SEQUENCE LISTING (128 sequences)

SEQ ID NO: 1 - DNA, 482 bp, synthetic construct
  source 1..482: mol_type = other DNA
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactacaatttggactgggtgcgacaggcccctggacaagggcttgagtggatgggagatattagtcctaactatggttatactatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgatgtctggggccaagggacaatggtcaccgtctcttca

SEQ ID NO: 2 - protein, 144 aa, synthetic construct
MDWTWVFLFLLSVTAGVHSQVQLVQSGAEVKKPGASVKVSCKASGYTITDYNLDWVRQAPGQGLEWMGDISPNYGYTIYNQKFKDRVTMTTDTSTSTAYMELRSLRSDDTAVYYCARRDIRYSGNSYKWYFDVWGQGTMVTVSS

SEQ ID NO: 3 - protein, 10 aa, synthetic construct
GYTITDYNLD

SEQ ID NO: 4 - protein, 16 aa, synthetic construct
DISPNYGYTIYNQKFK

SEQ ID NO: 5 - protein, 16 aa, synthetic construct
RDIRYSGNSYKWYFDV

SEQ ID NO: 6 - DNA, 428 bp, synthetic construct
  source 1..428: mol_type = other DNA
gtcagagccctggggaggaactgctcagttaggacccagagggaaccatggaagccccagctcagcttctcttcctcctgctactctggctcccagataccaccggagacatccagttgacccagtctccatccttcctgtctgcatctgtaggagacagagtcaccatcacttgcaggacaagtaagagcataagcaaatatttagcctggtatcagcaaaaaccagggaaagcccctaagctcctgatctattctggatccaccttgcaatctggggtcccatcaaggttcagcggcagtggatctgggacagaattcactctcacaatcagcagcctgcagcctgaagattttgcaacttattactgtcaacaacataatgaatacccgtacacgtttggccaggggaccaagctggagatcaaa

SEQ ID NO: 7 - protein, 127 aa, synthetic construct
MEAPAQLLFLLLLWLPDTTGDIQLTQSPSFLSASVGDRVTITCRTSKSISKYLAWYQQKPGKAPKLLIYSGSTLQSGVPSRFSGSGSGTEFTLTISSLQPEDFATYYCQQHNEYPYTFGQGTKLEIK

SEQ ID NO: 8 - protein, 11 aa, synthetic construct
RTSKSISKYLA

SEQ ID NO: 9 - protein, 7 aa, synthetic construct
SGSTLQS

SEQ ID NO: 10 - protein, 9 aa, synthetic construct
QQHNEYPYT

SEQ ID NO: 11 - protein, 9 aa, synthetic construct
HQHNEYPYT

SEQ ID NO: 12 - DNA, 428 bp, synthetic construct
  source 1..428: mol_type = other DNA
gtcagagccctggggaggaactgctcagttaggacccagagggaaccatggaagccccagctcagcttctcttcctcctgctactctggctcccagataccaccggagacatccagttgacccagtctccatccttcctgtctgcatctgtaggagacagagtcaccatcacttgcaggacaagtaagagcataagcaaatatttagcctggtatcagcaaaaaccagggaaagcccctaagctcctgatctattctggatccaccttgcaatctggggtcccatcaaggttcagcggcagtggatctgggacagaattcactctcacaatcagcagcctgcagcctgaagattttgcaacttattactgtcatcaacataatgaatacccgtacacgtttggccaggggaccaagctggagatcaaa

SEQ ID NO: 13 - protein, 127 aa, synthetic construct
MEAPAQLLFLLLLWLPDTTGDIQLTQSPSFLSASVGDRVTITCRTSKSISKYLAWYQQKPGKAPKLLIYSGSTLQSGVPSRFSGSGSGTEFTLTISSLQPEDFATYYCHQHNEYPYTFGQGTKLEIK

SEQ ID NO: 14 - protein, 11 aa, synthetic construct
RHSKSISKYLA

SEQ ID NO: 15 - DNA, 428 bp, synthetic construct
  source 1..428: mol_type = other DNA
gtcagagccctggggaggaactgctcagttaggacccagagggaaccatggaagccccagctcagcttctcttcctcctgctactctggctcccagataccaccggagacatccagttgacccagtctccatccttcctgtctgcatctgtaggagacagagtcaccatcacttgcaggcatagtaagagcataagcaaatatttagcctggtatcagcaaaaaccagggaaagcccctaagctcctgatctattctggatccaccttgcaatctggggtcccatcaaggttcagcggcagtggatctgggacagaattcactctcacaatcagcagcctgcagcctgaagattttgcaacttattactgtcaacaacataatgaatacccgtacacgtttggccaggggaccaagctggagatcaaa

SEQ ID NO: 16 - protein, 127 aa, synthetic construct
MEAPAQLLFLLLLWLPDTTGDIQLTQSPSFLSASVGDRVTITCRHSKSISKYLAWYQQKPGKAPKLLIYSGSTLQSGVPSRFSGSGSGTEFTLTISSLQPEDFATYYCQQHNEYPYTFGQGTKLEIK

SEQ ID NO: 17 - protein, 10 aa, synthetic construct
GYTHTDYNLD

SEQ ID NO: 18 - DNA, 482 bp, synthetic construct
  source 1..482: mol_type = other DNA
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacacatacagactacaatttggactgggtgcgacaggcccctggacaagggcttgagtggatgggagatattagtcctaactatggttatactatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgatgtctggggccaagggacaatggtcaccgtctcttca

SEQ ID NO: 19 - protein, 144 aa, synthetic construct
MDWTWVFLFLLSVTAGVHSQVQLVQSGAEVKKPGASVKVSCKASGYTHTDYNLDWVRQAPGQGLEWMGDISPNYGYTIYNQKFKDRVTMTTDTSTSTAYMELRSLRSDDTAVYYCARRDIRYSGNSYKWYFDVWGQGTMVTVSS

SEQ ID NO: 20 - protein, 10 aa, synthetic construct
GYTITDYHLD

SEQ ID NO: 21 - DNA, 481 bp, synthetic construct
  source 1..481: mol_type = other DNA
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatttggactgggtgcgacaggcccctggacaagggcttgagtggatgggagatattagtcctaactatggttatactatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgatgtctggggccaagggacaatggtcaccgtctcttc

SEQ ID NO: 22 - protein, 144 aa, synthetic construct
MDWTWVFLFLLSVTAGVHSQVQLVQSGAEVKKPGASVKVSCKASGYTITDYHLDWVRQAPGQGLEWMGDISPNYGYTIYNQKFKDRVTMTTDTSTSTAYMELRSLRSDDTAVYYCARRDIRYSGNSYKWYFDVWGQGTMVTVSS

SEQ ID NO: 23 - protein, 11 aa, synthetic construct
RTSKSISKHLA

SEQ ID NO: 24 - DNA, 428 bp, synthetic construct
  source 1..428: mol_type = other DNA
gtcagagccctggggaggaactgctcagttaggacccagagggaaccatggaagccccagctcagcttctcttcctcctgctactctggctcccagataccaccggagacatccagttgacccagtctccatccttcctgtctgcatctgtaggagacagagtcaccatcacttgcaggacaagtaagagcataagcaaacatttagcctggtatcagcaaaaaccagggaaagcccctaagctcctgatctattctggatccaccttgcaatctggggtcccatcaaggttcagcggcagtggatctgggacagaattcactctcacaatcagcagcctgcagcctgaagattttgcaacttattactgtcaacaacataatgaatacccgtacacgtttggccaggggaccaagctggagatcaaa

SEQ ID NO: 25 - protein, 127 aa, synthetic construct
MEAPAQLLFLLLLWLPDTTGDIQLTQSPSFLSASVGDRVTITCRTSKSISKHLAWYQQKPGKAPKLLIYSGSTLQSGVPSRFSGSGSGTEFTLTISSLQPEDFATYYCQQHNEYPYTFGQGTKLEIK

SEQ ID NO: 26 - protein, 17 aa, synthetic construct
DISPNHGYTIYNQKFKD

SEQ ID NO: 27 - DNA, 482 bp, synthetic construct
  source 1..482: mol_type = other DNA
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactacaatttggactgggtgcgacaggcccctggacaagggcttgagtggatgggagatattagtcctaaccatggttatactatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgatgtctggggccaagggacaatggtcaccgtctcttca

SEQ ID NO: 28 - protein, 144 aa, synthetic construct
MDWTWVFLFLLSVTAGVHSQVQLVQSGAEVKKPGASVKVSCKASGYTITDYNLDWVRQAPGQGLEWMGDISPNHGYTIYNQKFKDRVTMTTDTSTSTAYMELRSLRSDDTAVYYCARRDIRYSGNSYKWYFDVWGQGTMVTVSS

SEQ ID NO: 29 - protein, 9 aa, synthetic construct
QQHNHYPYT

SEQ ID NO: 30 - DNA, 428 bp, synthetic construct
  source 1..428: mol_type = other DNA
gtcagagccctggggaggaactgctcagttaggacccagagggaaccatggaagccccagctcagcttctcttcctcctgctactctggctcccagataccaccggagacatccagttgacccagtctccatccttcctgtctgcatctgtaggagacagagtcaccatcacttgcaggacaagtaagagcataagcaaatatttagcctggtatcagcaaaaaccagggaaagcccctaagctcctgatctattctggatccaccttgcaatctggggtcccatcaaggttcagcggcagtggatctgggacagaattcactctcacaatcagcagcctgcagcctgaagattttgcaacttattactgtcaacaacataatcattacccgtacacgtttggccaggggaccaagctggagatcaaa

SEQ ID NO: 31 - protein, 127 aa, synthetic construct
MEAPAQLLFLLLLWLPDTTGDIQLTQSPSFLSASVGDRVTITCRTSKSISKYLAWYQQKPGKAPKLLIYSGSTLQSGVPSRFSGSGSGTEFTLTISSLQPEDFATYYCQQHNHYPYTFGQGTKLEIK

SEQ ID NO: 32 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 17 aa, synthetic construct
DISPNYGYHIYNQKFKD

SEQ ID NO: 35 - DNA, 482 bp, synthetic construct
  source 1..482: mol_type = other DNA
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactacaatttggactgggtgcgacaggcccctggacaagggcttgagtggatgggagatattagtcctaactatggttatcatatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgatgtctggggccaagggacaatggtcaccgtctcttca

SEQ ID NO: 36 - protein, 144 aa, synthetic construct
MDWTWVFLFLLSVTAGVHSQVQLVQSGAEVKKPGASVKVSCKASGYTITDYNLDWVRQAPGQGLEWMGDISPNYGYHIYNQKFKDRVTMTTDTSTSTAYMELRSLRSDDTAVYYCARRDIRYSGNSYKWYFDVWGQGTMVTVSS

SEQ ID NO: 37 - protein, 10 aa, synthetic construct
GYTITDYHID

SEQ ID NO: 38 - protein, 17 aa, synthetic construct
DISWNYGYTIYNQKFKD

SEQ ID NO: 39 - protein, 16 aa, synthetic construct
RDIRYSGNSYKWYFDW

SEQ ID NO: 40 - DNA, 482 bp, synthetic construct
  source 1..482: mol_type = other DNA
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatatcgactgggtgcgacaggcccctggacaagggcttgagtggatgggagatattagttggaactatggttatactatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgattggtggggccaagggacaatggtcaccgtctcttca

SEQ ID NO: 41 - protein, 144 aa, synthetic construct
MDWTWVFLFLLSVTAGVHSQVQLVQSGAEVKKPGASVKVSCKASGYTITDYHIDWVRQAPGQGLEWMGDISWNYGYTIYNQKFKDRVTMTTDTSTSTAYMELRSLRSDDTAVYYCARRDIRYSGNSYKWYFDWWGQGTMVTVSS

SEQ ID NO: 42 - protein, 10 aa, synthetic construct
GYTITDYHID

SEQ ID NO: 43 - protein, 17 aa, synthetic construct
DISFNYGYTIYNQKFKD

SEQ ID NO: 44 - protein, 16 aa, synthetic construct
RDIRYSGNSYKWYFDW

SEQ ID NO: 45 - DNA, 482 bp, synthetic construct
  source 1..482: mol_type = other DNA
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatatcgactgggtgcgacaggcccctggacaagggcttgagtggatgggagatattagtttcaactatggttatactatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgattggtggggccaagggacaatggtcaccgtctcttca

SEQ ID NO: 46 - protein, 144 aa, synthetic construct
MDWTWVFLFLLSVTAGVHSQVQLVQSGAEVKKPGASVKVSCKASGYTITDYHIDWVRQAPGQGLEWMGDISFNYGYTIYNQKFKDRVTMTTDTSTSTAYMELRSLRSDDTAVYYCARRDIRYSGNSYKWYFDWWGQGTMVTVSS

SEQ ID NO: 47 - protein, 10 aa, synthetic construct
GYTITDYHFD

SEQ ID NO: 48 - protein, 17 aa, synthetic construct
DISMNYGYTIYNQKFKD

SEQ ID NO: 49 - protein, 16 aa, synthetic construct
RDIRYSGNSYKWYFDE

SEQ ID NO: 50 - DNA, 482 bp, synthetic construct
  source 1..482: mol_type = other DNA
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatttcgactgggtgcgacaggcccctggacaagggcttgagtggatgggagatattagtatgaactatggttatactatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgatgagtggggccaagggacaatggtcaccgtctcttca

SEQ ID NO: 51 - protein, 144 aa, synthetic construct
MDWTWVFLFLLSVTAGVHSQVQLVQSGAEVKKPGASVKVSCKASGYTITDYHFDWVRQAPGQGLEWMGDISMNYGYTIYNQKFKDRVTMTTDTSTSTAYMELRSLRSDDTAVYYCARRDIRYSGNSYKWYFDEWGQGTMVTVSS

SEQ ID NO: 52 - protein, 10 aa, synthetic construct
GYTITDYHFD

SEQ ID NO: 53 - protein, 17 aa, synthetic construct
DISMNYGYTIYNQKFKD

SEQ ID NO: 54 - protein, 16 aa, synthetic construct
RDIRYSGNSYKWYFDW

SEQ ID NO: 55 - DNA, 482 bp, synthetic construct
  source 1..482: mol_type = other DNA
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatttcgactgggtgcgacaggcccctggacaagggcttgagtggatgggagatattagtatgaactatggttatactatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgattggtggggccaagggacaatggtcaccgtctcttca

SEQ ID NO: 56 - protein, 144 aa, synthetic construct
MDWTWVFLFLLSVTAGVHSQVQLVQSGAEVKKPGASVKVSCKASGYTITDYHFDWVRQAPGQGLEWMGDISMNYGYTIYNQKFKDRVTMTTDTSTSTAYMELRSLRSDDTAVYYCARRDIRYSGNSYKWYFDWWGQGTMVTVSS

SEQ ID NO: 57 - protein, 17 aa, synthetic construct
DISMNYGYHIYNQKFKD

SEQ ID NO: 58 - DNA, 482 bp, synthetic construct
  source 1..482: mol_type = other DNA
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatttcgactgggtgcgacaggcccctggacaagggcttgagtggatgggagatattagtatgaactatggttatcatatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgatgagtggggccaagggacaatggtcaccgtctcttca

SEQ ID NO: 59 - protein, 144 aa, synthetic construct
MDWTWVFLFLLSVTAGVHSQVQLVQSGAEVKKPGASVKVSCKASGYTITDYHFDWVRQAPGQGLEWMGDISMNYGYHIYNQKFKDRVTMTTDTSTSTAYMELRSLRSDDTAVYYCARRDIRYSGNSYKWYFDEWGQGTMVTVSS

SEQ ID NO: 60 - DNA, 984 bp, synthetic construct
  source 1..984: mol_type = other DNA
gctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgctgcatgaggctctgcacgcccactacacacagaagagcctctccctgtctctgggtaaatga

SEQ ID NO: 61 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGK

SEQ ID NO: 62 - protein, 17 aa, synthetic construct
DISWNYGYHIYNQKFKD

SEQ ID NO: 63 - DNA, 482 bp, synthetic construct
  source 1..482: mol_type = other DNA
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatatcgactgggtgcgacaggcccctggacaagggcttgagtggatgggagatattagttggaactatggttatcatatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgattggtggggccaagggacaatggtcaccgtctcttca

SEQ ID NO: 64 - protein, 144 aa, synthetic construct
MDWTWVFLFLLSVTAGVHSQVQLVQSGAEVKKPGASVKVSCKASGYTITDYHIDWVRQAPGQGLEWMGDISWNYGYHIYNQKFKDRVTMTTDTSTSTAYMELRSLRSDDTAVYYCARRDIRYSGNSYKWYFDWWGQGTMVTVSS

SEQ ID NO: 65 - protein, 17 aa, synthetic construct
DISFNYGYHIYNQKFKD

SEQ ID NO: 66 - DNA, 482 bp, synthetic construct
  source 1..482: mol_type = other DNA
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatatcgactgggtgcgacaggcccctggacaagggcttgagtggatgggagatattagtttcaactatggttatcatatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgattggtggggccaagggacaatggtcaccgtctcttca

SEQ ID NO: 67 - protein, 144 aa, synthetic construct
MDWTWVFLFLLSVTAGVHSQVQLVQSGAEVKKPGASVKVSCKASGYTITDYHIDWVRQAPGQGLEWMGDISFNYGYHIYNQKFKDRVTMTTDTSTSTAYMELRSLRSDDTAVYYCARRDIRYSGNSYKWYFDWWGQGTMVTVSS

SEQ ID NO: 68 - protein, 17 aa, synthetic construct
DISMNYGYHIYNQKFKD

SEQ ID NO: 69 - DNA, 482 bp, synthetic construct
  source 1..482: mol_type = other DNA
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatttcgactgggtgcgacaggcccctggacaagggcttgagtggatgggagatattagtatgaactatggttatcatatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgattggtggggccaagggacaatggtcaccgtctcttca

SEQ ID NO: 70 - protein, 144 aa, synthetic construct
MDWTWVFLFLLSVTAGVHSQVQLVQSGAEVKKPGASVKVSCKASGYTITDYHFDWVRQAPGQGLEWMGDISMNYGYHIYNQKFKDRVTMTTDTSTSTAYMELRSLRSDDTAVYYCARRDIRYSGNSYKWYFDWWGQGTMVTVSS

SEQ ID NO: 71 - DNA, 2384 bp, synthetic construct
  source 1..2384: mol_type = other DNA
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatttcgactgggtgcgacaggcccctggacaagggcttgagtggatgggagatattagtatgaactatggttatcatatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgatgagtggggccaagggacaatggtcaccgtctcttcagctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgctgcatgaggctctgcacgcccactacacacagaagagcctctccctgtctctgggtaaagaggattgtaatgaactgcctccaagaaggaacacagaaattttgaccgggtcctggtccgatcaaacctaccctgaaggaactcaagctatatacaagtgcagacctggttatagatcactgggcaacattatcatggtatgtcgcaaaggagaatgggtggctctgaatcccctcagaaaatgccagaaacggccatgtggacaccccggcgataccccattcgggacatttaccttgactggaggcaatgtattcgagtatggcgtgaaagctgtctatacctgtaacgaaggttaccaattgttgggagaaataaattacagagaatgtgataccgatggatggaccaacgatattcccatatgcgaggttgttaagtgcttgcctgtcactgcaccagaaaacgggaaaatcgtatctagcgcaatggagccagaccgcgaataccatttcgggcaggcagtgaggtttgtttgcaattccggttataagatagaaggggatgaggagatgcactgtagcgacgatggtttttggtccaaggaaaagcccaagtgcgtcgaaataagttgcaagtcacctgacgtcataaacgggagccccatatcccaaaagataatttacaaggagaacgaacgatttcaatataagtgtaatatggggtatgaatactccgagagaggtgatgccgtctgtaccgaaagtggatggcgaccactcccctcatgcgaagagaagtcctgtgataatccatacatccctaatggtgattattccccccttcgaataaagcatcggacaggagacgagatcacatatcagtgtcgcaacggattctatccagccaccagaggcaacactgcaaagtgtacatctacaggatggatacctgccccacgatgtaccttgaagccctga

SEQ ID NO: 72 - protein, 777 aa, synthetic construct
MDWTWVFLFLLSVTAGVHSQVQLVQSGAEVKKPGASVKVSCKASGYTITDYHFDWVRQAPGQGLEWMGDISMNYGYHIYNQKFKDRVTMTTDTSTSTAYMELRSLRSDDTAVYYCARRDIRYSGNSYKWYFDEWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 73 - DNA, 752 bp, synthetic construct
  source 1..752: mol_type = other DNA
gtcagagccctggggaggaactgctcagttaggacccagagggaaccatggaagccccagctcagcttctcttcctcctgctactctggctcccagataccaccggagacatccagttgacccagtctccatccttcctgtctgcatctgtaggagacagagtcaccatcacttgcaggacaagtaagagcataagcaaacatttagcctggtatcagcaaaaaccagggaaagcccctaagctcctgatctattctggatccaccttgcaatctggggtcccatcaaggttcagcggcagtggatctgggacagaattcactctcacaatcagcagcctgcagcctgaagattttgcaacttattactgtcaacaacataatgaatacccgtacacgtttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 74 - protein, 234 aa, synthetic construct
MEAPAQLLFLLLLWLPDTTGDIQLTQSPSFLSASVGDRVTITCRTSKSISKHLAWYQQKPGKAPKLLIYSGSTLQSGVPSRFSGSGSGTEFTLTISSLQPEDFATYYCQQHNEYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - DNA, 2384 bp, synthetic construct
  source 1..2384: mol_type = other DNA
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatatcgactgggtgcgacaggcccctggacaagggcttgagtggatgggagatattagttggaactatggttatcatatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgattggtggggccaagggacaatggtcaccgtctcttcagctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgctgcatgaggctctgcacgcccactacacacagaagagcctctccctgtctctgggtaaagaggattgtaatgaactgcctccaagaaggaacacagaaattttgaccgggtcctggtccgatcaaacctaccctgaaggaactcaagctatatacaagtgcagacctggttatagatcactgggcaacattatcatggtatgtcgcaaaggagaatgggtggctctgaatcccctcagaaaatgccagaaacggccatgtggacaccccggcgataccccattcgggacatttaccttgactggaggcaatgtattcgagtatggcgtgaaagctgtctatacctgtaacgaaggttaccaattgttgggagaaataaattacagagaatgtgataccgatggatggaccaacgatattcccatatgcgaggttgttaagtgcttgcctgtcactgcaccagaaaacgggaaaatcgtatctagcgcaatggagccagaccgcgaataccatttcgggcaggcagtgaggtttgtttgcaattccggttataagatagaaggggatgaggagatgcactgtagcgacgatggtttttggtccaaggaaaagcccaagtgcgtcgaaataagttgcaagtcacctgacgtcataaacgggagccccatatcccaaaagataatttacaaggagaacgaacgatttcaatataagtgtaatatggggtatgaatactccgagagaggtgatgccgtctgtaccgaaagtggatggcgaccactcccctcatgcgaagagaagtcctgtgataatccatacatccctaatggtgattattccccccttcgaataaagcatcggacaggagacgagatcacatatcagtgtcgcaacggattctatccagccaccagaggcaacactgcaaagtgtacatctacaggatggatacctgccccacgatgtaccttgaagccctga

SEQ ID NO: 76 - protein, 777 aa, synthetic construct
MDWTWVFLFLLSVTAGVHSQVQLVQSGAEVKKPGASVKVSCKASGYTITDYHIDWVRQAPGQGLEWMGDISWNYGYHIYNQKFKDRVTMTTDTSTSTAYMELRSLRSDDTAVYYCARRDIRYSGNSYKWYFDWWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 77 - DNA, 2384 bp, synthetic construct
  source 1..2384: mol_type = other DNA
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatatcgactgggtgcgacaggcccctggacaagggcttgagtggatgggagatattagtttcaactatggttatcatatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgattggtggggccaagggacaatggtcaccgtctcttcagctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgctgcatgaggctctgcacgcccactacacacagaagagcctctccctgtctctgggtaaagaggattgtaatgaactgcctccaagaaggaacacagaaattttgaccgggtcctggtccgatcaaacctaccctgaaggaactcaagctatatacaagtgcagacctggttatagatcactgggcaacattatcatggtatgtcgcaaaggagaatgggtggctctgaatcccctcagaaaatgccagaaacggccatgtggacaccccggcgataccccattcgggacatttaccttgactggaggcaatgtattcgagtatggcgtgaaagctgtctatacctgtaacgaaggttaccaattgttgggagaaataaattacagagaatgtgataccgatggatggaccaacgatattcccatatgcgaggttgttaagtgcttgcctgtcactgcaccagaaaacgggaaaatcgtatctagcgcaatggagccagaccgcgaataccatttcgggcaggcagtgaggtttgtttgcaattccggttataagatagaaggggatgaggagatgcactgtagcgacgatggtttttggtccaaggaaaagcccaagtgcgtcgaaataagttgcaagtcacctgacgtcataaacgggagccccatatcccaaaagataatttacaaggagaacgaacgatttcaatataagtgtaatatggggtatgaatactccgagagaggtgatgccgtctgtaccgaaagtggatggcgaccactcccctcatgcgaagagaagtcctgtgataatccatacatccctaatggtgattattccccccttcgaataaagcatcggacaggagacgagatcacatatcagtgtcgcaacggattctatccagccaccagaggcaacactgcaaagtgtacatctacaggatggatacctgccccacgatgtaccttgaagccctga

SEQ ID NO: 78 - protein, 777 aa, synthetic construct
MDWTWVFLFLLSVTAGVHSQVQLVQSGAEVKKPGASVKVSCKASGYTITDYHIDWVRQAPGQGLEWMGDISFNYGYHIYNQKFKDRVTMTTDTSTSTAYMELRSLRSDDTAVYYCARRDIRYSGNSYKWYFDWWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 79 - DNA, 2384 bp, synthetic construct
  source 1..2384: mol_type = other DNA
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatttcgactgggtgcgacaggcccctggacaagggcttgagtggatgggagatattagtatgaactatggttatcatatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgattggtggggccaagggacaatggtcaccgtctcttcagctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgctgcatgaggctctgcacgcccactacacacagaagagcctctccctgtctctgggtaaagaggattgtaatgaactgcctccaagaaggaacacagaaattttgaccgggtcctggtccgatcaaacctaccctgaaggaactcaagctatatacaagtgcagacctggttatagatcactgggcaacattatcatggtatgtcgcaaaggagaatgggtggctctgaatcccctcagaaaatgccagaaacggccatgtggacaccccggcgataccccattcgggacatttaccttgactggaggcaatgtattcgagtatggcgtgaaagctgtctatacctgtaacgaaggttaccaattgttgggagaaataaattacagagaatgtgataccgatggatggaccaacgatattcccatatgcgaggttgttaagtgcttgcctgtcactgcaccagaaaacgggaaaatcgtatctagcgcaatggagccagaccgcgaataccatttcgggcaggcagtgaggtttgtttgcaattccggttataagatagaaggggatgaggagatgcactgtagcgacgatggtttttggtccaaggaaaagcccaagtgcgtcgaaataagttgcaagtcacctgacgtcataaacgggagccccatatcccaaaagataatttacaaggagaacgaacgatttcaatataagtgtaatatggggtatgaatactccgagagaggtgatgccgtctgtaccgaaagtggatggcgaccactcccctcatgcgaagagaagtcctgtgataatccatacatccctaatggtgattattccccccttcgaataaagcatcggacaggagacgagatcacatatcagtgtcgcaacggattctatccagccaccagaggcaacactgcaaagtgtacatctacaggatggatacctgccccacgatgtaccttgaagccctga

SEQ ID NO: 80 - protein, 777 aa, synthetic construct
MDWTWVFLFLLSVTAGVHSQVQLVQSGAEVKKPGASVKVSCKASGYTITDYHFDWVRQAPGQGLEWMGDISMNYGYHIYNQKFKDRVTMTTDTSTSTAYMELRSLRSDDTAVYYCARRDIRYSGNSYKWYFDWWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 81 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 82 - protein, 1213 aa, Homo sapiens
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 83 - protein, 449 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVSFTL

SEQ ID NO: 84 - protein, 431 aa, Homo sapiens
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVSFTL

SEQ ID NO: 85 - protein, 306 aa, Homo sapiens
EDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 86 - protein, 5 aa, synthetic construct
DYHFD

SEQ ID NO: 87 - protein, 125 aa, synthetic construct
QVQLVQSGAEVKKPGASVKVSCKASGYTITDYHFDWVRQAPGQGLEWMGDISMNYGYHIYNQKFKDRVTMTTDTSTSTAYMELRSLRSDDTAVYYCARRDIRYSGNSYKWYFDEWGQGTMVTVSS

SEQ ID NO: 88 - protein, 107 aa, synthetic construct
DIQLTQSPSFLSASVGDRVTITCRTSKSISKHLAWYQQKPGKAPKLLIYSGSTLQSGVPSRFSGSGSGTEFTLTISSLQPEDFATYYCQQHNEYPYTFGQGTKLEIK

SEQ ID NO: 89 - protein, 758 aa, synthetic construct
QVQLVQSGAEVKKPGASVKVSCKASGYTITDYHFDWVRQAPGQGLEWMGDISMNYGYHIYNQKFKDRVTMTTDTSTSTAYMELRSLRSDDTAVYYCARRDIRYSGNSYKWYFDEWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 90 - protein, 214 aa, synthetic construct
DIQLTQSPSFLSASVGDRVTITCRTSKSISKHLAWYQQKPGKAPKLLIYSGSTLQSGVPSRFSGSGSGTEFTLTISSLQPEDFATYYCQQHNEYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 19 aa, synthetic construct
MDWTWVFLFLLSVTAGVHS

SEQ ID NO: 92 - protein, 20 aa, synthetic construct
MEAPAQLLFLLLLWLPDTTG

SEQ ID NO: 93 - protein, 125 aa, synthetic construct
QVQLVQSGAEVKKPGASVKVSCKASGYTITDYNLDWVRQAPGQGLEWMGDISPNYGYTIYNQKFKDRVTMTTDTSTSTAYMELRSLRSDDTAVYYCARRDIRYSGNSYKWYFDVWGQGTMVTVSS

SEQ ID NO: 94 - protein, 5 aa, synthetic construct
DYNLD

SEQ ID NO: 95 - protein, 107 aa, synthetic construct
DIQLTQSPSFLSASVGDRVTITCRTSKSISKYLAWYQQKPGKAPKLLIYSGSTLQSGVPSRFSGSGSGTEFTLTISSLQPEDFATYYCQQHNEYPYTFGQGTKLEIK

SEQ ID NO: 96 - protein, 7 aa, synthetic construct
QQHNEYP

SEQ ID NO: 97 - protein, 7 aa, synthetic construct
HQHNEYP

SEQ ID NO: 98 - protein, 107 aa, synthetic construct
DIQLTQSPSFLSASVGDRVTITCRTSKSISKYLAWYQQKPGKAPKLLIYSGSTLQSGVPSRFSGSGSGTEFTLTISSLQPEDFATYYCHQHNEYPYTFGQGTKLEIK

SEQ ID NO: 99 - protein, 107 aa, synthetic construct
DIQLTQSPSFLSASVGDRVTITCRHSKSISKYLAWYQQKPGKAPKLLIYSGSTLQSGVPSRFSGSGSGTEFTLTISSLQPEDFATYYCQQHNEYPYTFGQGTKLEIK

SEQ ID NO: 100 - protein, 10 aa, synthetic construct
GYTHTDYNLD

SEQ ID NO: 101 - protein, 125 aa, synthetic construct
QVQLVQSGAEVKKPGASVKVSCKASGYTHTDYNLDWVRQAPGQGLEWMGDISPNYGYTIYNQKFKDRVTMTTDTSTSTAYMELRSLRSDDTAVYYCARRDIRYSGNSYKWYFDVWGQGTMVTVSS

SEQ ID NO: 102 - protein, 5 aa, synthetic construct
DYHLD

SEQ ID NO: 103 - protein, 125 aa, synthetic construct
QVQLVQSGAEVKKPGASVKVSCKASGYTITDYHLDWVRQAPGQGLEWMGDISPNYGYTIYNQKFKDRVTMTTDTSTSTAYMELRSLRSDDTAVYYCARRDIRYSGNSYKWYFDVWGQGTMVTVSS

SEQ ID NO: 104 - protein, 125 aa, synthetic construct
QVQLVQSGAEVKKPGASVKVSCKASGYTITDYNLDWVRQAPGQGLEWMGDISPNHGYTIYNQKFKDRVTMTTDTSTSTAYMELRSLRSDDTAVYYCARRDIRYSGNSYKWYFDVWGQGTMVTVSS

SEQ ID NO: 105 - protein, 7 aa, synthetic construct
QQHNHYP

SEQ ID NO: 106 - protein, 107 aa, synthetic construct
DIQLTQSPSFLSASVGDRVTITCRTSKSISKYLAWYQQKPGKAPKLLIYSGSTLQSGVPSRFSGSGSGTEFTLTISSLQPEDFATYYCQQHNHYPYTFGQGTKLEIK

SEQ ID NO: 107 - protein, 125 aa, synthetic construct
QVQLVQSGAEVKKPGASVKVSCKASGYTITDYNLDWVRQAPGQGLEWMGDISPNYGYHIYNQKFKDRVTMTTDTSTSTAYMELRSLRSDDTAVYYCARRDIRYSGNSYKWYFDVWGQGTMVTVSS

SEQ ID NO: 108 - protein, 5 aa, synthetic construct
DYHID

SEQ ID NO: 109 - protein, 125 aa, synthetic construct
QVQLVQSGAEVKKPGASVKVSCKASGYTITDYHIDWVRQAPGQGLEWMGDISWNYGYTIYNQKFKDRVTMTTDTSTSTAYMELRSLRSDDTAVYYCARRDIRYSGNSYKWYFDWWGQGTMVTVSS

SEQ ID NO: 110 - protein, 125 aa, synthetic construct
QVQLVQSGAEVKKPGASVKVSCKASGYTITDYHIDWVRQAPGQGLEWMGDISFNYGYTIYNQKFKDRVTMTTDTSTSTAYMELRSLRSDDTAVYYCARRDIRYSGNSYKWYFDWWGQGTMVTVSS

SEQ ID NO: 111 - protein, 125 aa, synthetic construct
QVQLVQSGAEVKKPGASVKVSCKASGYTITDYHFDWVRQAPGQGLEWMGDISMNYGYTIYNQKFKDRVTMTTDTSTSTAYMELRSLRSDDTAVYYCARRDIRYSGNSYKWYFDEWGQGTMVTVSS

SEQ ID NO: 112 - protein, 125 aa, synthetic construct
QVQLVQSGAEVKKPGASVKVSCKASGYTITDYHFDWVRQAPGQGLEWMGDISMNYGYTIYNQKFKDRVTMTTDTSTSTAYMELRSLRSDDTAVYYCARRDIRYSGNSYKWYFDWWGQGTMVTVSS

SEQ ID NO: 113 - protein, 125 aa, synthetic construct
QVQLVQSGAEVKKPGASVKVSCKASGYTITDYHIDWVRQAPGQGLEWMGDISWNYGYHIYNQKFKDRVTMTTDTSTSTAYMELRSLRSDDTAVYYCARRDIRYSGNSYKWYFDWWGQGTMVTVSS

SEQ ID NO: 114 - protein, 125 aa, synthetic construct
QVQLVQSGAEVKKPGASVKVSCKASGYTITDYHIDWVRQAPGQGLEWMGDISFNYGYHIYNQKFKDRVTMTTDTSTSTAYMELRSLRSDDTAVYYCARRDIRYSGNSYKWYFDWWGQGTMVTVSS

SEQ ID NO: 115 - protein, 125 aa, synthetic construct
QVQLVQSGAEVKKPGASVKVSCKASGYTITDYHFDWVRQAPGQGLEWMGDISMNYGYHIYNQKFKDRVTMTTDTSTSTAYMELRSLRSDDTAVYYCARRDIRYSGNSYKWYFDWWGQGTMVTVSS

SEQ ID NO: 116 - protein, 758 aa, synthetic construct
QVQLVQSGAEVKKPGASVKVSCKASGYTITDYHIDWVRQAPGQGLEWMGDISWNYGYHIYNQKFKDRVTMTTDTSTSTAYMELRSLRSDDTAVYYCARRDIRYSGNSYKWYFDWWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 117 - protein, 758 aa, synthetic construct
QVQLVQSGAEVKKPGASVKVSCKASGYTITDYHIDWVRQAPGQGLEWMGDISFNYGYHIYNQKFKDRVTMTTDTSTSTAYMELRSLRSDDTAVYYCARRDIRYSGNSYKWYFDWWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 118 - protein, 758 aa, synthetic construct
QVQLVQSGAEVKKPGASVKVSCKASGYTITDYHFDWVRQAPGQGLEWMGDISMNYGYHIYNQKFKDRVTMTTDTSTSTAYMELRSLRSDDTAVYYCARRDIRYSGNSYKWYFDWWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 119 - protein, 471 aa, synthetic construct
MDWTWVFLFLLSVTAGVHSQVQLVQSGAEVKKPGASVKVSCKASGYTITDYHFDWVRQAPGQGLEWMGDISMNYGYHIYNQKFKDRVTMTTDTSTSTAYMELRSLRSDDTAVYYCARRDIRYSGNSYKWYFDEWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGK

SEQ ID NO: 120 - protein, 452 aa, synthetic construct
QVQLVQSGAEVKKPGASVKVSCKASGYTITDYHFDWVRQAPGQGLEWMGDISMNYGYHIYNQKFKDRVTMTTDTSTSTAYMELRSLRSDDTAVYYCARRDIRYSGNSYKWYFDEWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGK

SEQ ID NO: 121 - protein, 471 aa, synthetic construct
MDWTWVFLFLLSVTAGVHSQVQLVQSGAEVKKPGASVKVSCKASGYTITDYHIDWVRQAPGQGLEWMGDISWNYGYHIYNQKFKDRVTMTTDTSTSTAYMELRSLRSDDTAVYYCARRDIRYSGNSYKWYFDWWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGK

SEQ ID NO: 122 - protein, 452 aa, synthetic construct
QVQLVQSGAEVKKPGASVKVSCKASGYTITDYHIDWVRQAPGQGLEWMGDISWNYGYHIYNQKFKDRVTMTTDTSTSTAYMELRSLRSDDTAVYYCARRDIRYSGNSYKWYFDWWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGK

SEQ ID NO: 123 - protein, 471 aa, synthetic construct
MDWTWVFLFLLSVTAGVHSQVQLVQSGAEVKKPGASVKVSCKASGYTITDYHIDWVRQAPGQGLEWMGDISFNYGYHIYNQKFKDRVTMTTDTSTSTAYMELRSLRSDDTAVYYCARRDIRYSGNSYKWYFDWWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGK

SEQ ID NO: 124 - protein, 452 aa, synthetic construct
QVQLVQSGAEVKKPGASVKVSCKASGYTITDYHIDWVRQAPGQGLEWMGDISFNYGYHIYNQKFKDRVTMTTDTSTSTAYMELRSLRSDDTAVYYCARRDIRYSGNSYKWYFDWWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGK

SEQ ID NO: 125 - protein, 471 aa, synthetic construct
MDWTWVFLFLLSVTAGVHSQVQLVQSGAEVKKPGASVKVSCKASGYTITDYHFDWVRQAPGQGLEWMGDISMNYGYHIYNQKFKDRVTMTTDTSTSTAYMELRSLRSDDTAVYYCARRDIRYSGNSYKWYFDWWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGK

SEQ ID NO: 126 - protein, 452 aa, synthetic construct
QVQLVQSGAEVKKPGASVKVSCKASGYTITDYHFDWVRQAPGQGLEWMGDISMNYGYHIYNQKFKDRVTMTTDTSTSTAYMELRSLRSDDTAVYYCARRDIRYSGNSYKWYFDWWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGK

SEQ ID NO: 127 - protein, 1231 aa, Homo sapiens [sequence omitted]

SEQ ID NO: 128 - protein, 1213 aa, Homo sapiens
EDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

(19) United States
(12) Patent Application Publication
(10) Pub. No.: US 2026/0176337 A1
(43) Pub. Date: Jun. 25, 2026

(54) ANTI-C5 ANTIBODY FUSED TO FACTOR H FOR USE IN THE TREATMENT OF COMPLEMENT-MEDIATED DISEASES

(71) Applicant: KIRA PHARMACEUTICALS (US) LLC, Cambridge, MA (US)

(72) Inventors: Wenru SONG, Arlington, MA (US); Richard LEE, Newton, MA (US); Qing Yu WENG, Brookline, MA (US); Jay MA, Bethany, CT (US)

(21) Appl. No.: 19/127,004

(22) PCT Filed: Nov. 1, 2023
(86) PCT No.: PCT/US2023/078410

§ 371 Date: May 2, 2025

Related U.S. Application Data
(60) Provisional application No. 63/421,993, filed on Nov. 2, 2022.
(60) Provisional application No. 63/486,949, filed on Feb. 24, 2023.
(60) Provisional application No. 63/501,264, filed on May 10, 2023.

(30) Foreign Application Priority Data
Feb. 26, 2023 (WO) PCT/US2023/063305

(51) Int. Cl. C07K 16/18

(52) U.S. Cl. CPC C07K 16/18; C07K 2317/24; C07K 2319/30

(57) ABSTRACT

The present application provides methods of treating a complement-mediated disease in a human individual, comprising administering to the individual an effective amount of a fusion protein comprising i) an antibody moiety that specifically binds to human C5 and ii) a Factor H (FH) or functional fragment thereof. The complement-mediated disease can be, for example, paroxysmal nocturnal hemoglobinuria (PNH) syndrome, C3 glomerulopathy (C3G), IgA nephropathy (IgAN), and thrombotic microangiopathy secondary to systemic lupus erythematosus (SLE-TMA).

### CROSS REFERENCE TO RELATED APPLICATIONS

This patent application claims priority benefits of U.S. Provisional Application No. 63/421,993 filed on Nov. 2, 2022, U.S. Provisional Application No. 63/486,949 filed on Feb. 24, 2023, International Patent Application No. PCT/US2023/063305 filed on Feb. 26, 2023, and U.S. Provisional Application No. 63/501,264 filed on May 10, 2023, the content of each of which is incorporated herein by reference in their entirety.

### REFERENCE TO AN ELECTRONIC SEQUENCE LISTING

The contents of the electronic sequence listing (792252001241seglist.xml; Size: 153,067 bytes; and Date of Creation: Oct. 16, 2023) is herein incorporated by reference in its entirety.

### FIELD OF THE INVENTION

The present invention relates to compositions and methods for treating complement-mediated diseases.

### BACKGROUND OF THE INVENTION

The complement system is part of innate immunity that plays a key role in host defense. It enhances (complements) the ability of antibodies and phagocytic cells to clear microbes and damaged cells from an organism, promote inflammation, and attack pathogen's cell membrane. However, activated complement also has the potential to cause significant tissue injury and destruction and dysregulated complement activity has been found to be associated with a number of rare and common diseases such as paroxysmal nocturnal hemoglobinuria (PNH), atypical hemolytic uremic syndrome (aHUS), rheumatoid arthritis (RA), age-related macular degeneration (AMD), etc. Thus, anti-complement therapy is a promising way of treating these human disorders.

Complement activation involves a cascade of target recognition and proteolytic cleavage. It can be activated via three different pathways, all of them converge at the C3 activation step. These pathways are the classical, alternative and lectin pathways. The Classical Pathway (CP) is activated by antigen-antibody complex and involves the sequential activation of C1 and C4/C2 before merging with other pathways at the C3 activation step. The lectin pathway (LP) is triggered by certain pattern recognition molecules, such as mannose-binding lectin (MBL), collectins and ficolins upon their binding to microbial surface sugar molecules. It involves the activation of mannan-binding lectin serine proteases (MASPs) which then cleave C4/C2 and join the other pathways at the C3 activation step. The alternative pathway (AP) is constitutively active at a low level due to spontaneous hydrolysis and activation of C3 to produce C3(H2O). The latter can associate with factor B, and upon proteolytic activation by factor D, produces the initial C3 cleaving enzyme complex C3(H2O)Bb. In the absence of regulatory proteins, the product of C3(H2O)Bb complex, C3b, can associate with factor B in the same way as C3(H2O) does, and thus starts another cycle of self-amplifying C3 activation.

Ongoing efforts to target C3 activation in complement-dependent diseases employ C3-inhibitory cyclic peptides or recombinant short variants of FH, which have very poor pharmacokinetics and require large and frequent (e.g., daily) dosing.

C3 activation also leads to the generation of C5-cleaving enzyme complexes and initiates the terminal complement activation pathway, culminating in the production of the potent pro-inflammatory mediator C5a and the membrane attack complex (MAC) C5b-9 which can cause cell lysis and death. To prevent complement from causing indiscriminate injury, host cells express a number of membrane-anchored regulators that function to block complement activation and amplification. Some of these regulators, including decay-accelerating factor (DAF, CD55) and MCP, work to inhibit C3 activation, while others such as CD59 work at other steps of the complement activation cascade. In addition to membrane-anchored complement regulators, there are also fluid phase regulators in the blood which act to preferentially protect the host tissues. The fluid phase inhibitors include factor H (FH) and factor I (FI), which are critical inhibitors of the alternative pathway of complement activation, and C4BP and C1 inhibitor (C1INH) which inhibit the classical pathway complement activation. Both fluid phase and membrane-anchored complement regulatory proteins are often composed of multiple conserved SCR domains. For example, FH is composed of 20 SCRs.

Complement C5 is a critical protein in the terminal pathway of complement activation and is the precursor protein for generating the potent pro-inflammatory mediator C5a, as well as the cytolytic membrane attack complex (MAC).

A number of human inflammatory and autoimmune diseases are mediated by C5a and/or MAC, and blocking C5 activation should prevent C5a and MAC generation and be of therapeutic value. A humanized mouse anti-human C5 mAb, eculizumab (e.g., Soliris®), has been used to treat two complement-mediated diseases paroxysmal nocturnal hemoglobinuria (PNH) and atypical hemolytic uremic syndrome (aHUS). However, not all PNH patients are responsive to eculizumab treatments and one of the reasons for non-responsiveness is genetic polymorphism of human C5 with loss of epitope binding to eculizumab. Additionally, due to high plasma concentration of C5 and targeted-mediated rapid removal of antibody, eculizumab has to be administered to patients at high doses and frequency. Thus, more effective and more convenient anti-complement drugs are needed, both in the treatment of PNH and aHUS, and in other complement-mediated diseases.

The disclosures of all publications, patents, patent applications and published patent applications referred to herein are hereby incorporated herein by reference in their entirety.

### BRIEF SUMMARY OF THE INVENTION

The present application in one aspect provides a method of treating a complement-mediated disease in a human individual, comprising administering to the human individual an effective amount of a fusion protein comprising i) an antibody moiety that specifically binds to human C5 (“anti-C5 antibody moiety”) and ii) a Factor H (FH) or fragment thereof.

In some embodiments according to any one of the methods described above, the complement-mediated disease is selected from the group consisting of paroxysmal nocturnal hemoglobinuria (PNH) syndrome, C3 glomerulopathy (C3G), IgA nephropathy (IgAN), and thrombotic microangiopathy secondary to systemic lupus erythematosus (SLE-TMA). In some embodiments, the complement-mediated disease is PNH. In some embodiments, the complement-mediated disease is C3G. In some embodiments, the complement-mediated disease is IgAN. In some embodiments, the complement-mediated disease is SLE-TMA.

In some embodiments according to any one of the methods described above, the fusion protein is administered intravenously (IV) or subcutaneously (SC).

In some embodiments according to any one of the methods described above, the fusion protein is administered at a dose of about 60 mg to about 3600 mg (e.g., about 60 mg to about 1200 mg, about 600 mg to 1200 mg, about 600 mg to about 2880 mg, about 600 mg to about 3600 mg, about 1200 mg to about 3600 mg, about 2400 mg to about 3600 mg, about 720 mg to about 1440 mg, about 1920 mg to about 2880 mg, or about 1800 mg to about 2400 mg).

In some embodiments according to any one of the methods described above, the fusion protein is administered at a single dose. In some embodiments, the fusion protein is administered at a dose of about 60 mg to about 1200 mg (e.g., about any of 60, 120, 180, 240, 300, 360, 420, 480, 540, 600, 660, 720, 780, 840, 900, 960, or 1200 mg). In some embodiments, the fusion protein is administered at a dose of: about 60 mg IV, about 180 mg IV, about 180 mg SC, about 360 mg IV, about 600 mg IV, about 720 mg SC, or about 1200 mg IV.

In some embodiments according to any one of the methods described above, the fusion protein is administered at multiple doses. In some embodiments, the fusion protein is administered weekly (QW). In some embodiments, the fusion protein is administered biweekly (Q2W). In some embodiments, the fusion protein is administered at a dose of about 600 mg to about 2880 mg (e.g., about 600 mg to 1200 mg, about 600 mg to about 2400 mg, about 1200 mg to about 2880 mg, about 2400 mg to about 2880 mg, about 720 mg to about 1440 mg, about 720 mg to about 1920 mg, about 1920 mg to about 2880 mg, or about 1800 mg to about 2400 mg). In some embodiments, the fusion protein is administered at multiple doses (e.g., QW or Q2W) of each of about any of 600 mg, 660 mg, 720 mg, 780 mg, 840 mg, 900 mg, 960 mg, 1200 mg, 1440 mg, 1800 mg, 1920 mg, 2400 mg, 2880 mg, 3000 mg, or 3600 mg. In some embodiments, the fusion protein is administered at a dose of about 600 mg IV weekly for 5 weeks.

In some embodiments according to any one of the methods described above, the fusion protein is administered with an initial phase comprising administering the fusion protein at one or more (e.g., 1, 2, 3, 4, 5, 6, 7, 8, 9, 10, 12, or more) initial doses, followed by a maintenance phase comprising administering the fusion protein (e.g., weekly or biweekly) for two or more doses (e.g., 2, 3, 4, 5, 6, 8, 10, 11, 12, 13, 14, 23, 24, 25, 26, 40, 44, 47, 48, 49, 50, or more doses). In some embodiments, the initial dose is about 600 mg to about 3600 mg, such as about 1200 mg to about 3600 mg (e.g., about any of 1200 mg, 1440 mg, 1800 mg, 1920 mg, 2400 mg, 2880 mg, 3000 mg, or 3600 mg). In some embodiments, the one or more initial doses of the fusion protein are administered IV. In some embodiments, the initial phase comprises administering the fusion protein at one initial dose. In some embodiments, the maintenance dose is about 600 mg to about 2880 mg (e.g., about any of 600 mg, 720 mg, 960 mg, 1200 mg, 1440 mg, 1800 mg, 1920 mg, 2400 mg, or 2880 mg). In some embodiments, the two or more maintenance doses of the fusion protein are administered weekly. In some embodiments, the maintenance dose (e.g., weekly) is about 600 mg to about 1440 mg (e.g., about any of 600 mg, 720 mg, 840 mg, 960 mg, 1080 mg, 1200 mg, 1320 mg, or 1440 mg). In some embodiments, the two or more maintenance doses (e.g., weekly) of the fusion protein are each administered at about 600 mg IV to about 1200 mg IV, or at about 720 mg SC to about 1440 mg SC (e.g., about any of 600 mg IV, 1200 mg IV, 720 mg SC, 960 mg SC, or 1440 mg SC). In some embodiments, the two or more maintenance doses of the fusion protein are administered biweekly. In some embodiments, the maintenance dose (e.g., biweekly) is about 960 mg to about 2880 mg (e.g., about any of 960 mg, 1800 mg, 1920 mg, 2000 mg, 2200 mg, 2400 mg, 2600 mg, or 2880 mg). In some embodiments, the two or more maintenance doses (e.g., biweekly) of the fusion protein are each administered at about 960 mg SC to about 1920 mg SC, at about 1920 mg SC to about 2880 mg SC, at about 1800 mg SC to about 2880 mg SC, or at about 1800 mg SC to about 2400 mg SC (e.g., about any of 960 mg SC, 1800 mg SC, 1920 mg SC, 2400 mg SC, or 2880 mg SC). In some embodiments, the maintenance phase is at least about 4 weeks (e.g., at least about any of 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 42, 44, 45, 46, 47, 48, 49, 50, 52 weeks or longer), such as at least about 12 weeks (e.g., about any of 12 weeks, 13 weeks, 24 weeks, 25 weeks, 48 weeks, or 49 weeks). In some embodiments, the initial phase comprises administering the fusion protein at an initial dose of about 1200 mg weekly for one or more (e.g., 1, 2, 3, 4, 5, 6, 7, 8, 9, 10, 12, or more) doses, and the maintenance phase comprises administering the fusion protein at a maintenance dose of about 720 mg weekly or biweekly for at least two doses (e.g., 2, 3, 4, 5, 6, 8, 10, 11, 12, 13, 14, 23, 24, 25, 26, 40, 44, 47, 48, 49, 50, or more doses). In some embodiments, the initial phase comprises administering the fusion protein at an initial dose of about 1200 mg IV on Day 1, and the maintenance phase comprises administering the fusion protein at a maintenance dose of about 720 mg SC weekly starting Day 8 for 4 weeks. In some embodiments, the maintenance phase comprises administering the fusion protein weekly or biweekly at a first maintenance dose for a first maintenance phase period, followed by administering the fusion protein weekly or biweekly at a second maintenance dose for a second maintenance phase period. In some embodiments, the second maintenance dose is higher than the first maintenance dose. In some embodiments, the second maintenance phase period is longer than the first maintenance phase period. In some embodiments, the first maintenance dose of the fusion protein is administered at about 720 mg SC or about 600 mg IV weekly, and the second maintenance dose of the fusion protein is administered at about 1440 mg SC or about 1200 mg IV weekly. In some embodiments, the first maintenance phase period is about 4 weeks, and the second maintenance phase period is at least about 44 weeks. In some embodiments, the complement-mediated disease is C3G or IgAN. In some embodiments, the initial phase comprises administering the fusion protein at an initial dose of about 1200 mg IV on Day 1, and the maintenance phase comprises: i) administering the fusion protein at a first maintenance dose of about 720 mg SC or about 600 mg IV weekly starting Day 8 for about 4 weeks, followed by ii) administering the fusion protein at a second maintenance dose of about 1440 mg SC or about 1200 mg IV weekly starting Day 36 for about 44 weeks.

In some embodiments according to any one of the methods described above, the initial phase comprises administering the fusion protein at an initial dose of about 600 mg IV to about 3600 mg IV on Day 1, and the maintenance phase comprises administering the fusion protein starting Day 8 at a maintenance dose of: i) about 600 mg IV to about 1200 mg IV weekly; ii) about 720 mg SC to about 1440 mg SC weekly; iii) about 1920 mg SC to about 2880 mg SC biweekly; iv) about 1800 mg SC to about 2400 mg SC biweekly; or v) about 960 mg SC to about 2880 mg SC biweekly. In some embodiments, the maintenance phase is at least about 12 weeks for weekly maintenance dosing, or at least about 13 weeks for biweekly maintenance dosing. In some embodiments, the maintenance phase is at least about 24 weeks for weekly maintenance dosing, or at least about 25 weeks for biweekly maintenance dosing. In some embodiments, the maintenance phase is at least about 48 weeks for weekly maintenance dosing, or at least about 49 weeks for biweekly maintenance dosing.

In some embodiments according to any one of the methods described above, the complement-mediated disease is PNH. In some embodiments, the initial phase comprises administering the fusion protein at an initial dose of about 1200 mg IV to about 3600 mg IV on Day 1, and the maintenance phase comprises administering the fusion protein starting Day 8 at a maintenance dose of: i) about 600 mg IV to about 1200 mg IV weekly for at least about 12 weeks; ii) about 720 mg SC to about 1440 mg SC weekly for at least about 12 weeks; iii) about 1920 mg SC to about 2880 mg SC biweekly for at least about 13 weeks; or iv) about 960 mg SC to about 2880 mg SC biweekly for at least about 13 weeks. In some embodiments, the initial phase comprises administering the fusion protein at an initial dose of about 1200 mg IV on Day 1, and the maintenance phase comprises administering the fusion protein starting Day 8 at a maintenance dose of about 720 mg SC or about 600 mg IV weekly for about 12 weeks. In some embodiments, the initial phase comprises administering the fusion protein at an initial dose of about 1200 mg IV on Day 1, and the maintenance phase comprises administering the fusion protein starting Day 8 at a maintenance dose of about 720 mg SC weekly for about 12 weeks. In some embodiments, the initial phase comprises administering the fusion protein at an initial dose of about 3600 mg IV on Day 1, and the maintenance phase comprises administering the fusion protein starting Day 8 at a maintenance dose of about 1920 mg SC to about 2880 mg SC (e.g., about 2880 mg SC) biweekly for about 13 weeks. In some embodiments, the initial phase comprises administering the fusion protein at an initial dose of about 1200 mg IV to about 2400 mg IV on Day 1, and the maintenance phase comprises administering the fusion protein starting Day 8 at a maintenance dose of about 960 mg SC to about 2880 mg SC biweekly for about 13 weeks. In some embodiments, the initial phase comprises administering the fusion protein at an initial dose of about 2400 mg IV on Day 1, and the maintenance phase comprises administering the fusion protein starting Day 8 at a maintenance dose of about 960 mg SC to about 2880 mg SC biweekly for about 13 weeks. In some embodiments, the initial phase comprises administering the fusion protein at an initial dose of about 1200 mg IV to about 2400 mg IV on Day 1, and the maintenance phase comprises administering the fusion protein starting Day 8 at a maintenance dose of about 1920 mg SC biweekly for about 13 weeks. In some embodiments, the initial phase comprises administering the fusion protein at an initial dose of about 2400 mg IV on Day 1, and the maintenance phase comprises administering the fusion protein starting Day 8 at a maintenance dose of about 1920 mg SC biweekly for about 13 weeks. In some embodiments, the fusion protein is further administered with an extension phase comprising administering the fusion protein at one or more extension doses after the maintenance phase. In some embodiments, the extension phase comprises administering the fusion protein at an extension dose of about 1920 mg SC biweekly for about 9 months.

In some embodiments according to any one of the methods described above, the complement-mediated disease is SLE-TMA. In some embodiments, the initial phase comprises administering the fusion protein at an initial dose of about 1200 mg IV to about 3600 mg IV on Day 1, and the maintenance phase comprises: i) administering the fusion protein starting Day 8 at a maintenance dose of about 720 mg SC to about 1440 mg SC weekly for at least about 24 weeks; ii) administering the fusion protein starting Day 8 at a maintenance dose of about 1920 mg SC to about 2880 mg SC biweekly for at least about 25 weeks; iii) administering the fusion protein starting Day 8 at a first maintenance dose of about 600 mg IV to about 1200 mg IV weekly for a first maintenance phase period, followed by administering the fusion protein at a second maintenance dose of about 720 mg SC to about 1440 mg SC weekly for a second maintenance phase period, and the maintenance phase is at least about 24 weeks; or iv) administering the fusion protein starting Day 8 at a first maintenance dose of about 600 mg IV to about 1200 mg IV weekly for a first maintenance phase period, followed by administering the fusion protein at a second maintenance dose of about 1920 mg SC to about 2880 mg SC biweekly for a second maintenance phase period, and the maintenance phase is at least about 25 weeks. In some embodiments, the first maintenance phase period is at least about 1 week, and the second maintenance phase period is no more than about 23 or about 24 weeks. In some embodiments, the initial phase comprises administering the fusion protein at an initial dose of about 1200 mg IV on Day 1, and wherein the maintenance phase comprises administering the fusion protein starting Day 8 at a maintenance dose of about 960 mg SC weekly for about 24 weeks.

In some embodiments according to any one of the methods described above, the complement-mediated disease is C3G or IgAN. In some embodiments, the initial phase comprises administering the fusion protein at an initial dose of about 600 mg IV to about 3600 mg IV (e.g., about 1200 mg IV to about 3600 mg IV) on Day 1, and the maintenance phase comprises administering the fusion protein starting Day 8 at a maintenance dose of: i) about 600 mg IV to about 1200 mg IV weekly for at least about 48 weeks; ii) about 720 mg SC to about 1440 mg SC weekly for at least about 48 weeks; iii) about 1920 mg SC to about 2880 mg SC biweekly for at least about 49 weeks; iv) about 1800 mg SC to about 2400 mg SC biweekly for at least about 49 weeks; or v) about 960 mg SC to about 2880 mg SC biweekly for about 49 weeks. In some embodiments, the initial phase comprises administering the fusion protein at an initial dose of about 1200 mg IV on Day 1, and the maintenance phase comprises administering the fusion protein starting Day 8 at a maintenance dose of about 720 mg SC or about 600 mg IV weekly for about 48 weeks. In some embodiments, the initial phase comprises administering the fusion protein at an initial dose of about 1200 mg IV on Day 1, and the maintenance phase comprises administering the fusion protein starting Day 8 at a maintenance dose of about 1440 mg SC or about 1200 mg IV weekly for about 48 weeks. In some embodiments, the initial phase comprises administering the fusion protein at an initial dose of about 2400 mg IV to about 3600 mg IV on Day 1, and wherein the maintenance phase comprises administering the fusion protein starting Day 8 at a maintenance dose of about 1920 mg SC to about 2880 mg SC biweekly for about 49 weeks. In some embodiments, the initial phase comprises administering the fusion protein at an initial dose of about 1200 mg IV to about 3600 mg IV on Day 1, and the maintenance phase comprises administering the fusion protein starting Day 8 at a maintenance dose of about 960 mg SC to about 2880 mg SC biweekly for about 49 weeks. In some embodiments, the initial phase comprises administering the fusion protein at an initial dose of about 600 mg IV on Day 1, and the maintenance phase comprises administering the fusion protein starting Day 8 at a maintenance dose of about 720 mg SC weekly for about 48 weeks

In some embodiments according to any one of the methods described above, the binding of the anti-C5 antibody moiety to human C5 is pH-dependent, and the anti-C5 antibody moiety binds more strongly to human C5 at a neutral pH (e.g., about pH 7.4; such as that found in the blood) than it does at an acidic pH (e.g., about pH 5.8; such as that found in the endosome).

In some embodiments according to any one of the methods described above, the anti-C5 antibody moiety is a full-length antibody, a Fab, a Fab′, a F(ab)₂, a F(ab′)₂, an scFv, or a combination thereof.

In some embodiments according to any one of the methods described above, the anti-C5 antibody moiety is a full-length antibody (“anti-C5 full-length antibody”). In some embodiments, the anti-C5 full-length antibody comprises an Fc fragment derived from human IgG4. In some embodiments, the Fc fragment comprises the amino acid sequence of any of SEQ ID NOs: 32, 33, and 61, such as SEQ ID NO: 61. In some embodiments, the anti-C5 full-length antibody comprises a heavy chain and a light chain, wherein (i) the heavy chain comprises the amino acid sequence of SEQ ID NO: 119, and the light chain comprises the amino acid sequence of SEQ ID NO: 74; (ii) the heavy chain comprises the amino acid sequence of SEQ ID NO: 121, and the light chain comprises the amino acid sequence of SEQ ID NO: 74; (iii) the heavy chain comprises the amino acid sequence of SEQ ID NO: 123, and the light chain comprises the amino acid sequence of SEQ ID NO: 74; (iv) the heavy chain comprises the amino acid sequence of SEQ ID NO: 125, and the light chain comprises the amino acid sequence of SEQ ID NO: 74; (v) the heavy chain comprises the amino acid sequence of SEQ ID NO: 120, and the light chain comprises the amino acid sequence of SEQ ID NO: 90; (vi) the heavy chain comprises the amino acid sequence of SEQ ID NO: 122, and the light chain comprises the amino acid sequence of SEQ ID NO: 90; (vii) the heavy chain comprises the amino acid sequence of SEQ ID NO: 124, and the light chain comprises the amino acid sequence of SEQ ID NO: 90; or (viii) the heavy chain comprises the amino acid sequence of SEQ ID NO: 126, and the light chain comprises the amino acid sequence of SEQ ID NO: 90.

In some embodiments according to any one of the methods described above, the fusion protein inhibits C3 activation.

In some embodiments according to any one of the methods described above, the functional fragment of FH comprises short consensus repeat (SCR) domains 1-5 of the FH protein. In some embodiments, the functional fragment of FH comprises the amino acid sequence of SEQ ID NO: 85.

In some embodiments according to any one of the methods described above, the fusion protein comprises a first FH or functional fragment thereof and a second FH or functional fragment thereof, the first FH or functional fragment thereof is fused to the C-terminus of a first heavy chain of the anti-C5 full-length antibody, and the second FH or functional fragment thereof is fused to the C-terminus of a second heavy chain of the anti-C5 full-length antibody. In some embodiments, (i) each heavy chain fused to FH or functional fragment thereof comprises the amino acid sequence of SEQ ID NO: 72, each light chain comprises the amino acid sequence of SEQ ID NO: 74; (ii) each heavy chain fused to FH or functional fragment thereof comprises the amino acid sequence of SEQ ID NO: 76, each light chain comprises the amino acid sequence of SEQ ID NO: 74; (iii) each heavy chain fused to FH or functional fragment thereof comprises the amino acid sequence of SEQ ID NO: 78, each light chain comprises the amino acid sequence of SEQ ID NO: 74; (iv) each heavy chain fused to FH or functional fragment thereof comprises the amino acid sequence of SEQ ID NO: 80, each light chain comprises the amino acid sequence of SEQ ID NO: 74; (v) each heavy chain fused to FH or functional fragment thereof comprises the amino acid sequence of SEQ ID NO: 89, each light chain comprises the amino acid sequence of SEQ ID NO: 90; (vi) each heavy chain fused to FH or functional fragment thereof comprises the amino acid sequence of SEQ ID NO: 116, each light chain comprises the amino acid sequence of SEQ ID NO: 90; (vii) each heavy chain fused to FH or functional fragment thereof comprises the amino acid sequence of SEQ ID NO: 117, each light chain comprises the amino acid sequence of SEQ ID NO: 90; or (viii) each heavy chain fused to FH or functional fragment thereof comprises the amino acid sequence of SEQ ID NO: 118, each light chain comprises the amino acid sequence of SEQ ID NO: 90.

In some embodiments according to any one of the methods described above, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0.

The methods described herein can use any of the fusion proteins described herein. Anti-C5 antibody moieties and the FH moieties are described in more details below.

### BRIEF DESCRIPTION OF THE DRAWINGS

FIG. 1A depicts the bifunctional structure of the anti-C5-FH fusion protein and the SDS-PAGE separation of humanized anti-C5 mAb and anti-C5-FH fusion protein. FIG. 1B depicts 2G1-3 binding to a different epitope from Eculizumab.

FIG. 2A depicts a C5 Inhibition Assay showing that anti-C5-FH fusion protein is as potent as Ravulizumab in inhibiting CP-triggered terminal pathway complement activation (sheep red blood cell (RBC) lysis). FIG. 2B depicts an LPS-based ELISA assay showing that anti-C5-FH fusion protein also inhibits AP complement. FIG. 2C depicts a rabbit RBCs lysis assay showing that anti-C5-FH fusion protein is more potent than anti-C5 mAb or FH SCR1-5-Fc, alone or combined, in inhibiting AP-triggered terminal pathway complement activation.

FIG. 3 depicts anti-C5-FH fusion protein is more potent than Ecu/Rav mAbs in inhibiting the lysis of human PNH RBCs and it differentiates from Ecu/Rav in inhibiting C3b fragment opsonization of non-lysed PNH RBCs.

FIG. 4 depicts anti-C5-FH fusion protein dose-dependently inhibited extravascular hemolysis (EVH) in a mouse model of EVH.

FIG. 5 depicts anti-C5-FH fusion protein possess tissue targeting property for cells with C5b-9 deposition.

FIG. 6A depicts a survival curve for FH^{m/m}P^{-/-} and factor D humanization in FH^{m/m}P^{-/-} mice (hFD-FH^{m/m}P^{-/-}). FIG. 6B depicts immunofluorescence staining of C3 in hFD-FH^{m/m}P^{-/-} mice. FIG. 6C depicts protein levels of C3 and C5 in FH^{m/m}P^{-/-} and hFD-FH^{m/m}P^{-/-} mice.

FIG. 7A depicts a novel bifunctional complement inhibitor comprising of an anti-C5 mAb (BB5.1) and mouse factor H SCR 1-5 fusion protein. FIG. 7B depicts a sheep red blood cell (RBC) lysis assay performed with 50% mouse plasma for the murine anti-C5-FH fusion protein and BB5.1. FIG. 7C depicts a rabbit RBC lysis assay performed with 50% mouse plasma for the murine anti-C5-FH fusion protein and BB5.1. FIG. 7D depicts an LPS-based ELISA assay for the murine anti-C5-FH fusion protein and BB5.1.

FIG. 8A depicts a survival curve of hFD-FH^{m/m}P^{-/-} mice treated with either the murine anti-C5-FH fusion protein or BB5.1. FIG. 8B depicts the protein levels of systemic C3 and factor B consumption in hFD-FH^{m/m}P^{-/-} mice injected with the murine anti-C5-FH fusion protein or BB5.1. FIG. 8C depicts the scores for proteinuria and hematuria in hFD-FH^{m/m}P^{-/-} mice injected with the murine anti-C5-FH fusion protein or BB5.1. FIG. 8D depicts the scores for crescents and fibrin deposition, endocapillary hypercellularity, and mesangial hypercellularity in hFD-FH^{m/m}P^{-/-} mice injected with the murine anti-C5-FH fusion protein or BB5.1. FIG. 8E depicts immunofluorescence staining and quantification of glomerular C3 and C9 deposition in hFD-FH^{m/m}P^{-/-} mice injected with the murine anti-C5-FH fusion protein or BB5.1.

FIG. 9 depicts a Phase 1 anti-C5-FH fusion protein clinical study schema.

FIGS. 10A-10B depict a table of demographic characteristics.

FIG. 11 depicts a table of most frequently reported treatment-emergent adverse events.

FIG. 12A depicts anti-C5-FH fusion protein concentration-time profiles (semi-logarithmic scale) for the SAD cohort. FIG. 12B depicts anti-C5-FH fusion protein concentration-time profiles (semi-logarithmic scale) for the MAD cohort.

FIG. 13A depicts mean (SD) serum rRBC versus time by dosing regimen in the MAD cohort. FIG. 13B depicts mean (SD) C3b versus time by dosing regimen for the MAD cohort.

FIG. 13C depicts mean (SD) free C5 versus time by dosing regimen for the MAD cohort.

FIG. 14A depicts a scatter plot of the percent change from baseline of serum rRBC levels versus anti-C5-FH fusion protein serum concentrations in all subjects. FIG. 14B depicts a scatter plot of the percent change from baseline of serum C3b levels versus anti-C5-FH fusion protein concentrations in all subjects. FIG. 14C depicts scatter plot of the percent change from baseline of free C5 levels versus anti-C5-FH fusion protein concentrations in all subjects.

FIG. 15 depicts a systemic lupus erythematosus (SLE)-Thrombotic microangiopathy (TMA) clinical study schema.

FIG. 16 depicts an IgA Nephropathy (IgAN) and Complement 3 Glomerulopathy (C3G) clinical study schema.

FIG. 17 depicts a Phase 2 paroxysmal nocturnal hemoglobinuria (PNH) clinical study schema.

FIG. 18 depicts a graph of the mean (±standard deviation) hemoglobin increase from baseline of complement inhibitor-naïve PNH patients administered the anti-C5-FH fusion protein across 17 weeks for Cohorts 1, 2, and 3. The horizonal dashed line represents a 2 g/dL of hemoglobin increase from baseline. The vertical dashed lines indicate the specified time in weeks. Mean (SD) hemoglobin levels increased by 4.9 (±1.7) g/dL, 5.8 (±1.6) g/dL, and 5.8 (±2.8) g/dL over baseline for Cohorts 1, 2, and 3, respectively.

FIG. 19 depicts a graph of the mean (±standard deviation) lactate dehydrogenase (LDH) levels of complement inhibitor-naïve PNH patients administered the anti-C5-FH fusion protein across 17 weeks for Cohorts 1, 2, and 3. The top, horizontal dashed line represents total LDH that is 1.5 times the upper limit of normal (ULN). The lower, horizonal dashed line represents the total LDH that is 1 times the upper limit of normal. The vertical dashed lines indicate the specified time in weeks. Mean (SD) LDH levels reduced by 88.0 (±3.67) %, 83.5 (±7.45) %, and 89.5 (±4.05) % over baseline for Cohorts 1, 2, and 3, respectively.

### DETAILED DESCRIPTION OF THE INVENTION

The present application in one aspect provides methods of treating complement-mediated diseases through inhibition of complement signaling using an anti-C5/factor H fusion protein (hereinafter referred to as “anti-C5-FH fusion protein”) comprising an anti-C5 antibody moiety and a factor H (FH) moiety. Anti-C5-FH fusion proteins used herein inhibit complement system activities via dual mechanisms: i) the anti-C5 antibody moiety functions as an anti-C5 antibody to block C5 activity; and ii) the FH moiety acts as a C3 complement activation inhibitor. In some embodiments, the anti-C5 antibody moiety exhibits pH-dependent binding to C5 (hereinafter referred to as “pH-dependent anti-C5 antibody moiety”). In some embodiments, the pH-dependent anti-C5 antibody moiety binds more strongly to C5 at a more neutral pH (e.g., about pH 7.4; such as that found in the blood) than it does at a more acidic pH (e.g., about pH 5.8; such as that found in the endosome). In some embodiments, the FH moiety is FH protein or fragment thereof, such as an FH fragment comprising short consensus repeat (SCR) domains 1-5 of a FH protein, which are domains involved in regulating C3 activation. Complement-mediated diseases include, but are not limited to, paroxysmal nocturnal hemoglobinuria (PNH) syndrome, C3 glomerulopathy (C3G), IgA nephropathy (IgAN), and thrombotic microangiopathy secondary to systemic lupus erythematosus (SLE-TMA). Anti-C5-FH fusion proteins and anti-C5 antibody moieties (e.g., pH-dependent anti-C5 antibody moieties) have been described in US Patent Publication No. 20220204602 and international patent application No. WO2020/219922, the entire contents of each of which is specifically incorporated herein by reference in their entirety. Treatment methods described herein provide more effective and more convenient ways to treat complement-dependent pathologies, such as by reducing dosing amount and/or frequency, and/or by more effectively blocking complement system activities, which addresses and meets needs previously unmet by other complement-mediated disease therapies (e.g., FDA-approved eculizumab).

### I. Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

The terms “inhibit” and “inhibition,” as used herein, means to reduce, suppress, diminish or block an activity or function by at least about 10% (e.g., at least about any of 20%, 30%, 40%, 50%, 60%, 70%, 80%, 90%, 95%, 99%, or 100%) relative to a control value. In some embodiments, the activity is suppressed or blocked by at least about 50% compared to a control value. In some embodiments, the activity is suppressed or blocked by at least about 75%. In some embodiments, the activity is suppressed or blocked by at least about 95%. In some embodiments, the activity is 100% blocked.

The terms “effective amount” and “pharmaceutically effective amount” refer to a sufficient amount of an agent to provide the desired biological result. That result can be reduction (e.g., reducing at least about any of 10%, 20%, 30%, 40%, 50%, 60%, 70%, 80%, 90%, 95%, 99%, or 100%) and/or alleviation of the signs, symptoms, or causes of a disease or disorder, or any other desired alteration of a biological system. An appropriate effective amount in any individual case may be determined by one of ordinary skill in the art using routine experimentation.

The terms “patient,” “subject,” “individual,” and the like are used interchangeably herein, and refer to any animal, in some embodiments a mammal, and in some embodiments a human, having a complement system, including a human in need of therapy for, or susceptible to, a condition or its sequelae. The individual may include, for example, dogs, cats, pigs, cows, sheep, goats, horses, rats, rabbits, hamsters, guinea pigs, monkeys, mice, and humans. In some embodiments, the individual is a human.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected/homeostatic) respective characteristic. Characteristics which are normal or expected for one cell, tissue type, or subject, might be abnormal for a different cell or tissue type.

A “disease” is a state of health of a subject wherein the subject cannot maintain homeostasis, and wherein if the disease is not ameliorated then the subject's health continues to deteriorate.

In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

A disease or disorder is “alleviated” if the severity of a sign or symptom of the disease or disorder, the frequency with which such a sign or symptom is experienced by a patient, or both, is reduced.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with disease or disorder are mitigated or eliminated, including, but not limited to, decreasing the frequency and/or severity of a sign and/or symptoms resulting from the disease, increasing the quality of life of those suffering from the disease, decreasing the dose of other medications required to treat the disease, and/or prolonging survival of individuals. Treatment may be prophylactic (to prevent or delay the onset of the disease, or to prevent the manifestation of clinical or subclinical symptoms thereof) or therapeutic suppression or alleviation of symptoms after the manifestation of the disease.

A subject who “may be suitable”, which includes a subject who “is suitable” for treatment(s) described herein, is a subject who is more likely than not to benefit from administration of said treatments. Conversely, a subject who “may not be suitable” or “may be unsuitable”, which includes a subject who is “unsuitable” for treatment(s) described herein, is a subject who is more likely than not to fail to benefit from administration of said treatments.

An “effective amount” or “therapeutically effective amount” of a compound is that amount of compound which is sufficient to provide a beneficial effect to the subject to which the compound is administered.

A “therapeutic treatment” is a treatment administered to a subject who exhibits signs of disease or disorder, for the purpose of diminishing or eliminating those signs.

The term “antibody,” as used herein, refers to an immunoglobulin molecule which is able to specifically bind to a specific epitope of an antigen. Antibodies can be intact immunoglobulins derived from natural sources, or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab, Fab′, F(ab)₂, and F(ab′)₂, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

As used herein, the term “heavy chain antibody” or “heavy chain antibodies” comprises immunoglobulin molecules derived from camelid species, either by immunization with a peptide and subsequent isolation of sera, or by the cloning and expression of nucleic acid sequences encoding such antibodies. The term “heavy chain antibody” or “heavy chain antibodies” further encompasses immunoglobulin molecules isolated from a subject with heavy chain disease, or prepared by the cloning and expression of VH (variable heavy chain immunoglobulin) genes from a subject.

A “chimeric antibody” refers to a type of engineered antibody which contains a naturally-occurring variable region (light chain and heavy chains) derived from a donor antibody in association with light and heavy chain constant regions derived from an acceptor antibody.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., 1989, Queen et al., Proc. Natl. Acad Sci USA, 86:10029-10032; 1991, Hodgson et al., Bio/Technology, 9:421). A suitable human acceptor antibody may be one selected from a conventional database, e.g., the KABAT database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies (see for example EP-A-0239400 and EP-A-054951).

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). The structure and protein folding of the antibody may mean that other residues are considered part of the antigen binding region and would be understood to be so by a skilled person. See for example Chothia et al., (1989) Conformations of immunoglobulin hypervariable regions; Nature 342, p 877-883.

The terms “native antibody,” “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region. Native antibodies are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs, also referred to as CDRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)).

The term “constant domain” refers to the portion of an immunoglobulin molecule having a more conserved amino acid sequence relative to the other portion of the immunoglobulin, the variable domain, which contains the antigen binding site. The constant domain contains the C_{H}1, C_{H}2 and C_{H}3 domains (collectively, CH) of the heavy chain and the CL domain of the light chain. The constant domains are not involved directly in the binding of an antibody to an antigen, but exhibit various effector functions, such as participation of the antibody in antibody-dependent cellular toxicity.

The “light chains” of antibodies (immunoglobulins) from any mammalian species can be assigned to one of two clearly distinct types, called kappa (“κ”) and lambda (“λ”), based on the amino acid sequences of their constant domains.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

“Antibody fragments” comprise a portion of an intact antibody, preferably comprising the antigen binding region thereof. In some embodiments, the antibody fragment described herein is an antigen binding fragment. Examples of antibody fragments or antigen binding fragments include Fab, Fab′, F(ab′)₂, and Fv fragments (such as single-chain variable fragment, scFv); diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment which contains a complete antigen-binding site. In some embodiments, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment has two polypeptide chains, containing the heavy- and light-chain variable domains (VH, VL), and also containing the constant domain of the light chain (CL) and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthün, The Pharmacology of Monoclonal Antibodies. Springer Berlin Heidelberg, 1994. 269-315.

The “Fc” fragment comprises the carboxy-terminal portions of both heavy chains held together by di-sulfides. The effector functions of antibodies are determined by sequences in the Fc region, which region is also the part recognized by Fc receptors (FcR) found on certain types of cells.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In some embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature 256:495-97 (1975); Hongo et al., Hybridoma 14 (3): 253-260 (1995), Harlow et al., Antibodies: A Laboratory Manual, (Cold Spring Harbor Laboratory Press, 2nd ed. 1988); Hammerling et al., Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004)), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995)).

The monoclonal antibodies (mAbs) herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATTZED® antibodies wherein the antigen-binding region of the antibody is derived from an antibody produced by, e.g., immunizing macaque monkeys with the antigen of interest.

The structures and locations of immunoglobulin variable regions may be determined by reference to Kabat, E. A. et al., Sequences of Proteins of Immunological Interest. 4th Edition. US Department of Health and Human Services. 1987, and updates thereof, now available on the Internet (immuno.bme.nwu.edu).

By the term “specifically binds,” as used herein with respect to an antibody, is meant an antibody which recognizes and binds to a specific target molecule, but does not substantially recognize or bind other molecules in a sample. In some instances, the terms “specific binding” or “specifically binding,” is used to mean that the recognition and binding is dependent upon the presence of a particular structure (e.g., an antigenic determinant or epitope) on the target molecule. If, for example, an antibody specifically binds to epitope “A,” the presence of an unlabelled molecule containing epitope A (or free, unlabeled A) in a reaction containing labeled “A” and the antibody, will reduce the amount of labeled A bound to the antibody. An antibody that binds to or specifically binds to a target (e.g., an epitope) is an antibody that binds this target with greater affinity, avidity, more readily, and/or with greater duration than it binds to other targets. In some embodiments, the extent of binding of an antibody to an unrelated target is less than about 10% of the binding of the antibody to the target as measured, e.g., by a radioimmunoassay (RIA). In some embodiments, an antibody that specifically binds to a target has a dissociation constant (K_{d}) of ≤1 μM, ≤100 nM, ≤10 nM, ≤1 nM, or ≤0.1 nM. In some embodiments, an antibody specifically binds to an epitope on a protein that is conserved among the protein from different species. In some embodiments, specific binding can include, but does not require exclusive binding.

As used herein, the term “affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity.

The term “multispecific” as used in conjunction with an antibody or antigen binding protein refers to an antibody or antigen binding protein having polyepitopic specificity (i.e., is capable of specifically binding to two, three, or more, different epitopes on one biological molecule or is capable of specifically binding to epitopes on two, three, or more, different biological molecules).

As used herein, “Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but is not limited to the replacement of one amino acid in a polypeptide with another amino acid. Exemplary substitutions are shown in Table A. Amino acid substitutions may be introduced into an antibody of interest and the products screened for a desired activity, e.g., retained/improved antigen binding, decreased immunogenicity, or improved ADCC or CDC.

TABLE A

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class.

The term “covalently linked” as used herein, refers to a direct linkage through one or more chemical bonds or an indirect linkage through one or more linkers. Any suitable chemical bond can be used to create a direct linkage, including but not limited to, a covalent bond such as a peptide bond and a disulfide bond, or a non-covalent bond such as a hydrogen bond, a hydrophobic bond, an ionic bond, or a van der Waals bond.

As used herein, the “C terminus” of a polypeptide refers to the last amino acid residue of the polypeptide which donates its amine group to form a peptide bond with the carboxyl group of its adjacent amino acid residue. “N terminus” of a polypeptide as used herein refers to the first amino acid of the polypeptide which donates its carboxyl group to form a peptide bond with the amine group of its adjacent amino acid residue.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

It is understood that embodiments of the invention described herein include “consisting of” and/or “consisting essentially of” embodiments.

As used herein, reference to “not” a value or parameter generally means and describes “other than” a value or parameter. For example, the method is not used to treat cancer of type X means the method is used to treat cancer of types other than X.

Reference to “about” a value or parameter herein includes (and describes) variations that are directed to that value or parameter per se. For example, description referring to “about X” includes description of “X”. The term “about X-Y” used herein has the same meaning as “about X to about Y.”

As used herein and in the appended claims, the singular forms “a,” “or,” and “the” include plural referents unless the context clearly dictates otherwise.

### II. Methods of Treatment

This invention relates to the inhibition of the complement signaling and complement-related diseases or disorders in an individual (e.g., human) using an anti-C5-FH fusion protein (e.g., FMEH-IgG4-PLA-FH) comprising i) an anti-C5 antibody moiety that specifically binds to C5 (e.g., human C5) and ii) an FH or functional fragment thereof. This invention also relates to the inhibition of the complement signaling and complement-related diseases or disorders in an individual (e.g., human) using any of the anti-C5 antibody moieties described herein. In some embodiments, the anti-C5 antibody moiety exhibits pH-dependent binding to C5 (e.g., human C5). In some embodiments, the pH-dependent anti-C5 antibody moiety binds more strongly to C5 at a more neutral pH (e.g., about pH 7.4; such as that found in the blood) than it does at a more acidic pH (e.g., about pH 5.8; such as that found in the endosome). Such pH-dependent binding provides for greater persistence of administered antibody molecules, because immune complexes (i.e., anti-C5 mAb bound to C5) taken up by cells will dissociate in the acidic environment of the endosome and allow the freed antibody to be recycled back out of the cell through the neonatal Fc receptor (FcRn) where it is available to bind to a new C5 molecule. In some embodiments, the individual is a complement inhibitor-naïve individual.

In some embodiments, the invention is directed to inhibiting the complement signaling cascade by specifically targeting complement component C5 protein, or a fragment of the protein C5a or C5b, such as by inhibiting (e.g., inhibiting at least about any of 10%, 20%, 30%, 40%, 50%, 60%, 70%, 80%, 90%, 95%, 99%, or 100%) C5a-mediated inflammation and cell activation, or inhibiting C5b-mediated cell lysis. In some embodiments, the invention is directed to inhibiting the complement signaling cascade by specifically targeting complement component C3b protein, such as for preventing (e.g., preventing at least about any of 10%, 20%, 30%, 40%, 50%, 60%, 70%, 80%, 90%, 95%, 99%, or 100%) complement C3b deposition and amplification. In some embodiments, the invention is directed to methods of treating and preventing inflammation and autoimmune diseases mediated by unwanted, uncontrolled, excessive complement activation. In some embodiments, the invention is directed towards the treatment of complement-mediated disease or complement-mediated disorder in an individual (e.g., human) by administering to the individual (e.g., intravenously or subcutaneously) an anti-C5-FH fusion protein (e.g., FMEH-IgG4-PLA-FH). In some embodiments, the invention is directed towards the treatment of complement-mediated disease or complement-mediated disorder in an individual (e.g., human) by administering to the individual (e.g., intravenously or subcutaneously) an anti-C5 antibody moiety (such as any of the anti-C5 antibodies or antigen-binding fragments thereof described herein).

Any complement-related diseases, such as diseases related to C3 (or C3b) and/or C5 (or C5a, C5b) activities, or AP and/or terminal pathway activities, can be treated using the methods described herein. Defective complement action is a cause of several human glomerular diseases including atypical hemolytic uremic syndrome (aHUS), anti-neutrophil cytoplasmic antibody mediated vasculitis (ANCA), C3 glomerulopathy, IgA nephropathy, immune complex membranoproliferative glomerulonephritis, renal ischemic reperfusion injury, lupus nephritis, membranous nephropathy, and chronic transplant mediated glomerulopathy. Aberrant complement component activation has also been proposed as markers in various types of cancers and their clinical outcomes. Lung cancer patients show significantly higher plasma levels of complement proteins and activation fragments than do control donors, and elevated complement levels are correlated with lung tumor size. Complement-related proteins are also elevated in biological fluids from patients with other types of tumor. See, for example, Pio et al. Semin Immunol. 2013 February; 25(1): 54-64. Inhibition of the complement cascade has been proposed for glomerular diseases and cancer treatment.

In some embodiments, the complement-related disease is selected from the group consisting of: macular degeneration (MD), age-related macular degeneration (AMD), ischemia reperfusion injury, arthritis, rheumatoid arthritis, asthma, allergic asthma, lupus, ulcerative colitis, stroke, post-surgery systemic inflammatory syndrome, chronic obstructive pulmonary disease (COPD), PNH syndrome, myasthenia gravis, neuromyelitis optica, (NMO), multiple sclerosis, delayed graft function, antibody-mediated rejection, aHUS, central retinal vein occlusion (CRVO), central retinal artery occlusion (CRAO), epidermolysis bullosa, sepsis, organ transplantation, inflammation (including, but not limited to, inflammation associated with cardiopulmonary bypass surgery and kidney dialysis), C3 glomerulopathy (C3G), membranous nephropathy, IgA nephropathy (IgAN), glomerulonephritis (including, but not limited to, anti-neutrophil cytoplasmic antibody (ANCA)-mediated glomerulonephritis, lupus nephritis, and combinations thereof), thrombotic microangiopathies secondary to systemic lupus erythematosus (SLE-TMA), ANCA-mediated vasculitis, shiga toxin induced HUS, and antiphospholipid antibody-induced pregnancy loss, or any combinations thereof. In some embodiments, the AP-mediated disease is C3G. In some embodiments, the AP-mediated disease is macular degeneration, such as AMD.

In some embodiments, the invention is a method of treating a complement-mediated disease or disorder in an individual (e.g., human), comprising the step of administering to said human individual an anti-C5 antibody moiety (e.g., any of anti-C5 antibodies or antigen-binding fragments thereof described herein), thereby inhibiting the generation of a C5a or C5b protein, and formation of MAC. Examples of complement-mediated diseases that can be treated using the methods of the invention include, but are not limited to PNH syndrome, C3G, IgAN, and SLE-TMA.

In some embodiments, there is provided a method of treating a complement-mediated disease in a human individual (e.g., complement inhibitor-naïve human individual), comprising administering to the human individual an effective amount of a fusion protein comprising i) an antibody moiety that specifically binds to a human C5 (anti-human C5 antibody moiety; e.g., FMEH-IgG4-PLA) and ii) an FH or fragment thereof (e.g., SCR1-5 domains of FH, such as SEQ ID NO: 85), such as any of the anti-C5-FH fusion proteins described herein (e.g., FMEH-IgG4-PLA-FH). In some embodiments, the complement-mediated disease is selected from the group consisting of PNH syndrome, C3G, IgAN, and SLE-TMA. In some embodiments, the fusion protein is administered intravenously (IV). In some embodiments, the fusion protein is administered subcutaneously (SC). In some embodiments, the fusion protein is administered at a dose of about 60 mg to about 3600 mg, such as any of about 60 mg to about 1200 mg, about 60 mg to about 3000 mg, about 60 mg to about 2880 mg, about 600 mg to about 1200 mg, about 600 mg to about 2880 mg, about 600 mg to about 3600 mg, about 1200 mg to about 3600 mg, about 720 mg to about 1440 mg, about 720 mg to about 3000 mg, about 1920 mg to about 2880 mg, about 1800 mg to about 3600 mg, or about 1800 mg to about 2400 mg. In some embodiments, the fusion protein is administered at a single dose. In some embodiments, the fusion protein is administered at a dose (e.g., single dose) of about 60 mg to about 1200 mg (e.g., about any of 60 mg, 120 mg, 180 mg, 240 mg, 300 mg, 360 mg, 420 mg, 480 mg, 540 mg, 600 mg, 660 mg, 720 mg, 780 mg, 840 mg, 900 mg, 960 mg, or 1200 mg). In some embodiments, the fusion protein is administered at a dose (e.g., single dose) of: about 60 mg IV, about 180 mg IV, about 180 mg SC, about 360 mg IV, about 600 mg IV, about 720 mg SC, or about 1200 mg IV. In some embodiments, the fusion protein is administered at multiple doses (e.g., 2, 3, 4, 5, 6, 8, 10, 11, 12, 13, 14, 23, 24, 25, 26, 40, 44, 47, 48, 49, 50, or more doses). In some embodiments, the fusion protein is administered weekly (QW). In some embodiments, the fusion protein is administered biweekly (Q2W). In some embodiments, the fusion protein is administered at multiple doses (e.g., QW or Q2W) of each of about 600 mg to about 2880 mg, such as any of about 600 mg to 1200 mg, about 1200 mg to about 2880 mg, about 720 mg to about 1440 mg, about 720 mg to about 2880 mg, about 960 mg to about 1920 mg, about 960 mg to about 2880 mg, about 1920 mg to about 2880 mg, about 1400 mg to about 2880 mg, about 1800 mg to about 2880 mg, or about 1800 mg to about 2400 mg. In some embodiments, the fusion protein is administered at multiple doses (e.g., QW or Q2W) of each of about any of 600 mg, 660 mg, 720 mg, 780 mg, 840 mg, 900 mg, 960 mg, 1200 mg, 1440 mg, 1800 mg, 1920 mg, 2400 mg, 2880 mg, 3000 mg, or 3600 mg. In some embodiments, the fusion protein is administered at a dose of about 600 mg IV weekly for 5 weeks. In some embodiments, the anti-C5 antibody moiety comprises a VH-CDR1 comprising the amino acid sequence of SEQ ID NO: 86, a VH-CDR2 comprising the amino acid sequence of SEQ ID NO: 57, a VH-CDR3 comprising the amino acid sequence of SEQ ID NO: 49, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96. In some embodiments, the anti-C5 antibody moiety comprises a VH comprising the amino acid sequence of SEQ ID NO: 87, and a VL comprising the amino acid sequence of SEQ ID NO: 88. In some embodiments, the anti-C5 antibody moiety is a full-length antibody comprising i) a heavy chain comprising the amino acid sequence of SEQ ID NO: 120, and ii) a light chain comprising the amino acid sequence of SEQ ID NO: 90. In some embodiments, the FH or functional fragment thereof is fused to the C-terminus of one or both (e.g., both) heavy chains of the anti-C5 full-length antibody. In some embodiments, the heavy chain fused to FH or functional fragment thereof comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating a complement-mediated disease in a human individual (e.g., complement inhibitor-naïve human individual), comprising administering to the human individual an effective amount of a fusion protein (e.g., any of the anti-C5-FH fusion proteins described herein, such as FMEH-IgG4-PLA-FH) comprising i) an anti-human C5 antibody moiety (e.g., anti-human C5 full-length antibody, such as FMEH-IgG4-PLA) and ii) an FH or fragment thereof (e.g., SCR1-5 domains of FH, such as SEQ ID NO: 85), wherein the fusion protein is administered with an initial phase comprising administering the fusion protein at one or more (e.g., one) initial doses, followed by a maintenance phase comprising administering the fusion protein (e.g., weekly or biweekly) for two or more (e.g., 2, 3, 4, 5, 6, 8, 10, 11, 12, 13, 14, 23, 24, 25, 26, 40, 44, 47, 48, 49, 50, or more) maintenance doses. In some embodiments, the complement-mediated disease is selected from the group consisting of PNH, C3G, IgAN, and SLE-TMA. In some embodiments, the initial dose is about 600 mg to about 3600 mg, such as any of about 1200 mg to about 3600 mg, about 600 mg to about 1200 mg, about 600 mg to about 2880 mg, about 1200 mg to about 3600 mg, about 720 mg to about 1440 mg, about 720 mg to about 3000 mg, about 1920 mg to about 2880 mg, about 1800 mg to about 3600 mg, or about 1800 mg to about 2400 mg. In some embodiments, the initial dose is about 1200 mg to about 3600 mg, such as about any of 1200 mg, 1440 mg, 1800 mg, 1920 mg, 2400 mg, 2880 mg, 3000 mg, or 3600 mg. In some embodiments, the one or more (e.g., one) initial doses of the fusion protein are administered IV. In some embodiments, the initial phase comprises administering the fusion protein at 1, 2, 3, 4, 5, 6, 10, 12, 14, 16, or more initial doses. In some embodiments, the initial phase comprises administering the fusion protein at one initial dose. In some embodiments, the maintenance dose is about 600 mg to about 2880 mg, such as any of about 1200 mg to about 2880 mg, about 600 mg to about 1200 mg, about 960 mg to about 1440 mg, about 960 mg to about 1920 mg, about 960 mg to about 2880 mg, about 600 mg to about 2000 mg, about 720 mg to about 2400 mg, about 720 mg to about 1440 mg, about 1920 mg to about 2880 mg, about 1800 mg to about 2880 mg, or about 1800 mg to about 2400 mg. In some embodiments, the maintenance dose is about any of 600 mg, 720 mg, 960 mg, 1200 mg, 1440 mg, 1800 mg, 1920 mg, 2400 mg, or 2880 mg. In some embodiments, the two or more maintenance doses of the fusion protein are administered weekly. In some embodiments, the maintenance dose (e.g., weekly) is about 600 mg to about 1440 mg, such as any of about 600 mg to about 1200 mg, about 720 mg to about 1440 mg, about 600 mg, about 720 mg, about 840 mg, about 960 mg, about 1080 mg, about 1200 mg, about 1320 mg, or about 1440 mg. In some embodiments, the two or more maintenance doses of the fusion protein are each administered (e.g., weekly) at about 600 mg IV to about 1200 mg IV, or at about 720 mg SC to about 1440 mg SC. For example, the two or more maintenance doses of the fusion protein are each administered (e.g., weekly) at any of about 600 mg IV, about 1200 mg IV, about 720 mg SC, about 960 mg SC, or about 1440 mg SC. In some embodiments, the two or more maintenance doses of the fusion protein are administered biweekly. In some embodiments, the maintenance dose (e.g., biweekly) is about 960 mg to about 2880 mg, such as any of about 960 mg to about 1920 mg, about 1920 mg to about 2880 mg, about 1800 mg to about 2400 mg, about 1800 mg to about 1920 mg, about 1800 mg to about 2880 mg, about 1800 mg, about 1920 mg, about 2000 mg, about 2200 mg, about 2400 mg, about 2600 mg, or about 2880 mg. In some embodiments, the two or more maintenance doses of the fusion protein are each administered (e.g., biweekly) at about 1920 mg SC to about 2880 mg SC, or at about 1800 mg SC to about 2400 mg SC, such as any of about 960 mg SC, about 1800 mg SC, about 1920 mg SC, about 2400 mg SC, or about 2880 mg SC. In some embodiments, the maintenance phase is at least about 4 weeks, such as at least about any of 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 42, 44, 45, 46, 47, 48, 49, 50, 52 weeks, or longer. In some embodiments, the maintenance phase is at least about 12 weeks, such as any of 12 weeks, 13 weeks, 24 weeks, 25 weeks, 48 weeks, or 49 weeks. In some embodiments, the initial phase comprises administering the fusion protein at an initial dose of about 1200 mg weekly for one or more (e.g., one) doses, and wherein the maintenance phase comprises administering the fusion protein at a maintenance dose of about 720 mg weekly or biweekly for at least two (e.g., at least any of 4, 5, 12, 13, 24, 25, 48, or 49) doses. In some embodiments, the initial phase comprises administering the fusion protein at an initial dose of about 1200 mg IV on Day 1, and the maintenance phase comprises administering the fusion protein at a maintenance dose of about 720 mg SC weekly starting Day 8 for 4 weeks. In some embodiments, the maintenance phase comprises administering the fusion protein (e.g., weekly or biweekly) at a first maintenance dose for a first maintenance phase period, followed by administering the fusion protein (e.g., weekly or biweekly) at a second maintenance dose for a second maintenance phase period. In some embodiments, the second maintenance dose is higher than the first maintenance dose, such as at least about any of 1.5, 2, 3, 4, 5, 10-fold or more higher. In some embodiments, the second maintenance phase period is longer than the first maintenance phase period, such as at least about any of 2, 4, 6, 8, 10, 12, 14, 16, 18, 20, 22, 24, 26, 28, 30, 32, 34, 36, 38, 40, 42, 44, 46, 48 weeks or more longer. In some embodiments, the first maintenance phase period is at least 1 week, and the second maintenance phase period is no more than about 23 or about 24 weeks. In some embodiments, the first maintenance dose of the fusion protein is administered at about 720 mg SC or about 600 mg IV weekly, and the second maintenance dose of the fusion protein is administered at about 1440 mg SC or about 1200 mg IV weekly. In some embodiments, the first maintenance phase period is about 4 weeks, and the second maintenance phase period is at least about 44 weeks. In some embodiments, the anti-C5 antibody moiety comprises a VH-CDR1 comprising the amino acid sequence of SEQ ID NO: 86, a VH-CDR2 comprising the amino acid sequence of SEQ ID NO: 57, a VH-CDR3 comprising the amino acid sequence of SEQ ID NO: 49, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96. In some embodiments, the anti-C5 antibody moiety comprises a VH comprising the amino acid sequence of SEQ ID NO: 87, and a VL comprising the amino acid sequence of SEQ ID NO: 88. In some embodiments, the anti-C5 antibody moiety is a full-length antibody comprising i) a heavy chain comprising the amino acid sequence of SEQ ID NO: 120, and ii) a light chain comprising the amino acid sequence of SEQ ID NO: 90. In some embodiments, the FH or functional fragment thereof is fused to the C-terminus of one or both (e.g., both) heavy chains of the anti-C5 full-length antibody. In some embodiments, the heavy chain fused to FH or functional fragment thereof comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating a complement-mediated disease in a human individual (e.g., complement inhibitor-naïve human individual), comprising administering to the human individual an effective amount of a fusion protein (e.g., any of the anti-C5-FH fusion proteins described herein, such as FMEH-IgG4-PLA-FH) comprising i) an anti-human C5 antibody moiety (e.g., anti-human C5 full-length antibody, such as FMEH-IgG4-PLA) and ii) an FH or fragment thereof (e.g., SCR1-5 domains of FH, such as SEQ ID NO: 85), wherein the fusion protein is administered at a single dose of: about 60 mg IV, about 180 mg IV, about 180 mg SC, about 360 mg IV, about 600 mg IV, about 720 mg SC, or about 1200 mg IV. In some embodiments, there is provided a method of treating a complement-mediated disease in a human individual, comprising administering to the human individual an effective amount of a fusion protein (e.g., FMEH-IgG4-PLA-FH) comprising i) an anti-human C5 antibody moiety (e.g., anti-human C5 full-length antibody, such as FMEH-IgG4-PLA) and ii) an FH or fragment thereof (e.g., SCR1-5 domains of FH, such as SEQ ID NO: 85), wherein the fusion protein is administered at a dose of about 600 mg IV weekly for 5 weeks. In some embodiments, there is provided a method of treating a complement-mediated disease in a human individual, comprising administering to the human individual an effective amount of a fusion protein (e.g., FMEH-IgG4-PLA-FH) comprising i) an anti-human C5 antibody moiety (e.g., anti-human C5 full-length antibody, such as FMEH-IgG4-PLA) and ii) an FH or fragment thereof (e.g., SCR1-5 domains of FH, such as SEQ ID NO: 85), wherein the fusion protein is administered with an initial phase comprising administering the fusion protein at an initial dose of about 1200 mg (e.g., IV) weekly for one or more doses (e.g., one dose only), followed by a maintenance phase comprising administering the fusion protein at a maintenance dose of about 720 mg (e.g., SC) weekly or biweekly for at least two (e.g., 2, 3, 4, 5, 6, 8, 10, 11, 12, 13, 14, 23, 24, 25, 26, 40, 44, 47, 48, 49, 50, or more) doses. In some embodiments, there is provided a method of treating a complement-mediated disease in a human individual, comprising administering to the human individual an effective amount of a fusion protein (e.g., FMEH-IgG4-PLA-FH) comprising i) an anti-human C5 antibody moiety (e.g., anti-human C5 full-length antibody, such as FMEH-IgG4-PLA) and ii) an FH or fragment thereof (e.g., SCR1-5 domains of FH, such as SEQ ID NO: 85), wherein the fusion protein is administered with an initial phase comprising administering the fusion protein at an initial dose of about 1200 mg IV on Day 1, followed by a maintenance phase comprising administering the fusion protein at a maintenance dose of about 720 mg SC weekly starting Day 8 for 4 weeks. In some embodiments, the complement-mediated disease is selected from the group consisting of PNH, C3G, IgAN, and SLE-TMA. In some embodiments, the anti-C5 antibody moiety comprises a VH-CDR1 comprising the amino acid sequence of SEQ ID NO: 86, a VH-CDR2 comprising the amino acid sequence of SEQ ID NO: 57, a VH-CDR3 comprising the amino acid sequence of SEQ ID NO: 49, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96. In some embodiments, the anti-C5 antibody moiety comprises a VH comprising the amino acid sequence of SEQ ID NO: 87, and a VL comprising the amino acid sequence of SEQ ID NO: 88. In some embodiments, the anti-C5 antibody moiety is a full-length antibody comprising i) a heavy chain comprising the amino acid sequence of SEQ ID NO: 120, and ii) a light chain comprising the amino acid sequence of SEQ ID NO: 90. In some embodiments, the FH or functional fragment thereof is fused to the C-terminus of one or both (e.g., both) heavy chains of the anti-C5 full-length antibody. In some embodiments, the heavy chain fused to FH or functional fragment thereof comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating a complement-mediated disease in a human individual (e.g., complement inhibitor-naïve human individual), comprising administering to the human individual an effective amount of a fusion protein (e.g., any of the anti-C5-FH fusion proteins described herein, such as FMEH-IgG4-PLA-FH) comprising (a) an anti-human C5 antibody moiety (e.g., anti-human C5 full-length antibody, such as FMEH-IgG4-PLA) and (b) an FH or fragment thereof (e.g., SCR1-5 domains of FH, such as SEQ ID NO: 85), wherein the fusion protein is administered with an initial phase comprising administering the fusion protein at an initial dose of about 600 mg IV to about 3600 mg IV on Day 1, followed by a maintenance phase comprising administering the fusion protein starting Day 8 at a maintenance dose of: i) about 600 mg IV to about 1200 mg IV weekly; ii) about 720 mg SC to about 1440 mg SC weekly; iii) about 1920 mg SC to about 2880 mg SC biweekly; iv) about 1800 mg SC to about 2400 mg SC biweekly; or v) about 960 mg SC to about 2880 mg SC biweekly. In some embodiments, the complement-mediated disease is selected from the group consisting of PNH, C3G, IgAN, and SLE-TMA. In some embodiments, the initial dose of the fusion protein is administered IV at any of about 600 mg to about 1200 mg, about 600 mg to about 2880 mg, about 720 mg to about 1440 mg, about 720 mg to about 3000 mg, about 1200 mg to about 3600 mg, about 1200 mg to about 2400 mg, about 1200 mg to about 1800 mg, about 2000 mg to about 2800 mg, about 1800 mg to about 3600 mg, or about 2400 mg to about 3600 mg; such as any of about 1200 mg, about 1600 mg, about 1800 mg, about 2000 mg, about 2400 mg, about 2800 mg, about 3000 mg, or about 3600 mg. In some embodiments, the maintenance phase comprises administering the fusion protein IV at a weekly maintenance dose of any of about 600 mg, about 720 mg, about 840 mg, about 960 mg, about 1080 mg, about 1200 mg, or any ranges in between. In some embodiments, the maintenance phase comprises administering the fusion protein SC at a weekly maintenance dose of any of about 720 mg, about 840 mg, about 960 mg, about 1080 mg, about 1200 mg, about 1320 mg, about 1440 mg, or any ranges in between. In some embodiments, the maintenance phase comprises administering the fusion protein SC at a biweekly maintenance dose of any of about 960 mg to about 1920 mg, about 960 mg to about 2880 mg, about 1920 mg to about 2880 mg, about 1800 mg to about 2400 mg, about 1800 mg to about 2880 mg, about 1800 mg to about 1920 mg, about 1920 mg to about 2400 mg, or about 2000 mg to about 2400 mg; such as any of about 1800 mg, about 1920 mg, about 2040 mg, about 2160 mg, about 2280 mg, about 2400 mg, about 2520 mg, about 2640 mg, about 2760 mg, about 2880 mg, or any ranges in between. In some embodiments, the maintenance phase is at least about 12 weeks (e.g., 12, 13, 14, 15, 16, 18, 20, 22, 23, 24, 25, 26, 28, 30, 34, 38, 40, 42, 44, 46, 47, 48, 49, 50 weeks or more, such as 12 weeks) for weekly maintenance dosing, or at least about 13 weeks (e.g., 13, 15, 17, 19, 21, 23, 25, 27, 29, 31, 33, 35, 37, 39, 41, 43, 45, 47, 49, 51 weeks or more, such as 13 weeks) for biweekly maintenance dosing. In some embodiments, the maintenance phase is at least about 24 weeks (e.g., 24 weeks) for weekly maintenance dosing, or at least about 25 weeks (e.g., 25 weeks) for biweekly maintenance dosing. In some embodiments, the maintenance phase is at least about 48 weeks (e.g., 48 weeks) for weekly maintenance dosing, or at least about 49 weeks (e.g., 49 weeks) for biweekly maintenance dosing. In some embodiments, the anti-C5 antibody moiety comprises a VH-CDR1 comprising the amino acid sequence of SEQ ID NO: 86, a VH-CDR2 comprising the amino acid sequence of SEQ ID NO: 57, a VH-CDR3 comprising the amino acid sequence of SEQ ID NO: 49, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96. In some embodiments, the anti-C5 antibody moiety comprises a VH comprising the amino acid sequence of SEQ ID NO: 87, and a VL comprising the amino acid sequence of SEQ ID NO: 88. In some embodiments, the anti-C5 antibody moiety is a full-length antibody comprising i) a heavy chain comprising the amino acid sequence of SEQ ID NO: 120, and ii) a light chain comprising the amino acid sequence of SEQ ID NO: 90. In some embodiments, the FH or functional fragment thereof is fused to the C-terminus of one or both (e.g., both) heavy chains of the anti-C5 full-length antibody. In some embodiments, the heavy chain fused to FH or functional fragment thereof comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, the method comprises administering the anti-C5-FH fusion protein at a high initial loading dose, such as about 600 mg to about 3600 mg (including for example any of 600, 1200, 1440, 1800, 1920, 2400, and 3600 mg, or any ranges in between), in order to mitigate the impact of target-mediated drug disposition (TMDD). Pharmacokinetics of mAbs have revealed that higher antigen mass is associated with higher mAb clearance, which lengthens the time to reach steady-state drug concentration (see, e.g., Ternant et al., Influence of Antigen Mass on the Pharmacokinetics of Therapeutic Antibodies in Humans. Clinical Pharmacokinetics, 2019 February; 58(2)). Use of high loading dose upfront can decrease the contribution of clearance due to the nonlinear TMDD to the total clearance (nonlinear clearance due to the TMDD+linear nonspecific clearance), so that the steady-state drug concentration can be reached sooner.

In some embodiments, the methods described herein further comprises selecting an individual suitable for such treatment. In some embodiments, the methods described herein further comprises excluding an individual not suitable for such treatment. In some embodiments, the human individual to be treated has been previously treated with an C5 inhibitor, e.g., anti-C5 antibody therapy. In some embodiments, the human individual to be treated has not been previously treated with an C5 inhibitor, e.g., anti-C5 antibody therapy. In some embodiments, the methods further comprise determining the subject's hemoglobin level, transfusion status, and/or FACIT Fatigue Scale Score at baseline and post-treatment. See, e.g., Examples 4-6 for exemplary selection/exclusion methods.

In some embodiments, the methods described herein further comprises measuring toxicity or side effects of the treatment methods, including but not limited to, treatment-emergent adverse events (TEAEs), treatment-emergent serious adverse events (TESAEs), and Adverse events of special interest (AESIs), such as based on National Cancer Institute Common Terminology Criteria for Adverse Events (NCI CTCAE). In some embodiments, the treatment methods described herein does not induce an adverse event of Grade ≥3 according to CTCAE v5.0. In some embodiments, the method also comprises measuring one or more of clinical laboratory values, electrocardiograms (ECGs), physical examinations, and vital signs. See, e.g., Examples 4-6 for exemplary methods.

### Methods of Treating PNH

In some embodiments, there is provided a method of treating PNH in a human individual (e.g., complement inhibitor-naïve human individual), comprising administering to the human individual an effective amount of a fusion protein (e.g., any of the anti-C5-FH fusion proteins described herein, such as FMEH-IgG4-PLA-FH) comprising i) an antibody moiety that specifically binds to a human C5 (e.g., anti-human C5 full-length antibody, such as FMEH-IgG4-PLA) and ii) an FH or functional fragment thereof (e.g., SCR1-5 domains of FH, such as SEQ ID NO: 85). In some embodiments, the fusion protein is administered intravenously. In some embodiments, the fusion protein is administered subcutaneously. In some embodiments, the fusion protein is administered at the dose of about 60 to about 3600 mg (including for example any of 60, 180, 360, 600, 720, 960, 1200, 1440, 1800, 1920, 2400, 1920, 2880, 3000, 3200, and 3600 mg). In some embodiments, the fusion protein is administered at a single dose, such as at a dose of about 60 mg to about 1200 mg. In some embodiments, the fusion protein is administered at a single dose of about 60 mg IV, about 180 mg IV, about 180 mg SC, about 360 mg IV, about 600 mg IV, about 720 mg SC, or about 1200 mg IV. In some embodiments, the fusion protein is administered at multiple doses. In some embodiments, the fusion protein is administered weekly or biweekly, such as administered at a dose of about 600 mg to about 2880 mg. In some embodiments, the fusion protein is administered with an initial phase comprising administering the fusion protein at one or more (e.g., one) initial doses (e.g., about 600 mg to about 3600 mg, or about 1200 mg to about 3600 mg per dose, such as IV administration), followed by a maintenance phase comprising administering the fusion protein (e.g., weekly or biweekly) for at least two (e.g., 2, 3, 4, 5, 6, 8, 10, 11, 12, 13, 14, 23, 24, 25, 26, 40, 44, 47, 48, 49, 50, or more) doses (e.g., about 600 mg to about 2880 mg per dose). In some embodiments, the two or more maintenance doses of the fusion protein are administered weekly, such as at about 600 mg to about 2880 mg per dose. In some embodiments, the two or more maintenance doses of the fusion protein are each administered weekly at about 600 mg IV to about 1200 mg IV, about 960 mg SC to about 2880 mg SC, or at about 720 mg SC to about 1440 mg SC. In some embodiments, the two or more maintenance doses of the fusion protein are administered biweekly, such as at about 960 mg to about 2880 mg. In some embodiments, the two or more maintenance doses of the fusion protein are each administered biweekly at about 960 mg SC to about 2880 mg SC, about 1200 mg SC to about 2880 mg SC, about 1400 mg SC to about 2880 mg SC, about 1600 mg SC to about 2880 mg SC, about 1800 mg SC to about 2880 mg SC, about 2000 mg SC to about 2800 mg SC, about 2200 mg SC to about 2600 mg SC, about 2300 mg SC to about 2500 mg SC, about 2320 mg SC to about 2480 mg SC, about 2340 mg SC to about 2460 mg SC, about 2360 mg SC to about 2440 mg SC, about 2380 mg SC to about 2420 mg SC, about 1500 mg SC to about 2300 mg SC, about 1700 mg SC to about 2100 mg SC, about 1800 mg SC to about 2000 mg SC, about 1820 mg SC to about 1980 mg SC, about 1840 mg SC to about 1960 mg SC, about 1860 mg SC to about 1940 mg SC, about 1880 mg SC to about 1940 mg SC, about 1900 mg SC to about 1940 mg SC, about 1920 mg SC to about 2880 mg SC, or at about 1800 mg SC to about 2400 mg SC. In some embodiments, the maintenance phase is at least about 12 weeks. In some embodiments, the initial dosing phase comprises administering to the individual a dose of about 1200 mg to about 3600 mg (e.g., IV) weekly for one or more doses (e.g., one dose only), followed by a maintenance phase comprising administering the fusion protein at about 720 mg to about 2880 mg (e.g., SC) or about 600 mg to about 1200 mg (e.g., IV) weekly or biweekly for at least two doses, e.g., at least about 12 weeks (e.g., 12, 13, 14, 15, 16, 18, 20, 22, 23, 24, 25, 26, 28, 30, 34, 38, 40, 42, 44, 46, 47, 48, 49, 50 weeks or more, such as 12 weeks) for weekly maintenance dosing, or at least about 13 weeks (e.g., 13, 15, 17, 19, 21, 23, 25, 27, 29, 31, 33, 35, 37, 39, 41, 43, 45, 47, 49, 51 weeks or more, such as 13 weeks) for biweekly maintenance dosing. In some embodiments, the fusion protein is further administered with an extension phase comprising administering the fusion protein at one or more extension doses after the maintenance phase. In some embodiments, the maintenance phase is followed by an extension phase of at least about 1 week, 2 weeks, 3 weeks, or 1, 2, 3, 4, 5, 6, 7, 8, 9, 10, 11, 12, 13, 14, 15 months or more, such as 9 months. In some embodiments, the extension dose is the same as the maintenance dose. In some embodiments, the extension dose is lower than the maintenance dose. In some embodiments, the extension dose is higher than the maintenance dose. In some embodiments, the extension dose is about 1920 mg SC. In some embodiments, the extension phase comprises administering the fusion protein at an extension dose of about 1920 mg SC biweekly for about 9 months. In some embodiments, the anti-C5 antibody moiety comprises a VH-CDR1 comprising the amino acid sequence of SEQ ID NO: 86, a VH-CDR2 comprising the amino acid sequence of SEQ ID NO: 57, a VH-CDR3 comprising the amino acid sequence of SEQ ID NO: 49, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96. In some embodiments, the anti-C5 antibody moiety comprises a VH comprising the amino acid sequence of SEQ ID NO: 87, and a VL comprising the amino acid sequence of SEQ ID NO: 88. In some embodiments, the anti-C5 antibody moiety is a full-length antibody comprising i) a heavy chain comprising the amino acid sequence of SEQ ID NO: 120, and ii) a light chain comprising the amino acid sequence of SEQ ID NO: 90. In some embodiments, the FH or functional fragment thereof is fused to the C-terminus of one or both (e.g., both) heavy chains of the anti-C5 full-length antibody. In some embodiments, the heavy chain fused to FH or functional fragment thereof comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. IOn some embodiments, for SC administration, the anti-C5-FH fusion protein is administered without further formulation and administered into the abdominal wall using standard techniques for SC administration.

In some embodiments, there is provided a method of treating PNH in a human individual (e.g., complement inhibitor-naïve human individual), comprising administering to the human individual an effective amount of a fusion protein (e.g., any of the anti-C5-FH fusion proteins described herein, such as FMEH-IgG4-PLA-FH) comprising (a) an anti-human C5 antibody moiety (e.g., anti-human C5 full-length antibody, such as FMEH-IgG4-PLA) and (b) an FH or functional fragment thereof (e.g., SCR1-5 domains of FH, such as SEQ ID NO: 85), wherein the fusion protein is administered with an initial phase comprising administering the fusion protein at an initial dose of about 1200 mg IV to about 3600 mg IV on Day 1, followed by a maintenance phase comprising administering the fusion protein starting Day 8 at a maintenance dose of: i) about 600 mg IV to about 1200 mg IV weekly for at least about 12 weeks; ii) about 720 mg SC to about 1440 mg SC weekly for at least about 12 weeks; iii) about 1920 mg SC to about 2880 mg SC biweekly for at least about 13 weeks; or iv) about 960 mg SC to about 2880 mg SC biweekly for at least about 13 weeks. In some embodiments, the initial phase comprises administering the fusion protein at an initial dose of about 1200 mg IV on Day 1, and wherein the maintenance phase comprises administering the fusion protein starting Day 8 at a maintenance dose of about 720 mg SC or about 600 mg IV weekly for about 12 weeks. In some embodiments, the initial phase comprises administering the fusion protein at an initial dose of about 1200 mg IV to about 2400 mg IV on Day 1, and wherein the maintenance phase comprises administering the fusion protein starting Day 8 at a maintenance dose of about 960 mg SC to about 2880 mg SC biweekly for about 13 weeks. In some embodiments, the initial phase comprises administering the fusion protein at an initial dose of about 2400 mg IV on Day 1, and wherein the maintenance phase comprises administering the fusion protein starting Day 8 at a maintenance dose of about 960 mg SC to about 2880 mg SC biweekly for about 13 weeks. In some embodiments, the initial phase comprises administering the fusion protein at an initial dose of about 1200 mg IV to about 2400 mg IV on Day 1, and wherein the maintenance phase comprises administering the fusion protein starting Day 8 at a maintenance dose of about 1920 mg SC biweekly for about 13 weeks. In some embodiments, the initial phase comprises administering the fusion protein at an initial dose of about 3600 mg IV on Day 1, and wherein the maintenance phase comprises administering the fusion protein starting Day 8 at a maintenance dose of about 1920 mg SC to about 2880 mg SC biweekly for about 13 weeks. In some embodiments, the initial phase comprises administering the fusion protein at an initial dose of about 1200 mg IV on Day 1, and wherein the maintenance phase comprises administering the fusion protein starting Day 8 at a maintenance dose of about 720 mg SC weekly for about 12 weeks. In some embodiments, the initial phase comprises administering the fusion protein at an initial dose of about 2400 mg IV on Day 1, and wherein the maintenance phase comprises administering the fusion protein starting Day 8 at a maintenance dose of about 1920 mg SC biweekly for about 13 weeks. In some embodiments, the initial phase comprises administering the fusion protein at an initial dose of about 3600 mg IV on Day 1, and wherein the maintenance phase comprises administering the fusion protein starting Day 8 at a maintenance dose of about 2880 mg SC biweekly for about 13 weeks. In some embodiments, the fusion protein is further administered with an extension phase comprising administering the fusion protein at one or more extension doses after the maintenance phase. In some embodiments, the maintenance phase is followed by an extension phase of at least about 1 week, 2 weeks, 3 weeks, or 1, 2, 3, 4, 5, 6, 7, 8, 9, 10, 11, 12, 13, 14, 15 months or more, e.g., about 9 months. In some embodiments, the extension dose is the same as the maintenance dose. In some embodiments, the extension dose is lower than the maintenance dose. In some embodiments, the extension dose is higher than the maintenance dose. In some embodiments, the extension phase comprises administering the fusion protein at an extension dose of about 1920 mg SC biweekly for about 9 months. In some embodiments, the anti-C5 antibody moiety comprises a VH-CDR1 comprising the amino acid sequence of SEQ ID NO: 86, a VH-CDR2 comprising the amino acid sequence of SEQ ID NO: 57, a VH-CDR3 comprising the amino acid sequence of SEQ ID NO: 49, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96. In some embodiments, the anti-C5 antibody moiety comprises a VH comprising the amino acid sequence of SEQ ID NO: 87, and a VL comprising the amino acid sequence of SEQ ID NO: 88. In some embodiments, the anti-C5 antibody moiety is a full-length antibody comprising i) a heavy chain comprising the amino acid sequence of SEQ ID NO: 120, and ii) a light chain comprising the amino acid sequence of SEQ ID NO: 90. In some embodiments, the FH or functional fragment thereof is fused to the C-terminus of one or both (e.g., both) heavy chains of the anti-C5 full-length antibody. In some embodiments, the heavy chain fused to FH or functional fragment thereof comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating PNH in a human individual (e.g., complement inhibitor-naïve human individual), comprising administering to the human individual an effective amount of a fusion protein comprising i) an anti-human C5 full-length antibody, ii) a first FH functional fragment, and iii) a second FH functional fragment; wherein the fusion protein is administered with an initial phase comprising administering the fusion protein at an initial dose of about 1200 mg IV on Day 1, followed by a maintenance phase comprising administering the fusion protein starting Day 8 at a maintenance dose of about 720 mg SC or about 600 mg IV weekly for about 12 weeks; wherein the first FH functional fragment is fused to the C-terminus of a first heavy chain of the anti-human C5 full-length antibody, and the second FH functional fragment is fused to the C-terminus of a second heavy chain of the anti-human C5 full-length antibody; wherein each heavy chain of the anti-human C5 full-length antibody comprises the amino acid sequence of SEQ ID NO: 120, and each light chain of the anti-human C5 full-length antibody comprises the amino acid sequence of SEQ ID NO: 90; and wherein each heavy chain fused to FH functional fragment comprises the amino acid sequence of SEQ ID NO: 89. In some embodiments, the fusion protein is further administered with an extension phase comprising administering the fusion protein at one or more extension doses after the maintenance phase. In some embodiments, the extension phase comprises administering the fusion protein at an extension dose of about 1920 mg SC biweekly for about 9 months. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating PNH in a human individual (e.g., complement inhibitor-naïve human individual), comprising administering to the human individual an effective amount of a fusion protein comprising i) an anti-human C5 full-length antibody, ii) a first FH functional fragment, and iii) a second FH functional fragment; wherein the fusion protein is administered with an initial phase comprising administering the fusion protein at an initial dose of about 1200 mg IV on Day 1, followed by a maintenance phase comprising administering the fusion protein starting Day 8 at a maintenance dose of about 720 mg SC weekly for about 12 weeks; wherein the first FH functional fragment is fused to the C-terminus of a first heavy chain of the anti-human C5 full-length antibody, and the second FH functional fragment is fused to the C-terminus of a second heavy chain of the anti-human C5 full-length antibody; wherein each heavy chain of the anti-human C5 full-length antibody comprises the amino acid sequence of SEQ ID NO: 120, and each light chain of the anti-human C5 full-length antibody comprises the amino acid sequence of SEQ ID NO: 90; and wherein each heavy chain fused to FH functional fragment comprises the amino acid sequence of SEQ ID NO: 89. In some embodiments, the fusion protein is further administered with an extension phase comprising administering the fusion protein at one or more extension doses after the maintenance phase. In some embodiments, the extension phase comprises administering the fusion protein at an extension dose of about 1920 mg SC biweekly for about 9 months. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating PNH in a human individual (e.g., complement inhibitor-naïve human individual), comprising administering to the human individual an effective amount of a fusion protein comprising i) an anti-human C5 full-length antibody, ii) a first FH functional fragment, and iii) a second FH functional fragment; wherein the fusion protein is administered with an initial phase comprising administering the fusion protein at an initial dose of about 3600 mg IV on Day 1, followed by a maintenance phase comprising administering the fusion protein starting Day 8 at a maintenance dose of about 2880 mg SC biweekly for about 13 weeks; wherein the first FH functional fragment is fused to the C-terminus of a first heavy chain of the anti-human C5 full-length antibody, and the second FH functional fragment is fused to the C-terminus of a second heavy chain of the anti-human C5 full-length antibody; wherein each heavy chain of the anti-human C5 full-length antibody comprises the amino acid sequence of SEQ ID NO: 120, and each light chain of the anti-human C5 full-length antibody comprises the amino acid sequence of SEQ ID NO: 90; and wherein each heavy chain fused to FH functional fragment comprises the amino acid sequence of SEQ ID NO: 89. In some embodiments, the fusion protein is further administered with an extension phase comprising administering the fusion protein at one or more extension doses after the maintenance phase. In some embodiments, the extension phase comprises administering the fusion protein at an extension dose of about 1920 mg SC biweekly for about 9 months. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating PNH in a human individual (e.g., complement inhibitor-naïve human individual), comprising administering to the human individual an effective amount of a fusion protein comprising i) an anti-human C5 full-length antibody, ii) a first FH functional fragment, and iii) a second FH functional fragment; wherein the fusion protein is administered with an initial phase comprising administering the fusion protein at an initial dose of about 1200 mg IV to about 2400 mg IV on Day 1, followed by a maintenance phase comprising administering the fusion protein starting Day 8 at a maintenance dose of about 960 mg SC to about 2880 mg SC biweekly for about 13 weeks; wherein the first FH functional fragment is fused to the C-terminus of a first heavy chain of the anti-human C5 full-length antibody, and the second FH functional fragment is fused to the C-terminus of a second heavy chain of the anti-human C5 full-length antibody; wherein each heavy chain of the anti-human C5 full-length antibody comprises the amino acid sequence of SEQ ID NO: 120, and each light chain of the anti-human C5 full-length antibody comprises the amino acid sequence of SEQ ID NO: 90; and wherein each heavy chain fused to FH functional fragment comprises the amino acid sequence of SEQ ID NO: 89. In some embodiments, the fusion protein is further administered with an extension phase comprising administering the fusion protein at one or more extension doses after the maintenance phase. In some embodiments, the extension phase comprises administering the fusion protein at an extension dose of about 1920 mg SC biweekly for about 9 months. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating PNH in a human individual (e.g., complement inhibitor-naïve human individual), comprising administering to the human individual an effective amount of a fusion protein comprising i) an anti-human C5 full-length antibody, ii) a first FH functional fragment, and iii) a second FH functional fragment; wherein the fusion protein is administered with an initial phase comprising administering the fusion protein at an initial dose of about 2400 mg IV on Day 1, followed by a maintenance phase comprising administering the fusion protein starting Day 8 at a maintenance dose of about 960 mg SC to about 2880 mg SC biweekly for about 13 weeks; wherein the first FH functional fragment is fused to the C-terminus of a first heavy chain of the anti-human C5 full-length antibody, and the second FH functional fragment is fused to the C-terminus of a second heavy chain of the anti-human C5 full-length antibody; wherein each heavy chain of the anti-human C5 full-length antibody comprises the amino acid sequence of SEQ ID NO: 120, and each light chain of the anti-human C5 full-length antibody comprises the amino acid sequence of SEQ ID NO: 90; and wherein each heavy chain fused to FH functional fragment comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, the fusion protein is further administered with an extension phase comprising administering the fusion protein at one or more extension doses after the maintenance phase. In some embodiments, the extension phase comprises administering the fusion protein at an extension dose of about 1920 mg SC biweekly for about 9 months. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating PNH in a human individual (e.g., complement inhibitor-naïve human individual), comprising administering to the human individual an effective amount of a fusion protein comprising i) an anti-human C5 full-length antibody, ii) a first FH functional fragment, and iii) a second FH functional fragment; wherein the fusion protein is administered with an initial phase comprising administering the fusion protein at an initial dose of about 1200 mg IV to about 2400 mg IV on Day 1, followed by a maintenance phase comprising administering the fusion protein starting Day 8 at a maintenance dose of about 1920 mg SC biweekly for about 13 weeks; wherein the first FH functional fragment is fused to the C-terminus of a first heavy chain of the anti-human C5 full-length antibody, and the second FH functional fragment is fused to the C-terminus of a second heavy chain of the anti-human C5 full-length antibody; wherein each heavy chain of the anti-human C5 full-length antibody comprises the amino acid sequence of SEQ ID NO: 120, and each light chain of the anti-human C5 full-length antibody comprises the amino acid sequence of SEQ ID NO: 90; and wherein each heavy chain fused to FH functional fragment comprises the amino acid sequence of SEQ ID NO: 89. In some embodiments, the fusion protein is further administered with an extension phase comprising administering the fusion protein at one or more extension doses after the maintenance phase. In some embodiments, the extension phase comprises administering the fusion protein at an extension dose of about 1920 mg SC biweekly for about 9 months. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating PNH in a human individual (e.g., complement inhibitor-naïve human individual), comprising administering to the human individual an effective amount of a fusion protein comprising i) an anti-human C5 full-length antibody, ii) a first FH functional fragment, and iii) a second FH functional fragment; wherein the fusion protein is administered with an initial phase comprising administering the fusion protein at an initial dose of about 2400 mg IV on Day 1, followed by a maintenance phase comprising administering the fusion protein starting Day 8 at a maintenance dose of about 1920 mg SC biweekly for about 13 weeks; wherein the first FH functional fragment is fused to the C-terminus of a first heavy chain of the anti-human C5 full-length antibody, and the second FH functional fragment is fused to the C-terminus of a second heavy chain of the anti-human C5 full-length antibody; wherein each heavy chain of the anti-human C5 full-length antibody comprises the amino acid sequence of SEQ ID NO: 120, and each light chain of the anti-human C5 full-length antibody comprises the amino acid sequence of SEQ ID NO: 90; and wherein each heavy chain fused to FH functional fragment comprises the amino acid sequence of SEQ ID NO: 89. In some embodiments, the fusion protein is further administered with an extension phase comprising administering the fusion protein at one or more extension doses after the maintenance phase. In some embodiments, the extension phase comprises administering the fusion protein at an extension dose of about 1920 mg SC biweekly for about 9 months. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating PNH in a human individual (e.g., complement inhibitor-naïve human individual), comprising administering to the human individual an effective amount of a fusion protein comprising i) an anti-human C5 full-length antibody, ii) a first FH functional fragment, and iii) a second FH functional fragment; wherein the fusion protein is administered with an initial phase comprising administering the fusion protein at an initial dose of about 3600 mg IV on Day 1, followed by a maintenance phase comprising administering the fusion protein starting Day 8 at a maintenance dose of about 1920 mg SC to about 2880 mg SC biweekly for about 13 weeks; wherein the first FH functional fragment is fused to the C-terminus of a first heavy chain of the anti-human C5 full-length antibody, and the second FH functional fragment is fused to the C-terminus of a second heavy chain of the anti-human C5 full-length antibody; wherein each heavy chain of the anti-human C5 full-length antibody comprises the amino acid sequence of SEQ ID NO: 120, and each light chain of the anti-human C5 full-length antibody comprises the amino acid sequence of SEQ ID NO: 90; and wherein each heavy chain fused to FH functional fragment comprises the amino acid sequence of SEQ ID NO: 89. In some embodiments, the fusion protein is further administered with an extension phase comprising administering the fusion protein at one or more extension doses after the maintenance phase. In some embodiments, the extension phase comprises administering the fusion protein at an extension dose of about 1920 mg SC biweekly for about 9 months. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

### PNH

Complement-mediated hemolytic anemia contributes to numerous disorders of the red blood cells, such as paroxysmal nocturnal hemoglobinuria (PNH). PNH is a hematological disorder characterized by the clonal expansion of one or a few hematopoietic stem cells which are incapable of glycosylphosphatidylinositol (GPI)-anchor biosynthesis, due to an acquired somatic mutation in the phosphatidylinositol glycan class A (PIG-A) gene. Affected progeny cells are deficient in all GPI-anchored surface proteins, including complement regulators CD55 and CD59. Thus, PNH red blood cells (RBCs) are vulnerable to activated complement, and particularly to the membrane attack complex (MAC), resulting in chronic intravascular hemolysis with recurrent exacerbations.

PNH is a condition in which uncontrolled complement activity leads to systemic complications, principally through intravascular hemolysis and platelet activation (see Socie G, et al., French Society of Haematology. Lancet. 1996; 348(9027):573-577 and Brodsky, R., Blood. 2014; 124(18):2804-2811). Persistent intravascular hemolysis may be triggered by various stressors, such as infection or physical exertion, and this leads to smooth muscle contraction (free hemoglobin), chronic anemia, and an increased risk of severe thromboembolism. Thromboembolism is the most common cause of mortality in patients with PNH, and pulmonary hypertension and end-organ damage of vital organs, such as the liver, kidneys, brain, and intestines, are sequelae of such events (Hillmen, P., et al, Am. J. Hematol. 2010; 85(8):553-559). Due to these adverse pathologic processes, patients with PNH have a decreased quality of life (QoL), which may include debilitating fatigue, chronic pain, poor physical function, shortness of breath, abdominal pain, erectile dysfunction, a need for anti-coagulation, blood transfusions and in some instances, need for dialysis (Weitz, I C., et al., Thromb Res. 2012; 130(3):361-368). Patients with PNH are at risk of substantial morbidity and mortality.

PNH patients can exhibit at least one of the following characteristics, which characteristics may be symptoms of residual anemia and/or complement-mediated extravascular hemolysis (EVH) and/or incomplete control of intravascular hemolysis: a) exhibits signs or symptoms continued loss of RBCs by ongoing or intermittent intravascular hemolysis and/or extravascular hemolysis; b) has RBCs opsonized by fragments of C3; c) requires periodic blood transfusions; d) has low normal or below normal levels of hemoglobin; e) has low normal or below normal levels of platelets; f) has high normal or above normal reticulocytes; g) has high normal or above normal bilirubin; or h) has iron overload or is at risk of iron overload.

The above characteristics can also be used to monitor the PNH patients' progress in response to treatment in accordance with the present invention, and to modify the dosage regime if deemed clinically appropriate. In certain embodiments, the subject having PNH has previously been treated with a terminal complement inhibitor, but persists in exhibiting at least one of the above characteristics.

In some embodiments, the method of treating PNH described herein can prevent (e.g., preventing at least about any of 10%, 20%, 30%, 40%, 50%, 60%, 70%, 80%, 90%, or 100% chance), delay (e.g., delaying at least about any of 1, 2, 3, 6, 12, 18, 24, 30, 36, 42, 48, 52 months or longer), or reduce (e.g., reducing at least about any of 10%, 20%, 30%, 40%, 50%, 60%, 70%, 80%, 90%, or 100%) the severity of one or more of PNH signs/symptoms/characteristics discussed above, including but not limited to, EVH, fatigue, abdominal pain, dyspnea, anemia, dysphagia, chest pain, pallor, jaundice, cytopenia, and erectile dysfunction. In some embodiments, the method of treating PNH described herein results in a reduction (e.g., reducing at least about any of 10%, 20%, 30%, 40%, 50%, 60%, 70%, 80%, 90%, or 100%) in one or more of the following in the subject: (a) persistent EVH; (b) anemia; (c) transfusion dependence; (d) intravascular hemolysis; (e) uncontrolled C3 activation and opsonization; and (f) the occurrence of “breakthrough” hemolytic crises observed in patients treated with terminal complement inhibitors. In some embodiments, the method of treating PNH described herein results in an improvement (e.g., improving at least about any of 10%, 20%, 30%, 40%, 50%, 60%, 70%, 80%, 90%, or 100%) in one or more of the following in the subject: i) FACIT-Fatigue Scale Score; ii) serum LDH and hemoglobin (HgB) levels; iii) quality of life; iv) absolute reticulocyte count; v) bilirubin levels, and vi) haptoglobin levels. In some embodiments, the method of treating PNH described herein reduces (e.g., reducing at least about any of 1%, 2%, 3%, 4%, 5%, 6%, 7%, 8%, 9%, 10%, 20%, 30%, 40%, 50%, 60%, 70%, 80%, 90%, or 100%) in one or more of the following: (a) C3b deposition and (b) plasma free C5 levels. In some embodiments, the method of treating PNH described herein reduces (e.g., reducing at least about any of 10%, 20%, 30%, 40%, 50%, 60%, 70%, 80%, 90%, or 100%) lactate dehydrogenase (LDH) levels compared to baseline. In some embodiments, the method of treating PNH described herein reduces LDH levels to below 0.5 times, 1.0 times, or 1.5 times the upper limit of normal (ULN). In some embodiments, the method of treating PNH described herein increases (e.g., increasing at least about any of 1%, 2%, 3%, 4%, 5%, 6%, 7%, 8%, 9%, 10%, 20%, 30%, 40%, 50%, 60%, 70%, 80%, 90%, or 100%) HgB levels compared to baseline. In some embodiments, the method of treating PNH described herein achieves an Hgb increase from baseline by at least about any of 2 g/dL, 3 g/dL, 4 g/dL, 5 g/dL, 6 g/dL, 7 g/dL, 8 g/dL, 9 g/dL, 10 g/dL, 11 g/dL, or 12 g/dL. In some embodiments, the method of treating PNH described herein increases (e.g., increasing at least about any of 10%, 20%, 30%, 40%, 50%, 60%, 70%, 80%, 90%, or 100%) the proportion of PNH red blood cells which are able to survive complement attack. In some embodiments, the human individual to be treated has extravascular hemolysis (EVH). Thus in some embodiments, the disclosure also relates to method of treating clinically-evident EVH in a human individual suffering from PNH.

In some embodiments, the efficacy of the method of treating PNH described herein can be assessed by one or more of: i) increase (e.g., ≥2 g/dL increase) in hemoglobin level from baseline (e.g., in the absence of transfusion); ii) decrease (e.g., decreasing at least about any of 10%, 20%, 30%, 40%, 50%, 60%, 70%, 80%, 90%, or 100%) from baseline in serum lactate dehydrogenase (LDH) levels; iii) proportion of subjects with breakthrough hemolysis defined as at least 1 new or worsening symptom or sign of intravascular hemolysis (fatigue, hemoglobinuria, abdominal pain, shortness of breath [dyspnea], anemia [hemoglobin <10 g/dL], MAVE including thrombosis, dysphagia, or erectile dysfunction) in the presence of elevated LDH ≥2×the upper limit of normal (ULN), after prior LDH reduction to <1.5×ULN on therapy; iv) change in the proportion of subjects with hemoglobin ≥12 g/dL; and v) increase (e.g., increasing at least about any of 10%, 20%, 30%, 40%, 50%, 60%, 70%, 80%, 90%, or 100%) in quality of life assessed by the Functional Assessment of Chronic Illness Therapy (FACIT)-Fatigue Score and EQ-5D-3L. The pharmacodynamics and biomarker changes can also be measured to reflect treatment efficacy, including but are not limited to: 1) decrease (e.g., decreasing at least about any of 1%, 2%, 3%, 4%, 5%, 6%, 7%, 8%, 9%, 10%, 20%, 30%, 40%, 50%, 60%, 70%, 80%, 90%, or 100%) from baseline in C3b activity assay; 2) decrease (e.g., decreasing at least about any of 1%, 2%, 3%, 4%, 5%, 6%, 7%, 8%, 9%, 10%, 20%, 30%, 40%, 50%, 60%, 70%, 80%, 90%, or 100%) from baseline in total and free serum C5 levels; 3) change from baseline in rabbit RBC assay; 4) change from baseline in Factor H serum level; 5) change from baseline in d-dimer; 6) increase (e.g., increasing at least about any of 1%, 2%, 3%, 4%, 5%, 6%, 7%, 8%, 9%, 10%, 20%, 30%, 40%, 50%, 60%, 70%, 80%, 90%, or 100%) from baseline in free hemoglobin; 7) increase (e.g., increasing at least about any of 10%, 20%, 30%, 40%, 50%, 60%, 70%, 80%, 90%, or 100%) from baseline in serum total and direct bilirubin; 8) increase (e.g., increasing at least about any of 10%, 20%, 30%, 40%, 50%, 60%, 70%, 80%, 90%, or 100%) from baseline in serum haptoglobin levels; and 9) increase (e.g., increasing at least about any of 10%, 20%, 30%, 40%, 50%, 60%, 70%, 80%, 90%, or 100%) from baseline in reticulocyte count.

In some embodiments, the method of treating PNH described herein further comprises selecting a human individual suitable for such treatment. In some embodiments, the human individual to be treated are complement inhibitor-naïve subjects with PNH. In some embodiments, the human individual to be treated are complement inhibitor-naïve subjects with PNH on LDH, hemoglobin and transfusion dependence. In some embodiments, the human individual further receives antibiotic prophylaxis during the treatment. In some embodiments, the human individual is at least about 18 years old. In some embodiments, the human individual meets one or more of the criteria: 1) diagnosis of PNH confirmed by flow cytometry evaluation of white blood cells and red blood cells, e.g., with granulocyte or monocyte clone size of ≥10% within 6 months of screening; 2) presence of 1 or more PNH-related signs or symptoms, e.g., within 3 months of screening; 3) LDH ≥2.0×ULN at screening; 4) hemoglobin ≤10.0 g/dL at screening; 5) practice effective contraception during treatment; 6) negative pregnancy test for females during treatment; 7) BMI of <35 kg/m²; 8) prior vaccination against Neisseria meningitidis at screening (subjects not providing evidence of prior vaccination should receive vaccination following initiation of administration of the anti-C5-FH fusion protein, but are required to receive treatment with appropriate antibiotic prophylaxis until 2 weeks after vaccination); and 9) vaccination against Streptococcus pneumoniae and Hemophilus influenzae (if administration of the anti-C5-FH fusion protein is initiated within 2 weeks of vaccination, appropriate antibiotics should be given for prophylaxis).

In some embodiments, the method of treating PNH described herein further comprises excluding a human individual not suitable for such treatment. In some embodiments, a human individual not suitable for the method of treatment described herein meets one or more of the criteria: 1) any clinically significant poorly controlled underlying illness other than PNH; 2) treatment of any infection with IV (within 30 days of Screening) or oral (within 14 days of Screening) antibiotics, antivirals, or antifungals; 3) history of meningococcal infection; 4) history of untreated tuberculosis; 5) history of splenectomy; 6) positive serology for Hepatitis C Virus (HCV) ribonucleic acid (RNA) or human immunodeficiency virus (HIV) at Screening; 7) history of bone marrow or stem cell transplantation; 8) absolute neutrophil count (ANC) <500 cells/μL; 9) reticulocyte count <100×10³ cells/μL; 10) platelet count <30,000 cells/μL; 11) history of systemic autoimmune disease; 12) estimated glomerular filtration rate (eGFR) <30 mL/min/1.73 m² calculated by Chronic Kidney Disease Epidemiology Collaboration (CKD-EPI) equation; 13) known allergy to penicillin antibiotics; 14) history of drug or alcohol abuse within 1 year of Screening; 15) received any type of live attenuated vaccine <1 month prior to Screening or is planning to receive any such live attenuated vaccine during treatment; 16) use of rituximab within the last 3 months or any complement inhibitors prior to screening; 17) use of steroids (topical use is allowed), EPO, iron supplements, folic acid, vitamin B12, androgen, HIF-PHIs within 4 weeks and immunosuppressive agents within 3 months prior to screening (if doses of above drugs could be maintained stable at this period, it is acceptable); 18) history of malignancy, except adequately treated basal cell carcinoma or in situ carcinoma of the uterine cervix; 19) bone marrow failure and/or candidate for bone marrow transplantation; 20) any female who is pregnant or breastfeeding, or any female who is planning to become pregnant during treatment; 21) a QT duration corrected for heart rate by Fridericia's formula (QTcF) >450 millisecond (msec) for males and >470 msec for females based on either single or averaged QTcF values of triplicate ECGs obtained over a 3-minute interval; and 22) previously treated with a complement inhibitor.

### Methods of Treating SLE-TMA

In some embodiments, there is provided a method of treating SLE-TMA in a human individual (e.g., complement inhibitor-naïve human individual), comprising administering to the human individual an effective amount of a fusion protein (e.g., any of the anti-C5-FH fusion proteins described herein, such as FMEH-IgG4-PLA-FH) comprising i) an antibody moiety that specifically binds to a human C5 (e.g., anti-human C5 full-length antibody, such as FMEH-IgG4-PLA) and ii) an FH or functional fragment thereof (e.g., SCR1-5 domains of FH, such as SEQ ID NO: 85). In some embodiments, the fusion protein is administered intravenously. In some embodiments, the fusion protein is administered subcutaneously. In some embodiments, the fusion protein is administered at the dose of about 60 to about 3600 mg (including for example any of 60, 180, 360, 600, 720, 960, 1200, 1440, 1800, 1920, 2400, 1920, 2880, 3000, 3200, and 3600 mg). In some embodiments, the fusion protein is administered at a single dose, such as at a dose of about 60 mg to about 1200 mg. In some embodiments, the fusion protein is administered at a single dose of about 60 mg IV, about 180 mg IV, about 180 mg SC, about 360 mg IV, about 600 mg IV, about 720 mg SC, or about 1200 mg IV. In some embodiments, the fusion protein is administered at multiple doses. In some embodiments, the fusion protein is administered weekly or biweekly, such as administered at a dose of about 600 mg to about 2880 mg. In some embodiments, the fusion protein is administered with an initial phase comprising administering the fusion protein at one or more (e.g., one) initial doses (e.g., about 600 mg to about 3600 mg, or about 1200 mg to about 3600 mg per dose, such as IV administration), followed by a maintenance phase comprising administering the fusion protein (e.g., weekly or biweekly) for at least two (e.g., 2, 3, 4, 5, 6, 8, 10, 11, 12, 13, 14, 23, 24, 25, 26, 40, 44, 47, 48, 49, 50, or more) doses (e.g., about 600 mg to about 2880 mg per dose). In some embodiments, the two or more maintenance doses of the fusion protein are administered weekly, such as at about 600 mg to about 1440 mg per dose, or about 600 mg to about 1200 mg per dose. In some embodiments, the two or more maintenance doses of the fusion protein are each administered weekly at about 600 mg IV to about 1200 mg IV, or at about 720 mg SC to about 1440 mg SC. In some embodiments, the two or more maintenance doses of the fusion protein are administered biweekly, such as at about 1800 mg to about 2880 mg. In some embodiments, the two or more maintenance doses of the fusion protein are each administered biweekly at about 1920 mg SC to about 2880 mg SC, or at about 1800 mg SC to about 2400 mg SC. In some embodiments, the maintenance phase is at least about 24 weeks. In some embodiments, the initial dosing phase comprises administering to the individual a dose of about 1200 mg to about 3600 mg (e.g., about 1200 mg to about 2400 mg, such as IV) weekly for one or more doses (e.g., one dose only), followed by a maintenance phase comprising administering the fusion protein at about 720 mg to about 2880 mg (e.g., SC) weekly or biweekly for at least two doses, e.g., at least about 24 weeks (e.g., 24, 25, 26, 28, 30, 34, 38, 40, 42, 44, 46, 47, 48, 49, 50 weeks or more, such as 24 weeks) for weekly maintenance dosing, or at least about 25 weeks (e.g., 25, 27, 29, 31, 33, 35, 37, 39, 41, 43, 45, 47, 49, 51 weeks or more, such as 25 weeks) for biweekly maintenance dosing. In some embodiments, the maintenance phase comprises administering the fusion protein (e.g., weekly or biweekly) at a first maintenance dose for a first maintenance phase period, followed by administering the fusion protein (e.g., weekly or biweekly) at a second maintenance dose for a second maintenance phase period. In some embodiments, the first maintenance dose and/or the second maintenance dose is about 600 mg to about 2880 mg, such as any of about 600 mg to about 1200 mg, about 720 mg to about 1440 mg, about 1800 mg to about 2880 mg, about 1920 mg to about 2880 mg, about 1800 mg to about 2400 mg, about 600 mg to about 2400 mg, or about 720 mg to about 2400 mg. In some embodiments, the first maintenance dose and/or the second maintenance dose is about any of 600 mg, 720 mg, 960 mg, 1200 mg, 1440 mg, 1800 mg, 1920 mg, 2400 mg, or 2880 mg. In some embodiments, the initial dose, the first maintenance dose, and/or the second maintenance dose of the fusion protein is administered IV or SC. In some embodiments, the first maintenance dose and/or the second maintenance dose of the fusion protein is administered at about 600 mg IV to about 1200 mg IV, about 720 mg SC to about 1440 mg SC, about 1920 mg SC to about 2880 mg SC, or at about 1800 mg SC to about 2400 mg SC. In some embodiments, the second maintenance dose is higher than the first maintenance dose, such as at least about any of 1.5, 2, 3, 4, 5, 10-fold or more higher. In some embodiments, the second maintenance phase period is longer than the first maintenance phase period, such as at least about any of 1, 2, 3, 4, 5, 6, 7, 8, 9, 10, 11, 12, 13, 14, 15, 16, 17, 18, 19, 20, 21, or 22 weeks or more longer. In some embodiments, the first maintenance phase period is at least about 1 week, and the second maintenance phase period is no more than about 23 or about 24 weeks. In some embodiments, the first maintenance dose of the fusion protein is administered weekly at about 600 mg IV to about 1200 mg IV for a first maintenance phase period, followed by a second maintenance dose of the fusion protein administered weekly at about 720 mg SC to about 1440 mg SC for a second maintenance phase period. In some embodiments, the first maintenance dose of the fusion protein is administered weekly at about 600 mg IV to about 1200 mg IV weekly for a first maintenance phase period, followed by a second maintenance dose of the fusion protein administered biweekly at about 1920 mg SC to about 2880 mg SC for a second maintenance phase period. In some embodiments, the first maintenance phase period is at least about 1 week (e.g., 1, 2, 3, 4, 5, 6, 7, 8, 9, 10, 11, 12, 13, 14, 15, 16, 17, 18, 19, 20, 21, 22, 23 weeks or more, such as 1 week), and the second maintenance phase period is no more than about 24 weeks (e.g., 23, 22, 21, 20, 19, 18, 17, 16, 15, 14, 13, 12, 11, 10, 9, 8, 7, 6, 5, 4, 3, 2, or 1 week, such as 23 or 24 weeks). In some embodiments, the maintenance phase is at least about 24 or about 25 weeks (including the first maintenance phase period and the second maintenance phase period). In some embodiments, the anti-C5 antibody moiety comprises a VH-CDR1 comprising the amino acid sequence of SEQ ID NO: 86, a VH-CDR2 comprising the amino acid sequence of SEQ ID NO: 57, a VH-CDR3 comprising the amino acid sequence of SEQ ID NO: 49, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96. In some embodiments, the anti-C5 antibody moiety comprises a VH comprising the amino acid sequence of SEQ ID NO: 87, and a VL comprising the amino acid sequence of SEQ ID NO: 88. In some embodiments, the anti-C5 antibody moiety is a full-length antibody comprising i) a heavy chain comprising the amino acid sequence of SEQ ID NO: 120, and ii) a light chain comprising the amino acid sequence of SEQ ID NO: 90. In some embodiments, the FH or functional fragment thereof is fused to the C-terminus of one or both (e.g., both) heavy chains of the anti-C5 full-length antibody. In some embodiments, the heavy chain fused to FH or functional fragment thereof comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating SLE-TMA in a human individual (e.g., complement inhibitor-naïve human individual), comprising administering to the human individual an effective amount of a fusion protein (e.g., any of the anti-C5-FH fusion proteins described herein, such as FMEH-IgG4-PLA-FH) comprising i) an anti-human C5 antibody moiety (e.g., anti-human C5 full-length antibody, such as FMEH-IgG4-PLA) and ii) an FH or functional fragment thereof (e.g., SCR1-5 domains of FH, such as SEQ ID NO: 85), wherein the fusion protein is administered with an initial phase comprising administering the fusion protein at an initial dose (e.g., about 600 mg to about 1200 mg, about 600 mg to about 2880 mg, about 720 mg to about 1440 mg, about 720 mg to about 3000 mg, about 1200 mg IV to about 3600 mg IV, about 1200 mg IV to about 2400 mg IV, or about 2400 mg IV to about 3600 mg IV) on Day 1, followed by a maintenance phase comprising administering the fusion protein starting Day 8 for two or more (e.g., 24, 25, 26, 27, 28, 29, 30 weeks or more) maintenance doses; and wherein the maintenance phase comprises administering the fusion protein (e.g., weekly or biweekly) at a first maintenance dose for a first maintenance phase period (e.g., 1, 2, 3, 4, 5, 6, 7, 8, 9, 10, 11, 12, 18, 23, 24 weeks or more), followed by administering the fusion protein (e.g., weekly or biweekly) at a second maintenance dose for a second maintenance phase period (e.g., 1, 2, 3, 4, 5, 6, 7, 8, 9, 10, 11, 12, 18, 24, 25 weeks or more). In some embodiments, the initial dose is about any of 600 mg, 720 mg, 1200 mg, 1440 mg, 1600 mg, 1800 mg, 1920 mg, 2400 mg, 2880 mg, 3000 mg, 3240 mg, or 3600 mg. In some embodiments, the first maintenance dose and/or the second maintenance dose is about 600 mg to about 2880 mg, such as any of about 600 mg to about 1200 mg, about 720 mg to about 1440 mg, about 1800 mg to about 2880 mg, about 1920 mg to about 2880 mg, about 1800 mg to about 2400 mg, about 600 mg to about 2400 mg, or about 720 mg to about 2400 mg. In some embodiments, the first maintenance dose and/or the second maintenance dose is about any of 600 mg, 720 mg, 960 mg, 1200 mg, 1440 mg, 1800 mg, 1920 mg, 2400 mg, or 2880 mg. In some embodiments, the initial dose, the first maintenance dose, and/or the second maintenance dose of the fusion protein is administered IV or SC. In some embodiments, the first maintenance dose and/or the second maintenance dose of the fusion protein is administered at about 600 mg IV to about 1200 mg IV, about 720 mg SC to about 1440 mg SC, about 1920 mg SC to about 2880 mg SC, or at about 1800 mg SC to about 2400 mg SC. In some embodiments, the second maintenance dose is higher than the first maintenance dose. In some embodiments, the second maintenance phase period is longer than the first maintenance phase period. In some embodiments, the first maintenance dose of the fusion protein is administered at about 600 mg IV to about 1200 mg IV weekly, and the second maintenance dose of the fusion protein is administered at about 720 mg SC to about 1440 mg SC weekly, or about 1920 mg SC to about 2880 mg SC biweekly. In some embodiments, the maintenance phase (including the first maintenance phase period and the second maintenance phase period) is at least about 24 weeks or about 25 weeks. In some embodiments, the anti-C5 antibody moiety comprises a VH-CDR1 comprising the amino acid sequence of SEQ ID NO: 86, a VH-CDR2 comprising the amino acid sequence of SEQ ID NO: 57, a VH-CDR3 comprising the amino acid sequence of SEQ ID NO: 49, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96. In some embodiments, the anti-C5 antibody moiety comprises a VH comprising the amino acid sequence of SEQ ID NO: 87, and a VL comprising the amino acid sequence of SEQ ID NO: 88. In some embodiments, the anti-C5 antibody moiety is a full-length antibody comprising i) a heavy chain comprising the amino acid sequence of SEQ ID NO: 120, and ii) a light chain comprising the amino acid sequence of SEQ ID NO: 90. In some embodiments, the FH or functional fragment thereof is fused to the C-terminus of one or both (e.g., both) heavy chains of the anti-C5 full-length antibody. In some embodiments, the heavy chain fused to FH or functional fragment thereof comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating SLE-TMA in a human individual (e.g., complement inhibitor-naïve human individual), comprising administering to the human individual an effective amount of a fusion protein (e.g., any of the anti-C5-FH fusion proteins described herein, such as FMEH-IgG4-PLA-FH) comprising (a) an anti-human C5 antibody moiety (e.g., anti-human C5 full-length antibody, such as FMEH-IgG4-PLA) and (b) an FH or functional fragment thereof (e.g., SCR1-5 domains of FH, such as SEQ ID NO: 85), wherein the fusion protein is administered with an initial phase comprising administering the fusion protein at an initial dose of about 1200 mg IV to about 3600 mg IV (e.g., about 1200 mg IV to about 2400 mg IV) on Day 1, followed by a maintenance phase comprising: i) administering the fusion protein starting Day 8 at a maintenance dose of about 720 mg SC to about 1440 mg SC weekly for at least about 24 weeks; ii) administering the fusion protein starting Day 8 at a maintenance dose of about 1920 mg SC to about 2880 mg SC biweekly for at least about 25 weeks; iii) administering the fusion protein starting Day 8 at a first maintenance dose of about 600 mg IV to about 1200 mg IV weekly for a first maintenance phase period, followed by administering the fusion protein at a second maintenance dose of about 720 mg SC to about 1440 mg SC weekly for a second maintenance phase period, and wherein the maintenance phase is at least about 24 weeks; or iv) administering the fusion protein starting Day 8 at a first maintenance dose of about 600 mg IV to about 1200 mg IV weekly for a first maintenance phase period, followed by administering the fusion protein at a second maintenance dose of about 1920 mg SC to about 2880 mg SC biweekly for a second maintenance phase period, and wherein the maintenance phase is at least about 25 weeks. In some embodiments, the initial phase comprises administering the fusion protein at an initial dose of about 1200 mg IV on Day 1, and wherein the maintenance phase comprises administering the fusion protein starting Day 8 at a maintenance dose of about 960 mg SC weekly for about 24 weeks. In some embodiments, the anti-C5 antibody moiety comprises a VH-CDR1 comprising the amino acid sequence of SEQ ID NO: 86, a VH-CDR2 comprising the amino acid sequence of SEQ ID NO: 57, a VH-CDR3 comprising the amino acid sequence of SEQ ID NO: 49, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96. In some embodiments, the anti-C5 antibody moiety comprises a VH comprising the amino acid sequence of SEQ ID NO: 87, and a VL comprising the amino acid sequence of SEQ ID NO: 88. In some embodiments, the anti-C5 antibody moiety is a full-length antibody comprising i) a heavy chain comprising the amino acid sequence of SEQ ID NO: 120, and ii) a light chain comprising the amino acid sequence of SEQ ID NO: 90. In some embodiments, the FH or functional fragment thereof is fused to the C-terminus of one or both (e.g., both) heavy chains of the anti-C5 full-length antibody. In some embodiments, the heavy chain fused to FH or functional fragment thereof comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating SLE-TMA in a human individual (e.g., complement inhibitor-naïve human individual), comprising administering to the human individual an effective amount of a fusion protein comprising i) an anti-human C5 full-length antibody, ii) a first FH functional fragment, and iii) a second FH functional fragment; wherein the fusion protein is administered with an initial phase comprising administering the fusion protein at an initial dose of about 1200 mg IV to about 3600 mg IV on Day 1, followed by a maintenance phase comprising administering the fusion protein starting Day 8 at a maintenance dose of about 720 mg SC to about 1440 mg SC weekly for at least about 24 weeks; wherein the first FH functional fragment is fused to the C-terminus of a first heavy chain of the anti-human C5 full-length antibody, and the second FH functional fragment is fused to the C-terminus of a second heavy chain of the anti-human C5 full-length antibody; wherein each heavy chain of the anti-human C5 full-length antibody comprises the amino acid sequence of SEQ ID NO: 120, and each light chain of the anti-human C5 full-length antibody comprises the amino acid sequence of SEQ ID NO: 90; and wherein each heavy chain fused to FH functional fragment comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating SLE-TMA in a human individual (e.g., complement inhibitor-naïve human individual), comprising administering to the human individual an effective amount of a fusion protein comprising i) an anti-human C5 full-length antibody, ii) a first FH functional fragment, and iii) a second FH functional fragment; wherein the fusion protein is administered with an initial phase comprising administering the fusion protein at an initial dose of about 1200 mg IV to about 3600 mg IV on Day 1, followed by a maintenance phase comprising administering the fusion protein starting Day 8 at a maintenance dose of about 1920 mg SC to about 2880 mg SC biweekly for at least about 25 weeks; wherein the first FH functional fragment is fused to the C-terminus of a first heavy chain of the anti-human C5 full-length antibody, and the second FH functional fragment is fused to the C-terminus of a second heavy chain of the anti-human C5 full-length antibody; wherein each heavy chain of the anti-human C5 full-length antibody comprises the amino acid sequence of SEQ ID NO: 120, and each light chain of the anti-human C5 full-length antibody comprises the amino acid sequence of SEQ ID NO: 90; and wherein each heavy chain fused to FH functional fragment comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating SLE-TMA in a human individual (e.g., complement inhibitor-naïve human individual), comprising administering to the human individual an effective amount of a fusion protein comprising i) an anti-human C5 full-length antibody, ii) a first FH functional fragment, and iii) a second FH functional fragment; wherein the fusion protein is administered with an initial phase comprising administering the fusion protein at an initial dose of about 1200 mg IV to about 3600 mg IV on Day 1, followed by a maintenance phase of at least about 24 weeks, wherein the maintenance phase comprises administering the fusion protein starting Day 8 at a first maintenance dose of about 600 mg IV to about 1200 mg IV weekly for a first maintenance phase period, followed by administering the fusion protein at a second maintenance dose of about 720 mg SC to about 1440 mg SC weekly for a second maintenance phase period; wherein the first FH functional fragment is fused to the C-terminus of a first heavy chain of the anti-human C5 full-length antibody, and the second FH functional fragment is fused to the C-terminus of a second heavy chain of the anti-human C5 full-length antibody; wherein each heavy chain of the anti-human C5 full-length antibody comprises the amino acid sequence of SEQ ID NO: 120, and each light chain of the anti-human C5 full-length antibody comprises the amino acid sequence of SEQ ID NO: 90; and wherein each heavy chain fused to FH functional fragment comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration. In some embodiments, the second maintenance dose is higher than the first maintenance dose. In some embodiments, the second maintenance phase period is longer than the first maintenance phase period. In some embodiments, the first maintenance phase period is at least about 1 week, and the second maintenance phase period is no more than about 23 weeks.

In some embodiments, there is provided a method of treating SLE-TMA in a human individual (e.g., complement inhibitor-naïve human individual), comprising administering to the human individual an effective amount of a fusion protein comprising i) an anti-human C5 full-length antibody, ii) a first FH functional fragment, and iii) a second FH functional fragment; wherein the fusion protein is administered with an initial phase comprising administering the fusion protein at an initial dose of about 1200 mg IV to about 3600 mg IV on Day 1, followed by a maintenance phase of at least about 25 weeks, wherein the maintenance phase comprises administering the fusion protein starting Day 8 at a first maintenance dose of about 600 mg IV to about 1200 mg IV weekly for a first maintenance phase period, followed by administering the fusion protein at a second maintenance dose of about 1920 mg SC to about 2880 mg SC biweekly for a second maintenance phase period; wherein the first FH functional fragment is fused to the C-terminus of a first heavy chain of the anti-human C5 full-length antibody, and the second FH functional fragment is fused to the C-terminus of a second heavy chain of the anti-human C5 full-length antibody; wherein each heavy chain of the anti-human C5 full-length antibody comprises the amino acid sequence of SEQ ID NO: 120, and each light chain of the anti-human C5 full-length antibody comprises the amino acid sequence of SEQ ID NO: 90; and wherein each heavy chain fused to FH functional fragment comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration. In some embodiments, the second maintenance dose is higher than the first maintenance dose. In some embodiments, the second maintenance phase period is longer than the first maintenance phase period. In some embodiments, the first maintenance phase period is at least about 1 week, and the second maintenance phase period is no more than about 24 weeks.

In some embodiments, there is provided a method of treating SLE-TMA in a human individual (e.g., complement inhibitor-naïve human individual), comprising administering to the human individual an effective amount of a fusion protein comprising i) an anti-human C5 full-length antibody, ii) a first FH functional fragment, and iii) a second FH functional fragment; wherein the fusion protein is administered with an initial phase comprising administering the fusion protein at an initial dose of about 1200 mg IV on Day 1, followed by a maintenance phase comprising administering the fusion protein starting Day 8 at a maintenance dose of about 960 mg SC weekly for about 24 weeks; wherein the first FH functional fragment is fused to the C-terminus of a first heavy chain of the anti-human C5 full-length antibody, and the second FH functional fragment is fused to the C-terminus of a second heavy chain of the anti-human C5 full-length antibody; wherein each heavy chain of the anti-human C5 full-length antibody comprises the amino acid sequence of SEQ ID NO: 120, and each light chain of the anti-human C5 full-length antibody comprises the amino acid sequence of SEQ ID NO: 90; and wherein each heavy chain fused to FH functional fragment comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

### SLE-TMA

Thrombotic microangiopathy (TMA) is a serious complication that may occur in patients with systemic lupus erythematosus (SLE), adversely affecting the prognosis and increasing mortality. SLE is a chronic autoimmune disease of unknown cause. Renal involvement is common usually due to immune complex-mediated glomerular disease, however, vascular disease can also occur and generally adversely affect the prognosis and increase mortality. TMA is a severe renal vascular injury presenting with progressive life-threatening thrombocytopenia, microangiopathic hemolytic anemia, and advanced renal failure. The differential diagnosis of TMA in a patient with SLE includes antiphospholipid antibody syndrome (APS), thrombocytopenic purpura, complement-mediated and infection-associated hemolytic uremic syndrome, drug-mediated TMA (particularly due to calcineurin inhibitor toxicity), and malignant hypertension. TMA is characterized by endothelial injury that leads to thrombosis in capillaries and arterioles and results in a Coombs negative hemolytic anemia, thrombocytopenia, and end-organ damage, frequently affecting the kidneys. TMA encompasses several entities: thrombotic thrombocytopenia purpura (TTP), hemolytic uremic syndrome (HUS), as well as complement-mediated TMA. Generally, renal complications are particularly predominant with Shiga-toxin-associated hemolytic uremic syndrome (STx-HUS) and atypical HUS, whereas neurologic complications are more likely with TTP. Individuals with milder forms of TTP may have recurrent symptomatic episodes, including seizures and vision loss. SLE and/or APS are common autoimmune disorders associated with secondary HUS. Dysregulated terminal complement activation resulting in tissue injury is the common thread in the pathophysiology of all forms of complement mediated TMA. The clinical presentation of TMA, although dependent on the type, typically includes: fever, microangiopathic hemolytic anemia, kidney failure, thrombocytopenia and neurological manifestations. Multi-organ failure or injury is possible as TMA progresses, as the hyaline thrombi can spread to and affect the brain, kidneys, heart, liver, and other major organs. Typical organ damage related to TMA includes malignant hypertension, kidney injury, abdominal pain, diarrhea, stroke, confusion, heart injury, and eye damage. See. e.g., Figueiredo et al., CEN Case Rep. 2022; 11(1):26-30; Kello et al., Semin Arthritis Rheum. 2019; 49(1):74-83.

In some embodiments, the method of treating SLE-TMA described herein can prevent (e.g., preventing at least about any of 10%, 20%, 30%, 40%, 50%, 60%, 70%, 80%, 90%, or 100% chance), delay (e.g., delaying at least about any of 1, 2, 3, 6, 12, 18, 24, 30, 36, 42, 48, 52 months or longer), or reduce (e.g., reducing at least about any of 10%, 20%, 30%, 40%, 50%, 60%, 70%, 80%, 90%, or 100%) the severity of one or more of SLE-TMA signs/symptoms/characteristics discussed above, including but not limited to, APS, thrombocytopenic purpura, complement-mediated and infection-associated hemolytic uremic syndrome, drug-mediated TMA, malignant hypertension, thrombocytopenia, microangiopathic hemolytic anemia, kidney injury, and advanced renal failure. In some embodiments, the method of treating SLE-TMA described herein results in a reduction (e.g., reducing at least about any of 10%, 20%, 30%, 40%, 50%, 60%, 70%, 80%, 90%, or 100%) in one or more of the following in the subject: i) proteinuria; ii) requirement of hemodialysis; iii) morality rate; iv) complement activity; and v) Systemic Lupus Erythematosus Disease Activity Index (SLEDAI) score. In some embodiments, the method of treating SLE-TMA described herein results in an improvement (e.g., improving at least about any of 10%, 20%, 30%, 40%, 50%, 60%, 70%, 80%, 90%, or 100%) in one or more of the following in the subject: i) FACIT-Fatigue Scale Score; ii) renal function; iii) eGFR; and iv) quality of life.

In some embodiments, the efficacy of the method of treating SLE-TMA described herein can be assessed by one or more of: 1) change from baseline in platelet count; 2) the percent change from baseline in serum lactate dehydrogenase (LDH) levels; 3) the percent change of estimated glomerular filtration rate (eGFR) from baseline; 4) the percent change in urine protein/creatinine ratio (UPCR) from baseline; 5) time to the first hematological response, e.g., platelet count >100,000/μL accompanied by normalized LDH; 6) time to improvement in platelet count of at least 25% (e.g., at least about any of 30%, 40%, 50%, 60%, 70%, 75%, 80%, 90%, or 100%) from baseline; 7) percent of subjects with improvement in platelet count of at least 25% (e.g., at least about any of 30%, 40%, 50%, 60%, 70%, 75%, 80%, 90%, or 100%) from Baseline; 8) change from baseline in haptoglobin, hemoglobin, albumin, 24-hour proteinuria, bilirubin, and/or reticulocyte count; 9) change in dialysis status throughout the course of treatment; 10) change in RBC or platelet transfusion requirement (e.g., number of transfusions and total units required); 11) overall survival; 12) rate of extra-renal complications related to TMA; 13) percentage of subjects with resolution of schistocytosis; 14) markers of healthcare utilization and morbidity including but not limited to: duration of inpatient hospitalization (days) and/or duration of ICU stay during the treatment, and quality of life (QoL; The Functional Assessment of Chronic Illness Therapy-Fatigue [FACIT-F]); 15) incidence of treatment failure as defined by any of the following conditions: doubling of creatinine from Baseline in the setting of renal-related SAEs or corresponding to >1 grade increase in Common Terminology Criteria for Adverse Events (CTCAE), worsening of renal functioning requiring new dialysis, worsening of renal or hematologic functioning requiring use of another complement inhibitor, worsening of renal, hematological, neurologic, or other functions as a result of TMA, requiring use of IVIG, belimumab, or rituximab, major extra-renal adverse event secondary to TMA, and subject death due to TMA, TMA-related complications, or the fusion protein; 16) change in SLEDAI score from Baseline; 17) renal biopsy status; 18) presence of autoantibodies to Factor H; 19) pharmacodynamics and biomarker changes of the anti-C5-FH fusion protein, including but not limited to: change from baseline in serum C3b and free and total C5, change from baseline in rabbit RBC assay (alternative complement pathway [AP] and terminal complement pathway [TP] activity), change in factor H endogenous serum level, and change in serum C5b-9; and 20) immunogenicity of the anti-C5-FH fusion protein.

In some embodiments, the subject receiving the anti-C5-FH fusion protein will continue to receive standard of care (SOC) therapy for SLE-TMA. SOC includes any combination of the following: IV or PO corticosteroids, cyclophosphamide induction with/without azathioprine maintenance therapy, calcineurin inhibitors, or mycophenolate mofetil. SOC will exclude other complement inhibitors, IVIG, and rituximab. In some embodiments, the individual can further receive rescue therapy via plasma exchange, plasmapheresis, and/or plasma infusion.

In some embodiments, the method of treating SLE-TMA described herein further comprises selecting a human individual (e.g., complement inhibitor-naïve human individual) suitable for such treatment. In some embodiments, the human individual to be treated has prior vaccination against one or all of Neisseria meningitidis, Streptococcus pneumoniae, and Hemophilus influenzae. In some embodiments, the human individual further receives antibiotic prophylaxis during the treatment. In some embodiments, individuals not vaccinated with above vaccination will receive prophylactic antibiotics starting no later than concurrently with the first dose of the anti-C5-FH fusion protein, and will continue to receive this antibiotic prophylaxis treatment until 2 weeks after vaccination. In some embodiments, the human individual is about 18 to about 65 years old. In some embodiments, the human individual meets one or more of the criteria: 1) meets criteria for SLE per the 2019 European League Against Rheumatism (EULAR)/American College of Rheumatology (ACR) criteria; 2) decrease in platelet count to <100,000/μL AND representing at least a 25% (e.g., at least about any of 30%, 40%, 50%, 60%, 70%, 75%, 80%, 90%, or 100%) decrease from pre-treatment platelet count. Pre-treatment platelet count is defined as the platelet count within 6 months prior to screening, or the median of all platelet counts if more than 1 measurement was taken during those 6 months (If no platelet values from before screening are available, a platelet count of <100,000/μL at screening AND a renal biopsy within 6 months with evidence of TMA will be sufficient.); 3) LDH ≥2× the upper limit of normal (ULN); 4) presence of schistocytes on peripheral blood smear within 14 days of Screening; 5) abnormal renal function as defined by creatinine above the ULN or proteinuria as defined below. Subjects requiring dialysis within 4 weeks of screening for acute kidney injury due to SLE-TMA are eligible (Urine protein ≥1.0 g/24h; OR UPCR ≥1.0 g/g (or ≥113 mg/mmol) on 2 separate assessments during the Screening Period; for example, these assessments should be separated by at least 3 days and should have a difference of <20% comparing the higher to the lower value); 6) practice effective contraception from Screening till end of treatment; 7) a negative pregnancy test for women at Screening and/or within 24 hours prior to first dosing of the anti-C5-FH fusion protein; 8) evidence of prior vaccination against Neisseria meningitidis, Streptococcus pneumoniae, and Hemophilus influenzae at Screening (subjects not providing evidence of prior vaccination should receive vaccination following initiation of administration of the anti-C5-FH fusion protein but are required to receive treatment with appropriate antibiotic prophylaxis until 2 weeks after vaccination); and 9) evidence of microangiopathic hemolytic anemia.

In some embodiments, the method of treating PNH described herein further comprises excluding a human individual not suitable for such treatment. In some embodiments, a human individual not suitable for the method of treatment described herein meets one or more of the criteria: 1) diagnosis of other TMA syndromes, including but not limited to ADAMTS13-deficiency-mediated TMA, metabolism-mediated TMA, Shiga-toxin-mediated TMA, coagulation-mediated TMA, hematopoietic stem cell transplantation-mediated TMA, and drug-mediated TMA; 2) a renal biopsy within 7 days of screening that shows exclusively chronic changes of TMA, such as defined by mucoid changes and onion skin lesions of arterioles and/or arteries, without any acute components as defined by at least 1 fibrin microthrombus in glomeruli, small arterioles, and/or arteries; 3) any history or sign in the 6 months prior to screening of significant chronic active or recurrent infection or screening laboratory evidence consistent with a significant chronic active or recurrent infection requiring treatment with antibiotics, antivirals, or antifungals; 4) positive Coombs test at the time of TMA diagnosis; 5) treatment of any infection with IV (within 30 days of Screening) or oral (within 14 days of Screening) antibiotics, antivirals, or antifungals; 6) positive nasopharyngeal swab for Neisseria meningitidis at Screening or a prior history of meningitis; 7) history of meningococcal infection; 8) untreated tuberculosis; 9) positive serology for HCV or HIV; 10) history of splenectomy; 11) known allergy to penicillin antibiotics; 12) known or suspected immunodeficiency disease, including hereditary complement deficiency; 13) history of transplant including heart, lung, small bowel, pancreas, liver, kidney, bone marrow, or stem cell transplant; 14) absolute neutrophil count <1000 cells/mm³; 15) eGFR <30 mL/min/1.73 m² with the exception of subjects requiring acute dialysis within 4 weeks of screening for the new diagnosis of SLE-TMA; 16) platelet count <30,000/mm³; 17) history of drug or alcohol abuse within 1 year of Screening; 18) received any type of live attenuated vaccine <1 month prior to Screening or is planning to receive any such live attenuated vaccine over the course of treatment; 19) use of any complement inhibitors; 20) use of IVIG within 7 days of anti-C5-FH fusion protein initiation; 21) use of rituximab within 3 months of anti-C5-FH fusion protein initiation; 22) use of belimumab within 3 months of anti-C5-FH fusion protein initiation; 23) history of malignancy, except adequately treated basal cell carcinoma or in situ carcinoma of the uterine cervix; 24) any female who is pregnant or breastfeeding, or any female who is planning to become pregnant during treatment or shortly after; 25) any condition such as active symptomatic COVID infection that may compromise treatment, present a safety risk to the subject, or may confound the interpretation of the treatment results; 26) a QT duration corrected for heart rate by Fridericia's formula (QTcF) >450 millisecond (msec) for males or >470 msec for females based on either single or averaged QTcF values of triplicate ECGs obtained over a 3-minute interval; and 27) unwilling to get vaccinated against Neisseria meningitidis, Streptococcus pneumoniae, and Hemophilus influenzae.

Methods of treating C3G and/or IgAN

In some embodiments, there is provided a method of treating C3G in a human individual (e.g., complement inhibitor-naïve human individual), comprising administering to the human individual an effective amount of a fusion protein (e.g., any of the anti-C5-FH fusion proteins described herein, such as FMEH-IgG4-PLA-FH) comprising i) an antibody moiety that specifically binds to a human C5 (e.g., anti-human C5 full-length antibody, such as FMEH-IgG4-PLA) and ii) an FH or functional fragment thereof (e.g., SCR1-5 domains of FH, such as SEQ ID NO: 85). In some embodiments, the fusion protein is administered intravenously. In some embodiments, the fusion protein is administered subcutaneously. In some embodiments, the fusion protein is administered at the dose of about 60 to about 3600 mg (including for example any of 60, 180, 360, 600, 720, 960, 1200, 1440, 1800, 1920, 2400, 1920, 2880, 3000, 3200, and 3600 mg). In some embodiments, the fusion protein is administered at a single dose, such as at a dose of about 60 mg to about 1200 mg. In some embodiments, the fusion protein is administered at a single dose of about 60 mg IV, about 180 mg IV, about 180 mg SC, about 360 mg IV, about 600 mg IV, about 720 mg SC, or about 1200 mg IV. In some embodiments, the fusion protein is administered at multiple doses. In some embodiments, the fusion protein is administered weekly or biweekly, such as administered at a dose of about 600 mg to about 2880 mg. In some embodiments, the fusion protein is administered with an initial phase comprising administering the fusion protein at one or more (e.g., one) initial doses (e.g., about 600 mg to about 3600 mg, or about 1200 mg to about 3600 mg per dose, such as IV administration), followed by a maintenance phase comprising administering the fusion protein (e.g., weekly or biweekly) for at least two (e.g., 2, 3, 4, 5, 6, 8, 10, 11, 12, 13, 14, 23, 24, 25, 26, 40, 44, 47, 48, 49, 50, or more) doses (e.g., about 600 mg to about 2880 mg per dose). In some embodiments, the two or more maintenance doses of the fusion protein are administered weekly, such as at about 600 mg to about 1440 mg per dose. In some embodiments, the two or more maintenance doses of the fusion protein are each administered weekly at about 600 mg IV to about 1200 mg IV, or at about 720 mg SC to about 1440 mg SC. In some embodiments, the two or more maintenance doses of the fusion protein are administered biweekly, such as at about 1800 mg to about 2880 mg. In some embodiments, the two or more maintenance doses of the fusion protein are each administered biweekly at about 1920 mg SC to about 2880 mg SC, or at about 1800 mg SC to about 2400 mg SC. In some embodiments, the maintenance phase is at least about 48 weeks. In some embodiments, the initial dosing phase comprises administering to the individual a dose of about 600 mg to about 3600 mg (e.g., IV) weekly for one or more doses (e.g., one dose only), followed by a maintenance phase comprising administering the fusion protein at about 720 mg to about 2880 mg (e.g., SC) or about 600 mg to about 1200 mg (e.g., IV) weekly or biweekly for at least two doses, e.g., at least about 48 weeks (e.g., 48, 49, 50, 51, 52, 54, 56, 58, 60 weeks or more, such as 48 weeks) for weekly maintenance dosing, or at least about 49 weeks (e.g., 49, 51, 53, 55, 57, 59, 61, 63, 65 weeks or more, such as 49 weeks) for biweekly maintenance dosing. In some embodiments, the anti-C5 antibody moiety comprises a VH-CDR1 comprising the amino acid sequence of SEQ ID NO: 86, a VH-CDR2 comprising the amino acid sequence of SEQ ID NO: 57, a VH-CDR3 comprising the amino acid sequence of SEQ ID NO: 49, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96. In some embodiments, the anti-C5 antibody moiety comprises a VH comprising the amino acid sequence of SEQ ID NO: 87, and a VL comprising the amino acid sequence of SEQ ID NO: 88. In some embodiments, the anti-C5 antibody moiety is a full-length antibody comprising i) a heavy chain comprising the amino acid sequence of SEQ ID NO: 120, and ii) a light chain comprising the amino acid sequence of SEQ ID NO: 90. In some embodiments, the FH or functional fragment thereof is fused to the C-terminus of one or both (e.g., both) heavy chains of the anti-C5 full-length antibody. In some embodiments, the heavy chain fused to FH or functional fragment thereof comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating IgAN in a human individual (e.g., complement inhibitor-naïve human individual), comprising administering to the human individual an effective amount of a fusion protein (e.g., any of the anti-C5-FH fusion proteins described herein, such as FMEH-IgG4-PLA-FH) comprising i) an antibody moiety that specifically binds to a human C5 (e.g., anti-human C5 full-length antibody, such as FMEH-IgG4-PLA) and ii) an FH or functional fragment thereof (e.g., SCR1-5 domains of FH, such as SEQ ID NO: 85). In some embodiments, the fusion protein is administered intravenously. In some embodiments, the fusion protein is administered subcutaneously. In some embodiments, the fusion protein is administered at the dose of about 60 to about 3600 mg (including for example any of 60, 180, 360, 600, 720, 960, 1200, 1440, 1800, 1920, 2400, 1920, 2880, 3000, 3200, and 3600 mg). In some embodiments, the fusion protein is administered at a single dose, such as at a dose of about 60 mg to about 1200 mg. In some embodiments, the fusion protein is administered at a single dose of about 60 mg IV, about 180 mg IV, about 180 mg SC, about 360 mg IV, about 600 mg IV, about 720 mg SC, or about 1200 mg IV. In some embodiments, the fusion protein is administered at multiple doses. In some embodiments, the fusion protein is administered weekly or biweekly, such as administered at a dose of about 600 mg to about 2880 mg. In some embodiments, the fusion protein is administered with an initial phase comprising administering the fusion protein at one or more (e.g., one) initial doses (e.g., about 600 mg to about 3600 mg, or about 1200 mg to about 3600 mg per dose, such as IV administration), followed by a maintenance phase comprising administering the fusion protein (e.g., weekly or biweekly) for at least two (e.g., 2, 3, 4, 5, 6, 8, 10, 11, 12, 13, 14, 23, 24, 25, 26, 40, 44, 47, 48, 49, 50, or more) doses (e.g., about 600 mg to about 2880 mg per dose). In some embodiments, the two or more maintenance doses of the fusion protein are administered weekly, such as at about 600 mg to about 1440 mg per dose. In some embodiments, the two or more maintenance doses of the fusion protein are each administered weekly at about 600 mg IV to about 1200 mg IV, or at about 720 mg SC to about 1440 mg SC. In some embodiments, the two or more maintenance doses of the fusion protein are administered biweekly, such as at about 1800 mg to about 2880 mg. In some embodiments, the two or more maintenance doses of the fusion protein are each administered biweekly at about 1920 mg SC to about 2880 mg SC, or at about 1800 mg SC to about 2400 mg SC. In some embodiments, the maintenance phase is at least about 48 weeks. In some embodiments, the initial dosing phase comprises administering to the individual a dose of about 600 mg to about 3600 mg (e.g., IV) weekly for one or more doses (e.g., one dose only), followed by a maintenance phase comprising administering the fusion protein at about 720 mg to about 2880 mg (e.g., SC) or about 600 mg to about 1200 mg (e.g., IV) weekly or biweekly for at least two doses, e.g., at least about 48 weeks (e.g., 48, 49, 50, 51, 52, 54, 56, 58, 60 weeks or more, such as 48 weeks) for weekly maintenance dosing, or at least about 49 weeks (e.g., 49, 51, 53, 55, 57, 59, 61, 63, 65 weeks or more, such as 49 weeks) for biweekly maintenance dosing. In some embodiments, the anti-C5 antibody moiety comprises a VH-CDR1 comprising the amino acid sequence of SEQ ID NO: 86, a VH-CDR2 comprising the amino acid sequence of SEQ ID NO: 57, a VH-CDR3 comprising the amino acid sequence of SEQ ID NO: 49, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96. In some embodiments, the anti-C5 antibody moiety comprises a VH comprising the amino acid sequence of SEQ ID NO: 87, and a VL comprising the amino acid sequence of SEQ ID NO: 88. In some embodiments, the anti-C5 antibody moiety is a full-length antibody comprising i) a heavy chain comprising the amino acid sequence of SEQ ID NO: 120, and ii) a light chain comprising the amino acid sequence of SEQ ID NO: 90. In some embodiments, the FH or functional fragment thereof is fused to the C-terminus of one or both (e.g., both) heavy chains of the anti-C5 full-length antibody. In some embodiments, the heavy chain fused to FH or functional fragment thereof comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating C3G or IgAN in a human individual (e.g., complement inhibitor-naïve human individual), comprising administering to the human individual an effective amount of a fusion protein (e.g., any of the anti-C5-FH fusion proteins described herein, such as FMEH-IgG4-PLA-FH) comprising i) an anti-human C5 antibody moiety (e.g., anti-human C5 full-length antibody, such as FMEH-IgG4-PLA) and ii) an FH or functional fragment thereof (e.g., SCR1-5 domains of FH, such as SEQ ID NO: 85), wherein the fusion protein is administered with an initial phase comprising administering the fusion protein at an initial dose (e.g., about 600 mg to about 1200 mg, about 600 mg to about 2880 mg, about 720 mg to about 1440 mg, about 720 mg to about 3000 mg, about 1200 mg IV to about 3600 mg IV, about 1200 mg IV to about 2400 mg IV, or about 2400 mg IV to about 3600 mg IV) on Day 1, followed by a maintenance phase comprising administering the fusion protein starting Day 8 for two or more (e.g., 48, 49, 50, 51, 52, 53, 54, 55, 56, 57, 58, 59, 60, 61, 62 weeks or more) maintenance doses; and wherein the maintenance phase comprises administering the fusion protein (e.g., weekly or biweekly) at a first maintenance dose for a first maintenance phase period (e.g., 1, 2, 3, 4, 5, 6, 7, 8, 9, 10, 11, 12, 18, 24, 30, 40, 42, 44 weeks or more), followed by administering the fusion protein (e.g., weekly or biweekly) at a second maintenance dose for a second maintenance phase period (e.g., 1, 2, 3, 4, 5, 6, 7, 8, 9, 10, 11, 12, 18, 24, 30, 40, 42, 44, 46, 48, 50 weeks or more). In some embodiments, the initial dose is about any of 600 mg, 720 mg, 1200 mg, 1440 mg, 1600 mg, 1800 mg, 1920 mg, 2400 mg, 2880 mg, 3000 mg, 3240 mg, or 3600 mg. In some embodiments, the first maintenance dose and/or the second maintenance dose is about 600 mg to about 2880 mg, such as any of about 600 mg to about 1200 mg, about 720 mg to about 1440 mg, about 1800 mg to about 2880 mg, about 1920 mg to about 2880 mg, about 1800 mg to about 2400 mg, about 600 mg to about 2400 mg, or about 720 mg to about 2400 mg. In some embodiments, the first maintenance dose and/or the second maintenance dose is about any of 600 mg, 720 mg, 960 mg, 1200 mg, 1440 mg, 1800 mg, 1920 mg, 2400 mg, or 2880 mg. In some embodiments, the initial dose, the first maintenance dose, and/or the second maintenance dose of the fusion protein is administered IV or SC. In some embodiments, the first maintenance dose and/or the second maintenance dose of the fusion protein is administered at about 600 mg IV to about 1200 mg IV, about 720 mg SC to about 1440 mg SC, about 1920 mg SC to about 2880 mg SC, or at about 1800 mg SC to about 2400 mg SC. In some embodiments, the second maintenance dose is higher than the first maintenance dose. In some embodiments, the second maintenance phase period is longer than the first maintenance phase period. In some embodiments, the first maintenance dose of the fusion protein is administered at about 720 mg SC or about 600 mg IV weekly, and the second maintenance dose of the fusion protein is administered at about 1440 mg SC or about 1200 mg IV weekly. In some embodiments, the first maintenance phase period is about 4 weeks, and the second maintenance phase period is at least about 44 weeks. In some embodiments, the initial phase comprises administering the fusion protein at an initial dose of about 600 mg IV to about 1200 mg IV (e.g., about 1200 mg IV) on Day 1, and wherein the maintenance phase comprises: i) administering the fusion protein at a first maintenance dose of about 720 mg SC or about 600 mg IV weekly starting Day 8 for about 4 weeks, followed by ii) administering the fusion protein at a second maintenance dose of about 1440 mg SC or about 1200 mg IV weekly starting Day 36 for about 44 weeks. In some embodiments, the anti-C5 antibody moiety comprises a VH-CDR1 comprising the amino acid sequence of SEQ ID NO: 86, a VH-CDR2 comprising the amino acid sequence of SEQ ID NO: 57, a VH-CDR3 comprising the amino acid sequence of SEQ ID NO: 49, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96. In some embodiments, the anti-C5 antibody moiety comprises a VH comprising the amino acid sequence of SEQ ID NO: 87, and a VL comprising the amino acid sequence of SEQ ID NO: 88. In some embodiments, the anti-C5 antibody moiety is a full-length antibody comprising i) a heavy chain comprising the amino acid sequence of SEQ ID NO: 120, and ii) a light chain comprising the amino acid sequence of SEQ ID NO: 90. In some embodiments, the FH or functional fragment thereof is fused to the C-terminus of one or both (e.g., both) heavy chains of the anti-C5 full-length antibody. In some embodiments, the heavy chain fused to FH or functional fragment thereof comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating C3G or IgAN in a human individual (e.g., complement inhibitor-naïve human individual), comprising administering to the human individual an effective amount of a fusion protein comprising i) an anti-human C5 full-length antibody, ii) a first FH functional fragment, and iii) a second FH functional fragment; wherein the fusion protein is administered with an initial phase comprising administering the fusion protein at an initial dose of about 600 mg IV to about 1200 mg IV (e.g., about 1200 mg IV) on Day 1, followed by a maintenance phase comprising (a) administering the fusion protein at a first maintenance dose of about 720 mg SC or about 600 mg IV weekly starting Day 8 for about 4 weeks, followed by (b) administering the fusion protein at a second maintenance dose of about 1440 mg SC or about 1200 mg IV weekly starting Day 36 for about 44 weeks; wherein the first FH functional fragment is fused to the C-terminus of a first heavy chain of the anti-human C5 full-length antibody, and the second FH functional fragment is fused to the C-terminus of a second heavy chain of the anti-human C5 full-length antibody; wherein each heavy chain of the anti-human C5 full-length antibody comprises the amino acid sequence of SEQ ID NO: 120, and each light chain of the anti-human C5 full-length antibody comprises the amino acid sequence of SEQ ID NO: 90; and wherein each heavy chain fused to FH functional fragment comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating C3G or IgAN in a human individual (e.g., complement inhibitor-naïve human individual), comprising administering to the human individual an effective amount of a fusion protein (e.g., any of the anti-C5-FH fusion proteins described herein, such as FMEH-IgG4-PLA-FH) comprising i) an anti-human C5 antibody moiety (e.g., anti-human C5 full-length antibody, such as FMEH-IgG4-PLA) and ii) an FH or functional fragment thereof (e.g., SCR1-5 domains of FH, such as SEQ ID NO: 85), wherein the fusion protein is administered with an initial phase comprising administering the fusion protein at an initial dose of about 600 mg IV to about 3600 mg IV (e.g., about 600 mg to about 1200 mg, about 600 mg to about 2880 mg, about 720 mg to about 1440 mg, about 720 mg to about 3000 mg, about 1200 mg IV to about 2400 mg IV, or about 2400 mg IV to about 3600 mg IV) on Day 1, followed by a maintenance phase comprising administering the fusion protein starting Day 8 at a maintenance dose of: (a) about 600 mg IV to about 1200 mg IV weekly for at least about 48 weeks; (b) about 720 mg SC to about 1440 mg SC weekly for at least about 48 weeks; (c) about 1920 mg SC to about 2880 mg SC biweekly for at least about 49 weeks; or (d) about 1800 mg SC to about 2400 mg SC biweekly for at least about 49 weeks. In some embodiments, the initial phase comprises administering the fusion protein at an initial dose of about 600 mg IV to about 1200 mg IV (e.g., about 1200 mg IV) on Day 1, and wherein the maintenance phase comprises administering the fusion protein starting Day 8 at a maintenance dose of about 720 mg SC or about 600 mg IV weekly for about 48 weeks. In some embodiments, the initial phase comprises administering the fusion protein at an initial dose of about 600 mg IV to about 1200 mg IV (e.g., about 1200 mg IV) on Day 1, and wherein the maintenance phase comprises administering the fusion protein starting Day 8 at a maintenance dose of about 1440 mg SC or about 1200 mg IV weekly for about 48 weeks. In some embodiments, the initial phase comprises administering the fusion protein at an initial dose of about 2400 mg IV to about 3600 mg IV on Day 1, and wherein the maintenance phase comprises administering the fusion protein starting Day 8 at a maintenance dose of about 1920 mg SC to about 2880 mg SC biweekly for about 49 weeks. In some embodiments, the initial phase comprises administering the fusion protein at an initial dose of about 600 mg IV on Day 1, and wherein the maintenance phase comprises administering the fusion protein starting Day 8 at a maintenance dose of about 720 mg SC weekly for about 48 weeks. In some embodiments, the anti-C5 antibody moiety comprises a VH-CDR1 comprising the amino acid sequence of SEQ ID NO: 86, a VH-CDR2 comprising the amino acid sequence of SEQ ID NO: 57, a VH-CDR3 comprising the amino acid sequence of SEQ ID NO: 49, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96. In some embodiments, the anti-C5 antibody moiety comprises a VH comprising the amino acid sequence of SEQ ID NO: 87, and a VL comprising the amino acid sequence of SEQ ID NO: 88. In some embodiments, the anti-C5 antibody moiety is a full-length antibody comprising i) a heavy chain comprising the amino acid sequence of SEQ ID NO: 120, and ii) a light chain comprising the amino acid sequence of SEQ ID NO: 90. In some embodiments, the FH or functional fragment thereof is fused to the C-terminus of one or both (e.g., both) heavy chains of the anti-C5 full-length antibody. In some embodiments, the heavy chain fused to FH or functional fragment thereof comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating C3G or IgAN in a human individual (e.g., complement inhibitor-naïve human individual), comprising administering to the human individual an effective amount of a fusion protein comprising i) an anti-human C5 full-length antibody, ii) a first FH functional fragment, and iii) a second FH functional fragment; wherein the fusion protein is administered with an initial phase comprising administering the fusion protein at an initial dose of about 1200 mg IV on Day 1, followed by a maintenance phase comprising administering the fusion protein starting Day 8 at a maintenance dose of about 720 mg SC or about 600 mg IV weekly for about 48 weeks; wherein the first FH functional fragment is fused to the C-terminus of a first heavy chain of the anti-human C5 full-length antibody, and the second FH functional fragment is fused to the C-terminus of a second heavy chain of the anti-human C5 full-length antibody; wherein each heavy chain of the anti-human C5 full-length antibody comprises the amino acid sequence of SEQ ID NO: 120, and each light chain of the anti-human C5 full-length antibody comprises the amino acid sequence of SEQ ID NO: 90; and wherein each heavy chain fused to FH functional fragment comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating C3G or IgAN in a human individual (e.g., complement inhibitor-naïve human individual), comprising administering to the human individual an effective amount of a fusion protein comprising i) an anti-human C5 full-length antibody, ii) a first FH functional fragment, and iii) a second FH functional fragment; wherein the fusion protein is administered with an initial phase comprising administering the fusion protein at an initial dose of about 600 mg IV on Day 1, followed by a maintenance phase comprising administering the fusion protein starting Day 8 at a maintenance dose of about 720 mg SC weekly for about 48 weeks; wherein the first FH functional fragment is fused to the C-terminus of a first heavy chain of the anti-human C5 full-length antibody, and the second FH functional fragment is fused to the C-terminus of a second heavy chain of the anti-human C5 full-length antibody; wherein each heavy chain of the anti-human C5 full-length antibody comprises the amino acid sequence of SEQ ID NO: 120, and each light chain of the anti-human C5 full-length antibody comprises the amino acid sequence of SEQ ID NO: 90; and wherein each heavy chain fused to FH functional fragment comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating C3G or IgAN in a human individual (e.g., complement inhibitor-naïve human individual), comprising administering to the human individual an effective amount of a fusion protein comprising i) an anti-human C5 full-length antibody, ii) a first FH functional fragment, and iii) a second FH functional fragment; wherein the fusion protein is administered with an initial phase comprising administering the fusion protein at an initial dose of about 1200 mg IV on Day 1, followed by a maintenance phase comprising administering the fusion protein starting Day 8 at a maintenance dose of about 1440 mg SC or about 1200 mg IV weekly for about 48 weeks; wherein the first FH functional fragment is fused to the C-terminus of a first heavy chain of the anti-human C5 full-length antibody, and the second FH functional fragment is fused to the C-terminus of a second heavy chain of the anti-human C5 full-length antibody; wherein each heavy chain of the anti-human C5 full-length antibody comprises the amino acid sequence of SEQ ID NO: 120, and each light chain of the anti-human C5 full-length antibody comprises the amino acid sequence of SEQ ID NO: 90; and wherein each heavy chain fused to FH functional fragment comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

In some embodiments, there is provided a method of treating C3G or IgAN in a human individual (e.g., complement inhibitor-naïve human individual), comprising administering to the human individual an effective amount of a fusion protein comprising i) an anti-human C5 full-length antibody, ii) a first FH functional fragment, and iii) a second FH functional fragment; wherein the fusion protein is administered with an initial phase comprising administering the fusion protein at an initial dose of about 2400 mg IV to about 3600 mg IV on Day 1, followed by a maintenance phase comprising administering the fusion protein starting Day 8 at a maintenance dose of about 1920 mg SC to about 2880 mg SC biweekly for about 49 weeks; wherein the first FH functional fragment is fused to the C-terminus of a first heavy chain of the anti-human C5 full-length antibody, and the second FH functional fragment is fused to the C-terminus of a second heavy chain of the anti-human C5 full-length antibody; wherein each heavy chain of the anti-human C5 full-length antibody comprises the amino acid sequence of SEQ ID NO: 120, and each light chain of the anti-human C5 full-length antibody comprises the amino acid sequence of SEQ ID NO: 90; and wherein each heavy chain fused to FH functional fragment comprises the amino acid sequence of SEQ ID NO: 89. In some embodiments, the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0. In some embodiments, for IV administration, the anti-C5-FH fusion protein is diluted in an infusion bag containing 100 mL of 0.9% sodium chloride for infusion. In some embodiments, for SC administration, the anti-C5-FH fusion protein is administered without further formulation and administered into the abdominal wall using standard techniques for SC administration.

### C3G

Complement 3 glomerulopathy (C3G) is a rare kidney disease (estimated at 2-3 per 1,000,000 people) that has two forms: dense deposit disease (DDD) and C3 glomerulonephritis (C3GN). C3G is characterized by deposition of C3 in the filtration units (the glomeruli) of the kidney, indicating complement involvement in causing kidney damage. C3 glomerulopathy is characterized by evidence of alternative complement activation based on C3 deposition in the glomeruli. Genetic lesions leading to defective complement regulation, including mutations in complement factor H have been described in these patients. Common signs and symptoms of C3G (DDD or C3GN) that are related to a loss of normal kidney function include the following: blood in the urine (hematuria), excess protein in the urine (proteinuria), acute nephritic syndrome or nephrotic syndrome, low levels of the complement component C3, swelling (edema), gout, recurrent infections, less urine made (oliguria), hypertension, fatigue and reduced alertness, drusen, abnormal distribution of fat under the skin (acquired partial lipodystrophy), and any combinations thereof. C3G (DDD or C3GN) can also lead to kidney failure, the signs and symptoms of which include: lack of appetite, nausea and vomiting, difficulty sleeping, dry and itchy skin, and nighttime muscle cramps. There is no approved treatment for patients with C3 glomerulopathy, including C3GN. Without treatment, C3G invariably leads to kidney failure, and kidney transplant is frequently the only option. Even after transplantation, the new kidney will frequently fail due to recurrence of the disease.

In some embodiments, the method of treating C3G described herein can prevent (e.g., preventing at least about any of 10%, 20%, 30%, 40%, 50%, 60%, 70%, 80%, 90%, or 100% chance), delay (e.g., delaying at least about any of 1, 2, 3, 6, 12, 18, 24, 30, 36, 42, 48, 52 months or longer), or reduce (e.g., reducing at least about any of 10%, 20%, 30%, 40%, 50%, 60%, 70%, 80%, 90%, or 100%) the severity of one or more of C3G signs and symptoms discussed above, including but not limited to, hematuria, proteinuria, nephritic syndrome, kidney failure, drusen. In some embodiments, the method of treating C3G described herein can ameliorate (e.g., reducing at least about any of 10%, 20%, 30%, 40%, 50%, 60%, 70%, 80%, 90%, or 100%) one or more renal pathologies, including but not limited to, proteinuria, hematuria, crescents and fibrin deposition, C3 deposition, endocapillary hypercellularity, and mesangial hypercellularity. In some embodiments, the method of treating C3G described herein can reduce (e.g., reducing at least about any of 10%, 20%, 30%, 40%, 50%, 60%, 70%, 80%, 90%, or 100%) one or more of: C3 deposit score, C3b activity, free serum C5 level, eGFR, urinary protein creatinine ratio (UPCR; calculated as percent change in protein (Pr)/Creatinine (Cr)), and RBC lysis. In some embodiments, the method of treating C3G described herein can improve (e.g., improving at least about any of 10%, 20%, 30%, 40%, 50%, 60%, 70%, 80%, 90%, or 100%) quality of life, such as assessed by the Functional Assessment of Chronic Illness Therapy (FACIT)-Fatigue Score and Kidney Disease Quality of Life (KDQoL) scale, and/or histology and histopathology of renal biopsy.

### IgAN

IgA nephropathy (IgAN) is the most common primary glomerulonephritis worldwide.

Aberrant glycosylation of IgAQ1 results in increased serum levels of galactose-deficient IgAQ1 (Gd-IgAQ1) that are recognized by glycan-specific IgA and IgG autoantibodies. Aggregates of the immune complexes are formed in situ and/or deposited in the glomerular mesangium. This promotes proliferation of mesangial cells, increased synthesis of extracellular matrix proteins, cytokines, chemokines, and infiltration of immune cells into the surrounding tissue. Accordingly, disease progression involves (1) production of Gd-IgAQ1; and (2) its recognition by antiglycan autoantibodies; which (3) form immune complexes in the kidney; and (4) activate mesangial cells. See, e.g., Penfold et al., Int. J. Nephrol. and Renovascular Dis. 11, pp. 137-148 (2017).

IgAN occurs primarily in subjects in their 20s and 30s. Patients present with a range of symptoms, typically including micro- or macro-hematuria and increased protein excretion in the urine. Patients may also present with hypertension as a result of sustained renal damage. Current therapeutic approaches merely provide supportive care, including administration of the maximum tolerable dose of an angiotensin converting enzyme inhibitor or angiotensin-receptor blocker, or administration of immunosuppressive drugs, whose benefits are largely outweighed by adverse reactions. Ultimately, 30-40% of patients will develop end-stage renal disease (ESRD) within 20-30 years of diagnosis of IgAN. In the interim, patients experience numerous symptoms that significantly degrade their quality of life, in addition to declining renal function. Patients with IgAN often exhibit significantly increased expressions of endothelin 1 (ET-1) and ET-RA in the kidney. Increased expression of endothelins positively correlates with proteinuria, one of the hallmark symptoms of IgAN.

In some embodiments, the method of treating IgAN described herein can prevent (e.g., preventing at least about any of 10%, 20%, 30%, 40%, 50%, 60%, 70%, 80%, 90%, or 100% chance), delay (e.g., delaying at least about any of 1, 2, 3, 6, 12, 18, 24, 30, 36, 42, 48, 52 months or longer), or reduce (e.g., reducing at least about any of 10%, 20%, 30%, 40%, 50%, 60%, 70%, 80%, 90%, or 100%) the severity of one or more of IgAN signs and symptoms discussed above, including but not limited to, proteinuria, hematuria, pain in back, edema, high blood pressure, as well as complications like high cholesterol, acute kidney failure, chronic kidney failure, nephrotic syndrome. In some embodiments, the method of treating IgAN described herein can achieve one or more of following effects: i) decreasing (e.g., decreasing at least about any of 10%, 20%, 30%, 40%, 50%, 60%, 70%, 80%, 90%, or 100%) renal inflammation and/or fibrosis; ii) reducing (e.g., reducing at least about any of 10%, 20%, 30%, 40%, 50%, 60%, 70%, 80%, 90%, or 100%) production of Gd-IgA1; iii) decreasing (e.g., decreasing at least about any of 10%, 20%, 30%, 40%, 50%, 60%, 70%, 80%, 90%, or 100%) the occurrence of hematuria; iv) stabilizing (e.g., not varying more than about 30%, 20%, 10%, 5% or less) eGFR; v) delaying (e.g., delaying at least about any of 1, 2, 3, 6, 12, 18, 24, 30, 36, 42, 48, 52 months or longer) the onset of ESRD; vi) decreasing (e.g., decreasing at least about any of 10%, 20%, 30%, 40%, 50%, 60%, 70%, 80%, 90%, or 100%) the number of IgA-nephropathy associated disease flares; vii) decreasing (e.g., decreasing at least about any of 10%, 20%, 30%, 40%, 50%, 60%, 70%, 80%, 90%, or 100%) proteinuria; viii) decreasing (e.g., decreasing at least about any of 10%, 20%, 30%, 40%, 50%, 60%, 70%, 80%, 90%, or 100%) fatigue; ix) inhibiting (e.g., inhibiting at least about any of 10%, 20%, 30%, 40%, 50%, 60%, 70%, 80%, 90%, or 100%) mesangial cell activation; x) reducing (e.g., reducing at least about any of 10%, 20%, 30%, 40%, 50%, 60%, 70%, 80%, 90%, or 100%) activation of a mesangial cell in contact with an IgA immune complex; xi) reducing (e.g., reducing at least about any of 10%, 20%, 30%, 40%, 50%, 60%, 70%, 80%, 90%, or 100%) expressions of endothelin 1 (ET-1) and ET-RA in the kidney; and xii) improving (e.g., improving at least about any of 10%, 20%, 30%, 40%, 50%, 60%, 70%, 80%, 90%, or 100%) renal function and/or renal pathology score. In some embodiments, the method of treating IgAN described herein can reduce (e.g., reducing at least about any of 10%, 20%, 30%, 40%, 50%, 60%, 70%, 80%, 90%, or 100%) one or more of: C3 deposit score, C3b activity, free serum C5 level, eGFR, UPCR, and RBC lysis. In some embodiments, the method of treating IgAN described herein can improve (e.g., improving at least about any of 10%, 20%, 30%, 40%, 50%, 60%, 70%, 80%, 90%, or 100%) quality of life, such as assessed by the FACIT-Fatigue Score and KDQoL scale, and/or histology and histopathology of renal biopsy.

In some embodiments, the efficacy of the method of treating C3G or IgAN described herein can be assessed by one or more of: i) the percent change from baseline in 24-hour UPCR; ii) change from baseline in Rabbit RBC assay, change from baseline in C3b activity assay, change from baseline in serum and urine Factor H level, change from baseline in urine MCP-1, C3a, C5a, properdin, or C5b-9 level, and/or change from baseline in free serum C5 levels; iii) change in eGFR; iv) change in quality of life assessed by the FACIT-Fatigue Score and KDQoL scale; and v) change in histology and histopathology in subjects undergoing repeat renal biopsy. Such methods are known in the art. In some embodiments, changes from baseline in markers of alternative complement pathway involvement, e.g., C3, C3d, C3c, C3adesArg, C5, C5a, C5b-9, C5adesArg, and other markers of inflammation, may be assessed in plasma/serum or urine over the course of the treatment period.

In some embodiments, the method of treating C3G or IgAN described herein further comprises selecting a human individual (e.g., complement inhibitor-naïve human individual) suitable for such treatment. In some embodiments, the human individual to be treated has prior vaccination against one or all of Neisseria meningitidis, Streptococcus pneumoniae, and Hemophilus influenzae. In some embodiments, the human individual further receives antibiotic prophylaxis during the treatment. In some embodiments, individuals not vaccinated with above vaccination will receive prophylactic antibiotics starting no later than concurrently with the first dose of the anti-C5-FH fusion protein, and will continue to receive this antibiotic prophylaxis treatment until 2 weeks after vaccination. In some embodiments, the human individual is between ages of about 18 years to about 75 years. In some embodiments, the human individual meets one or more of the criteria: 1) weight of >35 kilograms (kg) at Screening; 2) body mass index (BMI) of <35 kilograms per square meter (kg/m²); 3) UPCR >1.5 grams per gram (g/g) by 24-hour urine collection at Screening; 4) documented diagnosis and clinical status of IgAN or C3G; 5) tested for negative pregnancy, and effective contraception during entire treatment period; 6) vaccination; and 7) able to provide informed consent. In some embodiments, C3G or IgAN is verified by biopsy in the human individual to be treated. In some embodiments, the human individual is on stable regimen of angiotensin converting enzyme or angiotensin blocking agents for 12 weeks and/or sodium-glucose cotransporter-2 (SGLT2) inhibitors for 6 weeks at Screening.

In some embodiments, the method of treating C3G or IgAN described herein further comprises excluding a human individual not suitable for such treatment. In some embodiments, a human individual not suitable for the method of treatment described herein meets one or more of the criteria: 1) any clinically significant, poorly controlled underlying illness other than IgAN or C3G; 2) any history or sign of significant chronic active or recurrent infection, such as those requiring treatment or being treated with antibiotics, antivirals, or antifungals; 3) history of infections with encapsulated organisms; 4) history of untreated tuberculosis; 5) known allergy to penicillin antibiotics; 6) known or suspected immunodeficiency disease, including hereditary complement deficiency; 7) positive serology for hepatitis C virus (HCV) ribonucleic acid (RNA) or human immunodeficiency virus (HIV) at Screening; 8) history of bone marrow or stem cell transplantation; 9) absolute neutrophil count (ANC) <500 cells per microliter (cells/μL); 10) eGFR <30 milliliters per minute per 1.73 square meter (mL/min/1.73 m²) using the Chronic Kidney Disease Epidemiology Collaboration (CKD-EPI) formula; 11) presence of crescent formation in >50 percent (%) of glomeruli assessed on renal biopsy; 12) nephrotic syndrome; 13) rapidly progressive glomerulonephritis, defined as a fall in eGFR of >30 mL/min/1.73 m² within 24 weeks prior to the Screening Visit; 14) receiving renal replacement therapy or anticipated to require renal replacement therapy during the duration of the treatment; 15) history of drug or alcohol abuse within 1 year of screening; 16) received any type of live attenuated vaccine <4 weeks prior to screening or is planning to receive any such live attenuated vaccine over the course of the treatment; 17) use of rituximab within the last 12 weeks or any prior use of complement inhibitors; 18) use of systemic corticosteroids >10 mg per day (prednisone or equivalent) within 4 weeks prior to screening or immunosuppressive agents (e.g., mycophenolate mofetil, hydroxychloroquine, cyclosporin etc.) within 3 months prior to screening; 19) history of malignancy, except adequately treated basal cell carcinoma or in situ carcinoma of the uterine cervix; 20) any female who is pregnant or breastfeeding, or any female who is planning to become pregnant during the treatment; 21) a QT duration corrected for heart rate by Fridericia's formula (QTcF) >450 millisecond (msec) for males and >470 msec for females based on either single or averaged QTcF values of triplicate ECGs obtained over a 3-minute interval; 22) diagnosed with secondary forms of IgAN (e.g., Henoch Schönlein purpura, IgA vasculitis); and 23) use of any complement inhibitors.

The anti-C5-FH fusion proteins and components thereof are described in more details below.

### Anti-C5-fH Fusion Protein

Any anti-C5-FH fusion proteins and anti-C5 antibody moieties (e.g., pH-dependent anti-C5 antibody moieties) described in US20220204602, US20220177556, U.S. Ser. No. 11/578,137, and WO2020219922 can be used herein, the content of each of which is incorporated herein by reference in their entirety.

The methods descried herein utilize a fusion protein comprising an anti-C5 antibody moiety (such as any one of the anti-C5 antibodies or antigen-binding fragments thereof described herein) and an FH or a functional fragment thereof. In some embodiments, the anti-C5 antibody moiety is a full-length antibody (hereinafter referred to as “anti-C5 full-length antibody”). In some embodiments, the FH or functional fragment thereof is fused to one or both of the heavy chains of the anti-C5 full-length antibody (for example at the C-terminal end of the heavy chain(s)). In some embodiments, the anti-C5 full-length antibody comprises an Fc fragment derived from human IgG4 (such as an IgG4 Fc fragment comprising an PLA mutation (S228P/M428L/N434A)). In some embodiments, the IgG4 Fc fragment comprises the amino acid sequence of any of SEQ ID NOs: 32, 33, and 61. In some embodiments, the IgG4 Fc fragment comprising an PLA mutation comprises an amino acid sequence set forth in SEQ ID NO: 61. In some embodiments, the fragment of FH inhibits C3 activation. In some embodiments, the functional fragment of FH comprises short consensus repeat (SCR) domains 1-5 of FH. In some embodiments, the functional fragment of FH comprises the amino acid sequence of SEQ ID NO: 85. In some embodiments, the nucleic acid that encodes an IgG4 Fc comprising the amino acid sequence of SEQ ID NO: 61 comprises the nucleic acid sequence of SEQ ID NO: 60.

In some embodiments, the fusion protein comprises an anti-C5 antibody moiety (such as any one of the anti-C5 antibodies or antigen-binding fragments thereof described herein) and an FH or a functional fragment thereof comprising SCR1-5 domains of FH. In some embodiments, the fusion protein comprises i) an anti-C5 antibody moiety (such as any one of the anti-C5 antibodies described herein) comprising an IgG4 Fc fragment (e.g., an IgG4 Fc fragment comprising a PLA mutation), and ii) an FH or a functional fragment thereof comprising SCR1-5 domains of FH. In some embodiments, the functional fragment of FH comprises the amino acid sequence of SEQ ID NO: 85. In some embodiments, the IgG4 Fc fragment comprises the amino acid sequence of SEQ ID NO: 61.

In some embodiments, the fusion protein comprises an anti-C5 antibody moiety and an FH or a functional fragment thereof, wherein the anti-C5 antibody moiety is pH-sensitive or pH-dependent (such as any one of the pH-sensitive or pH-dependent anti-C5 antibodies or antigen-binding fragments thereof described herein). For example, in some embodiments, the pH-sensitive anti-C5 antibody moiety is an anti-C5 antibody moiety that binds to C5 at a higher affinity at a neutral pH (e.g., about pH 7.4) than at an acidic pH (e.g., about pH 5.8). In some embodiments, the binding affinity of the pH-dependent anti-C5 antibody moiety to C5 (e.g., human C5) at about pH 7.4 is at least about 3 times (such as at least about any of 4, 5, 6, 7, 8, 9, 10, or more) times higher than the binding affinity of the pH-dependent anti-C5 antibody moiety to C5 (e.g., human C5) at about pH 5.8. In some embodiments, the pH-dependent anti-C5 antibody moiety comprises an Fc fragment derived from human IgG4 (e.g., an IgG4 Fc fragment comprising a PLA mutation). In some embodiments, the functional fragment of FH comprises the amino acid sequence of SEQ ID NO: 85. In some embodiments, the IgG4 Fc fragment comprises the amino acid sequence of SEQ ID NO: 61.

In some embodiments, the fusion protein comprises an anti-C5 antibody moiety and an FH or functional fragment thereof, wherein the anti-C5 antibody moiety is pH-sensitive or pH-dependent (such as any one of the pH-sensitive or pH-dependent anti-C5 antibodies described herein), for example binding to C5 (e.g., human C5) at a higher affinity at pH 7.4 than at pH 5.8, wherein the anti-C5 antibody moiety comprising an IgG4 Fc fragment (e.g., an IgG4 Fc fragment comprising a PLA mutation). In some embodiments, the fusion protein comprises an anti-C5 antibody moiety and an FH or functional fragment thereof, wherein the anti-C5 antibody moiety is pH-sensitive or pH-dependent (such as any one of the pH-sensitive or pH-dependent anti-C5 antibodies or antigen-binding fragments thereof described herein), for example binding to C5 (e.g., human C5) at a higher affinity at pH 7.4 than at pH 5.8, and wherein the FH or functional fragment thereof comprises SCR1-5 domains of FH. In some embodiments, the fusion protein comprises an anti-C5 antibody moiety and an FH or functional fragment thereof, wherein the anti-C5 antibody moiety is pH-sensitive or pH-dependent (such as any one of the pH-sensitive or pH-dependent anti-C5 antibodies described herein), for example binding to C5 (e.g., human C5) at a higher affinity at pH 7.4 than at pH 5.8, wherein the anti-C5 antibody moiety comprises an IgG4 Fc fragment (e.g., an IgG4 Fc fragment comprising a PLA mutation), and wherein the FH or functional fragment thereof comprises SCR1-5 domains of FH. In some embodiments, the functional fragment of FH comprises the amino acid sequence of SEQ ID NO: 85. In some embodiments, the IgG4 Fc fragment comprises the amino acid sequence of SEQ ID NO: 61.

In some embodiments, the fusion protein comprises an anti-C5 antibody moiety (such as any one of the anti-C5 antibodies or antigen-binding fragments thereof described herein) and an FH or functional fragment thereof, wherein the anti-C5 antibody moiety comprises a histidine substitution in one or more of its CDR regions (such as any one of the histidine-containing anti-C5 antibodies or antigen-binding fragments thereof described herein). In some embodiments, the fusion protein comprises an anti-C5 antibody moiety and an FH or functional fragment thereof, wherein the anti-C5 antibody moiety comprises a histidine substitution in one or more of its CDR regions (such as any one of the histidine-containing anti-C5 antibodies described herein), and wherein the anti-C5 antibody moiety comprises an IgG4 Fc fragment (e.g., an IgG4 Fc fragment comprising a PLA mutation). In some embodiments, the fusion protein comprises an anti-C5 antibody moiety and an FH or functional fragment thereof, wherein the anti-C5 antibody moiety comprises a histidine substitution in one or more of its CDR regions (such as any one of the histidine-containing anti-C5 antibodies or antigen-binding fragments thereof described herein), and wherein the FH or functional fragment thereof comprises SCR1-5 domains of FH. In some embodiments, the fusion protein comprises an anti-C5 antibody moiety and an FH or functional fragment thereof, wherein the anti-C5 moiety comprises a histidine substitution in one or more of its CDR regions (such as any one of the histidine-containing anti-C5 antibodies described herein), wherein the anti-C5 antibody moiety comprises an IgG4 Fc fragment (e.g., an IgG4 Fc fragment comprising a PLA mutation), and wherein the FH or functional fragment thereof comprises SCR1-5 domains of FH. In some embodiments, the functional fragment of FH comprises the amino acid sequence of SEQ ID NO: 85. In some embodiments, the IgG4 Fc fragment comprises the amino acid sequence of SEQ ID NO: 61.

In some embodiments, the FH or functional fragment thereof (e.g., SEQ ID NO: 85) is fused to the anti-C5 antibody moiety (e.g., any one of the anti-C5 antibodies or antigen-binding fragments thereof described herein) via a linker (e.g., peptide linker), such as fused to the C-terminus of the anti-C5 antibody moiety via a linker. Any suitable peptide linker can be used herein, including but not limited to, a GS linker. In some embodiments, the FH or functional fragment thereof (e.g., SEQ ID NO: 85) is directly fused to the anti-C5 antibody moiety (e.g., any one of the anti-C5 antibodies or antigen-binding fragments thereof described herein), such as directly fused to the C-terminus of the anti-C5 antibody moiety. In some embodiments, the anti-C5 antibody moiety is a full-length antibody. In some embodiments, the FH or functional fragment thereof is fused to the C-terminus of one or both heavy chains of the anti-C5 full-length antibody, either directly or via a peptide linker, such as fusing directly. In some embodiments, the fusion protein comprises a first FH or functional fragment thereof and a second FH or functional fragment thereof, wherein the first FH or functional fragment thereof is fused to the C-terminus of a first heavy chain of the anti-C5 full-length antibody, and the second FH or functional fragment thereof is fused to the C-terminus of a second heavy chain of the anti-C5 full-length antibody.

In some embodiments, the fusion protein further comprises a signal peptide at the N-terminus of one or more of its polypeptide chain(s). Any suitable signal peptide for protein/polypeptide export or expression can be used herein, including but not limited to SEQ ID NO: 91 or 92. In some embodiments, the anti-C5 antibody moiety is a full-length antibody, the FH or functional fragment thereof is fused to (e.g., direct fusion) the C-terminus of one or both (e.g., both) heavy chains of the anti-C5 full-length antibody, and the fusion protein comprises one or more signal peptides fused to the N-terminus of one or both VHs or heavy chains, and/or one or both VLs or light chains (e.g., both heavy chains and both light chains) of the anti-C5 full-length antibody. In some embodiments, the signal peptide fused to the VH(s) or the heavy chain(s) of the anti-C5 antibody or antigen-binding fragment thereof comprises the amino acid sequence of SEQ ID NO: 91. In some embodiments, the signal peptide fused to the VL(s) or the light chain(s) of the anti-C5 antibody or antigen-binding fragment thereof comprises the amino acid sequence of SEQ ID NO: 92. In some embodiments, the signal peptide is cleaved in the mature or secreted fusion protein format. In some embodiments, the fusion protein does not comprise any signal peptide.

In some embodiments, there is provided a fusion protein comprising an anti-C5 antibody moiety (e.g., anti-C5 full-length antibody) and an FH or functional fragment thereof (e.g., SCR1-5 domains of FH, such as SEQ ID NO: 85), wherein the anti-C5 antibody moiety comprises: (i) a VH-CDR1 comprising the amino acid sequence of SEQ ID NO: 3, a VH-CDR2 comprising the amino acid sequence of SEQ ID NO: 4, a VH-CDR3 comprising the amino acid sequence of SEQ ID NO: 5, a VL-CDR1 comprising the amino acid sequence of SEQ ID NO: 8, a VL-CDR2 comprising the amino acid sequence of SEQ ID NO: 9, and a VL-CDR3 comprising the amino acid sequence of SEQ ID NO: 11; (ii) a VH-CDR1 comprising the amino acid sequence of SEQ ID NO: 3, a VH-CDR2 comprising the amino acid sequence of SEQ ID NO: 4, a VH-CDR3 comprising the amino acid sequence of SEQ ID NO: 5, a VL-CDR1 comprising the amino acid sequence of SEQ ID NO: 14, a VL-CDR2 comprising the amino acid sequence of SEQ ID NO: 9, and a VL-CDR3 comprising the amino acid sequence of SEQ ID NO: 10; (iii) a VH-CDR1 comprising the amino acid sequence of SEQ ID NO: 17, a VH-CDR2 comprising the amino acid sequence of SEQ ID NO: 4, a VH-CDR3 comprising the amino acid sequence of SEQ ID NO: 5, a VL-CDR1 comprising the amino acid sequence of SEQ ID NO: 8, a VL-CDR2 comprising the amino acid sequence of SEQ ID NO: 9, and a VL-CDR3 comprising the amino acid sequence of SEQ ID NO: 10; (iv) a VH-CDR1 comprising the amino acid sequence of SEQ ID NO: 20, a VH-CDR2 comprising the amino acid sequence of SEQ ID NO: 4, a VH-CDR3 comprising the amino acid sequence of SEQ ID NO: 5, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 10; (v) a VH-CDR1 comprising the amino acid sequence of SEQ ID NO: 3, a VH-CDR2 comprising the amino acid sequence of SEQ ID NO: 26, a VH-CDR3 comprising the amino acid sequence of SEQ ID NO: 5, a VL-CDR1 comprising the amino acid sequence of SEQ ID NO: 8, a VL-CDR2 comprising the amino acid sequence of SEQ ID NO: 9, and a VL-CDR3 comprising the amino acid sequence of SEQ ID NO: 29; (vi) a VH-CDR1 comprising the amino acid sequence of SEQ ID NO: 3, a VH-CDR2 comprising the amino acid sequence of SEQ ID NO: 34, a VH-CDR3 comprising the amino acid sequence of SEQ ID NO: 5, a VL-CDR1 comprising the amino acid sequence of SEQ ID NO: 8, a VL-CDR2 comprising the amino acid sequence of SEQ ID NO: 9, and a VL-CDR3 comprising the amino acid sequence of SEQ ID NO: 10; (vii) a VH-CDR1 comprising the amino acid sequence of SEQ ID NO: 37, a VH-CDR2 comprising the amino acid sequence of SEQ ID NO: 38, a VH-CDR3 comprising the amino acid sequence of SEQ ID NO: 39, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 10; (viii) a VH-CDR1 comprising the amino acid sequence of SEQ ID NO: 42, a VH-CDR2 comprising the amino acid sequence of SEQ ID NO: 43, a VH-CDR3 comprising the amino acid sequence of SEQ ID NO: 44, a VL-CDR1 comprising the amino acid sequence of SEQ ID NO: 23, a VL-CDR2 comprising the amino acid sequence of SEQ ID NO: 9; and a VL-CDR3 comprising the amino acid sequence of SEQ ID NO: 10; (ix) a VH-CDR1 comprising the amino acid sequence of SEQ ID NO: 47, a VH-CDR2 comprising the amino acid sequence of SEQ ID NO: 48, a VH-CDR3 comprising the amino acid sequence of SEQ ID NO: 49, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 10; (x) a VH-CDR1 comprising the amino acid sequence of SEQ ID NO: 52, a VH-CDR2 comprising the amino acid sequence of SEQ ID NO: 53, a VH-CDR3 comprising the amino acid sequence of SEQ ID NO: 54, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 10; (xi) a VH-CDR1 comprising the amino acid sequence of SEQ ID NO: 47, a VH-CDR2 comprising the amino acid sequence of SEQ ID NO: 57, a VH-CDR3 comprising the amino acid sequence of SEQ ID NO: 49, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 10; (xii) a VH-CDR1 comprising the amino acid sequence of SEQ ID NO: 86, a VH-CDR2 comprising the amino acid sequence of SEQ ID NO: 57, a VH-CDR3 comprising the amino acid sequence of SEQ ID NO: 49, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96; (xiii) a VH-CDR1 comprising the amino acid sequence of SEQ ID NO: 37, a VH-CDR2 comprising the amino acid sequence of SEQ ID NO: 62, a VH-CDR3 comprising the amino acid sequence of SEQ ID NO: 39, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 10; (xiv) a VH-CDR1 comprising the amino acid sequence of SEQ ID NO: 42, a VH-CDR2 comprising the amino acid sequence of SEQ ID NO: 65, a VH-CDR3 comprising the amino acid sequence of SEQ ID NO: 44, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 10; (xv) a VH-CDR1 comprising the amino acid sequence of SEQ ID NO: 52, a VH-CDR2 comprising the amino acid sequence of SEQ ID NO: 68, a VH-CDR3 comprising the amino acid sequence of SEQ ID NO: 54, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 10; (xvi) a VH-CDR1 comprising the amino acid sequence of SEQ ID NO: 3, a VH-CDR2 comprising the amino acid sequence of SEQ ID NO: 4, a VH-CDR3 comprising the amino acid sequence of SEQ ID NO: 5, a VL-CDR1 comprising the amino acid sequence of SEQ ID NO: 8, a VL-CDR2 comprising the amino acid sequence of SEQ ID NO: 9, and a VL-CDR3 comprising the amino acid sequence of SEQ ID NO: 10; (xvii) a VH-CDR1 comprising the amino acid sequence of SEQ ID NO: 94, a VH-CDR2 comprising the amino acid sequence of SEQ ID NO: 4, a VH-CDR3 comprising the amino acid sequence of SEQ ID NO: 5, a VL-CDR1 comprising the amino acid sequence of SEQ ID NO: 8, a VL-CDR2 comprising the amino acid sequence of SEQ ID NO: 9, and a VL-CDR3 comprising the amino acid sequence of SEQ ID NO: 96; (xviii) a VH-CDR1 comprising the amino acid sequence of SEQ ID NO: 94, a VH-CDR2 comprising the amino acid sequence of SEQ ID NO: 4, a VH-CDR3 comprising the amino acid sequence of SEQ ID NO: 5, a VL-CDR1 comprising the amino acid sequence of SEQ ID NO: 8, a VL-CDR2 comprising the amino acid sequence of SEQ ID NO: 9, and a VL-CDR3 comprising the amino acid sequence of SEQ ID NO: 97; (xix) a VH-CDR1 comprising the amino acid sequence of SEQ ID NO: 94, a VH-CDR2 comprising the amino acid sequence of SEQ ID NO: 4, a VH-CDR3 comprising the amino acid sequence of SEQ ID NO: 5, a VL-CDR1 comprising the amino acid sequence of SEQ ID NO: 14, a VL-CDR2 comprising the amino acid sequence of SEQ ID NO: 9, and a VL-CDR3 comprising the amino acid sequence of SEQ ID NO: 96; (xx) a VH-CDR1 comprising the amino acid sequence of SEQ ID NO: 17, a VH-CDR2 comprising the amino acid sequence of SEQ ID NO: 4, a VH-CDR3 comprising the amino acid sequence of SEQ ID NO: 5, a VL-CDR1 comprising the amino acid sequence of SEQ ID NO: 8, a VL-CDR2 comprising the amino acid sequence of SEQ ID NO: 9, and a VL-CDR3 comprising the amino acid sequence of SEQ ID NO: 96; (xxi) a VH-CDR1 comprising the amino acid sequence of SEQ ID NO: 102, a VH-CDR2 comprising the amino acid sequence of SEQ ID NO: 4, a VH-CDR3 comprising the amino acid sequence of SEQ ID NO: 5, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96; (xxii) a VH-CDR1 comprising the amino acid sequence of SEQ ID NO: 94, a VH-CDR2 comprising the amino acid sequence of SEQ ID NO: 26, a VH-CDR3 comprising the amino acid sequence of SEQ ID NO: 5, a VL-CDR1 comprising the amino acid sequence of SEQ ID NO: 8, a VL-CDR2 comprising the amino acid sequence of SEQ ID NO: 9, and a VL-CDR3 comprising the amino acid sequence of SEQ ID NO: 105; (xxiii) a VH-CDR1 comprising the amino acid sequence of SEQ ID NO: 94, a VH-CDR2 comprising the amino acid sequence of SEQ ID NO: 34, a VH-CDR3 comprising the amino acid sequence of SEQ ID NO: 5, a VL-CDR1 comprising the amino acid sequence of SEQ ID NO: 8, a VL-CDR2 comprising the amino acid sequence of SEQ ID NO: 9, and a VL-CDR3 comprising the amino acid sequence of SEQ ID NO: 96; (xxiv) a VH-CDR1 comprising the amino acid sequence of SEQ ID NO: 108, a VH-CDR2 comprising the amino acid sequence of SEQ ID NO: 38, a VH-CDR3 comprising the amino acid sequence of SEQ ID NO: 39, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96; (xxv) a VH-CDR1 comprising the amino acid sequence of SEQ ID NO: 108, a VH-CDR2 comprising the amino acid sequence of SEQ ID NO: 43, a VH-CDR3 comprising the amino acid sequence of SEQ ID NO: 44, a VL-CDR1 comprising the amino acid sequence of SEQ ID NO: 23, a VL-CDR2 comprising the amino acid sequence of SEQ ID NO: 9; and a VL-CDR3 comprising the amino acid sequence of SEQ ID NO: 96; (xxvi) a VH-CDR1 comprising the amino acid sequence of SEQ ID NO: 86, a VH-CDR2 comprising the amino acid sequence of SEQ ID NO: 48, a VH-CDR3 comprising the amino acid sequence of SEQ ID NO: 49, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96; (xxvii) a VH-CDR1 comprising the amino acid sequence of SEQ ID NO: 86, a VH-CDR2 comprising the amino acid sequence of SEQ ID NO: 53, a VH-CDR3 comprising the amino acid sequence of SEQ ID NO: 54, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96; (xxviii) a VH-CDR1 comprising the amino acid sequence of SEQ ID NO: 108, a VH-CDR2 comprising the amino acid sequence of SEQ ID NO: 62, a VH-CDR3 comprising the amino acid sequence of SEQ ID NO: 39, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96; (xxix) a VH-CDR1 comprising the amino acid sequence of SEQ ID NO: 108, a VH-CDR2 comprising the amino acid sequence of SEQ ID NO: 65, a VH-CDR3 comprising the amino acid sequence of SEQ ID NO: 44, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96; or (xxx) a VH-CDR1 comprising the amino acid sequence of SEQ ID NO: 86, a VH-CDR2 comprising the amino acid sequence of SEQ ID NO: 68, a VH-CDR3 comprising the amino acid sequence of SEQ ID NO: 54, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96. In some embodiments, the anti-C5 antibody moiety comprises: (i) a VH comprising the amino acid sequence of SEQ ID NO: 93, and a VL comprising the amino acid sequence of SEQ ID NO: 98; (ii) a VH comprising the amino acid sequence of SEQ ID NO: 93, and a VL comprising the amino acid sequence of SEQ ID NO: 99; (iii) a VH comprising the amino acid sequence of SEQ ID NO: 101, and a VL comprising the amino acid sequence of SEQ ID NO: 95; (iv) a VH comprising the amino acid sequence of SEQ ID NO: 103, and a VL comprising the amino acid sequence of SEQ ID NO: 88; (v) a VH comprising the amino acid sequence of SEQ ID NO: 104, and a VL comprising the amino acid sequence of SEQ ID NO: 106; (vi) a VH comprising the amino acid sequence of SEQ ID NO: 107, and a VL comprising the amino acid sequence of SEQ ID NO: 95; (vii) a VH comprising the amino acid sequence of SEQ ID NO: 109, and a VL comprising the amino acid sequence of SEQ ID NO: 88; (viii) a VH comprising the amino acid sequence of SEQ ID NO: 110, and a VL comprising the amino acid sequence of SEQ ID NO: 88; (ix) a VH comprising the amino acid sequence of SEQ ID NO: 111, and a VL comprising the amino acid sequence of SEQ ID NO: 88; (x) a VH comprising the amino acid sequence of SEQ ID NO: 112, and a VL comprising the amino acid sequence of SEQ ID NO: 88; (xi) a VH comprising the amino acid sequence of SEQ ID NO: 87, and a VL comprising the amino acid sequence of SEQ ID NO: 88; (xii) a VH comprising the amino acid sequence of SEQ ID NO: 113, and a VL comprising the amino acid sequence of SEQ ID NO: 88; (xiii) a VH comprising the amino acid sequence of SEQ ID NO: 114, and a VL comprising the amino acid sequence of SEQ ID NO: 88; (xiv) a VH comprising the amino acid sequence of SEQ ID NO: 115, and a VL comprising the amino acid sequence of SEQ ID NO: 88; or (xv) a VH comprising the amino acid sequence of SEQ ID NO: 93, and a VL comprising the amino acid sequence of SEQ ID NO: 95. In some embodiments, there is provided a fusion protein comprising an anti-C5 antibody moiety (e.g., anti-C5 full-length antibody) and an FH or functional fragment thereof (e.g., SCR1-5 domains of FH, such as SEQ ID NO: 85), wherein the anti-C5 antibody moiety comprises: (i) a VH-CDR1 comprising the amino acid sequence of SEQ ID NO: 47, a VH-CDR2 comprising the amino acid sequence of SEQ ID NO: 57, a VH-CDR3 comprising the amino acid sequence of SEQ ID NO: 49, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 10; or (ii) a VH-CDR1 comprising the amino acid sequence of SEQ ID NO: 86, a VH-CDR2 comprising the amino acid sequence of SEQ ID NO: 57, a VH-CDR3 comprising the amino acid sequence of SEQ ID NO: 49, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96. In some embodiments, there is provided a fusion protein comprising an anti-C5 antibody moiety (e.g., anti-C5 full-length antibody) and an FH or functional fragment thereof (e.g., SCR1-5 domains of FH, such as SEQ ID NO: 85), wherein the anti-C5 antibody moiety comprises a VH comprising the amino acid sequence of SEQ ID NO: 87, and a VL comprising the amino acid sequence of SEQ ID NO: 88. In some embodiments, the FH or functional fragment thereof (e.g., SCR1-5 domains of FH, such as SEQ ID NO: 85) is fused to the C-terminus of the anti-C5 antibody moiety. In some embodiments, the anti-C5 antibody moiety is a full-length antibody. In some embodiments, the anti-C5 full-length antibody comprises an Fc fragment derived from human IgG4, such as an Fc fragment comprising the amino acid sequence of any of SEQ ID NOs: 32, 33, and 61, e.g., SEQ ID NO: 61. In some embodiments, the FH or functional fragment thereof (e.g., SCR1-5 domains of FH, such as SEQ ID NO: 85) is fused to (e.g., direct fusion) the C-terminus of one or both (e.g., both) heavy chains of the anti-C5 full-length antibody. In some embodiments, the anti-C5 full-length antibody comprises a heavy chain comprising the amino acid sequence of any of SEQ ID NOs: 120, 122, 124, and 126, and a light chain comprising the amino acid sequence of SEQ ID NO: 90. In some embodiments, the anti-C5 full-length antibody comprises a heavy chain comprising the amino acid sequence of SEQ ID NO: 120, and a light chain comprising the amino acid sequence of SEQ ID NO: 90.

In some embodiments, there is provided a fusion protein comprising an anti-C5 full-length antibody and an FH or functional fragment thereof (e.g., SCR1-5 domains of FH, such as SEQ ID NO: 85); wherein the FH or functional fragment thereof is fused to (e.g., direct fusion) the C-terminus of one or both (e.g., both) of the heavy chains of the anti-C5 full-length antibody; and wherein the anti-C5 full-length antibody comprises: (i) a VH-CDR1 comprising the amino acid sequence of SEQ ID NO: 47, a VH-CDR2 comprising the amino acid sequence of SEQ ID NO: 57, a VH-CDR3 comprising the amino acid sequence of SEQ ID NO: 49, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 10; or (ii) a VH-CDR1 comprising the amino acid sequence of SEQ ID NO: 86, a VH-CDR2 comprising the amino acid sequence of SEQ ID NO: 57, a VH-CDR3 comprising the amino acid sequence of SEQ ID NO: 49, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96. In some embodiments, there is provided a fusion protein comprising an anti-C5 full-length antibody and an FH or functional fragment thereof (e.g., SCR1-5 domains of FH, such as SEQ ID NO: 85); wherein the FH or functional fragment thereof is fused to (e.g., direct fusion) the C-terminus of one or both (e.g., both) of the heavy chains of the anti-C5 full-length antibody; and wherein the anti-C5 full-length antibody comprises a VH comprising the amino acid sequence of SEQ ID NO: 87, and a VL comprising the amino acid sequence of SEQ ID NO: 88. In some embodiments, the anti-C5 full-length antibody comprises an Fc fragment comprising the amino acid sequence of SEQ ID NO: 61. In some embodiments, there is provided a fusion protein comprising an anti-C5 full-length antibody and an FH or functional fragment thereof (e.g., SCR1-5 domains of FH, such as SEQ ID NO: 85); wherein the FH or functional fragment thereof is fused to (e.g., direct fusion) the C-terminus of one or both (e.g., both) of the heavy chains of the anti-C5 full-length antibody; and wherein the anti-C5 full-length antibody comprises a heavy chain comprising the amino acid sequence of SEQ ID NO: 120, and a light chain comprising the amino acid sequence of SEQ ID NO: 90.

In some embodiments, the anti-C5 antibody moiety is a full-length antibody. In some embodiments, the anti-C5 full-length antibody comprises an Fc fragment comprising the amino acid sequence of SEQ ID NO: 61. In some embodiments, the FH or functional fragment thereof (e.g., SCR1-5 domains of FH, such as SEQ ID NO: 85) is fused to (e.g., direct fusion) the C-terminus of one or both (e.g., both) heavy chains of the anti-C5 full-length antibody. In some embodiments, there is provided a fusion protein comprising an anti-C5 full-length antibody (such as any of the anti-C5 full-length antibodies described herein) and an FH or functional fragment thereof (e.g., SCR1-5 domains of FH, such as SEQ ID NO: 85); wherein the FH or functional fragment thereof is fused to (e.g., direct fusion) the C-terminus of one of the heavy chains of the anti-C5 full-length antibody. In some embodiments, there is provided a fusion protein comprising an anti-C5 full-length antibody and an FH or functional fragment thereof (e.g., SCR1-5 domains of FH, such as SEQ ID NO: 85); wherein the FH or functional fragment thereof is fused to (e.g., direct fusion) the C-terminus of one of the heavy chains of the anti-C5 full-length antibody; and wherein the anti-C5 full-length antibody comprises a heavy chain comprising the amino acid sequence of any of SEQ ID NOs: 120, 122, 124, and 126, and a light chain comprising the amino acid sequence of SEQ ID NO: 90. In some embodiments, there is provided a fusion protein comprising a first FH or functional fragment thereof (e.g., SCR1-5 domains of FH, such as SEQ ID NO: 85), a second FH or functional fragment thereof (e.g., SCR1-5 domains of FH, such as SEQ ID NO: 85), and an anti-C5 full-length antibody (such as any of the anti-C5 full-length antibodies described herein); wherein the first FH or functional fragment thereof is fused to (e.g., direct fusion) the C-terminus of a first heavy chain of the anti-C5 full-length antibody, and the second FH or functional fragment thereof is fused to (e.g., direct fusion) the C-terminus of a second heavy chain of the anti-C5 full-length antibody. In some embodiments, there is provided a fusion protein comprising a first FH or functional fragment thereof (e.g., SCR1-5 domains of FH, such as SEQ ID NO: 85), a second FH or functional fragment thereof (e.g., SCR1-5 domains of FH, such as SEQ ID NO: 85), and an anti-C5 full-length antibody; wherein the first FH or functional fragment thereof is fused to (e.g., direct fusion) the C-terminus of a first heavy chain of the anti-C5 full-length antibody, and the second FH or functional fragment thereof is fused to (e.g., direct fusion) the C-terminus of a second heavy chain of the anti-C5 full-length antibody; and wherein the anti-C5 full-length antibody comprises a heavy chain comprising the amino acid sequence of any of SEQ ID NOs: 120, 122, 124, and 126, and a light chain comprising the amino acid sequence of SEQ ID NO: 90.

In some embodiments, there is provided a fusion protein comprising i) an anti-C5 full-length antibody, ii) a first functional fragment of FH fused to the C-terminus of a first heavy chain of the anti-C5 full-length antibody, and iii) a second functional fragment of FH fused to the C-terminus of a second heavy chain of the anti-C5 full-length antibody; wherein the anti-C5 full-length antibody comprises two heavy chains each comprising the amino acid sequence of SEQ ID NO: 120, and two light chains each comprising the amino acid sequence of SEQ ID NO: 90; and wherein each heavy chain fused to the functional fragment of FH comprises the amino acid sequence of SEQ ID NO: 89. This fusion protein is hereinafter referred to as “FMEH-IgG4-PLA-FH”. In some embodiments, the fusion protein further comprises a signal peptide (e.g., SEQ ID NO: 91 or 92) at the N-terminus of one or both heavy chains and/or one or both light chains of the anti-C5 full-length antibody, such as both heavy chains and both light chains of the anti-C5 full-length antibody. In some embodiments, there is provided a fusion protein comprising i) an anti-C5 full-length antibody, ii) a first functional fragment of FH fused to the C-terminus of a first heavy chain of the anti-C5 full-length antibody, and iii) a second functional fragment of FH fused to the C-terminus of a second heavy chain of the anti-C5 full-length antibody; wherein the anti-C5 full-length antibody comprises two heavy chains each comprising the amino acid sequence of SEQ ID NO: 119, and two light chains each comprising the amino acid sequence of SEQ ID NO: 74; and wherein each heavy chain fused to the functional fragment of FH comprises the amino acid sequence of SEQ ID NO: 72.

In some embodiments, there is provided a fusion protein comprising i) an anti-C5 full-length antibody, ii) a first functional fragment of FH fused to the C-terminus of a first heavy chain of the anti-C5 full-length antibody, and iii) a second functional fragment of FH fused to the C-terminus of a second heavy chain of the anti-C5 full-length antibody; wherein the anti-C5 full-length antibody comprises two heavy chains each comprising the amino acid sequence of SEQ ID NO: 122, and two light chains each comprising the amino acid sequence of SEQ ID NO: 90; and wherein each heavy chain fused to the functional fragment of FH comprises the amino acid sequence of SEQ ID NO: 116. In some embodiments, the fusion protein further comprises a signal peptide (e.g., SEQ ID NO: 91 or 92) at the N-terminus of one or both heavy chains and/or one or both light chains of the anti-C5 full-length antibody, such as both heavy chains and both light chains of the anti-C5 full-length antibody. In some embodiments, there is provided a fusion protein comprising i) an anti-C5 full-length antibody, ii) a first functional fragment of FH fused to the C-terminus of a first heavy chain of the anti-C5 full-length antibody, and iii) a second functional fragment of FH fused to the C-terminus of a second heavy chain of the anti-C5 full-length antibody; wherein the anti-C5 full-length antibody comprises two heavy chains each comprising the amino acid sequence of SEQ ID NO: 121, and two light chains each comprising the amino acid sequence of SEQ ID NO: 74; and wherein each heavy chain fused to the functional fragment of FH comprises the amino acid sequence of SEQ ID NO: 76.

In some embodiments, there is provided a fusion protein comprising i) an anti-C5 full-length antibody, ii) a first functional fragment of FH fused to the C-terminus of a first heavy chain of the anti-C5 full-length antibody, and iii) a second functional fragment of FH fused to the C-terminus of a second heavy chain of the anti-C5 full-length antibody; wherein the anti-C5 full-length antibody comprises two heavy chains each comprising the amino acid sequence of SEQ ID NO: 124, and two light chains each comprising the amino acid sequence of SEQ ID NO: 90; and wherein each heavy chain fused to the functional fragment of FH comprises the amino acid sequence of SEQ ID NO: 117. In some embodiments, the fusion protein further comprises a signal peptide (e.g., SEQ ID NO: 91 or 92) at the N-terminus of one or both heavy chains and/or one or both light chains of the anti-C5 full-length antibody, such as both heavy chains and both light chains of the anti-C5 full-length antibody. In some embodiments, there is provided a fusion protein comprising i) an anti-C5 full-length antibody, ii) a first functional fragment of FH fused to the C-terminus of a first heavy chain of the anti-C5 full-length antibody, and iii) a second functional fragment of FH fused to the C-terminus of a second heavy chain of the anti-C5 full-length antibody; wherein the anti-C5 full-length antibody comprises two heavy chains each comprising the amino acid sequence of SEQ ID NO: 123, and two light chains each comprising the amino acid sequence of SEQ ID NO: 74; and wherein each heavy chain fused to the functional fragment of FH comprises the amino acid sequence of SEQ ID NO: 78.

In some embodiments, there is provided a fusion protein comprising i) an anti-C5 full-length antibody, ii) a first functional fragment of FH fused to the C-terminus of a first heavy chain of the anti-C5 full-length antibody, and iii) a second functional fragment of FH fused to the C-terminus of a second heavy chain of the anti-C5 full-length antibody; wherein the anti-C5 full-length antibody comprises two heavy chains each comprising the amino acid sequence of SEQ ID NO: 126, and two light chains each comprising the amino acid sequence of SEQ ID NO: 90; and wherein each heavy chain fused to the functional fragment of FH comprises the amino acid sequence of SEQ ID NO: 118. In some embodiments, the fusion protein further comprises a signal peptide (e.g., SEQ ID NO: 91 or 92) at the N-terminus of one or both heavy chains and/or one or both light chains of the anti-C5 full-length antibody, such as both heavy chains and both light chains of the anti-C5 full-length antibody. In some embodiments, there is provided a fusion protein comprising i) an anti-C5 full-length antibody, ii) a first functional fragment of FH fused to the C-terminus of a first heavy chain of the anti-C5 full-length antibody, and iii) a second functional fragment of FH fused to the C-terminus of a second heavy chain of the anti-C5 full-length antibody; wherein the anti-C5 full-length antibody comprises two heavy chains each comprising the amino acid sequence of SEQ ID NO: 125, and two light chains each comprising the amino acid sequence of SEQ ID NO: 74; and wherein each heavy chain fused to the functional fragment of FH comprises the amino acid sequence of SEQ ID NO: 80.

### Anti-C5 Antibody Moiety

The fusion protein descried herein comprises an antibody moiety that specifically binds to C5. In some embodiments, the anti-C5 antibody moiety is a chimeric antibody. In further embodiments, the anti-C5 antibody moiety is a humanized antibody. In some embodiments, the anti-C5 antibody moiety is a full-length antibody, a Fab, a Fab′, a F(ab)₂, a F(ab′)₂, an scFv, or a combination thereof. In some embodiments, the anti-C5 antibody moiety is an antibody fragment (e.g., Fab, scFv). In some embodiments, the anti-C5 antibody moiety is a full-length antibody, such as a full-length antibody comprising an Fc fragment derived from IgG4 (e.g., an IgG4 Fc comprising a PLA mutation). In some embodiments, the IgG4 Fc fragment comprises the amino acid sequence of any of SEQ ID NOs: 32, 33, and 61, such as SEQ ID NO: 61. In some embodiments, the binding of the anti-C5 antibody moiety to C5 is pH-dependent, and wherein the anti-C5 antibody moiety binds more strongly to C5 at a neutral pH (e.g., about pH 7.4) than it does at an acidic pH (e.g., about pH 5.8). In some embodiments, the C5 is human C5. In some embodiments, an anti-C5 antibody moiety comprises (or consists of, or consists essentially of) an anti-C5 antibody or antigen-binding fragment thereof. In some embodiments, the anti-C5 antibody moiety consists of any of the anti-C5 antibodies or antigen-binding fragments thereof described herein.

In some embodiments, the anti-C5 antibody or antigen-binding fragment thereof binds to an epitope in the α-chain of C5, and/or an epitope in the j-chain of C5.

It is to be understood that the anti-C5 antibody moiety described herein can derive from any of the anti-C5 antibodies or antibody fragments thereof discussed below in more detail.

In some embodiments, the anti-C5 antibody moiety exhibits pH-dependent binding to C5. In some embodiments, the pH-dependent anti-C5 antibody moiety binds more strongly to C5 at a more neutral pH (e.g., about pH 7.4; such as that found in the blood) than it does at a more acidic pH (e.g., about pH 5.8; such as that found in the endosome). In some embodiments, the pH-dependent anti-C5 antibody moiety is a variant (e.g., comprising one or more amino acid substitutions, such as conserved substitution) of humanized anti-C5 antibody comprising a VH comprising the amino acid sequence of SEQ ID NO: 2 and a VL comprising the amino acid sequence of SEQ ID NO: 7.

In some embodiments, binding of the anti-C5 antibody or fragment of the antibody to human-C5 is associated with a reduction in the generation of C5a or C5b and the formation of MAC in the complement activation pathway in an intact organism. In some embodiments, the anti-C5 antibody moiety is capable of binding to human C5. In some embodiments, the anti-C5 antibody or antibody fragment binds to a relevant portion or fraction or epitope of the human-C5; and the binding of the anti-C5 antibody or antibody fragment thereof to the relevant portion of the human-C5 is associated with a reduction in the generation of C5a or C5b and the formation of MAC in an intact organism.

In some embodiments, the anti-C5 antibody or antibody fragment thereof is further conjugated to a protein, a peptide or another compound. In some embodiments, the anti-C5 antibody or antibody fragment thereof, is conjugated to a protein, a peptide, or other compound. In some embodiments, the protein, peptide, or other compound to which the anti-C5 antibody or antibody fragment thereof is conjugated is a targeting moiety (i.e., the targeting moiety specifically binds to a molecule other than human-C5). In some embodiments, the protein, peptide, or other compound to which the anti-C5 antibody or antibody fragment thereof is conjugated to is an effector molecule (e.g., a cytotoxic molecule).

In various embodiments, any of the anti-C5 antibodies described herein, having any of the variable regions described herein, may comprise an Fc fragment or Fc domain. For example, in some embodiments, an anti-C5 antibody described herein, comprises an Fc fragment of an immunoglobulin. Exemplary immunoglobulins include, but is not limited to, IgG1, IgG2, IgG3, IgG4, IgM, IgA, IgE, and IgD. In some embodiments, the anti-C5 antibody comprises an Fc of human IgG4. SEQ ID NO: 32 is an example amino acid sequence of a human IgG4 Fc fragment. In some embodiments, the anti-C5 antibody comprises a human IgG4 Fc fragment having an S108P mutation relative to SEQ ID NO: 32. SEQ ID NO: 33 is an exemplary amino acid sequence of a human IgG4 Fc fragment having an S108P mutation relative to SEQ ID NO: 32. In some embodiments, the anti-C5 antibody comprises a human IgG4 Fc fragment having one or more of: an S108P mutation, a M308L mutation, and a N314A mutation, relative to SEQ ID NO: 32. In some embodiments, the anti-C5 antibody comprises a human IgG4 Fc fragment having an S108P mutation, a M308L mutation, and a N314A mutation, relative to SEQ ID NO: 32 (also referred to herein as having an IgG4 Fc “PLA” mutation). SEQ ID NO: 61 is an exemplary amino acid sequence of a human IgG4 Fc fragment having an S108P mutation, a M308L mutation, and a N314A mutation relative to SEQ ID NO: 32.

In some embodiments, the anti-C5 antibody or antigen-binding fragment thereof comprises: a VH-CDR1 comprising the amino acid sequence of SEQ ID NO: 3; a VH-CDR2 comprising the amino acid sequence of SEQ ID NO: 4; a VH-CDR3 comprising the amino acid sequence of SEQ ID NO: 5; a VL-CDR1 comprising the amino acid sequence of SEQ ID NO: 8; a VL-CDR2 comprising the amino acid sequence of SEQ ID NO: 9; and a VL-CDR3 comprising the amino acid sequence of SEQ ID NO: 10, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 3, or a variant thereof comprising up to about 3 (such as about any of 1, 2, or 3) amino acid substitutions (e.g., conserved substitution); a VH-CDR2 comprising the amino acid sequence of SEQ ID NO: 4, or a variant thereof comprising up to about 3 (such as about any of 1, 2, or 3) amino acid substitutions (e.g., conserved substitution); a VH-CDR3 comprising the amino acid sequence of SEQ ID NO: 5, or a variant thereof comprising up to about 3 (such as about any of 1, 2, or 3) amino acid substitutions (e.g., conserved substitution); a VL-CDR1 comprising the amino acid sequence of SEQ ID NO: 8, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 10, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 3; a VH-CDR2 comprising the amino acid sequence of SEQ ID NO: 4; a VH-CDR3 comprising the amino acid sequence of SEQ ID NO: 5; a VL-CDR1 comprising the amino acid sequence of SEQ ID NO: 8; a VL-CDR2 comprising the amino acid sequence of SEQ ID NO: 9; and a VL-CDR3 comprising the amino acid sequence of SEQ ID NO: 10.

In some embodiments, the anti-C5 antibody or antigen-binding fragment thereof comprises: a VH-CDR1 comprising the amino acid sequence of SEQ ID NO: 94; a VH-CDR2 comprising the amino acid sequence of SEQ ID NO: 4; a VH-CDR3 comprising the amino acid sequence of SEQ ID NO: 5; a VL-CDR1 comprising the amino acid sequence of SEQ ID NO: 8; a VL-CDR2 comprising the amino acid sequence of SEQ ID NO: 9; and a VL-CDR3 comprising the amino acid sequence of SEQ ID NO: 96, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 94, or a variant thereof comprising up to about 3 (such as about any of 1, 2, or 3) amino acid substitutions (e.g., conserved substitution); a VH-CDR2 comprising the amino acid sequence of SEQ ID NO: 4, or a variant thereof comprising up to about 3 (such as about any of 1, 2, or 3) amino acid substitutions (e.g., conserved substitution); a VH-CDR3 comprising the amino acid sequence of SEQ ID NO: 5, or a variant thereof comprising up to about 3 (such as about any of 1, 2, or 3) amino acid substitutions (e.g., conserved substitution); a VL-CDR1 comprising the amino acid sequence of SEQ ID NO: 8, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 96, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 94; a VH-CDR2 comprising the amino acid sequence of SEQ ID NO: 4; a VH-CDR3 comprising the amino acid sequence of SEQ ID NO: 5; a VL-CDR1 comprising the amino acid sequence of SEQ ID NO: 8; a VL-CDR2 comprising the amino acid sequence of SEQ ID NO: 9; and a VL-CDR3 comprising the amino acid sequence of SEQ ID NO: 96.

In some embodiments, the anti-C5 antibody or antigen-binding fragment thereof comprises a VH comprising the amino acid sequence of SEQ ID NO: 2, or a variant thereof comprising at least about 80% (e.g., at least about any of 85%, 90%, 95%, 96%, 97%, 98%, 99% or more) sequence identity to SEQ ID NO: 2, and a VL comprising the amino acid sequence of SEQ ID NO: 7, or a variant thereof comprising at least about 80% (e.g., at least about any of 85%, 90%, 95%, 96%, 97%, 98%, 99% or more) sequence identity to SEQ ID NO: 7. In some embodiments, the anti-C5 antibody or antigen-binding fragment thereof comprises a VH comprising the amino acid sequence of SEQ ID NO: 2, and a VL comprising the amino acid sequence of SEQ ID NO: 7. In some embodiments, the anti-C5 antibody or antigen-binding fragment thereof comprises a VH comprising the amino acid sequence of SEQ ID NO: 93, or a variant thereof comprising at least about 80% (e.g., at least about any of 85%, 90%, 95%, 96%, 97%, 98%, 99% or more) sequence identity to SEQ ID NO: 93, and a VL comprising the amino acid sequence of SEQ ID NO: 95, or a variant thereof comprising at least about 80% (e.g., at least about any of 85%, 90%, 95%, 96%, 97%, 98%, 99% or more) sequence identity to SEQ ID NO: 95. In some embodiments, the anti-C5 antibody or antigen-binding fragment thereof comprises a VH comprising the amino acid sequence of SEQ ID NO: 93, and a VL comprising the amino acid sequence of SEQ ID NO: 95. In some embodiments, the VH and VL do not comprise a signal peptide at the N-terminus.

In some embodiments, the anti-C5 antibody comprises a heavy chain comprising the amino acid sequence of SEQ ID NO: 2, or a variant thereof comprising at least about 80% (e.g., at least about any of 85%, 90%, 95%, 96%, 97%, 98%, 99% or more) sequence identity to SEQ ID NO: 2. In some embodiments, the anti-C5 antibody comprises a light chain comprising the amino acid sequence of SEQ ID NO: 7, or a variant thereof comprising at least about 80% (e.g., at least about any of 85%, 90%, 95%, 96%, 97%, 98%, 99% or more) sequence identity to SEQ ID NO: 7. In some embodiments, the anti-C5 antibody is humanized. In some embodiments, the anti-C5 antibody is a chimeric antibody. In some embodiments, the anti-C5 antibody is mAb 2G1, or a variant thereof (e.g., humanized 2G1). In some embodiments, the anti-C5 antibody mAb 2G1 comprises i) a heavy chain comprising the amino acid sequence of SEQ ID NO: 2 and a light chain comprising the amino acid sequence of SEQ ID NO: 7; or ii) a VH comprising the amino acid sequence of SEQ ID NO: 93 and a VL comprising the amino acid sequence of SEQ ID NO: 95. In some embodiments, the anti-C5 antibody comprises an Fc fragment. In some embodiments, the anti-C5 antibody comprises a human IgG4 Fc fragment or variant thereof. In some embodiments, the anti-C5 antibody mAb 2G1 comprises i) a heavy chain comprising the amino acid sequence of SEQ ID NO: 2 and a light chain comprising the amino acid sequence of SEQ ID NO: 7; or ii) a VH comprising the amino acid sequence of SEQ ID NO: 93 and a VL comprising the amino acid sequence of SEQ ID NO: 95; and a human IgG4 Fc fragment comprising the amino acid sequence of SEQ ID NO: 32. In some embodiments, the anti-C5 antibody comprises a human IgG4 Fc fragment having an S108P mutation relative to SEQ ID NO: 32, or a variant thereof. In some embodiments, the anti-C5 antibody mAb 2G1 comprises i) a heavy chain comprising the amino acid sequence of SEQ ID NO: 2 and a light chain comprising the amino acid sequence of SEQ ID NO: 7; or ii) a VH comprising the amino acid sequence of SEQ ID NO: 93 and a VL comprising the amino acid sequence of SEQ ID NO: 95; and a human IgG4 Fc fragment having an S108P mutation relative to SEQ ID NO: 32, comprising the amino acid sequence of SEQ ID NO: 33. In some embodiments, the anti-C5 antibody comprises a human IgG4 Fc fragment having an S108P mutation, a M308L mutation, and a N314A mutation relative to SEQ ID NO: 32, or a variant thereof. In some embodiments, the anti-C5 antibody mAb 2G1 comprises i) a heavy chain comprising the amino acid sequence of SEQ ID NO: 2 and a light chain comprising the amino acid sequence of SEQ ID NO: 7; or ii) a VH comprising the amino acid sequence of SEQ ID NO: 93 and a VL comprising the amino acid sequence of SEQ ID NO: 95; and a human IgG4 Fc fragment having an S108P mutation, a M308L mutation, and a N314A mutation relative to SEQ ID NO: 32, comprising the amino acid sequence of SEQ ID NO: 61.

In some embodiments, the anti-C5 antibody or antigen-binding fragment thereof comprises: a VH-CDR1 comprising the amino acid sequence of SEQ ID NO: 3; a VH-CDR2 comprising the amino acid sequence of SEQ ID NO: 4; a VH-CDR3 comprising the amino acid sequence of SEQ ID NO: 5; a VL-CDR1 comprising the amino acid sequence of SEQ ID NO: 8; a VL-CDR2 comprising the amino acid sequence of SEQ ID NO: 9; and a VL-CDR3 comprising the amino acid sequence of SEQ ID NO: 11, or a variant thereof comprising up to 5 (e.g., 5, 4, 3, 2, or 1) amino acid variations (e.g., insertion, deletion, substitution, e.g., conserved substitution) in one or more of the CDRs.

In some embodiments, the anti-C5 antibody or antigen-binding fragment thereof comprises: a VH-CDR1 comprising the amino acid sequence of SEQ ID NO: 3, or a variant thereof comprising up to about 3 (such as about any of 1, 2, or 3) amino acid substitutions (e.g., conserved substitution); a VH-CDR2 comprising the amino acid sequence of SEQ ID NO: 4, or a variant thereof comprising up to about 3 (such as about any of 1, 2, or 3) amino acid substitutions (e.g., conserved substitution); a VH-CDR3 comprising the amino acid sequence of SEQ ID NO: 5, or a variant thereof comprising up to about 3 (such as about any of 1, 2, or 3) amino acid substitutions (e.g., conserved substitution); a VL-CDR1 comprising the amino acid sequence of SEQ ID NO: 8, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 11, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 3; a VH-CDR2 comprising the amino acid sequence of SEQ ID NO: 4; a VH-CDR3 comprising the amino acid sequence of SEQ ID NO: 5; a VL-CDR1 comprising the amino acid sequence of SEQ ID NO: 8; a VL-CDR2 comprising the amino acid sequence of SEQ ID NO: 9; and a VL-CDR3 comprising the amino acid sequence of SEQ ID NO: 11.

In some embodiments, the anti-C5 antibody or antigen-binding fragment thereof comprises: a VH-CDR1 comprising the amino acid sequence of SEQ ID NO: 94; a VH-CDR2 comprising the amino acid sequence of SEQ ID NO: 4; a VH-CDR3 comprising the amino acid sequence of SEQ ID NO: 5; a VL-CDR1 comprising the amino acid sequence of SEQ ID NO: 8; a VL-CDR2 comprising the amino acid sequence of SEQ ID NO: 9; and a VL-CDR3 comprising the amino acid sequence of SEQ ID NO: 97, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 94, or a variant thereof comprising up to about 3 (such as about any of 1, 2, or 3) amino acid substitutions (e.g., conserved substitution); a VH-CDR2 comprising the amino acid sequence of SEQ ID NO: 4, or a variant thereof comprising up to about 3 (such as about any of 1, 2, or 3) amino acid substitutions (e.g., conserved substitution); a VH-CDR3 comprising the amino acid sequence of SEQ ID NO: 5, or a variant thereof comprising up to about 3 (such as about any of 1, 2, or 3) amino acid substitutions (e.g., conserved substitution); a VL-CDR1 comprising the amino acid sequence of SEQ ID NO: 8, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 97, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 94; a VH-CDR2 comprising the amino acid sequence of SEQ ID NO: 4; a VH-CDR3 comprising the amino acid sequence of SEQ ID NO: 5; a VL-CDR1 comprising the amino acid sequence of SEQ ID NO: 8; a VL-CDR2 comprising the amino acid sequence of SEQ ID NO: 9; and a VL-CDR3 comprising the amino acid sequence of SEQ ID NO: 97.

In some embodiments, the anti-C5 antibody or antigen-binding fragment thereof comprises a VH comprising the amino acid sequence of SEQ ID NO: 2, or a variant thereof comprising at least about 80% (e.g., at least about any of 85%, 90%, 95%, 96%, 97%, 98%, 99% or more) sequence identity to SEQ ID NO: 2, and a VL comprising the amino acid sequence of SEQ ID NO: 13, or a variant thereof comprising at least about 80% (e.g., at least about any of 85%, 90%, 95%, 96%, 97%, 98%, 99% or more) sequence identity to SEQ ID NO: 13. In some embodiments, the anti-C5 antibody or antigen-binding fragment thereof comprises a VH comprising the amino acid sequence of SEQ ID NO: 2, and a VL comprising the amino acid sequence of SEQ ID NO: 13. In some embodiments, the anti-C5 antibody or antigen-binding fragment thereof comprises a VH comprising the amino acid sequence of SEQ ID NO: 93, or a variant thereof comprising at least about 80% (e.g., at least about any of 85%, 90%, 95%, 96%, 97%, 98%, 99% or more) sequence identity to SEQ ID NO: 93, and a VL comprising the amino acid sequence of SEQ ID NO: 98, or a variant thereof comprising at least about 80% (e.g., at least about any of 85%, 90%, 95%, 96%, 97%, 98%, 99% or more) sequence identity to SEQ ID NO: 98. In some embodiments, the anti-C5 antibody or antigen-binding fragment thereof comprises a VH comprising the amino acid sequence of SEQ ID NO: 93, and a VL comprising the amino acid sequence of SEQ ID NO: 98. In some embodiments, the VH and VL do not comprise a signal peptide at the N-terminus.

In some embodiments, the anti-C5 antibody comprises a heavy chain comprising the amino acid sequence of SEQ ID NO: 2, or a variant thereof comprising at least about 80% (e.g., at least about any of 85%, 90%, 95%, 96%, 97%, 98%, 99% or more) sequence identity to SEQ ID NO: 2. In some embodiments, the anti-C5 antibody comprises a light chain comprising the amino acid sequence of SEQ ID NO: 13, or a variant thereof comprising at least about 80% (e.g., at least about any of 85%, 90%, 95%, 96%, 97%, 98%, 99% or more) sequence identity to SEQ ID NO: 13. In some embodiments, the anti-C5 antibody is humanized. In some embodiments, the anti-C5 antibody is a chimeric antibody. In some embodiments, the anti-C5 antibody is mAb L3-1, or a variant thereof. In some embodiments, the anti-C5 antibody mAb L3-1 comprises i) a heavy chain comprising the amino acid sequence of SEQ ID NO: 2 and a light chain comprising the amino acid sequence of SEQ ID NO: 13; or ii) a VH comprising the amino acid sequence of SEQ ID NO: 93 and a VL comprising the amino acid sequence of SEQ ID NO: 98. In some embodiments, the anti-C5 antibody comprises an Fc fragment. In some embodiments, the anti-C5 antibody comprises a human IgG4 Fc fragment or variant thereof. In some embodiments, the anti-C5 antibody mAb L3-1 comprises i) a heavy chain comprising the amino acid sequence of SEQ ID NO: 2 and a light chain comprising the amino acid sequence of SEQ ID NO: 13; or ii) a VH comprising the amino acid sequence of SEQ ID NO: 93 and a VL comprising the amino acid sequence of SEQ ID NO: 98; and a human IgG4 Fc fragment comprising the amino acid sequence of SEQ ID NO: 32. In some embodiments, the anti-C5 antibody comprises a human IgG4 Fc fragment having an S108P mutation relative to SEQ ID NO: 32, or a variant thereof. In some embodiments, the anti-C5 antibody mAb L3-1 comprises i) a heavy chain comprising the amino acid sequence of SEQ ID NO: 2 and a light chain comprising the amino acid sequence of SEQ ID NO: 13; or ii) a VH comprising the amino acid sequence of SEQ ID NO: 93 and a VL comprising the amino acid sequence of SEQ ID NO: 98; and a human IgG4 Fc fragment having an S108P mutation relative to SEQ ID NO: 32, comprising the amino acid sequence of SEQ ID NO: 33. In some embodiments, the anti-C5 antibody comprises a human IgG4 Fc fragment having an S108P mutation, a M308L mutation, and a N314A mutation relative to SEQ ID NO: 32, or a variant thereof. In some embodiments, the anti-C5 antibody mAb L3-1 comprises i) a heavy chain comprising the amino acid sequence of SEQ ID NO: 2 and a light chain comprising the amino acid sequence of SEQ ID NO: 13; or ii) a VH comprising the amino acid sequence of SEQ ID NO: 93 and a VL comprising the amino acid sequence of SEQ ID NO: 98; and a human IgG4 Fc fragment having an S108P mutation, a M308L mutation, and a N314A mutation relative to SEQ ID NO: 32, comprising the amino acid sequence of SEQ ID NO: 61.

In some embodiments, the anti-C5 antibody or antigen-binding fragment thereof comprises: a VH-CDR1 comprising the amino acid sequence of SEQ ID NO: 3; a VH-CDR2 comprising the amino acid sequence of SEQ ID NO: 4; a VH-CDR3 comprising the amino acid sequence of SEQ ID NO: 5; a VL-CDR1 comprising the amino acid sequence of SEQ ID NO: 14; a VL-CDR2 comprising the amino acid sequence of SEQ ID NO: 9; and a VL-CDR3 comprising the amino acid sequence of SEQ ID NO: 10, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 3, or a variant thereof comprising up to about 3 (such as about any of 1, 2, or 3) amino acid substitutions (e.g., conserved substitution); a VH-CDR2 comprising the amino acid sequence of SEQ ID NO: 4, or a variant thereof comprising up to about 3 (such as about any of 1, 2, or 3) amino acid substitutions (e.g., conserved substitution); a VH-CDR3 comprising the amino acid sequence of SEQ ID NO: 5, or a variant thereof comprising up to about 3 (such as about any of 1, 2, or 3) amino acid substitutions (e.g., conserved substitution); a VL-CDR1 comprising the amino acid sequence of SEQ ID NO: 14, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 10, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 3; a VH-CDR2 comprising the amino acid sequence of SEQ ID NO: 4; a VH-CDR3 comprising the amino acid sequence of SEQ ID NO: 5; a VL-CDR1 comprising the amino acid sequence of SEQ ID NO: 14; a VL-CDR2 comprising the amino acid sequence of SEQ ID NO: 9; and a VL-CDR3 comprising the amino acid sequence of SEQ ID NO: 10.

In some embodiments, the anti-C5 antibody or antigen-binding fragment thereof comprises: a VH-CDR1 comprising the amino acid sequence of SEQ ID NO: 94; a VH-CDR2 comprising the amino acid sequence of SEQ ID NO: 4; a VH-CDR3 comprising the amino acid sequence of SEQ ID NO: 5; a VL-CDR1 comprising the amino acid sequence of SEQ ID NO: 14; a VL-CDR2 comprising the amino acid sequence of SEQ ID NO: 9; and a VL-CDR3 comprising the amino acid sequence of SEQ ID NO: 96, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 94, or a variant thereof comprising up to about 3 (such as about any of 1, 2, or 3) amino acid substitutions (e.g., conserved substitution); a VH-CDR2 comprising the amino acid sequence of SEQ ID NO: 4, or a variant thereof comprising up to about 3 (such as about any of 1, 2, or 3) amino acid substitutions (e.g., conserved substitution); a VH-CDR3 comprising the amino acid sequence of SEQ ID NO: 5, or a variant thereof comprising up to about 3 (such as about any of 1, 2, or 3) amino acid substitutions (e.g., conserved substitution); a VL-CDR1 comprising the amino acid sequence of SEQ ID NO: 14, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 96, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 94; a VH-CDR2 comprising the amino acid sequence of SEQ ID NO: 4; a VH-CDR3 comprising the amino acid sequence of SEQ ID NO: 5; a VL-CDR1 comprising the amino acid sequence of SEQ ID NO: 14; a VL-CDR2 comprising the amino acid sequence of SEQ ID NO: 9; and a VL-CDR3 comprising the amino acid sequence of SEQ ID NO: 96.

In some embodiments, the anti-C5 antibody or antigen-binding fragment thereof comprises a VH comprising the amino acid sequence of SEQ ID NO: 2, or a variant thereof comprising at least about 80% (e.g., at least about any of 85%, 90%, 95%, 96%, 97%, 98%, 99% or more) sequence identity to SEQ ID NO: 2, and a VL comprising the amino acid sequence of SEQ ID NO: 16, or a variant thereof comprising at least about 80% (e.g., at least about any of 85%, 90%, 95%, 96%, 97%, 98%, 99% or more) sequence identity to SEQ ID NO: 16. In some embodiments, the anti-C5 antibody or antigen-binding fragment thereof comprises a VH comprising the amino acid sequence of SEQ ID NO: 2, and a VL comprising the amino acid sequence of SEQ ID NO: 16. In some embodiments, the anti-C5 antibody or antigen-binding fragment thereof comprises a VH comprising the amino acid sequence of SEQ ID NO: 93, or a variant thereof comprising at least about 80% (e.g., at least about any of 85%, 90%, 95%, 96%, 97%, 98%, 99% or more) sequence identity to SEQ ID NO: 93, and a VL comprising the amino acid sequence of SEQ ID NO: 99, or a variant thereof comprising at least about 80% (e.g., at least about any of 85%, 90%, 95%, 96%, 97%, 98%, 99% or more) sequence identity to SEQ ID NO: 99. In some embodiments, the anti-C5 antibody or antigen-binding fragment thereof comprises a VH comprising the amino acid sequence of SEQ ID NO: 93, and a VL comprising the amino acid sequence of SEQ ID NO: 99. In some embodiments, the VH and VL do not comprise a signal peptide at the N-terminus.

In some embodiments, the anti-C5 antibody comprises a heavy chain comprising the amino acid sequence of SEQ ID NO: 2, or a variant thereof comprising at least about 80% (e.g., at least about any of 85%, 90%, 95%, 96%, 97%, 98%, 99% or more) sequence identity to SEQ ID NO: 2. In some embodiments, the anti-C5 antibody comprises a light chain comprising the amino acid sequence of SEQ ID NO: 16, or a variant thereof comprising at least about 80% (e.g., at least about any of 85%, 90%, 95%, 96%, 97%, 98%, 99% or more) sequence identity to SEQ ID NO: 16. In some embodiments, the anti-C5 antibody is humanized. In some embodiments, the anti-C5 antibody is a chimeric antibody. In some embodiments, the anti-C5 antibody is mAb L1-2, or a variant thereof. In some embodiments, the anti-C5 antibody mAb L1-2 comprises i) a heavy chain comprising the amino acid sequence of SEQ ID NO: 2 and a light chain comprising the amino acid sequence of SEQ ID NO: 16; or ii) a VH comprising the amino acid sequence of SEQ ID NO: 93, and a VL comprising the amino acid sequence of SEQ ID NO: 99. In some embodiments, the anti-C5 antibody comprises an Fc fragment. In some embodiments, the anti-C5 antibody comprises a human IgG4 Fc fragment or variant thereof. In some embodiments, the anti-C5 antibody mAb L1-2 comprises i) a heavy chain comprising the amino acid sequence of SEQ ID NO: 2 and a light chain comprising the amino acid sequence of SEQ ID NO: 16; or ii) a VH comprising the amino acid sequence of SEQ ID NO: 93, and a VL comprising the amino acid sequence of SEQ ID NO: 99; and a human IgG4 Fc fragment comprising the amino acid sequence of SEQ ID NO: 32. In some embodiments, the anti-C5 antibody comprises a human IgG4 Fc fragment having an S108P mutation relative to SEQ ID NO: 32, or a variant thereof. In some embodiments, the anti-C5 antibody mAb L1-2 comprises i) a heavy chain comprising the amino acid sequence of SEQ ID NO: 2 and a light chain comprising the amino acid sequence of SEQ ID NO: 16; or ii) a VH comprising the amino acid sequence of SEQ ID NO: 93, and a VL comprising the amino acid sequence of SEQ ID NO: 99; and a human IgG4 Fc fragment having an S108P mutation relative to SEQ ID NO: 32, comprising the amino acid sequence of SEQ ID NO: 33. In some embodiments, the anti-C5 antibody comprises a human IgG4 Fc fragment having an S108P mutation, a M308L mutation, and a N314A mutation relative to SEQ ID NO: 32, or a variant thereof. In some embodiments, the anti-C5 antibody mAb L1-2 comprises i) a heavy chain comprising the amino acid sequence of SEQ ID NO: 2 and a light chain comprising the amino acid sequence of SEQ ID NO: 16; or ii) a VH comprising the amino acid sequence of SEQ ID NO: 93, and a VL comprising the amino acid sequence of SEQ ID NO: 99; and a human IgG4 Fc fragment having an S108P mutation, a M308L mutation, and a N314A mutation relative to SEQ ID NO: 32, comprising the amino acid sequence of SEQ ID NO: 61.

In some embodiments, the anti-C5 antibody or antigen-binding fragment thereof comprises: a VH-CDR1 comprising the amino acid sequence of SEQ ID NO: 17; a VH-CDR2 comprising the amino acid sequence of SEQ ID NO: 4; a VH-CDR3 comprising the amino acid sequence of SEQ ID NO: 5; a VL-CDR1 comprising the amino acid sequence of SEQ ID NO: 8; a VL-CDR2 comprising the amino acid sequence of SEQ ID NO: 9; and a VL-CDR3 comprising the amino acid sequence of SEQ ID NO: 10, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 17, or a variant thereof comprising up to about 3 (such as about any of 1, 2, or 3) amino acid substitutions (e.g., conserved substitution); a VH-CDR2 comprising the amino acid sequence of SEQ ID NO: 4, or a variant thereof comprising up to about 3 (such as about any of 1, 2, or 3) amino acid substitutions (e.g., conserved substitution); a VH-CDR3 comprising the amino acid sequence of SEQ ID NO: 5, or a variant thereof comprising up to about 3 (such as about any of 1, 2, or 3) amino acid substitutions (e.g., conserved substitution); a VL-CDR1 comprising the amino acid sequence of SEQ ID NO: 8, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 10, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 17; a VH-CDR2 comprising the amino acid sequence of SEQ ID NO: 4; a VH-CDR3 comprising the amino acid sequence of SEQ ID NO: 5; a VL-CDR1 comprising the amino acid sequence of SEQ ID NO: 8; a VL-CDR2 comprising the amino acid sequence of SEQ ID NO: 9; and a VL-CDR3 comprising the amino acid sequence of SEQ ID NO: 10.

In some embodiments, the anti-C5 antibody or antigen-binding fragment thereof comprises: a VH-CDR1 comprising the amino acid sequence of SEQ ID NO: 17; a VH-CDR2 comprising the amino acid sequence of SEQ ID NO: 4; a VH-CDR3 comprising the amino acid sequence of SEQ ID NO: 5; a VL-CDR1 comprising the amino acid sequence of SEQ ID NO: 8; a VL-CDR2 comprising the amino acid sequence of SEQ ID NO: 9; and a VL-CDR3 comprising the amino acid sequence of SEQ ID NO: 96, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 17, or a variant thereof comprising up to about 3 (such as about any of 1, 2, or 3) amino acid substitutions (e.g., conserved substitution); a VH-CDR2 comprising the amino acid sequence of SEQ ID NO: 4, or a variant thereof comprising up to about 3 (such as about any of 1, 2, or 3) amino acid substitutions (e.g., conserved substitution); a VH-CDR3 comprising the amino acid sequence of SEQ ID NO: 5, or a variant thereof comprising up to about 3 (such as about any of 1, 2, or 3) amino acid substitutions (e.g., conserved substitution); a VL-CDR1 comprising the amino acid sequence of SEQ ID NO: 8, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 96, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 17; a VH-CDR2 comprising the amino acid sequence of SEQ ID NO: 4; a VH-CDR3 comprising the amino acid sequence of SEQ ID NO: 5; a VL-CDR1 comprising the amino acid sequence of SEQ ID NO: 8; a VL-CDR2 comprising the amino acid sequence of SEQ ID NO: 9; and a VL-CDR3 comprising the amino acid sequence of SEQ ID NO: 96.

In some embodiments, the anti-C5 antibody or antigen-binding fragment thereof comprises a VH comprising the amino acid sequence of SEQ ID NO: 19, or a variant thereof comprising at least about 80% (e.g., at least about any of 85%, 90%, 95%, 96%, 97%, 98%, 99% or more) sequence identity to SEQ ID NO: 19, and a VL comprising the amino acid sequence of SEQ ID NO: 7, or a variant thereof comprising at least about 80% (e.g., at least about any of 85%, 90%, 95%, 96%, 97%, 98%, 99% or more) sequence identity to SEQ ID NO: 7. In some embodiments, the anti-C5 antibody or antigen-binding fragment thereof comprises a VH comprising the amino acid sequence of SEQ ID NO: 19, and a VL comprising the amino acid sequence of SEQ ID NO: 7. In some embodiments, the anti-C5 antibody or antigen-binding fragment thereof comprises a VH comprising the amino acid sequence of SEQ ID NO: 101, or a variant thereof comprising at least about 80% (e.g., at least about any of 85%, 90%, 95%, 96%, 97%, 98%, 99% or more) sequence identity to SEQ ID NO: 101, and a VL comprising the amino acid sequence of SEQ ID NO: 95, or a variant thereof comprising at least about 80% (e.g., at least about any of 85%, 90%, 95%, 96%, 97%, 98%, 99% or more) sequence identity to SEQ ID NO: 95. In some embodiments, the anti-C5 antibody or antigen-binding fragment thereof comprises a VH comprising the amino acid sequence of SEQ ID NO: 101, and a VL comprising the amino acid sequence of SEQ ID NO: 95. In some embodiments, the VH and VL do not comprise a signal peptide at the N-terminus.

In some embodiments, the anti-C5 antibody comprises a heavy chain comprising the amino acid sequence of SEQ ID NO: 19, or a variant thereof comprising at least about 80% (e.g., at least about any of 85%, 90%, 95%, 96%, 97%, 98%, 99% or more) sequence identity to SEQ ID NO: 19. In some embodiments, the anti-C5 antibody comprises a light chain comprising the amino acid sequence of SEQ ID NO: 7, or a variant thereof comprising at least about 80% (e.g., at least about any of 85%, 90%, 95%, 96%, 97%, 98%, 99% or more) sequence identity to SEQ ID NO: 7. In some embodiments, the anti-C5 antibody is humanized. In some embodiments, the anti-C5 antibody is a chimeric antibody. In some embodiments, the anti-C5 antibody is mAb H1-4, or a variant thereof. In some embodiments, the anti-C5 antibody mAb H1-4 comprises i) a heavy chain comprising the amino acid sequence of SEQ ID NO: 19 and a light chain comprising the amino acid sequence of SEQ ID NO: 7; or ii) a VH comprising the amino acid sequence of SEQ ID NO: 101, and a VL comprising the amino acid sequence of SEQ ID NO: 95. In some embodiments, the anti-C5 antibody comprises an Fc fragment. In some embodiments, the anti-C5 antibody comprises a human IgG4 Fc fragment or variant thereof. In some embodiments, the anti-C5 antibody mAb H1-4 comprises i) a heavy chain comprising the amino acid sequence of SEQ ID NO: 19 and a light chain comprising the amino acid sequence of SEQ ID NO: 7; or ii) a VH comprising the amino acid sequence of SEQ ID NO: 101, and a VL comprising the amino acid sequence of SEQ ID NO: 95; and a human IgG4 Fc fragment comprising the amino acid sequence of SEQ ID NO: 32. In some embodiments, the anti-C5 antibody comprises a human IgG4 Fc fragment having an S108P mutation relative to SEQ ID NO: 32, or a variant thereof. In some embodiments, the anti-C5 antibody mAb H1-4 comprises i) a heavy chain comprising the amino acid sequence of SEQ ID NO: 19 and a light chain comprising the amino acid sequence of SEQ ID NO: 7; or ii) a VH comprising the amino acid sequence of SEQ ID NO: 101, and a VL comprising the amino acid sequence of SEQ ID NO: 95; and a human IgG4 Fc fragment having an S108P mutation relative to SEQ ID NO: 32, comprising the amino acid sequence of SEQ ID NO: 33. In some embodiments, the anti-C5 antibody comprises a human IgG4 Fc fragment having an S108P mutation, a M308L mutation, and a N314A mutation relative to SEQ ID NO: 32, or a variant thereof. In some embodiments, the anti-C5 antibody mAb H1-4 comprises i) a heavy chain comprising the amino acid sequence of SEQ ID NO: 19 and a light chain comprising the amino acid sequence of SEQ ID NO: 7; or ii) a VH comprising the amino acid sequence of SEQ ID NO: 101, and a VL comprising the amino acid sequence of SEQ ID NO: 95; and a human IgG4 Fc fragment having an S108P mutation, a M308L mutation, and a N314A mutation relative to SEQ ID NO: 32, comprising the amino acid sequence of SEQ ID NO: 61.

In some embodiments, the anti-C5 antibody or antigen-binding fragment thereof comprises: a VH-CDR1 comprising the amino acid sequence of SEQ ID NO: 20; a VH-CDR2 comprising the amino acid sequence of SEQ ID NO: 4; a VH-CDR3 comprising the amino acid sequence of SEQ ID NO: 5; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 10, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 20, or a variant thereof comprising up to about 3 (such as about any of 1, 2, or 3) amino acid substitutions (e.g., conserved substitution); a VH-CDR2 comprising the amino acid sequence of SEQ ID NO: 4, or a variant thereof comprising up to about 3 (such as about any of 1, 2, or 3) amino acid substitutions (e.g., conserved substitution); a VH-CDR3 comprising the amino acid sequence of SEQ ID NO: 5, or a variant thereof comprising up to about 3 (such as about any of 1, 2, or 3) amino acid substitutions (e.g., conserved substitution); a VL-CDR1 comprising the amino acid sequence of SEQ ID NO: 23, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 10, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 20; a VH-CDR2 comprising the amino acid sequence of SEQ ID NO: 4; a VH-CDR3 comprising the amino acid sequence of SEQ ID NO: 5; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 10.

In some embodiments, the anti-C5 antibody or antigen-binding fragment thereof comprises: a VH-CDR1 comprising the amino acid sequence of SEQ ID NO: 102; a VH-CDR2 comprising the amino acid sequence of SEQ ID NO: 4; a VH-CDR3 comprising the amino acid sequence of SEQ ID NO: 5; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 96, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 102, or a variant thereof comprising up to about 3 (such as about any of 1, 2, or 3) amino acid substitutions (e.g., conserved substitution); a VH-CDR2 comprising the amino acid sequence of SEQ ID NO: 4, or a variant thereof comprising up to about 3 (such as about any of 1, 2, or 3) amino acid substitutions (e.g., conserved substitution); a VH-CDR3 comprising the amino acid sequence of SEQ ID NO: 5, or a variant thereof comprising up to about 3 (such as about any of 1, 2, or 3) amino acid substitutions (e.g., conserved substitution); a VL-CDR1 comprising the amino acid sequence of SEQ ID NO: 23, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 96, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 102; a VH-CDR2 comprising the amino acid sequence of SEQ ID NO: 4; a VH-CDR3 comprising the amino acid sequence of SEQ ID NO: 5; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 96.

In some embodiments, the anti-C5 antibody or antigen-binding fragment thereof comprises a VH comprising the amino acid sequence of SEQ ID NO: 22, or a variant thereof comprising at least about 80% (e.g., at least about any of 85%, 90%, 95%, 96%, 97%, 98%, 99% or more) sequence identity to SEQ ID NO: 22, and a VL comprising the amino acid sequence of SEQ ID NO: 25, or a variant thereof comprising at least about 80% (e.g., at least about any of 85%, 90%, 95%, 96%, 97%, 98%, 99% or more) sequence identity to SEQ ID NO: 25. In some embodiments, the anti-C5 antibody or antigen-binding fragment thereof comprises a VH comprising the amino acid sequence of SEQ ID NO: 22, and a VL comprising the amino acid sequence of SEQ ID NO: 25. In some embodiments, the anti-C5 antibody or antigen-binding fragment thereof comprises a VH comprising the amino acid sequence of SEQ ID NO: 103, or a variant thereof comprising at least about 80% (e.g., at least about any of 85%, 90%, 95%, 96%, 97%, 98%, 99% or more) sequence identity to SEQ ID NO: 103, and a VL comprising the amino acid sequence of SEQ ID NO: 88, or a variant thereof comprising at least about 80% (e.g., at least about any of 85%, 90%, 95%, 96%, 97%, 98%, 99% or more) sequence identity to SEQ ID NO: 88. In some embodiments, the anti-C5 antibody or antigen-binding fragment thereof comprises a VH comprising the amino acid sequence of SEQ ID NO: 103, and a VL comprising the amino acid sequence of SEQ ID NO: 88. In some embodiments, the VH and VL do not comprise a signal peptide at the N-terminus.

In some embodiments, the anti-C5 antibody comprises a heavy chain comprising the amino acid sequence of SEQ ID NO: 22, or a variant thereof comprising at least about 80% (e.g., at least about any of 85%, 90%, 95%, 96%, 97%, 98%, 99% or more) sequence identity to SEQ ID NO: 22. In some embodiments, the anti-C5 antibody comprises a light chain comprising the amino acid sequence of SEQ ID NO: 25, or a variant thereof comprising at least about 80% (e.g., at least about any of 85%, 90%, 95%, 96%, 97%, 98%, 99% or more) sequence identity to SEQ ID NO: 25. In some embodiments, the anti-C5 antibody is humanized. In some embodiments, the anti-C5 antibody is a chimeric antibody. In some embodiments, the anti-C5 antibody is mAb H1-8/L1-9, or a variant thereof. In some embodiments, the anti-C5 antibody mAb H1-8/L1-9 comprises i) a heavy chain comprising the amino acid sequence of SEQ ID NO: 22 and a light chain comprising the amino acid sequence of SEQ ID NO: 25; or ii) a VH comprising the amino acid sequence of SEQ ID NO: 103, and a VL comprising the amino acid sequence of SEQ ID NO: 88. In some embodiments, the anti-C5 antibody comprises an Fc fragment. In some embodiments, the anti-C5 antibody comprises a human IgG4 Fc fragment or variant thereof. In some embodiments, the anti-C5 antibody mAb H1-8/L1-9 comprises i) a heavy chain comprising the amino acid sequence of SEQ ID NO: 22 and a light chain comprising the amino acid sequence of SEQ ID NO: 25; or ii) a VH comprising the amino acid sequence of SEQ ID NO: 103, and a VL comprising the amino acid sequence of SEQ ID NO: 88; and a human IgG4 Fc fragment comprising the amino acid sequence of SEQ ID NO: 32. In some embodiments, the anti-C5 antibody comprises a human IgG4 Fc fragment having an S108P mutation relative to SEQ ID NO: 32, or a variant thereof. In some embodiments, the anti-C5 antibody mAb H1-8/L1-9 comprises i) a heavy chain comprising the amino acid sequence of SEQ ID NO: 22 and a light chain comprising the amino acid sequence of SEQ ID NO: 25; or ii) a VH comprising the amino acid sequence of SEQ ID NO: 103, and a VL comprising the amino acid sequence of SEQ ID NO: 88; and a human IgG4 Fc fragment having an S108P mutation relative to SEQ ID NO: 32, comprising the amino acid sequence of SEQ ID NO: 33. In some embodiments, the anti-C5 antibody comprises a human IgG4 Fc fragment having an S108P mutation, a M308L mutation, and a N314A mutation relative to SEQ ID NO: 32, or a variant thereof. In some embodiments, the anti-C5 antibody mAb H1-8/L1-9 comprises i) a heavy chain comprising the amino acid sequence of SEQ ID NO: 22 and a light chain comprising the amino acid sequence of SEQ ID NO: 25; or ii) a VH comprising the amino acid sequence of SEQ ID NO: 103, and a VL comprising the amino acid sequence of SEQ ID NO: 88; and a human IgG4 Fc fragment having an S108P mutation, a M308L mutation, and a N314A mutation relative to SEQ ID NO: 32, comprising the amino acid sequence of SEQ ID NO: 61.

In some embodiments, the anti-C5 antibody or antigen-binding fragment thereof comprises: a VH-CDR1 comprising the amino acid sequence of SEQ ID NO: 3; a VH-CDR2 comprising the amino acid sequence of SEQ ID NO: 26; a VH-CDR3 comprising the amino acid sequence of SEQ ID NO: 5; a VL-CDR1 comprising the amino acid sequence of SEQ ID NO: 8; a VL-CDR2 comprising the amino acid sequence of SEQ ID NO: 9; and a VL-CDR3 comprising the amino acid sequence of SEQ ID NO: 29, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 3, or a variant thereof comprising up to about 3 (such as about any of 1, 2, or 3) amino acid substitutions (e.g., conserved substitution); a VH-CDR2 comprising the amino acid sequence of SEQ ID NO: 26, or a variant thereof comprising up to about 3 (such as about any of 1, 2, or 3) amino acid substitutions (e.g., conserved substitution); a VH-CDR3 comprising the amino acid sequence of SEQ ID NO: 5, or a variant thereof comprising up to about 3 (such as about any of 1, 2, or 3) amino acid substitutions (e.g., conserved substitution); a VL-CDR1 comprising the amino acid sequence of SEQ ID NO: 8, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 29, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 3; a VH-CDR2 comprising the amino acid sequence of SEQ ID NO: 26; a VH-CDR3 comprising the amino acid sequence of SEQ ID NO: 5; a VL-CDR1 comprising the amino acid sequence of SEQ ID NO: 8; a VL-CDR2 comprising the amino acid sequence of SEQ ID NO: 9; and a VL-CDR3 comprising the amino acid sequence of SEQ ID NO: 29.

In some embodiments, the anti-C5 antibody or antigen-binding fragment thereof comprises: a VH-CDR1 comprising the amino acid sequence of SEQ ID NO: 94; a VH-CDR2 comprising the amino acid sequence of SEQ ID NO: 26; a VH-CDR3 comprising the amino acid sequence of SEQ ID NO: 5; a VL-CDR1 comprising the amino acid sequence of SEQ ID NO: 8; a VL-CDR2 comprising the amino acid sequence of SEQ ID NO: 9; and a VL-CDR3 comprising the amino acid sequence of SEQ ID NO: 105, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 94, or a variant thereof comprising up to about 3 (such as about any of 1, 2, or 3) amino acid substitutions (e.g., conserved substitution); a VH-CDR2 comprising the amino acid sequence of SEQ ID NO: 26, or a variant thereof comprising up to about 3 (such as about any of 1, 2, or 3) amino acid substitutions (e.g., conserved substitution); a VH-CDR3 comprising the amino acid sequence of SEQ ID NO: 5, or a variant thereof comprising up to about 3 (such as about any of 1, 2, or 3) amino acid substitutions (e.g., conserved substitution); a VL-CDR1 comprising the amino acid sequence of SEQ ID NO: 8, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 105, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 94; a VH-CDR2 comprising the amino acid sequence of SEQ ID NO: 26; a VH-CDR3 comprising the amino acid sequence of SEQ ID NO: 5; a VL-CDR1 comprising the amino acid sequence of SEQ ID NO: 8; a VL-CDR2 comprising the amino acid sequence of SEQ ID NO: 9; and a VL-CDR3 comprising the amino acid sequence of SEQ ID NO: 105.

In some embodiments, the anti-C5 antibody or antigen-binding fragment thereof comprises a VH comprising the amino acid sequence of SEQ ID NO: 28, or a variant thereof comprising at least about 80% (e.g., at least about any of 85%, 90%, 95%, 96%, 97%, 98%, 99% or more) sequence identity to SEQ ID NO: 28, and a VL comprising the amino acid sequence of SEQ ID NO: 31, or a variant thereof comprising at least about 80% (e.g., at least about any of 85%, 90%, 95%, 96%, 97%, 98%, 99% or more) sequence identity to SEQ ID NO: 31. In some embodiments, the anti-C5 antibody or antigen-binding fragment thereof comprises a VH comprising the amino acid sequence of SEQ ID NO: 28, and a VL comprising the amino acid sequence of SEQ ID NO: 31. In some embodiments, the anti-C5 antibody or antigen-binding fragment thereof comprises a VH comprising the amino acid sequence of SEQ ID NO: 104, or a variant thereof comprising at least about 80% (e.g., at least about any of 85%, 90%, 95%, 96%, 97%, 98%, 99% or more) sequence identity to SEQ ID NO: 104, and a VL comprising the amino acid sequence of SEQ ID NO: 106, or a variant thereof comprising at least about 80% (e.g., at least about any of 85%, 90%, 95%, 96%, 97%, 98%, 99% or more) sequence identity to SEQ ID NO: 106. In some embodiments, the anti-C5 antibody or antigen-binding fragment thereof comprises a VH comprising the amino acid sequence of SEQ ID NO: 104, and a VL comprising the amino acid sequence of SEQ ID NO: 106. In some embodiments, the VH and VL do not comprise a signal peptide at the N-terminus.

In some embodiments, the anti-C5 antibody comprises a heavy chain comprising the amino acid sequence of SEQ ID NO: 28, or a variant thereof comprising at least about 80% (e.g., at least about any of 85%, 90%, 95%, 96%, 97%, 98%, 99% or more) sequence identity to SEQ ID NO: 28. In some embodiments, the anti-C5 antibody comprises a light chain comprising the amino acid sequence of SEQ ID NO: 31, or a variant thereof comprising at least about 80% (e.g., at least about any of 85%, 90%, 95%, 96%, 97%, 98%, 99% or more) sequence identity to SEQ ID NO: 31. In some embodiments, the anti-C5 antibody is humanized. In some embodiments, the anti-C5 antibody is a chimeric antibody. In some embodiments, the anti-C5 antibody is mAb H2-6/L3-5, or a variant thereof. In some embodiments, the anti-C5 antibody mAb H2-6/L3-5 comprises i) a heavy chain comprising the amino acid sequence of SEQ ID NO: 28 and a light chain comprising the amino acid sequence of SEQ ID NO: 31; or ii) a VH comprising the amino acid sequence of SEQ ID NO: 104, and a VL comprising the amino acid sequence of SEQ ID NO: 106. In some embodiments, the anti-C5 antibody comprises an Fc fragment. In some embodiments, the anti-C5 antibody comprises a human IgG4 Fc fragment or variant thereof. In some embodiments, the anti-C5 antibody mAb H2-5/L3-5 comprises i) a heavy chain comprising the amino acid sequence of SEQ ID NO: 28 and a light chain comprising the amino acid sequence of SEQ ID NO: 31; or ii) a VH comprising the amino acid sequence of SEQ ID NO: 104, and a VL comprising the amino acid sequence of SEQ ID NO: 106; and a human IgG4 Fc fragment comprising the amino acid sequence of SEQ ID NO: 32. In some embodiments, the anti-C5 antibody comprises a human IgG4 Fc fragment having an S108P mutation relative to SEQ ID NO: 32, or a variant thereof. In some embodiments, the anti-C5 antibody mAb H2-6/L3-5 comprises i) a heavy chain comprising the amino acid sequence of SEQ ID NO: 28 and a light chain comprising the amino acid sequence of SEQ ID NO: 31; or ii) a VH comprising the amino acid sequence of SEQ ID NO: 104, and a VL comprising the amino acid sequence of SEQ ID NO: 106; and a human IgG4 Fc fragment having an S108P mutation relative to SEQ ID NO: 32, comprising the amino acid sequence of SEQ ID NO: 33. In some embodiments, the anti-C5 antibody comprises a human IgG4 Fc fragment having an S108P mutation, a M308L mutation, and a N314A mutation relative to SEQ ID NO: 32, or a variant thereof. In some embodiments, the anti-C5 antibody mAb H2-61L3-5 comprises i) a heavy chain comprising the amino acid sequence of SEQ ID NO: 28 and a light chain comprising the amino acid sequence of SEQ ID NO: 31; or ii) a VH comprising the amino acid sequence of SEQ ID NO: 104, and a VL comprising the amino acid sequence of SEQ ID NO: 106; and a human IgG4 Fc fragment having an S108P mutation, a M308L mutation, and a N314A mutation relative to SEQ ID NO: 32, comprising the amino acid sequence of SEQ ID NO: 61.

In some embodiments, the anti-C5 antibody or antigen-binding fragment thereof comprises: a VH-CDR1 comprising the amino acid sequence of SEQ ID NO: 3; a VH-CDR2 comprising the amino acid sequence of SEQ ID NO: 34; a VH-CDR3 comprising the amino acid sequence of SEQ ID NO: 5; a VL-CDR1 comprising the amino acid sequence of SEQ ID NO: 8; a VL-CDR2 comprising the amino acid sequence of SEQ ID NO: 9; and a VL-CDR3 comprising the amino acid sequence of SEQ ID NO: 10, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 3, or a variant thereof comprising up to about 3 (such as about any of 1, 2, or 3) amino acid substitutions (e.g., conserved substitution); a VH-CDR2 comprising the amino acid sequence of SEQ ID NO: 34, or a variant thereof comprising up to about 3 (such as about any of 1, 2, or 3) amino acid substitutions (e.g., conserved substitution); a VH-CDR3 comprising the amino acid sequence of SEQ ID NO: 5, or a variant thereof comprising up to about 3 (such as about any of 1, 2, or 3) amino acid substitutions (e.g., conserved substitution); a VL-CDR1 comprising the amino acid sequence of SEQ ID NO: 8, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 10, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 3; a VH-CDR2 comprising the amino acid sequence of SEQ ID NO: 34; a VH-CDR3 comprising the amino acid sequence of SEQ ID NO: 5; a VL-CDR1 comprising the amino acid sequence of SEQ ID NO: 8; a VL-CDR2 comprising the amino acid sequence of SEQ ID NO: 9; and a VL-CDR3 comprising the amino acid sequence of SEQ ID NO: 10.

In some embodiments, the anti-C5 antibody or antigen-binding fragment thereof comprises: a VH-CDR1 comprising the amino acid sequence of SEQ ID NO: 94; a VH-CDR2 comprising the amino acid sequence of SEQ ID NO: 34; a VH-CDR3 comprising the amino acid sequence of SEQ ID NO: 5; a VL-CDR1 comprising the amino acid sequence of SEQ ID NO: 8; a VL-CDR2 comprising the amino acid sequence of SEQ ID NO: 9; and a VL-CDR3 comprising the amino acid sequence of SEQ ID NO: 96, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 94, or a variant thereof comprising up to about 3 (such as about any of 1, 2, or 3) amino acid substitutions (e.g., conserved substitution); a VH-CDR2 comprising the amino acid sequence of SEQ ID NO: 34, or a variant thereof comprising up to about 3 (such as about any of 1, 2, or 3) amino acid substitutions (e.g., conserved substitution); a VH-CDR3 comprising the amino acid sequence of SEQ ID NO: 5, or a variant thereof comprising up to about 3 (such as about any of 1, 2, or 3) amino acid substitutions (e.g., conserved substitution); a VL-CDR1 comprising the amino acid sequence of SEQ ID NO: 8, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 96, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 94; a VH-CDR2 comprising the amino acid sequence of SEQ ID NO: 34; a VH-CDR3 comprising the amino acid sequence of SEQ ID NO: 5; a VL-CDR1 comprising the amino acid sequence of SEQ ID NO: 8; a VL-CDR2 comprising the amino acid sequence of SEQ ID NO: 9; and a VL-CDR3 comprising the amino acid sequence of SEQ ID NO: 96.

In some embodiments, the anti-C5 antibody or antigen-binding fragment thereof comprises a VH comprising the amino acid sequence of SEQ ID NO: 36, or a variant thereof comprising at least about 80% (e.g., at least about any of 85%, 90%, 95%, 96%, 97%, 98%, 99% or more) sequence identity to SEQ ID NO: 36, and a VL comprising the amino acid sequence of SEQ ID NO: 7, or a variant thereof comprising at least about 80% (e.g., at least about any of 85%, 90%, 95%, 96%, 97%, 98%, 99% or more) sequence identity to SEQ ID NO: 7. In some embodiments, the anti-C5 antibody or antigen-binding fragment thereof comprises a VH comprising the amino acid sequence of SEQ ID NO: 36, and a VL comprising the amino acid sequence of SEQ ID NO: 7. In some embodiments, the anti-C5 antibody or antigen-binding fragment thereof comprises a VH comprising the amino acid sequence of SEQ ID NO: 107, or a variant thereof comprising at least about 80% (e.g., at least about any of 85%, 90%, 95%, 96%, 97%, 98%, 99% or more) sequence identity to SEQ ID NO: 107, and a VL comprising the amino acid sequence of SEQ ID NO: 95, or a variant thereof comprising at least about 80% (e.g., at least about any of 85%, 90%, 95%, 96%, 97%, 98%, 99% or more) sequence identity to SEQ ID NO: 95. In some embodiments, the anti-C5 antibody or antigen-binding fragment thereof comprises a VH comprising the amino acid sequence of SEQ ID NO: 107, and a VL comprising the amino acid sequence of SEQ ID NO: 95. In some embodiments, the VH and VL do not comprise a signal peptide at the N-terminus.

In some embodiments, the anti-C5 antibody comprises a heavy chain comprising the amino acid sequence of SEQ ID NO: 36, or a variant thereof comprising at least about 80% (e.g., at least about any of 85%, 90%, 95%, 96%, 97%, 98%, 99% or more) sequence identity to SEQ ID NO: 36. In some embodiments, the anti-C5 antibody comprises a light chain comprising the amino acid sequence of SEQ ID NO: 7, or a variant thereof comprising at least about 80% (e.g., at least about any of 85%, 90%, 95%, 96%, 97%, 98%, 99% or more) sequence identity to SEQ ID NO: 7. In some embodiments, the anti-C5 antibody is humanized. In some embodiments, the anti-C5 antibody is a chimeric antibody. In some embodiments, the anti-C5 antibody comprises i) a heavy chain comprising the amino acid sequence of SEQ ID NO: 36 and a light chain comprising the amino acid sequence of SEQ ID NO: 7; or ii) a VH comprising the amino acid sequence of SEQ ID NO: 107, and a VL comprising the amino acid sequence of SEQ ID NO: 95. In some embodiments, the anti-C5 antibody comprises an Fc fragment. In some embodiments, the anti-C5 antibody comprises a human IgG4 Fc fragment or variant thereof. In some embodiments, the anti-C5 antibody comprises i) a heavy chain comprising the amino acid sequence of SEQ ID NO: 36 and a light chain comprising the amino acid sequence of SEQ ID NO: 7; or ii) a VH comprising the amino acid sequence of SEQ ID NO: 107, and a VL comprising the amino acid sequence of SEQ ID NO: 95; and a human IgG4 Fc fragment comprising the amino acid sequence of SEQ ID NO: 32. In some embodiments, the anti-C5 antibody comprises a human IgG4 Fc fragment having an S108P mutation relative to SEQ ID NO: 32, or a variant thereof. In some embodiments, the anti-C5 antibody comprises i) a heavy chain comprising the amino acid sequence of SEQ ID NO: 36 and a light chain comprising the amino acid sequence of SEQ ID NO: 7; or ii) a VH comprising the amino acid sequence of SEQ ID NO: 107, and a VL comprising the amino acid sequence of SEQ ID NO: 95; and a human IgG4 Fc fragment having an S108P mutation relative to SEQ ID NO: 32, comprising the amino acid sequence of SEQ ID NO: 33. In some embodiments, the anti-C5 antibody comprises a human IgG4 Fc fragment having an S108P mutation, a M308L mutation, and a N314A mutation relative to SEQ ID NO: 32, or a variant thereof. In some embodiments, the anti-C5 antibody comprises i) a heavy chain comprising the amino acid sequence of SEQ ID NO: 36 and a light chain comprising the amino acid sequence of SEQ ID NO: 7; or ii) a VH comprising the amino acid sequence of SEQ ID NO: 107, and a VL comprising the amino acid sequence of SEQ ID NO: 95; and a human IgG4 Fc fragment having an S108P mutation, a M308L mutation, and a N314A mutation relative to SEQ ID NO: 32, comprising the amino acid sequence of SEQ ID NO: 61.

In some embodiments, the anti-C5 antibody or antigen-binding fragment thereof comprises: a VH-CDR1 comprising the amino acid sequence of SEQ ID NO: 37; a VH-CDR2 comprising the amino acid sequence of SEQ ID NO: 38; a VH-CDR3 comprising the amino acid sequence of SEQ ID NO: 39; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 10, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 37, or a variant thereof comprising up to about 3 (such as about any of 1, 2, or 3) amino acid substitutions (e.g., conserved substitution); a VH-CDR2 comprising the amino acid sequence of SEQ ID NO: 38, or a variant thereof comprising up to about 3 (such as about any of 1, 2, or 3) amino acid substitutions (e.g., conserved substitution); a VH-CDR3 comprising the amino acid sequence of SEQ ID NO: 39, or a variant thereof comprising up to about 3 (such as about any of 1, 2, or 3) amino acid substitutions (e.g., conserved substitution); a VL-CDR1 comprising the amino acid sequence of SEQ ID NO: 23, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 10, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 37; a VH-CDR2 comprising the amino acid sequence of SEQ ID NO: 38; a VH-CDR3 comprising the amino acid sequence of SEQ ID NO: 39; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 10.

In some embodiments, the anti-C5 antibody or antigen-binding fragment thereof comprises: a VH-CDR1 comprising the amino acid sequence of SEQ ID NO: 108; a VH-CDR2 comprising the amino acid sequence of SEQ ID NO: 38; a VH-CDR3 comprising the amino acid sequence of SEQ ID NO: 39; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 96, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 108, or a variant thereof comprising up to about 3 (such as about any of 1, 2, or 3) amino acid substitutions (e.g., conserved substitution); a VH-CDR2 comprising the amino acid sequence of SEQ ID NO: 38, or a variant thereof comprising up to about 3 (such as about any of 1, 2, or 3) amino acid substitutions (e.g., conserved substitution); a VH-CDR3 comprising the amino acid sequence of SEQ ID NO: 39, or a variant thereof comprising up to about 3 (such as about any of 1, 2, or 3) amino acid substitutions (e.g., conserved substitution); a VL-CDR1 comprising the amino acid sequence of SEQ ID NO: 23, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 96, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 108; a VH-CDR2 comprising the amino acid sequence of SEQ ID NO: 38; a VH-CDR3 comprising the amino acid sequence of SEQ ID NO: 39; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 96.

In some embodiments, the anti-C5 antibody or antigen-binding fragment thereof comprises a VH comprising the amino acid sequence of SEQ ID NO: 41, or a variant thereof comprising at least about 80% (e.g., at least about any of 85%, 90%, 95%, 96%, 97%, 98%, 99% or more) sequence identity to SEQ ID NO: 41, and a VL comprising the amino acid sequence of SEQ ID NO: 25, or a variant thereof comprising at least about 80% (e.g., at least about any of 85%, 90%, 95%, 96%, 97%, 98%, 99% or more) sequence identity to SEQ ID NO: 25. In some embodiments, the anti-C5 antibody or antigen-binding fragment thereof comprises a VH comprising the amino acid sequence of SEQ ID NO: 41, and a VL comprising the amino acid sequence of SEQ ID NO: 25. In some embodiments, the anti-C5 antibody or antigen-binding fragment thereof comprises a VH comprising the amino acid sequence of SEQ ID NO: 109, or a variant thereof comprising at least about 80% (e.g., at least about any of 85%, 90%, 95%, 96%, 97%, 98%, 99% or more) sequence identity to SEQ ID NO: 109, and a VL comprising the amino acid sequence of SEQ ID NO: 88, or a variant thereof comprising at least about 80% (e.g., at least about any of 85%, 90%, 95%, 96%, 97%, 98%, 99% or more) sequence identity to SEQ ID NO: 88. In some embodiments, the anti-C5 antibody or antigen-binding fragment thereof comprises a VH comprising the amino acid sequence of SEQ ID NO: 109, and a VL comprising the amino acid sequence of SEQ ID NO: 88. In some embodiments, the VH and VL do not comprise a signal peptide at the N-terminus.

In some embodiments, the anti-C5 antibody comprises a heavy chain comprising the amino acid sequence of SEQ ID NO: 41, or a variant thereof comprising at least about 80% (e.g., at least about any of 85%, 90%, 95%, 96%, 97%, 98%, 99% or more) sequence identity to SEQ ID NO: 41. In some embodiments, the anti-C5 antibody comprises a light chain comprising the amino acid sequence of SEQ ID NO: 25, or a variant thereof comprising at least about 80% (e.g., at least about any of 85%, 90%, 95%, 96%, 97%, 98%, 99% or more) sequence identity to SEQ ID NO: 25. In some embodiments, the anti-C5 antibody is humanized. In some embodiments, the anti-C5 antibody is a chimeric antibody. In some embodiments, the anti-C5 antibody is mAb H1-8/L1-9 variant IWW, or a variant thereof. In some embodiments, the anti-C5 antibody mAb H1-8/L1-9 variant IWW comprises i) a heavy chain comprising the amino acid sequence of SEQ ID NO: 41 and a light chain comprising the amino acid sequence of SEQ ID NO: 25; or ii) a VH comprising the amino acid sequence of SEQ ID NO: 109, and a VL comprising the amino acid sequence of SEQ ID NO: 88. In some embodiments, the anti-C5 antibody comprises an Fc fragment. In some embodiments, the anti-C5 antibody comprises a human IgG4 Fc fragment or variant thereof. In some embodiments, the anti-C5 antibody mAb H1-8/L1-9 variant IWW comprises i) a heavy chain comprising the amino acid sequence of SEQ ID NO: 41 and a light chain comprising the amino acid sequence of SEQ ID NO: 25; or ii) a VH comprising the amino acid sequence of SEQ ID NO: 109, and a VL comprising the amino acid sequence of SEQ ID NO: 88; and a human IgG4 Fc fragment comprising the amino acid sequence of SEQ ID NO: 32. In some embodiments, the anti-C5 antibody comprises a human IgG4 Fc fragment having an S108P mutation, or a variant thereof. In some embodiments, the anti-C5 antibody mAb H1-8/L1-9 variant IWW comprises i) a heavy chain comprising the amino acid sequence of SEQ ID NO: 41 and a light chain comprising the amino acid sequence of SEQ ID NO: 25; or ii) a VH comprising the amino acid sequence of SEQ ID NO: 109, and a VL comprising the amino acid sequence of SEQ ID NO: 88; and a human IgG4 Fc fragment having an S108P mutation relative to SEQ ID NO: 32, comprising the amino acid sequence of SEQ ID NO: 33. In some embodiments, the anti-C5 antibody comprises a human IgG4 Fc fragment having an S108P mutation, a M308L mutation, and a N314A mutation relative to SEQ ID NO: 32, or a variant thereof. In some embodiments, the anti-C5 antibody mAb H1-8/L1-9 variant IWW comprises i) a heavy chain comprising the amino acid sequence of SEQ ID NO: 41 and a light chain comprising the amino acid sequence of SEQ ID NO: 25; or ii) a VH comprising the amino acid sequence of SEQ ID NO: 109, and a VL comprising the amino acid sequence of SEQ ID NO: 88; and a human IgG4 Fc fragment having an S108P mutation, a M308L mutation, and a N314A mutation relative to SEQ ID NO: 32, comprising the amino acid sequence of SEQ ID NO: 61.

In some embodiments, the anti-C5 antibody or antigen-binding fragment thereof comprises: a VH-CDR1 comprising the amino acid sequence of SEQ ID NO: 42; a VH-CDR2 comprising the amino acid sequence of SEQ ID NO: 43; a VH-CDR3 comprising the amino acid sequence of SEQ ID NO: 44; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 10, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 42, or a variant thereof comprising up to about 3 (such as about any of 1, 2, or 3) amino acid substitutions (e.g., conserved substitution); a VH-CDR2 comprising the amino acid sequence of SEQ ID NO: 43, or a variant thereof comprising up to about 3 (such as about any of 1, 2, or 3) amino acid substitutions (e.g., conserved substitution); a VH-CDR3 comprising the amino acid sequence of SEQ ID NO: 44, or a variant thereof comprising up to about 3 (such as about any of 1, 2, or 3) amino acid substitutions (e.g., conserved substitution); a VL-CDR1 comprising the amino acid sequence of SEQ ID NO: 23, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 10, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 42; a VH-CDR2 comprising the amino acid sequence of SEQ ID NO: 43; a VH-CDR3 comprising the amino acid sequence of SEQ ID NO: 44; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 10.

In some embodiments, the anti-C5 antibody or antigen-binding fragment thereof comprises: a VH-CDR1 comprising the amino acid sequence of SEQ ID NO: 108; a VH-CDR2 comprising the amino acid sequence of SEQ ID NO: 43; a VH-CDR3 comprising the amino acid sequence of SEQ ID NO: 44; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 96, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 108, or a variant thereof comprising up to about 3 (such as about any of 1, 2, or 3) amino acid substitutions (e.g., conserved substitution); a VH-CDR2 comprising the amino acid sequence of SEQ ID NO: 43, or a variant thereof comprising up to about 3 (such as about any of 1, 2, or 3) amino acid substitutions (e.g., conserved substitution); a VH-CDR3 comprising the amino acid sequence of SEQ ID NO: 44, or a variant thereof comprising up to about 3 (such as about any of 1, 2, or 3) amino acid substitutions (e.g., conserved substitution); a VL-CDR1 comprising the amino acid sequence of SEQ ID NO: 23, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 96, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 108; a VH-CDR2 comprising the amino acid sequence of SEQ ID NO: 43; a VH-CDR3 comprising the amino acid sequence of SEQ ID NO: 44; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 96.

In some embodiments, the anti-C5 antibody or antigen-binding fragment thereof comprises a VH comprising the amino acid sequence of SEQ ID NO: 46, or a variant thereof comprising at least about 80% (e.g., at least about any of 85%, 90%, 95%, 96%, 97%, 98%, 99% or more) sequence identity to SEQ ID NO: 46, and a VL comprising the amino acid sequence of SEQ ID NO: 25, or a variant thereof comprising at least about 80% (e.g., at least about any of 85%, 90%, 95%, 96%, 97%, 98%, 99% or more) sequence identity to SEQ ID NO: 25. In some embodiments, the anti-C5 antibody or antigen-binding fragment thereof comprises a VH comprising the amino acid sequence of SEQ ID NO: 46, and a VL comprising the amino acid sequence of SEQ ID NO: 25. In some embodiments, the anti-C5 antibody or antigen-binding fragment thereof comprises a VH comprising the amino acid sequence of SEQ ID NO: 110, or a variant thereof comprising at least about 80% (e.g., at least about any of 85%, 90%, 95%, 96%, 97%, 98%, 99% or more) sequence identity to SEQ ID NO: 110, and a VL comprising the amino acid sequence of SEQ ID NO: 88, or a variant thereof comprising at least about 80% (e.g., at least about any of 85%, 90%, 95%, 96%, 97%, 98%, 99% or more) sequence identity to SEQ ID NO: 88. In some embodiments, the anti-C5 antibody or antigen-binding fragment thereof comprises a VH comprising the amino acid sequence of SEQ ID NO: 110, and a VL comprising the amino acid sequence of SEQ ID NO: 88. In some embodiments, the VH and VL do not comprise a signal peptide at the N-terminus.

In some embodiments, the anti-C5 antibody comprises a heavy chain comprising the amino acid sequence of SEQ ID NO: 46, or a variant thereof comprising at least about 80% (e.g., at least about any of 85%, 90%, 95%, 96%, 97%, 98%, 99% or more) sequence identity to SEQ ID NO: 46. In some embodiments, the anti-C5 antibody comprises a light chain comprising the amino acid sequence of SEQ ID NO: 25, or a variant thereof comprising at least about 80% (e.g., at least about any of 85%, 90%, 95%, 96%, 97%, 98%, 99% or more) sequence identity to SEQ ID NO: 25. In some embodiments, the anti-C5 antibody is humanized. In some embodiments, the anti-C5 antibody is a chimeric antibody. In some embodiments, the anti-C5 antibody is mAb H1-8/L1-9 variant IFW, or a variant thereof. In some embodiments, the anti-C5 antibody mAb H1-8/L1-9 variant IFW comprises i) a heavy chain comprising the amino acid sequence of SEQ ID NO: 46 and a light chain comprising the amino acid sequence of SEQ ID NO: 25; or ii) a VH comprising the amino acid sequence of SEQ ID NO: 110, and a VL comprising the amino acid sequence of SEQ ID NO: 88. In some embodiments, the anti-C5 antibody comprises an Fc fragment. In some embodiments, the anti-C5 antibody comprises a human IgG4 Fc fragment or variant thereof. In some embodiments, the anti-C5 antibody mAb H1-8/L1-9 variant IFW comprises i) a heavy chain comprising the amino acid sequence of SEQ ID NO: 46 and a light chain comprising the amino acid sequence of SEQ ID NO: 25; or ii) a VH comprising the amino acid sequence of SEQ ID NO: 110, and a VL comprising the amino acid sequence of SEQ ID NO: 88; and a human IgG4 Fc fragment comprising the amino acid sequence of SEQ ID NO: 32. In some embodiments, the anti-C5 antibody comprises a human IgG4 Fc fragment having an S108P mutation relative to SEQ ID NO: 32, or a variant thereof. In some embodiments, the anti-C5 antibody mAb H1-8/L1-9 variant IFW comprises i) a heavy chain comprising the amino acid sequence of SEQ ID NO: 46 and a light chain comprising the amino acid sequence of SEQ ID NO: 25; or ii) a VH comprising the amino acid sequence of SEQ ID NO: 110, and a VL comprising the amino acid sequence of SEQ ID NO: 88; and a human IgG4 Fc fragment having an S108P mutation relative to SEQ ID NO: 32, comprising the amino acid sequence of SEQ ID NO: 33. In some embodiments, the anti-C5 antibody comprises a human IgG4 Fc fragment having an S108P mutation, a M308L mutation, and a N314A mutation relative to SEQ ID NO: 32, or a variant thereof. In some embodiments, the anti-C5 antibody mAb H1-8/L1-9 variant IFW comprises i) a heavy chain comprising the amino acid sequence of SEQ ID NO: 46 and a light chain comprising the amino acid sequence of SEQ ID NO: 25; or ii) a VH comprising the amino acid sequence of SEQ ID NO: 110, and a VL comprising the amino acid sequence of SEQ ID NO: 88; and a human IgG4 Fc fragment having an S108P mutation, a M308L mutation, and a N314A mutation relative to SEQ ID NO: 32, comprising the amino is acid sequence of SEQ ID NO: 61.

In some embodiments, the anti-C5 antibody or antigen-binding fragment thereof comprises: a VH-CDR1 comprising the amino acid sequence of SEQ ID NO: 47; a VH-CDR2 comprising the amino acid sequence of SEQ ID NO: 48; a VH-CDR3 comprising the amino acid sequence of SEQ ID NO: 49; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 10, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 47, or a variant thereof comprising up to about 3 (such as about any of 1, 2, or 3) amino acid substitutions (e.g., conserved substitution); a VH-CDR2 comprising the amino acid sequence of SEQ ID NO: 48, or a variant thereof comprising up to about 3 (such as about any of 1, 2, or 3) amino acid substitutions (e.g., conserved substitution); a VH-CDR3 comprising the amino acid sequence of SEQ ID NO: 49, or a variant thereof comprising up to about 3 (such as about any of 1, 2, or 3) amino acid substitutions (e.g., conserved substitution); a VL-CDR1 comprising the amino acid sequence of SEQ ID NO: 23, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 10, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 47; a VH-CDR2 comprising the amino acid sequence of SEQ ID NO: 48; a VH-CDR3 comprising the amino acid sequence of SEQ ID NO: 49; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 10.

In some embodiments, the anti-C5 antibody or antigen-binding fragment thereof comprises: a VH-CDR1 comprising the amino acid sequence of SEQ ID NO: 86; a VH-CDR2 comprising the amino acid sequence of SEQ ID NO: 48; a VH-CDR3 comprising the amino acid sequence of SEQ ID NO: 49; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 96, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 86, or a variant thereof comprising up to about 3 (such as about any of 1, 2, or 3) amino acid substitutions (e.g., conserved substitution); a VH-CDR2 comprising the amino acid sequence of SEQ ID NO: 48, or a variant thereof comprising up to about 3 (such as about any of 1, 2, or 3) amino acid substitutions (e.g., conserved substitution); a VH-CDR3 comprising the amino acid sequence of SEQ ID NO: 49, or a variant thereof comprising up to about 3 (such as about any of 1, 2, or 3) amino acid substitutions (e.g., conserved substitution); a VL-CDR1 comprising the amino acid sequence of SEQ ID NO: 23, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 96, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 86; a VH-CDR2 comprising the amino acid sequence of SEQ ID NO: 48; a VH-CDR3 comprising the amino acid sequence of SEQ ID NO: 49; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 96.

In some embodiments, the anti-C5 antibody or antigen-binding fragment thereof comprises a VH comprising the amino acid sequence of SEQ ID NO: 51, or a variant thereof comprising at least about 80% (e.g., at least about any of 85%, 90%, 95%, 96%, 97%, 98%, 99% or more) sequence identity to SEQ ID NO: 51, and a VL comprising the amino acid sequence of SEQ ID NO: 25, or a variant thereof comprising at least about 80% (e.g., at least about any of 85%, 90%, 95%, 96%, 97%, 98%, 99% or more) sequence identity to SEQ ID NO: 25. In some embodiments, the anti-C5 antibody or antigen-binding fragment thereof comprises a VH comprising the amino acid sequence of SEQ ID NO: 51, and a VL comprising the amino acid sequence of SEQ ID NO: 25. In some embodiments, the anti-C5 antibody or antigen-binding fragment thereof comprises a VH comprising the amino acid sequence of SEQ ID NO: 111, or a variant thereof comprising at least about 80% (e.g., at least about any of 85%, 90%, 95%, 96%, 97%, 98%, 99% or more) sequence identity to SEQ ID NO: 111, and a VL comprising the amino acid sequence of SEQ ID NO: 88, or a variant thereof comprising at least about 80% (e.g., at least about any of 85%, 90%, 95%, 96%, 97%, 98%, 99% or more) sequence identity to SEQ ID NO: 88. In some embodiments, the anti-C5 antibody or antigen-binding fragment thereof comprises a VH comprising the amino acid sequence of SEQ ID NO: 111, and a VL comprising the amino acid sequence of SEQ ID NO: 88. In some embodiments, the VH and VL do not comprise a signal peptide at the N-terminus.

In some embodiments, the anti-C5 antibody comprises a heavy chain comprising the amino acid sequence of SEQ ID NO: 51, or a variant thereof comprising at least about 80% (e.g., at least about any of 85%, 90%, 95%, 96%, 97%, 98%, 99% or more) sequence identity to SEQ ID NO: 51. In some embodiments, the anti-C5 antibody comprises a light chain comprising the amino acid sequence of SEQ ID NO: 25, or a variant thereof comprising at least about 80% (e.g., at least about any of 85%, 90%, 95%, 96%, 97%, 98%, 99% or more) sequence identity to SEQ ID NO: 25. In some embodiments, the anti-C5 antibody is humanized. In some embodiments, the anti-C5 antibody is a chimeric antibody. In some embodiments, the anti-C5 antibody is mAb H1-8/L1-9 variant FME, or a variant thereof. In some embodiments, the anti-C5 antibody mAb H1-8/L1-9 variant FME comprises i) a heavy chain comprising the amino acid sequence of SEQ ID NO: 51 and a light chain comprising the amino acid sequence of SEQ ID NO: 25; or ii) a VH comprising the amino acid sequence of SEQ ID NO: 111, and a VL comprising the amino acid sequence of SEQ ID NO: 88. In some embodiments, the anti-C5 antibody comprises an Fc fragment. In some embodiments, the anti-C5 antibody comprises a human IgG4 Fc fragment or variant thereof. In some embodiments, the anti-C5 antibody mAb H1-8/L1-9 variant FME comprises i) a heavy chain comprising the amino acid sequence of SEQ ID NO: 51 and a light chain comprising the amino acid sequence of SEQ ID NO: 25; or ii) a VH comprising the amino acid sequence of SEQ ID NO: 111, and a VL comprising the amino acid sequence of SEQ ID NO: 88; and a human IgG4 Fc fragment comprising the amino acid sequence of SEQ ID NO: 32. In some embodiments, the anti-C5 antibody comprises a human IgG4 Fc fragment having an S108P mutation relative to SEQ ID NO: 32, or a variant thereof. In some embodiments, the anti-C5 antibody mAb H1-8/L1-9 variant FME comprises i) a heavy chain comprising the amino acid sequence of SEQ ID NO: 51 and a light chain comprising the amino acid sequence of SEQ ID NO: 25; or ii) a VH comprising the amino acid sequence of SEQ ID NO: 111, and a VL comprising the amino acid sequence of SEQ ID NO: 88; and a human IgG4 Fc fragment having an S108P mutation relative to SEQ ID NO: 32, comprising the amino acid sequence of SEQ ID NO: 33. In some embodiments, the anti-C5 antibody comprises a human IgG4 Fc fragment having an S108P mutation, a M308L mutation, and a N314A mutation relative to SEQ ID NO: 32, or a variant thereof. In some embodiments, the anti-C5 antibody mAb H1-8/L1-9 variant FME comprises i) a heavy chain comprising the amino acid sequence of SEQ ID NO: 51 and a light chain comprising the amino acid sequence of SEQ ID NO: 25; or ii) a VH comprising the amino acid sequence of SEQ ID NO: 111, and a VL comprising the amino acid sequence of SEQ ID NO: 88; and a human IgG4 Fc fragment having an S108P mutation, a M308L mutation, and a N314A mutation relative to SEQ ID NO: 32, comprising the amino acid sequence of SEQ ID NO: 61.

In some embodiments, the anti-C5 antibody or antigen-binding fragment thereof comprises: a VH-CDR1 comprising the amino acid sequence of SEQ ID NO: 52; a VH-CDR2 comprising the amino acid sequence of SEQ ID NO: 53; a VH-CDR3 comprising the amino acid sequence of SEQ ID NO: 54; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 10, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 52, or a variant thereof comprising up to about 3 (such as about any of 1, 2, or 3) amino acid substitutions (e.g., conserved substitution); a VH-CDR2 comprising the amino acid sequence of SEQ ID NO: 53, or a variant thereof comprising up to about 3 (such as about any of 1, 2, or 3) amino acid substitutions (e.g., conserved substitution); a VH-CDR3 comprising the amino acid sequence of SEQ ID NO: 54, or a variant thereof comprising up to about 3 (such as about any of 1, 2, or 3) amino acid substitutions (e.g., conserved substitution); a VL-CDR1 comprising the amino acid sequence of SEQ ID NO: 23, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 10, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 52; a VH-CDR2 comprising the amino acid sequence of SEQ ID NO: 53; a VH-CDR3 comprising the amino acid sequence of SEQ ID NO: 54; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 10.

In some embodiments, the anti-C5 antibody or antigen-binding fragment thereof comprises: a VH-CDR1 comprising the amino acid sequence of SEQ ID NO: 86; a VH-CDR2 comprising the amino acid sequence of SEQ ID NO: 53; a VH-CDR3 comprising the amino acid sequence of SEQ ID NO: 54; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 96, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 86, or a variant thereof comprising up to about 3 (such as about any of 1, 2, or 3) amino acid substitutions (e.g., conserved substitution); a VH-CDR2 comprising the amino acid sequence of SEQ ID NO: 53, or a variant thereof comprising up to about 3 (such as about any of 1, 2, or 3) amino acid substitutions (e.g., conserved substitution); a VH-CDR3 comprising the amino acid sequence of SEQ ID NO: 54, or a variant thereof comprising up to about 3 (such as about any of 1, 2, or 3) amino acid substitutions (e.g., conserved substitution); a VL-CDR1 comprising the amino acid sequence of SEQ ID NO: 23, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 96, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 86; a VH-CDR2 comprising the amino acid sequence of SEQ ID NO: 53; a VH-CDR3 comprising the amino acid sequence of SEQ ID NO: 54; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 96.

In some embodiments, the anti-C5 antibody or antigen-binding fragment thereof comprises a VH comprising the amino acid sequence of SEQ ID NO: 56, or a variant thereof comprising at least about 80% (e.g., at least about any of 85%, 90%, 95%, 96%, 97%, 98%, 99% or more) sequence identity to SEQ ID NO: 56, and a VL comprising the amino acid sequence of SEQ ID NO: 25, or a variant thereof comprising at least about 80% (e.g., at least about any of 85%, 90%, 95%, 96%, 97%, 98%, 99% or more) sequence identity to SEQ ID NO: 25. In some embodiments, the anti-C5 antibody or antigen-binding fragment thereof comprises a VH comprising the amino acid sequence of SEQ ID NO: 56, and a VL comprising the amino acid sequence of SEQ ID NO: 25. In some embodiments, the anti-C5 antibody or antigen-binding fragment thereof comprises a VH comprising the amino acid sequence of SEQ ID NO: 112, or a variant thereof comprising at least about 80% (e.g., at least about any of 85%, 90%, 95%, 96%, 97%, 98%, 99% or more) sequence identity to SEQ ID NO: 112, and a VL comprising the amino acid sequence of SEQ ID NO: 88, or a variant thereof comprising at least about 80% (e.g., at least about any of 85%, 90%, 95%, 96%, 97%, 98%, 99% or more) sequence identity to SEQ ID NO: 88. In some embodiments, the anti-C5 antibody or antigen-binding fragment thereof comprises a VH comprising the amino acid sequence of SEQ ID NO: 112, and a VL comprising the amino acid sequence of SEQ ID NO: 88. In some embodiments, the VH and VL do not comprise a signal peptide at the N-terminus.

In some embodiments, the anti-C5 antibody comprises a heavy chain comprising the amino acid sequence of SEQ ID NO: 56, or a variant thereof comprising at least about 80% (e.g., at least about any of 85%, 90%, 95%, 96%, 97%, 98%, 99% or more) sequence identity to SEQ ID NO: 56. In some embodiments, the anti-C5 antibody comprises a light chain comprising the amino acid sequence of SEQ ID NO: 25, or a variant thereof comprising at least about 80% (e.g., at least about any of 85%, 90%, 95%, 96%, 97%, 98%, 99% or more) sequence identity to SEQ ID NO: 25. In some embodiments, the anti-C5 antibody is humanized. In some embodiments, the anti-C5 antibody is a chimeric antibody. In some embodiments, the anti-C5 antibody is mAb H1-8/L1-9 variant FMW, or a variant thereof. In some embodiments, the anti-C5 antibody mAb H1-8/L1-9 variant FMW comprises i) a heavy chain comprising the amino acid sequence of SEQ ID NO: 56 and a light chain comprising the amino acid sequence of SEQ ID NO: 25; or ii) a VH comprising the amino acid sequence of SEQ ID NO: 112, and a VL comprising the amino acid sequence of SEQ ID NO: 88. In some embodiments, the anti-C5 antibody comprises an Fc fragment. In some embodiments, the anti-C5 antibody comprises a human IgG4 Fc fragment or variant thereof. In some embodiments, the anti-C5 antibody mAb H1-8/L1-9 variant FMW comprises i) a heavy chain comprising the amino acid sequence of SEQ ID NO: 56 and a light chain comprising the amino acid sequence of SEQ ID NO: 25; or ii) a VH comprising the amino acid sequence of SEQ ID NO: 112, and a VL comprising the amino acid sequence of SEQ ID NO: 88; and a human IgG4 Fc fragment comprising the amino acid sequence of SEQ ID NO: 32. In some embodiments, the anti-C5 antibody comprises a human IgG4 Fc fragment having an S108P mutation relative to SEQ ID NO: 32, or a variant thereof. In some embodiments, the anti-C5 antibody mAb H1-8/L1-9 variant FMW comprises i) a heavy chain comprising the amino acid sequence of SEQ ID NO: 56 and a light chain comprising the amino acid sequence of SEQ ID NO: 25; or ii) a VH comprising the amino acid sequence of SEQ ID NO: 112, and a VL comprising the amino acid sequence of SEQ ID NO: 88; and a human IgG4 Fc fragment having an S108P mutation relative to SEQ ID NO: 32, comprising the amino acid sequence of SEQ ID NO: 33. In some embodiments, the anti-C5 antibody comprises a human IgG4 Fc fragment having an S108P mutation, a M308L mutation, and a N314A mutation relative to SEQ ID NO: 32, or variant thereof. In some embodiments, the anti-C5 antibody mAb H1-8/L1-9 variant FMW comprises i) a heavy chain comprising the amino acid sequence of SEQ ID NO: 56 and a light chain comprising the amino acid sequence of SEQ ID NO: 25; or ii) a VH comprising the amino acid sequence of SEQ ID NO: 112, and a VL comprising the amino acid sequence of SEQ ID NO: 88; and a human IgG4 Fc fragment having an S108P mutation, a M308L mutation, and a N314A mutation relative to SEQ ID NO: 32, comprising the amino acid sequence of SEQ ID NO: 61.

In some embodiments, the anti-C5 antibody or antigen-binding fragment thereof comprises at least one of the CDRs selected from the group consisting of: a VH-CDR1 comprising the amino acid sequence of SEQ ID NO: 47; a VH-CDR2 comprising the amino acid sequence of SEQ ID NO: 57; a VH-CDR3 comprising the amino acid sequence of SEQ ID NO: 49; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 10, or a variant thereof comprising up to 5 (e.g., 5, 4, 3, 2, or 1) amino acid variations (e.g., insertion, deletion, substitution, such as conserved substitution) in one or more of the CDRs. In some embodiments, the anti-C5 antibody or antigen-binding fragment thereof comprises: a VH-CDR1 comprising the amino acid sequence of SEQ ID NO: 47; a VH-CDR2 comprising the amino acid sequence of SEQ ID NO: 57; a VH-CDR3 comprising the amino acid sequence of SEQ ID NO: 49; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 10, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 47, or a variant thereof comprising up to about 3 (such as about any of 1, 2, or 3) amino acid substitutions (e.g., conserved substitution); a VH-CDR2 comprising the amino acid sequence of SEQ ID NO: 57, or a variant thereof comprising up to about 3 (such as about any of 1, 2, or 3) amino acid substitutions (e.g., conserved substitution); a VH-CDR3 comprising the amino acid sequence of SEQ ID NO: 49, or a variant thereof comprising up to about 3 (such as about any of 1, 2, or 3) amino acid substitutions (e.g., conserved substitution); a VL-CDR1 comprising the amino acid sequence of SEQ ID NO: 23, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 10, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 47, or a variant thereof comprising up to about 3 (such as about any of 1, 2, or 3) amino acid substitutions (e.g., conserved substitution); a VH-CDR2 comprising the amino acid sequence of SEQ ID NO: 57, or a variant thereof comprising up to about 3 (such as about any of 1, 2, or 3) amino acid substitutions (e.g., conserved substitution); a VH-CDR3 comprising the amino acid sequence of SEQ ID NO: 49; a VL-CDR1 comprising the amino acid sequence of SEQ ID NO: 23, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 10.

In some embodiments, the anti-C5 antibody or antigen-binding fragment thereof comprises: a VH-CDR1 comprising the amino acid sequence of SEQ ID NO: 47; a VH-CDR2 comprising the amino acid sequence of SEQ ID NO: 57; a VH-CDR3 comprising the amino acid sequence of SEQ ID NO: 49; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 10.

In some embodiments, the anti-C5 antibody or antigen-binding fragment thereof comprises at least one of the CDRs selected from the group consisting of: a VH-CDR1 comprising the amino acid sequence of SEQ ID NO: 86; a VH-CDR2 comprising the amino acid sequence of SEQ ID NO: 57; a VH-CDR3 comprising the amino acid sequence of SEQ ID NO: 49; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 96, or a variant thereof comprising up to 5 (e.g., 5, 4, 3, 2, or 1) amino acid variations (e.g., insertion, deletion, substitution, such as conserved substitution) in one or more of the CDRs. In some embodiments, the anti-C5 antibody or antigen-binding fragment thereof comprises: a VH-CDR1 comprising the amino acid sequence of SEQ ID NO: 86; a VH-CDR2 comprising the amino acid sequence of SEQ ID NO: 57; a VH-CDR3 comprising the amino acid sequence of SEQ ID NO: 49; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 96, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 86, or a variant thereof comprising up to about 3 (such as about any of 1, 2, or 3) amino acid substitutions (e.g., conserved substitution); a VH-CDR2 comprising the amino acid sequence of SEQ ID NO: 57, or a variant thereof comprising up to about 3 (such as about any of 1, 2, or 3) amino acid substitutions (e.g., conserved substitution); a VH-CDR3 comprising the amino acid sequence of SEQ ID NO: 49, or a variant thereof comprising up to about 3 (such as about any of 1, 2, or 3) amino acid substitutions (e.g., conserved substitution); a VL-CDR1 comprising the amino acid sequence of SEQ ID NO: 23, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 96, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 86, or a variant thereof comprising up to about 3 (such as about any of 1, 2, or 3) amino acid substitutions (e.g., conserved substitution); a VH-CDR2 comprising the amino acid sequence of SEQ ID NO: 57, or a variant thereof comprising up to about 3 (such as about any of 1, 2, or 3) amino acid substitutions (e.g., conserved substitution); a VH-CDR3 comprising the amino acid sequence of SEQ ID NO: 49; a VL-CDR1 comprising the amino acid sequence of SEQ ID NO: 23, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 96.

In some embodiments, the anti-C5 antibody or antigen-binding fragment thereof comprises: a VH-CDR1 comprising the amino acid sequence of SEQ ID NO: 86; a VH-CDR2 comprising the amino acid sequence of SEQ ID NO: 57; a VH-CDR3 comprising the amino acid sequence of SEQ ID NO: 49; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 96.

In some embodiments, the anti-C5 antibody or antigen-binding fragment thereof comprises a VH comprising the amino acid sequence of SEQ ID NO: 59, or a variant thereof comprising at least about 80% (e.g., at least about any of 85%, 90%, 95%, 96%, 97%, 98%, 99% or more) sequence identity to SEQ ID NO: 59, and a VL comprising the amino acid sequence of SEQ ID NO: 25, or a variant thereof comprising at least about 80% (e.g., at least about any of 85%, 90%, 95%, 96%, 97%, 98%, 99% or more) sequence identity to SEQ ID NO: 25. In some embodiments, the anti-C5 antibody or antigen-binding fragment thereof comprises a VH comprising the amino acid sequence of SEQ ID NO: 59, and a VL comprising the amino acid sequence of SEQ ID NO: 25. In some embodiments, the anti-C5 antibody or antigen-binding fragment thereof comprises a VH comprising the amino acid sequence of SEQ ID NO: 87, or a variant thereof comprising at least about 80% (e.g., at least about any of 85%, 90%, 95%, 96%, 97%, 98%, 99% or more) sequence identity to SEQ ID NO: 87, and a VL comprising the amino acid sequence of SEQ ID NO: 88, or a variant thereof comprising at least about 80% (e.g., at least about any of 85%, 90%, 95%, 96%, 97%, 98%, 99% or more) sequence identity to SEQ ID NO: 88. In some embodiments, the anti-C5 antibody or antigen-binding fragment thereof comprises a VH comprising the amino acid sequence of SEQ ID NO: 87, and a VL comprising the amino acid sequence of SEQ ID NO: 88. In some embodiments, the VH and VL do not comprise a signal peptide at the N-terminus.

In some embodiments, the anti-C5 antibody comprises a heavy chain comprising the amino acid sequence of SEQ ID NO: 59, or a variant thereof comprising at least about 80% (e.g., at least about any of 85%, 90%, 95%, 96%, 97%, 98%, 99% or more) sequence identity to SEQ ID NO: 59. In some embodiments, the anti-C5 antibody comprises a light chain comprising the amino acid sequence of SEQ ID NO: 25, or a variant thereof comprising at least about 80% (e.g., at least about any of 85%, 90%, 95%, 96%, 97%, 98%, 99% or more) sequence identity to SEQ ID NO: 25. In some embodiments, the anti-C5 antibody is humanized. In some embodiments, the anti-C5 antibody is a chimeric antibody. In some embodiments, the anti-C5 antibody is mAb H1-8/L1-9 variant FMEH, or a variant thereof. In some embodiments, the anti-C5 antibody mAb H1-8L1-9 variant FMEH comprises i) a heavy chain comprising the amino acid sequence of SEQ ID NO: 59 and a light chain comprising the amino acid sequence of SEQ ID NO: 25; or ii) a VH comprising the amino acid sequence of SEQ ID NO: 87, and a VL comprising the amino acid sequence of SEQ ID NO: 88. In some embodiments, the anti-C5 antibody comprises an Fc fragment. In some embodiments, the anti-C5 antibody comprises a human IgG4 Fc fragment or variant thereof. In some embodiments, the anti-C5 antibody mAb H1-8/L1-9 variant FMEH comprises i) a heavy chain comprising the amino acid sequence of SEQ ID NO: 59 and a light chain comprising the amino acid sequence of SEQ ID NO: 25; or ii) a VH comprising the amino acid sequence of SEQ ID NO: 87, and a VL comprising the amino acid sequence of SEQ ID NO: 88; and a human IgG4 Fc fragment comprising the amino acid sequence of SEQ ID NO: 32. In some embodiments, the anti-C5 antibody comprises a human IgG4 Fc fragment having an S108P mutation relative to SEQ ID NO: 32, or a variant thereof. In some embodiments, the anti-C5 antibody mAb H1-8/L1-9 variant FMEH comprises i) a heavy chain comprising the amino acid sequence of SEQ ID NO: 59 and a light chain comprising the amino acid sequence of SEQ ID NO: 25; or ii) a VH comprising the amino acid sequence of SEQ ID NO: 87, and a VL comprising the amino acid sequence of SEQ ID NO: 88; and a human IgG4 Fc fragment having an S108P mutation relative to SEQ ID NO: 32, comprising the amino acid sequence of SEQ ID NO: 33. In some embodiments, the anti-C5 antibody comprises a human IgG4 Fc fragment having an S108P mutation, a M308L mutation, and a N314A mutation relative to SEQ ID NO: 32, or a variant thereof. In some embodiments, the anti-C5 antibody mAb H1-8/L1-9 variant FMEH comprises i) a heavy chain comprising the amino acid sequence of SEQ ID NO: 59 and a light chain comprising the amino acid sequence of SEQ ID NO: 25; or ii) a VH comprising the amino acid sequence of SEQ ID NO: 87, and a VL comprising the amino acid sequence of SEQ ID NO: 88; and a human IgG4 Fc fragment having an S108P mutation, a M308L mutation, and a N314A mutation relative to SEQ ID NO: 32, comprising the amino acid sequence of SEQ ID NO: 61. In some embodiments, the anti-C5 antibody mAb H1-8/L1-9 variant FMEH comprises a heavy chain comprising the amino acid sequence of SEQ ID NO: 119, and a light chain comprising the amino acid sequence of SEQ ID NO: 74. In some embodiments, the heavy chain and light chain do not comprise a signal peptide at the N-terminus. In some embodiments, the anti-C5 antibody mAb H1-8/L1-9 variant FMEH comprises a heavy chain comprising the amino acid sequence of SEQ ID NO: 120, and a light chain comprising the amino acid sequence of SEQ ID NO: 90 (hereinafter also referred to as “FMEH-IgG4-PLA”).

In some embodiments, the anti-C5 antibody or antigen-binding fragment thereof comprises: a VH-CDR1 comprising the amino acid sequence of SEQ ID NO: 37; a VH-CDR2 comprising the amino acid sequence of SEQ ID NO: 62; a VH-CDR3 comprising the amino acid sequence of SEQ ID NO: 39; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 10, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 37, or a variant thereof comprising up to about 3 (such as about any of 1, 2, or 3) amino acid substitutions (e.g., conserved substitution); a VH-CDR2 comprising the amino acid sequence of SEQ ID NO: 62, or a variant thereof comprising up to about 3 (such as about any of 1, 2, or 3) amino acid substitutions (e.g., conserved substitution); a VH-CDR3 comprising the amino acid sequence of SEQ ID NO: 39, or a variant thereof comprising up to about 3 (such as about any of 1, 2, or 3) amino acid substitutions (e.g., conserved substitution); a VL-CDR1 comprising the amino acid sequence of SEQ ID NO: 23, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 10, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 37; a VH-CDR2 comprising the amino acid sequence of SEQ ID NO: 62; a VH-CDR3 comprising the amino acid sequence of SEQ ID NO: 39; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 10.

In some embodiments, the anti-C5 antibody or antigen-binding fragment thereof comprises: a VH-CDR1 comprising the amino acid sequence of SEQ ID NO: 108; a VH-CDR2 comprising the amino acid sequence of SEQ ID NO: 62; a VH-CDR3 comprising the amino acid sequence of SEQ ID NO: 39; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 96, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 108, or a variant thereof comprising up to about 3 (such as about any of 1, 2, or 3) amino acid substitutions (e.g., conserved substitution); a VH-CDR2 comprising the amino acid sequence of SEQ ID NO: 62, or a variant thereof comprising up to about 3 (such as about any of 1, 2, or 3) amino acid substitutions (e.g., conserved substitution); a VH-CDR3 comprising the amino acid sequence of SEQ ID NO: 39, or a variant thereof comprising up to about 3 (such as about any of 1, 2, or 3) amino acid substitutions (e.g., conserved substitution); a VL-CDR1 comprising the amino acid sequence of SEQ ID NO: 23, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 96, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 108; a VH-CDR2 comprising the amino acid sequence of SEQ ID NO: 62; a VH-CDR3 comprising the amino acid sequence of SEQ ID NO: 39; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 96.

In some embodiments, the anti-C5 antibody or antigen-binding fragment thereof comprises a VH comprising the amino acid sequence of SEQ ID NO: 64, or a variant thereof comprising at least about 80% (e.g., at least about any of 85%, 90%, 95%, 96%, 97%, 98%, 99% or more) sequence identity to SEQ ID NO: 64, and a VL comprising the amino acid sequence of SEQ ID NO: 25, or a variant thereof comprising at least about 80% (e.g., at least about any of 85%, 90%, 95%, 96%, 97%, 98%, 99% or more) sequence identity to SEQ ID NO: 25. In some embodiments, the anti-C5 antibody or antigen-binding fragment thereof comprises a VH comprising the amino acid sequence of SEQ ID NO: 64, and a VL comprising the amino acid sequence of SEQ ID NO: 25. In some embodiments, the anti-C5 antibody or antigen-binding fragment thereof comprises a VH comprising the amino acid sequence of SEQ ID NO: 113, or a variant thereof comprising at least about 80% (e.g., at least about any of 85%, 90%, 95%, 96%, 97%, 98%, 99% or more) sequence identity to SEQ ID NO: 113, and a VL comprising the amino acid sequence of SEQ ID NO: 88, or a variant thereof comprising at least about 80% (e.g., at least about any of 85%, 90%, 95%, 96%, 97%, 98%, 99% or more) sequence identity to SEQ ID NO: 88. In some embodiments, the anti-C5 antibody or antigen-binding fragment thereof comprises a VH comprising the amino acid sequence of SEQ ID NO: 113, and a VL comprising the amino acid sequence of SEQ ID NO: 88. In some embodiments, the VH and VL do not comprise a signal peptide at the N-terminus.

In some embodiments, the anti-C5 antibody comprises a heavy chain comprising the amino acid sequence of SEQ ID NO: 64, or a variant thereof comprising at least about 80% (e.g., at least about any of 85%, 90%, 95%, 96%, 97%, 98%, 99% or more) sequence identity to SEQ ID NO: 64. In some embodiments, the anti-C5 antibody comprises a light chain comprising the amino acid sequence of SEQ ID NO: 25, or a variant thereof comprising at least about 80% (e.g., at least about any of 85%, 90%, 95%, 96%, 97%, 98%, 99% or more) sequence identity to SEQ ID NO: 25. In some embodiments, the anti-C5 antibody is humanized. In some embodiments, the anti-C5 antibody is a chimeric antibody. In some embodiments, the anti-C5 antibody is mAb H1-8/L1-9 variant IWWH, or a variant thereof. In some embodiments, the anti-C5 antibody mAb H1-8/L1-9 variant IWWH comprises i) a heavy chain comprising the amino acid sequence of SEQ ID NO: 64 and a light chain comprising the amino acid sequence of SEQ ID NO: 25; or ii) a VH comprising the amino acid sequence of SEQ ID NO: 113, and a VL comprising the amino acid sequence of SEQ ID NO: 88. In some embodiments, the anti-C5 antibody comprises an Fc fragment. In some embodiments, the anti-C5 antibody comprises a human IgG4 Fc fragment or variant thereof. In some embodiments, the anti-C5 antibody mAb H1-8/L1-9 variant IWWH comprises i) a heavy chain comprising the amino acid sequence of SEQ ID NO: 64 and a light chain comprising the amino acid sequence of SEQ ID NO: 25; or ii) a VH comprising the amino acid sequence of SEQ ID NO: 113, and a VL comprising the amino acid sequence of SEQ ID NO: 88; and a human IgG4 Fc fragment comprising the amino acid sequence of SEQ ID NO: 32. In some embodiments, the anti-C5 antibody comprises a human IgG4 Fc fragment having an S108P mutation relative to SEQ ID NO: 32, or a variant thereof. In some embodiments, the anti-C5 antibody mAb H1-8/L1-9 variant IWWH comprises i) a heavy chain comprising the amino acid sequence of SEQ ID NO: 64 and a light chain comprising the amino acid sequence of SEQ ID NO: 25; or ii) a VH comprising the amino acid sequence of SEQ ID NO: 113, and a VL comprising the amino acid sequence of SEQ ID NO: 88; and a human IgG4 Fc fragment having an S108P mutation relative to SEQ ID NO: 32, comprising the amino acid sequence of SEQ ID NO: 33. In some embodiments, the anti-C5 antibody comprises a human IgG4 Fc fragment having an S108P mutation, a M308L mutation, and a N314A mutation relative to SEQ ID NO: 32, or a variant thereof. In some embodiments, the anti-C5 antibody mAb H1-8/L1-9 variant IWWH comprises i) a heavy chain comprising the amino acid sequence of SEQ ID NO: 64 and a light chain comprising the amino acid sequence of SEQ ID NO: 25; or ii) a VH comprising the amino acid sequence of SEQ ID NO: 113, and a VL comprising the amino acid sequence of SEQ ID NO: 88; and a human IgG4 Fc fragment having an S108P mutation, a M308L mutation, and a N314A mutation relative to SEQ ID NO: 32, comprising the amino acid sequence of SEQ ID NO: 61. In some embodiments, the anti-C5 antibody mAb H1-8/L1-9 variant IWWH comprises a heavy chain comprising the amino acid sequence of SEQ ID NO: 121, and a light chain comprising the amino acid sequence of SEQ ID NO: 74. In some embodiments, the heavy chain and light chain do not comprise a signal peptide at the N-terminus. In some embodiments, the anti-C5 antibody mAb H1-8/L1-9 variant IWWH comprises a heavy chain comprising the amino acid sequence of SEQ ID NO: 122, and a light chain comprising the amino acid sequence of SEQ ID NO: 90.

In some embodiments, the anti-C5 antibody or antigen-binding fragment thereof comprises: a VH-CDR1 comprising the amino acid sequence of SEQ ID NO: 42; a VH-CDR2 comprising the amino acid sequence of SEQ ID NO: 65; a VH-CDR3 comprising the amino acid sequence of SEQ ID NO: 44; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 10, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 42, or a variant thereof comprising up to about 3 (such as about any of 1, 2, or 3) amino acid substitutions (e.g., conserved substitution); a VH-CDR2 comprising the amino acid sequence of SEQ ID NO: 65, or a variant thereof comprising up to about 3 (such as about any of 1, 2, or 3) amino acid substitutions (e.g., conserved substitution); a VH-CDR3 comprising the amino acid sequence of SEQ ID NO: 44, or a variant thereof comprising up to about 3 (such as about any of 1, 2, or 3) amino acid substitutions (e.g., conserved substitution); a VL-CDR1 comprising the amino acid sequence of SEQ ID NO: 23, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 10, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 42; a VH-CDR2 comprising the amino acid sequence of SEQ ID NO: 65; a VH-CDR3 comprising the amino acid sequence of SEQ ID NO: 44; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 10.

In some embodiments, the anti-C5 antibody or antigen-binding fragment thereof comprises: a VH-CDR1 comprising the amino acid sequence of SEQ ID NO: 108; a VH-CDR2 comprising the amino acid sequence of SEQ ID NO: 65; a VH-CDR3 comprising the amino acid sequence of SEQ ID NO: 44; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 96, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 108, or a variant thereof comprising up to about 3 (such as about any of 1, 2, or 3) amino acid substitutions (e.g., conserved substitution); a VH-CDR2 comprising the amino acid sequence of SEQ ID NO: 65, or a variant thereof comprising up to about 3 (such as about any of 1, 2, or 3) amino acid substitutions (e.g., conserved substitution); a VH-CDR3 comprising the amino acid sequence of SEQ ID NO: 44, or a variant thereof comprising up to about 3 (such as about any of 1, 2, or 3) amino acid substitutions (e.g., conserved substitution); a VL-CDR1 comprising the amino acid sequence of SEQ ID NO: 23, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 96, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 108; a VH-CDR2 comprising the amino acid sequence of SEQ ID NO: 65; a VH-CDR3 comprising the amino acid sequence of SEQ ID NO: 44; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 96.

In some embodiments, the anti-C5 antibody or antigen-binding fragment thereof comprises a VH comprising the amino acid sequence of SEQ ID NO: 67, or a variant thereof comprising at least about 80% (e.g., at least about any of 85%, 90%, 95%, 96%, 97%, 98%, 99% or more) sequence identity to SEQ ID NO: 67, and a VL comprising the amino acid sequence of SEQ ID NO: 25, or a variant thereof comprising at least about 80% (e.g., at least about any of 85%, 90%, 95%, 96%, 97%, 98%, 99% or more) sequence identity to SEQ ID NO: 25. In some embodiments, the anti-C5 antibody or antigen-binding fragment thereof comprises a VH comprising the amino acid sequence of SEQ ID NO: 67, and a VL comprising the amino acid sequence of SEQ ID NO: 25. In some embodiments, the anti-C5 antibody or antigen-binding fragment thereof comprises a VH comprising the amino acid sequence of SEQ ID NO: 114, or a variant thereof comprising at least about 80% (e.g., at least about any of 85%, 90%, 95%, 96%, 97%, 98%, 99% or more) sequence identity to SEQ ID NO: 114, and a VL comprising the amino acid sequence of SEQ ID NO: 88, or a variant thereof comprising at least about 80% (e.g., at least about any of 85%, 90%, 95%, 96%, 97%, 98%, 99% or more) sequence identity to SEQ ID NO: 88. In some embodiments, the anti-C5 antibody or antigen-binding fragment thereof comprises a VH comprising the amino acid sequence of SEQ ID NO: 114, and a VL comprising the amino acid sequence of SEQ ID NO: 88. In some embodiments, the VH and VL do not comprise a signal peptide at the N-terminus.

In some embodiments, the anti-C5 antibody comprises a heavy chain comprising the amino acid sequence of SEQ ID NO: 67, or a variant thereof comprising at least about 80% (e.g., at least about any of 85%, 90%, 95%, 96%, 97%, 98%, 99% or more) sequence identity to SEQ ID NO: 67. In some embodiments, the anti-C5 antibody comprises a light chain comprising the amino acid sequence of SEQ ID NO: 25, or a variant thereof comprising at least about 80% (e.g., at least about any of 85%, 90%, 95%, 96%, 97%, 98%, 99% or more) sequence identity to SEQ ID NO: 25. In some embodiments, the anti-C5 antibody is humanized. In some embodiments, the anti-C5 antibody is a chimeric antibody. In some embodiments, the anti-C5 antibody is mAb H1-8/L1-9 variant IFWH, or a variant thereof. In some embodiments, the anti-C5 antibody mAb H1-8/L1-9 variant IFWH comprises i) a heavy chain comprising the amino acid sequence of SEQ ID NO: 67 and a light chain comprising the amino acid sequence of SEQ ID NO: 25; or ii) a VH comprising the amino acid sequence of SEQ ID NO: 114, and a VL comprising the amino acid sequence of SEQ ID NO: 88. In some embodiments, the anti-C5 antibody comprises an Fc fragment. In some embodiments, the anti-C5 antibody comprises a human IgG4 Fc fragment or variant thereof. In some embodiments, the anti-C5 antibody mAb H1-8/L1-9 variant IFWH comprises i) a heavy chain comprising the amino acid sequence of SEQ ID NO: 67 and a light chain comprising the amino acid sequence of SEQ ID NO: 25; or ii) a VH comprising the amino acid sequence of SEQ ID NO: 114, and a VL comprising the amino acid sequence of SEQ ID NO: 88; and a human IgG4 Fc fragment comprising the amino acid sequence of SEQ ID NO: 32. In some embodiments, the anti-C5 antibody comprises a human IgG4 Fc fragment having an S108P mutation relative to SEQ ID NO: 32, or a variant thereof. In some embodiments, the anti-C5 antibody mAb H1-8/L1-9 variant IFWH comprises i) a heavy chain comprising the amino acid sequence of SEQ ID NO: 67 and a light chain comprising the amino acid sequence of SEQ ID NO: 25; or ii) a VH comprising the amino acid sequence of SEQ ID NO: 114, and a VL comprising the amino acid sequence of SEQ ID NO: 88; and a human IgG4 Fc fragment having an S108P mutation relative to SEQ ID NO: 32, comprising the amino acid sequence of SEQ ID NO: 33. In some embodiments, the anti-C5 antibody comprises a human IgG4 Fc fragment having an S108P mutation, a M308L mutation, and a N314A mutation relative to SEQ ID NO: 32, or a variant thereof. In some embodiments, the anti-C5 antibody mAb H1-8/L1-9 variant IFWH comprises i) a heavy chain comprising the amino acid sequence of SEQ ID NO: 67 and a light chain comprising the amino acid sequence of SEQ ID NO: 25; or ii) a VH comprising the amino acid sequence of SEQ ID NO: 114, and a VL comprising the amino acid sequence of SEQ ID NO: 88; and a human IgG4 Fc fragment having an S108P mutation, a M308L mutation, and a N314A mutation relative to SEQ ID NO: 32, comprising the amino acid sequence of SEQ ID NO: 61. In some embodiments, the anti-C5 antibody mAb H1-8/L1-9 variant IFWH comprises a heavy chain comprising the amino acid sequence of SEQ ID NO: 123, and a light chain comprising the amino acid sequence of SEQ ID NO: 74. In some embodiments, the heavy chain and light chain do not comprise a signal peptide at the N-terminus. In some embodiments, the anti-C5 antibody mAb H1-8/L1-9 variant IFWH comprises a heavy chain comprising the amino acid sequence of SEQ ID NO: 124, and a light chain comprising the amino acid sequence of SEQ ID NO: 90.

In some embodiments, the anti-C5 antibody or antigen-binding fragment thereof comprises: a VH-CDR1 comprising the amino acid sequence of SEQ ID NO: 52; a VH-CDR2 comprising the amino acid sequence of SEQ ID NO: 68; a VH-CDR3 comprising the amino acid sequence of SEQ ID NO: 54; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 10, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 52, or a variant thereof comprising up to about 3 (such as about any of 1, 2, or 3) amino acid substitutions (e.g., conserved substitution); a VH-CDR2 comprising the amino acid sequence of SEQ ID NO: 68, or a variant thereof comprising up to about 3 (such as about any of 1, 2, or 3) amino acid substitutions (e.g., conserved substitution); a VH-CDR3 comprising the amino acid sequence of SEQ ID NO: 54, or a variant thereof comprising up to about 3 (such as about any of 1, 2, or 3) amino acid substitutions (e.g., conserved substitution); a VL-CDR1 comprising the amino acid sequence of SEQ ID NO: 23, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 10, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 52; a VH-CDR2 comprising the amino acid sequence of SEQ ID NO: 68; a VH-CDR3 comprising the amino acid sequence of SEQ ID NO: 54; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 10.

In some embodiments, the anti-C5 antibody or antigen-binding fragment thereof comprises: a VH-CDR1 comprising the amino acid sequence of SEQ ID NO: 86; a VH-CDR2 comprising the amino acid sequence of SEQ ID NO: 68; a VH-CDR3 comprising the amino acid sequence of SEQ ID NO: 54; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 96, or a variant thereof comprising up to 5 (e.g., 5, 4, 3, 2, or 1) amino acid variations (e.g., insertion, deletion, substitution, such as conserved substitution) in one or more of the CDRs.

In some embodiments, the anti-C5 antibody or antigen-binding fragment thereof comprises: a VH-CDR1 comprising the amino acid sequence of SEQ ID NO: 86, or a variant thereof comprising up to about 3 (such as about any of 1, 2, or 3) amino acid substitutions (e.g., conserved substitution); a VH-CDR2 comprising the amino acid sequence of SEQ ID NO: 68, or a variant thereof comprising up to about 3 (such as about any of 1, 2, or 3) amino acid substitutions (e.g., conserved substitution); a VH-CDR3 comprising the amino acid sequence of SEQ ID NO: 54, or a variant thereof comprising up to about 3 (such as about any of 1, 2, or 3) amino acid substitutions (e.g., conserved substitution); a VL-CDR1 comprising the amino acid sequence of SEQ ID NO: 23, or a variant thereof comprising up to about 3 (such as about any of 1, 2, or 3) amino acid substitutions (e.g., conserved substitution); a VL-CDR2 comprising the amino acid sequence of SEQ ID NO: 9, or a variant thereof comprising up to about 3 (such as about any of 1, 2, or 3) amino acid substitutions (e.g., conserved substitution); and a VL-CDR3 comprising the amino acid sequence of SEQ ID NO: 96, or a variant thereof comprising up to about 3 (such as about any of 1, 2, or 3) amino acid substitutions (e.g., conserved substitution).

In some embodiments, the anti-C5 antibody or antigen-binding fragment thereof comprises: a VH-CDR1 comprising the amino acid sequence of SEQ ID NO: 86; a VH-CDR2 comprising the amino acid sequence of SEQ ID NO: 68; a VH-CDR3 comprising the amino acid sequence of SEQ ID NO: 54; a VL-CDR1 comprising the amino acid sequence of SEQ ID NO: 23; a VL-CDR2 comprising the amino acid sequence of SEQ ID NO: 9; and a VL-CDR3 comprising the amino acid sequence of SEQ ID NO: 96.

In some embodiments, the anti-C5 antibody or antigen-binding fragment thereof comprises a VH comprising the amino acid sequence of SEQ ID NO: 70, or a variant thereof comprising at least about 80% (e.g., at least about any of 85%, 90%, 95%, 96%, 97%, 98%, 99% or more) sequence identity to SEQ ID NO: 70, and a VL comprising the amino acid sequence of SEQ ID NO: 25, or a variant thereof comprising at least about 80% (e.g., at least about any of 85%, 90%, 95%, 96%, 97%, 98%, 99% or more) sequence identity to SEQ ID NO: 25. In some embodiments, the anti-C5 antibody or antigen-binding fragment thereof comprises a VH comprising the amino acid sequence of SEQ ID NO: 70, and a VL comprising the amino acid sequence of SEQ ID NO: 25. In some embodiments, the anti-C5 antibody or antigen-binding fragment thereof comprises a VH comprising the amino acid sequence of SEQ ID NO: 115, or a variant thereof comprising at least about 80% (e.g., at least about any of 85%, 90%, 95%, 96%, 97%, 98%, 99% or more) sequence identity to SEQ ID NO: 115, and a VL comprising the amino acid sequence of SEQ ID NO: 88, or a variant thereof comprising at least about 80% (e.g., at least about any of 85%, 90%, 95%, 96%, 97%, 98%, 99% or more) sequence identity to SEQ ID NO: 88. In some embodiments, the anti-C5 antibody or antigen-binding fragment thereof comprises a VH comprising the amino acid sequence of SEQ ID NO: 115, and a VL comprising the amino acid sequence of SEQ ID NO: 88. In some embodiments, the VH and VL do not comprise a signal peptide at the N-terminus.

In some embodiments, the anti-C5 antibody comprises a heavy chain comprising the amino acid sequence of SEQ ID NO: 70, or a variant thereof comprising at least about 80% (e.g., at least about any of 85%, 90%, 95%, 96%, 97%, 98%, 99% or more) sequence identity to SEQ ID NO: 70. In some embodiments, the anti-C5 antibody comprises a light chain comprising the amino acid sequence of SEQ ID NO: 25, or a variant thereof comprising at least about 80% (e.g., at least about any of 85%, 90%, 95%, 96%, 97%, 98%, 99% or more) sequence identity to SEQ ID NO: 25. In some embodiments, the anti-C5 antibody is humanized. In some embodiments, the anti-C5 antibody is a chimeric antibody. In some embodiments, the anti-C5 antibody is mAb H1-8/L1-9 variant FMWH, or a variant thereof. In some embodiments, the anti-C5 antibody mAb H1-8L1-9 variant FMWH comprises i) a heavy chain comprising the amino acid sequence of SEQ ID NO: 70 and a light chain comprising the amino acid sequence of SEQ ID NO: 25; or ii) a VH comprising the amino acid sequence of SEQ ID NO: 115, and a VL comprising the amino acid sequence of SEQ ID NO: 88. In some embodiments, the anti-C5 antibody comprises an Fc fragment. In some embodiments, the anti-C5 antibody comprises a human IgG4 Fc fragment or variant thereof. In some embodiments, the anti-C5 antibody mAb H1-8/L1-9 variant FMWH comprises i) a heavy chain comprising the amino acid sequence of SEQ ID NO: 70 and a light chain comprising the amino acid sequence of SEQ ID NO: 25; or ii) a VH comprising the amino acid sequence of SEQ ID NO: 115, and a VL comprising the amino acid sequence of SEQ ID NO: 88; and a human IgG4 Fc fragment comprising the amino acid sequence of SEQ ID NO: 32. In some embodiments, the anti-C5 antibody comprises a human IgG4 Fc fragment having an S108P mutation relative to SEQ ID NO: 32, or a variant thereof. In some embodiments, the anti-C5 antibody mAb H1-8/L1-9 variant FMWH comprises i) a heavy chain comprising the amino acid sequence of SEQ ID NO: 70 and a light chain comprising the amino acid sequence of SEQ ID NO: 25; or ii) a VH comprising the amino acid sequence of SEQ ID NO: 115, and a VL comprising the amino acid sequence of SEQ ID NO: 88; and a human IgG4 Fc fragment having an S108P mutation relative to SEQ ID NO: 32, comprising the amino acid sequence of SEQ ID NO: 33. In some embodiments, the anti-C5 antibody comprises a human IgG4 Fc fragment having an S108P mutation, a M308L mutation, and a N314A mutation relative to SEQ ID NO: 32, or a variant thereof. In some embodiments, the anti-C5 antibody mAb H1-8/L1-9 variant FMWH comprises i) a heavy chain comprising the amino acid sequence of SEQ ID NO: 70 and a light chain comprising the amino acid sequence of SEQ ID NO: 25; or ii) a VH comprising the amino acid sequence of SEQ ID NO: 115, and a VL comprising the amino acid sequence of SEQ ID NO: 88; and a human IgG4 Fc fragment having an S108P mutation, a M308L mutation, and a N314A mutation relative to SEQ ID NO: 32, comprising the amino acid sequence of SEQ ID NO: 61. In some embodiments, the anti-C5 antibody mAb H1-8/L1-9 variant FMWH comprises a heavy chain comprising the amino acid sequence of SEQ ID NO: 125, and a light chain comprising the amino acid sequence of SEQ ID NO: 74. In some embodiments, the heavy chain and light chain do not comprise a signal peptide at the N-terminus. In some embodiments, the anti-C5 antibody mAb H1-8/L1-9 variant FMWH comprises a heavy chain comprising the amino acid sequence of SEQ ID NO: 126, and a light chain comprising the amino acid sequence of SEQ ID NO: 90.

In some embodiments, the anti-C5 antibody or antigen-binding fragment thereof comprises a substitution (e.g., conserved substitution) of asparagine at position #8 (i.e., N8) in VH-CDR1 relative to SEQ ID NO: 3. In some embodiments, the substitution at N8 is N8→H8 (i.e., N8H), N8→W8 (i.e., N8W), N8→18 (i.e., N8I), N8→V8 (i.e., N8V), N8→Y8 (i.e., N8Y), or N8→F8 (i.e., N8F).

In some embodiments, the anti-C5 antibody or antigen-binding fragment thereof comprises a substitution (e.g., conserved substitution) of leucine at position #9 (i.e., L9) in VH-CDR1 relative to SEQ ID NO: 20. In some embodiments, the substitution at L9 is L9→W9 (i.e., L9W), L9→I9 (i.e., L9I), L9→V9 (i.e., L9V), L9→Y9 (i.e., L9Y), or L9→F9 (i.e., L9F).

In some embodiments, the anti-C5 antibody or antigen-binding fragment thereof comprises a substitution (e.g., conserved substitution) of proline at position #4 (i.e., P4) in VH-CDR2 relative to SEQ ID NO: 4. In some embodiments, the substitution at P4 is P4→F4 (i.e., P4F), P4→L4 (i.e., P4L), P4→M4 (i.e., P4M), P4→W4 (i.e., P4W), or P4→I4 (i.e., P4I).

In some embodiments, the anti-C5 antibody or antigen-binding fragment thereof comprises a substitution (e.g., conserved substitution) of threonine at position #9 (i.e., T9) in VH-CDR2 relative to SEQ ID NO: 4. In some embodiments, the substitution at T9 is T9→H9 (i.e., T9H), T9→F9 (i.e., T9F), T9→L9 (i.e., T9L), T9→M9 (i.e., T9M), T9→W9 (i.e., T9W), or T9→I9 (i.e., T9I).

In some embodiments, the anti-C5 antibody or antigen-binding fragment thereof comprises a substitution (e.g., conserved substitution) of P4 in VH-CDR2 relative to SEQ ID NO: 4; and a substitution (e.g., conserved substitution) of T9 in VH-CDR2 relative to SEQ ID NO: 4. In some embodiments, the substitution at P4 is P4F, P4L, P4M, P4W, or P41; and the substitution at T9 is T9H, T9F, T9L, T9M, T9W, or T9I.

In some embodiments, the anti-C5 antibody or antigen-binding fragment thereof comprises a substitution (e.g., conserved substitution) of valine at position #16 (i.e., V16) in VH-CDR3 relative to SEQ ID NO: 5. In some embodiments, the substitution at V16 is V16→F16 (i.e., V16F), V16→E16 (i.e., V16E) or V16→W16 (i.e., V16W).

In some embodiments, the anti-C5 antibody or antigen-binding fragment thereof comprises a substitution (e.g., conserved substitution) at two or more amino acid residues selected from the group consisting of: L9 in VH-CDR1 relative to SEQ ID NO: 20, P4 in VH-CDR2 relative to SEQ ID NO: 4, T9 in VH-CDR2 relative to SEQ ID NO: 4, and V16 in VH-CDR3 relative to SEQ ID NO: 5. In some embodiments, the anti-C5 antibody or antigen-binding fragment thereof comprising a substitution at two or more amino acid residues selected from the group consisting of L9 in VH-CDR1 relative to SEQ ID NO: 20, P4 in VH-CDR2 relative to SEQ ID NO: 4, T9 in VH-CDR2 relative to SEQ ID NO: 4, and V16 in VH-CDR3 relative to SEQ ID NO: 5 comprises two or more substitutions selected from the group consisting of: L9I/P4M, L9I/P4W, L9I/P4F, L9F/P4M, L9F/P4W, L9F/P4F, L9I/P4M/V16W, L9I/P4W/V16W (hereinafter referred to as “IWW” mutation), L9I/P4F/V16W (hereinafter referred to as “IFW” mutation), L9F/P4M/V16W (hereinafter referred to as “FMW” mutation), L9F/P4W/V16W, L9F/P4F/V16W, L9I/P4M/V16E, L9I/P4W/V16E, L9I/P4F/V6E, L9F/P4M/V16E (hereinafter referred to as “FME” mutation), L9F/P4W/V16E, L9F/P4F/V16E, L9I/P4M/T9H/V16W, L9I/P4W/T9H/V16W (hereinafter referred to as “IWWH” mutation), L9I/P4F/T9H/V16W (hereinafter referred to as “IFWH” mutation), L9F/P4M/T9H/V16W (hereinafter referred to as “FMWH” mutation), L9F/P4W/T9H/V16W, L9F/P4F/T9H/V16W, L9I/P4M/T9H/V16E, L9I/P4W/T9H/V16E, L9I/P4F/T9H/V16E, L9F/P4M/T9H/V16E (hereinafter referred to as “FMEH” mutation), L9F/P4W/T9H/V16E, and L9F/P4F/T9H/V16E. In some embodiments, the anti-C5 antibody or antigen-binding fragment thereof comprises triple mutations (e.g., IWW, IFW, FME, or FMW mutations). In some embodiments, the anti-C5 antibody or antigen-binding fragment thereof comprises quadruple mutations (e.g., IWWH, IFWH, FMEH, or FMWH mutations). In some embodiments, the anti-C5 antibody or antigen-binding fragment thereof comprises FMEH mutation.

In some embodiments, the anti-C5 antibody is a chimeric antibody. In some embodiments, the anti-human C5 antibody comprises human light chain and human heavy chain constant regions in combination with the variable region CDR sequences described above. One of ordinary skill in the art would be able to prepare and obtain a chimeric antibody using known techniques of swapping relevant domains of specific antibodies of interest. Such an antibody is easily prepared by grafting heterogeneous antibody domains, incorporating one or more CDR sequences described herein. Using known recombinant technology, it is possible to obtain and prepare a recombinant antibody comprising heavy chain and light chain constant regions encoded by nucleic acid sequences of human heavy chain and light chain constant regions; and the heavy chain and light chain variable regions comprising CDRs encoded by nucleic acid sequences corresponding to the CDR sequences described herein. One of ordinary skill in the art can prepare an anti-human C5 antibody comprising one or more CDR sequences described herein, wherein portions of the light chain alone or portions of the heavy chain alone are replaced with regions from an antibody belonging to another species, such as, for example, human, monkey, or mouse. An anti-human-C5 antibody comprising variable regions having one or more CDR sequences selected from SEQ ID NOs: 3-5, 8-11, 14, 17, 20, 23, 26, 29, 34, 37-39, 42-44, 47-49, 52-54, 57, 62, 65, 68, 86, 94, 96, 97, 100, 102, 105, and 108, or a variant thereof, in combination with murine or non-murine (e.g., human) antibody structural elements outside the CDR regions can be prepared by routine methods known in the art. In some embodiments, the anti-C5 antibodies or antibody fragments thereof are further humanized using known techniques in the art.

One of ordinary skill in the art would also be able to prepare anti-C5 scFvs or Fabs, comprising one or more CDRs selected from any of SEQ ID NOs: 3-5, 8-11, 14, 17, 20, 23, 26, 29, 34, 37-39, 42-44, 47-49, 52-54, 57, 62, 65, 68, 86, 94, 96, 97, 100, 102, 105, and 108, or a variant thereof having at least about 85% (e.g., at least about any of 86%, 87%, 88%, 89%, 90%, 91%, 92%, 93%, 94%, 95%, 96%, 97%, 98%, 99% or more) sequence identity to any of SEQ ID NOs: 3-5, 8-11, 14, 17, 20, 23, 26, 29, 34, 37-39, 42-44, 47-49, 52-54, 57, 62, 65, 68, 86, 94, 96, 97, 100, 102, 105, and 108.

In some embodiments, the anti-C5 antibody or antigen-binding fragment thereof comprises one or more CDRs having at least about 85% (such as at least about any of 86%, 87%, 88%, 89%, 90%, 91%, 92%, 93%, 94%, 95%, 96%, 97%, 98%, 99%, or 100%) amino acid identity with the CDR sequence of any of SEQ ID NOs: 3-5, 8-11, 14, 17, 20, 23, 26, 29, 34, 37-39, 42-44, 47-49, 52-54, 57, 62, 65, 68, 86, 94, 96, 97, 100, 102, 105, and 108.

In some embodiments, the anti-C5 antibody or antigen-binding fragment thereof (e.g., scFv, Fab) comprises a VH region and a VL region, wherein the VH region comprises an amino acid sequence that is more than about 90% (such as more than about any of 90%, 91%, 92%, 93%, 94%, 95%, 96%, 97%, 98%, 99%, or 100%) identical to an amino acid sequence of any of SEQ ID NOs: 2, 19, 22, 28, 36, 41, 46, 51, 56, 59, 64, 67, and 70, and wherein the VL region comprises an amino acid sequence that is more than about 90% (such as more than about any of 90%, 91%, 92%, 93%, 94%, 95%, 96%, 97%, 98%, 99%, or 100%) identical to an amino acid sequence of any of SEQ ID NOs: 7, 13, 16, 17, 25, and 31. In some embodiments, the anti-C5 antibody or antigen-binding fragment thereof (e.g., scFv, Fab) comprises a VH comprising an amino acid sequence that is more than about 90% (such as more than about any of 90%, 91%, 92%, 93%, 94%, 95%, 96%, 97%, 98%, 99%, or 100%) identical to an amino acid sequence of any of SEQ ID NOs: 87, 93, 101, 103, 104, 107, and 109-115, and a VL comprising an amino acid sequence that is more than about 90% (such as more than about any of 90%, 91%, 92%, 93%, 94%, 95%, 96%, 97%, 98%, 99%, or 100%) identical to an amino acid sequence of any of SEQ ID NOs: 88, 95, 98, 99, and 106.

In some embodiments, the anti-C5 antibody or antibody fragment thereof is modified. In some embodiments, the modification(s) includes fusion of the anti-C5 antibody or antigen-binding fragment thereof with another protein or fragment thereof. In some embodiments, the anti-C5 antibody or antibody fragment thereof is modified to increase the circulating half-life of in vivo. For example, the anti-C5 antibody or antibody fragment thereof may be fused with an FcRn molecule, which is also known as neonatal Fc receptor to stabilize the antibody or antibody fragment thereof in vivo. (Nature Reviews Immunology 7:715-725). In some embodiments, the anti-C5 antibody or antigen-binding fragment thereof is conjugated (e.g., fused) to an effector molecule and/or another targeting moiety (such as an antibody or antibody fragment thereof recognizing a different molecule, different antigen, or a different epitope).

### FH Moiety

FH inhibits alternative pathway complement activation by serving as a cofactor for FI-mediated cleavage of C3b and by accelerating the decay of C3bBb complex, both mechanisms prevent the amplification loop of the AP.

In various embodiments, the FH or functional fragment thereof comprises a complement FH. Complement FH is a single polypeptide chain plasma glycoprotein. The protein is composed of 20 repetitive SCR domains of approximately 60 amino acids, arranged in a continuous fashion like a string of 20 beads. In some embodiments, the FH binds to C3b, accelerates the decay of the alternative pathway C3-convertase (C3Bb), and acts as a cofactor for the proteolytic inactivation of C3b. In the presence of FH, C3b proteolysis results in the cleavage of C3b. In some embodiments, the FH has at least three distinct binding domains for C3b, which are located within SCR 1-4, SCR 5-8, and SCR 19-20. In some embodiments, each site of FH binds to a distinct region within the C3b protein: the N-terminal sites bind to native C3b; the second site, located in the middle region of FH, binds to the C3c fragment, and the site located within SCR19 and 20 binds to the C3d region. In some embodiments, the FH additionally contains binding sites for heparin, which are located within SCR 7, SCR 5-12, and SCR20 of FH and overlap with that of the C3b binding sites. For example, structural and functional analyses have shown that the domains for the complement inhibitory activity of FH are located within the first four N-terminal SCR domains.

FH or functional fragment thereof can be derived from any source, such as any organism that has a complement system, including but not limited to, dogs, cats, pigs, cows, sheep, goats, horses, rats, rabbits, hamsters, guinea pigs, monkeys, mice, and humans. In some embodiments, the FH or functional fragment thereof is a mouse FH or functional fragment thereof. In some embodiments, the FH or functional fragment thereof is a human FH or functional fragment thereof.

The FH or functional fragment thereof described herein refers to any portion of a FH protein having some (e.g., at least about any of 10%, 20%, 30%, 40%, 50%, 60%, 70%, 80%, 90%, 95%, 99%, or more) or all of the complement inhibitory activity of the FH protein, such as inhibiting (e.g., inhibiting at least about any of 10%, 20%, 30%, 40%, 50%, 60%, 70%, 80%, 90%, 95%, 99%, or 100%) C3 activation, and includes, but is not limited to, full-length FH proteins, biologically active fragments of FH proteins, an FH fragment comprising SCR1-4, or any homologue of a naturally occurring FH or functional fragment thereof, as described in detail below.

In various embodiments, the FH is a full-length human FH protein comprising an amino acid sequence set forth in any of SEQ ID NOs: 81, 83, and 127. Amino acids 1-18 correspond to the leader peptide, amino acids 21-80 correspond to SCR1, amino acids 85-141 correspond to SCR2, amino acids 146-205 correspond to SCR3, amino acids 210-262 correspond to SCR4, and amino acids 267-320 correspond to SCR5. In some embodiments, the FH or functional fragment thereof does not comprise the leader peptide, i.e., the FH or functional fragment thereof is mature form. In some embodiments, the FH comprises the amino acid sequence of any of SEQ ID NOs: 82, 84, and 128. In some embodiments, the functional fragment of FH is derived from SEQ ID NO: 127 or 128.

In some embodiments, the FH or functional fragment thereof has one or more of the following properties: (1) binding to C-reactive protein (CRP), (2) binding to C3b, (3) binding to heparin, (4) binding to sialic acid, (5) binding to endothelial cell surfaces, (6) binding to cellular integrin receptor, (7) binding to pathogens, (8) C3b co-factor activity, (9) C3b decay-acceleration activity, and (10) inhibiting the alternative complement pathway. In some embodiments, the FH moiety comprises the first four N-terminal SCR domains of FH (SCR 1-4). In some embodiments, the FH moiety comprises the first five N-terminal SCR domains of FH (SCR 1-5). In some embodiments, the FH moiety comprises the first six N-terminal SCR domains of FH (SCR 1-6). In some embodiments, the FH moiety comprises (and in some embodiments consists of or consisting essentially of) at least the first four N-terminal SCR domains of FH, including for example, at least any of the first 5, 6, 7, 8, 9, 10, 11, 12, 13, 14, or more N-terminal SCR domains of FH.

In some embodiments, the FH is a wildtype FH. In some embodiments, the FH is a variant of a naturally occurring FH or a fragment thereof. A variant of a naturally occurring FH protein or a fragment thereof includes proteins which differ from a naturally occurring FH or a fragment thereof in that at least one or a few, but not limited to one or a few, amino acids have been deleted (e.g., a truncated version of the protein, such as a peptide or fragment), inserted, inverted, substituted and/or derivatized (e.g., by glycosylation, phosphorylation, acetylation, myristoylation, prenylation, palmitation, amidation and/or addition of glycosylphosphatidyl inositol). For example, a FH variant may have an amino acid sequence that is at least about 70% identical to the amino acid sequence of a naturally occurring FH (e.g., any of SEQ ID NOs: 81-84), for example at least about any of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of a naturally occurring FH (e.g., any of SEQ ID NOs: 81-84). In some embodiment, the FH variant or a fragment thereof retains all the complement inhibition activity of a naturally occurring FH or a fragment thereof. In some embodiments, the FH variant or a fragment thereof retains at least about 50%, for example, at least about any of 60%, 70%, 80%, 90%, or 95% of the complement inhibition activity of a naturally occurring FH or a fragment thereof. In some embodiments, the FH or functional fragment thereof comprises at least the first five N-terminal SCR domains of a human FH, such as a FH portion having an amino acid sequence containing at least amino acids 21 through 320 of the human FH (e.g., any of SEQ ID NOs: 81-84). In some embodiments, the FH or functional fragment thereof comprises at least the first five N-terminal SCR domains of human FH having an amino acid sequence that is at least about any of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to amino acids 21 through 320 of the human FH (e.g., any of SEQ ID NOs: 81-84).

In some embodiments, the functional fragment of FH comprises SCR domains 1-5 of human FH. In some embodiments, the functional fragment of FH comprises the amino acid sequence of SEQ ID NO: 85.

### Anti-C5-4H Fusion Protein and Anti-C5 Antibody Moiety Properties

In some embodiments, the anti-C5-FH fusion protein blocks (e.g., blocking at least about any of 10%, 20%, 30%, 40%, 50%, 60%, 70%, 80%, 90%, or 100%) activated C3 fragment opsonization of paroxysmal nocturnal hemoglobinuria (PNH) RBCs. In some embodiments, the anti-C5-FH fusion protein inhibits (e.g., inhibiting at least about any of 10%, 20%, 30%, 40%, 50%, 60%, 70%, 80%, 90%, or 100%) classical pathway (CP)-triggered terminal pathway complement activation, e.g., inhibiting C5 activity. In some embodiments, the anti-C5-FH fusion protein inhibits (e.g., inhibiting at least about any of 10%, 20%, 30%, 40%, 50%, 60%, 70%, 80%, 90%, or 100%) alternative pathway (AP) complement activation, e.g., inhibiting C3 activity. In some embodiments, the anti-C5-FH fusion protein has higher (e.g., at least about any of 10%, 20%, 30%, 40%, 50%, 60%, 70%, 80%, 90%, 1-fold, 2-fold, 5-fold, 10-fold, 20-fold, or higher) complement pathway inhibition activity compared to that induced by a same anti-C5 antibody moiety alone, a same FH or fragment thereof (e.g., fused to an Fc fragment) alone, and/or a combination of a same anti-C5 antibody moiety and a same FH or fragment thereof (e.g., fused to an Fc fragment). In some embodiments, the anti-C5-FH fusion protein inhibits (e.g., inhibiting at least about any of 10%, 20%, 30%, 40%, 50%, 60%, 70%, 80%, 90%, or 100%) the lysis of human PNH RBCs. In some embodiments, the anti-C5-FH fusion protein inhibits (e.g., inhibiting at least about any of 10%, 20%, 30%, 40%, 50%, 60%, 70%, 80%, 90%, or 100%) extravascular hemolysis (EVH). In some embodiments, the anti-C5-FH fusion protein can target cells with C5b-9 deposition on cell surface. In some embodiments, the anti-C5-FH fusion protein inhibits (e.g., inhibiting at least about any of 10%, 20%, 30%, 40%, 50%, 60%, 70%, 80%, 90%, or 100%) C3b deposition via AP complement activation. In some embodiments, the anti-C5-FH fusion protein prevents (e.g., preventing at least about any of 10%, 20%, 30%, 40%, 50%, 60%, 70%, 80%, 90%, or 100%) both glomerular C3 and C9 deposition. In some embodiments, administration of the fusion protein inhibits (e.g., inhibiting at least about any of 10%, 20%, 30%, 40%, 50%, 60%, 70%, 80%, 90%, or 100%) the generation of a C3a or C3b protein. In some embodiments, administration of the fusion protein inhibits (e.g., inhibiting at least about any of 10%, 20%, 30%, 40%, 50%, 60%, 70%, 80%, 90%, or 100%) the generation of a C5a or C5b protein. In some embodiments, administration of the anti-C5 antibody or fusion protein inhibits (e.g., inhibiting at least about any of 10%, 20%, 30%, 40%, 50%, 60%, 70%, 80%, 90%, or 100%) the generation of a C3a or C3b protein, the generation of a C5a or C5b protein, or any combinations thereof.

Binding affinity and specificity of the anti-C5-FH fusion proteins and anti-C5 antibody moieties described herein can be determined experimentally by methods known in the art. For example, the binding of an antibody to a protein antigen can be detected and/or quantified using a variety of techniques such as, but not limited to, Western blot, dot blot, surface plasmon resonance (SPR) method (e.g., BIAcore system; Pharmacia Biosensor AB, Uppsala, Sweden and Piscataway, N.J.), Bio-Layer Interferometry (BLI) (e.g. Octet system, ForteBio), RIA, ECL, IRMA, EIA, peptide scans, and enzyme-linked immunosorbent assay (ELISA). See, e.g., Benny K. C. Lo (2004) “Antibody Engineering: Methods and Protocols.” Humana Press (ISBN: 1588290921); Borrebaek (1992) “Antibody Engineering, A Practical Guide.” W.H. Freeman and Co., N.Y.; Borrebaek (1995) “Antibody Engineering.” 2nd Edition, Oxford University Press, N.Y., Oxford; Johne et al. (1993). J Immunol Meth 160:191-198; Jonsson et al. (1993) Ann Biol Clin 51:19-26; and Jonsson et al. (1991) Biotechniques 11:620-627. Also see Example 1 herein, US20220204602, US20220177556, and U.S. Ser. No. 11/578,137 for exemplary methods, the content of each of which is incorporated herein by reference in their entirety.

The activities of the fusion proteins can be analyzed by any methods suitable for measuring factor H or anti-C5 antibody activities. Methods for determining whether a particular anti-C5 antibody moiety or anti-C5-FH fusion protein described herein inhibits human C5 and/or C3 activation are known in the art. Methods for determining the inhibitory activity of anti-C5 antibodies or anti-C5-FH fusion proteins on the classical pathway (CP) or terminal pathway are known in the art and described in, e.g., Zelek et al, (2020) Immunology. 161(2):103-113. Inhibition of C5 prevents the generation of C5a and C5b, which subsequently inhibits leukocyte and platelet activation. For example, anti-C5 complement inhibition during extracorporeal circulation can be measured by methods described in, e.g., Rinder et al. (1995) J Clin Invest. 96(3): 1564-1572. Methods for determining the inhibitory activity of FH or functional fragment thereof (or anti-C5-FH fusion protein) on the alternative pathway (AP) are known in the art and described in, e.g., Raina et al, (2022) Front Immunol. 13:931210 and Ferreira et al. (2011) Mol Immunol. 47(13): 2187-2197. FH or functional fragment thereof prevents the progression of C3b deposition and can be measured by methods known in the art. See, for example, DeLillo et al. (2006) Mol Immunol and “Instructions for Use WIESLAB® Complement System Alternative Pathway” Document No. LABEL-DOC-0034, 2.0, December 2018. The concentration and/or physiologic activity of C5 (or C5a, C5b) and C3 (or C3b) in a body fluid can be measured by methods well known in the art. For example, generation of C5a can be measured by methods described in Volokina et al. (2015) Blood. 126(2): 278-279. Methods for measuring C5 (or C5a, C5b) and C3 (or C3b) concentration or activity include, e.g., biolayer interferometry, RIAs, ELISAs (see, e.g., Corvillo et al. (2021) Int J Mol Sci 22(12):6608). The activities of the fusion proteins described herein can also be analyzed by investigating C5a-mediated inflammation and cell activation, or C5b-mediated cell lysis. Also see Examples 1 and 2 herein, as well as US20220204602, US20220177556, and U.S. Ser. No. 11/578,137 for exemplary methods.

Hemolytic assays can be used to determine the inhibitory activity of an anti-C5-FH fusion protein or an anti-C5 antibody moiety on complement activation, and are useful for determining potential off-target binding of anti-C5-FH fusion proteins or anti-C5 antibody moieties. These assays include but are not limited to, sheep red blood cell (RBC) lysis test, rabbit RBC lysis test, chicken RBC cell lysis test, or paroxysmal nocturnal hemoglobinuria (PNH) RBC lysis assay. Detailed methods can be found in, e.g., US20220204602, US20220177556, and U.S. Ser. No. 11/578,137. In order to determine the effect of the anti-C5-FH fusion protein or anti-C5 antibody moiety on complement pathway-mediated hemolysis in a serum test solution in vitro, for example, a sheep RBC lysis assay may be used to examine the inhibition of CP-triggered terminal pathway complement activation, a rabbit RBC lysis assay may be used to examine AP regulation, or an LPS-based ELISA assay may be used to examine AP complement inhibition, or a PNH RBC lysis assay may be used to examine AP-mediated complement inhibition. C3 inhibition can be measured by methods known in the art, including, e.g., flow cytometry and commercial kits (e.g., Wieslab® assays). See, for example, Mannes et al, (2021) Blood. 137(4):443-455 and “Instructions for Use WIESLAB® Complement System Alternative Pathway” Document No. LABEL-DOC-0034, 2.0, December 2018.

### Methods of Making Fusion Proteins

The fusion proteins described herein can be made by introducing a nucleic acid into a host cell, allowing expression of the protein, and purifying the protein from the host cell extract or supernatant. Methods of making proteins comprising antibody moieties and constructing nucleic acids or vectors encoding thereof, as well as purifying proteins comprising antibody moieties are known in the art. Also see US20220204602 for exemplary methods.

In some embodiments, there is also provided isolated nucleic acids encoding any of the anti-C5 antibody moieties and anti-C5-FH fusion proteins described herein. Also provided are vectors (e.g., expression vectors, viral vectors) comprising such isolated nucleic acids. Further provided are host cells (e.g., CHO cells, or E. coli) expressing such anti-C5 antibody moieties or anti-C5-FH fusion proteins, such as host cells comprising a vector or nucleic acid encoding thereof. In some embodiments, the isolated nucleic acid encoding a VH or a heavy chain (e.g., including a signal peptide at the N-terminus) of any of the anti-C5 antibody moieties or anti-C5-FH fusion proteins described herein comprises a nucleic acid sequence of any of SEQ ID NOs: 1, 18, 21, 27, 35, 40, 45, 50, 55, 58, 63, 66, and 69. In some embodiments, the isolated nucleic acid encoding a VL or a light chain (e.g., including a signal peptide at the N-terminus) of any of the anti-C5 antibody moieties or anti-C5-FH fusion proteins described herein comprises a nucleic acid sequence of any of SEQ ID NOs: 6, 12, 15, 24, and 30. In some embodiments, the isolated nucleic acid encoding a FH or functional fragment thereof fused to an anti-C5 antibody moiety (e.g., fused to the C-terminus of one or both heavy chains of an anti-C5 full-length antibody) comprises a nucleic acid sequence of any of SEQ ID NOs: 71, 75, 77, and 79.

### Pharmaceutical Compositions

Further provided are pharmaceutical compositions comprising i) any one of the anti-C5-FH fusion proteins described herein and ii) a pharmaceutically acceptable carrier. Pharmaceutical compositions can be prepared by mixing the anti-C5-FH fusion protein having the desired degree of purity with pharmaceutically acceptable carriers, excipients or stabilizers (Remington's Pharmaceutical Sciences 16th edition, Osol, A. Ed. (1980)), in the form of aqueous solutions.

In some embodiments, the pharmaceutical composition further comprises additional ingredients. Additional ingredients include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference. Additional excipients include agents which can serve as one or more of the following: (1) bulking agents, (2) solubility enhancers, (3) stabilizers and (4) and agents preventing denaturation or adherence to the container wall (e.g., surfactant, such as polysorbate (e.g., polysorbate 80) or poloxamer).

In order for the pharmaceutical compositions to be used for in vivo administration, they must be sterile. The pharmaceutical composition may be rendered sterile by filtration through sterile filtration membranes. The pharmaceutical compositions herein generally are placed into a container having a sterile access port, for example, an intravenous solution bag or vial having a stopper pierceable by a hypodermic injection needle.

The pharmaceutical compositions herein may also contain more than one active compound as necessary for the particular indication being treated, preferably those with complementary activities that do not adversely affect each other. Alternatively, or in addition, the composition may comprise a cytotoxic agent, chemotherapeutic agent, cytokine, immunosuppressive agent, or growth inhibitory agent. Such molecules are suitably present in combination in amounts that are effective for the purpose intended.

The pharmaceutical compositions may be prepared by any method known or hereafter developed in the art of pharmacology. Preparations include but are not limited to, bringing the active ingredient into association with a carrier or one or more other accessory ingredients, and then, if necessary or desirable, shaping or packaging the product into a desired single- or multi-dose unit. The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Pharmaceutical compositions that are useful in the methods of the invention may be prepared, packaged, or sold in formulations suitable for parenteral, intramuscular, intradermal, subcutaneous, or intravenous routes of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. A unit dose is discrete amount of the pharmaceutical composition comprising a predetermined amount of the active ingredient. The amount of the active ingredient is generally equal to the dosage of the active ingredient which would be administered to an individual or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the individual treated and further depending upon the route by which the composition is to be administered. By way of example, the composition may comprise between 0.1% and 100% (w/w) active ingredient. In various embodiments, the composition comprises at least about any of 1%, 2%, 3%, 4%, 5%, 6%, 7%, 8%, 9%, 10%, 15%, 20%, 30%, 40%, 50%, 60%, 70%, 80%, 90%, 95 99%, or 100% (w/w) active ingredient.

Parenteral administration of a pharmaceutical composition includes any route of administration characterized by physical breaching of a tissue of an individual and administration of the pharmaceutical composition through the breach in the tissue. Parental administration can be local, regional or systemic. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, intraocular, intravitreal, subcutaneous, intraperitoneal, intramuscular, intradermal, intrasternal injection, and intratumoral.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, and implantable sustained-release or biodegradable formulations. Such formulations may further comprise one or more additional ingredients including, but not limited to, suspending, stabilizing, or dispersing agents. In some embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

In some embodiments, there is also provided a pharmaceutical composition (e.g., a liquid solution) comprising a fusion protein (e.g., any of the anti-C5-FH fusion proteins described herein, such as FMEH-IgG4-PLA-FH) (e.g., at a concentration of about 120 mg/mL) comprising i) an anti-human C5 antibody moiety (e.g., anti-human C5 full-length antibody, such as FMEH-IgG4-PLA) and ii) an FH or functional fragment thereof (e.g., SCR1-5 domains of FH, such as SEQ ID NO: 85) in a phosphate buffer comprising sodium phosphate, sodium chloride, L-Lys-HCL, and PS80 (e.g., at about pH of 6.0). In some embodiments, the anti-C5-FH fusion protein or pharmaceutical composition thereof is provided in a 2 mL vial, e.g., with a fill volume of 1.85 mL for a deliverable volume of 1.5 mL. In some embodiments, the anti-C5-FH fusion protein or pharmaceutical composition thereof is stored at 2° C. to 8° C., e.g., protected from light. In some embodiments, for IV administration, the anti-C5-FH fusion protein or pharmaceutical composition thereof is diluted in an infusion bag, e.g., containing ˜100 mL of 0.9% sodium chloride for infusion. In some embodiments, for SC administration, the anti-C5-FH fusion protein or pharmaceutical composition thereof is directly injected (e.g., manually or via a syringe pump) to the abdomen. In some embodiments, the SC injection is not administered close to (e.g., within about 3 cm) of the umbilicus. In some embodiments, each dose of the anti-C5-FH fusion protein or pharmaceutical composition thereof is rotated around the 4 abdominal quadrants. Modes of Administration

The pharmaceutical compositions comprising the anti-C5-FH fusion protein useful for practicing the invention may be administered to deliver a dose of at least about any of 1 ng/kg, 5 ng/kg, 10 ng/kg, 25 ng/kg, 50 ng/kg, 100 ng/kg, 500 ng/kg, 1 μg/kg, 5 μg/kg, 10 μg/kg, 25 μg/kg, 50 μg/kg, 100 μg/kg, 500 μg/kg, 1 mg/kg, 5 mg/kg, 10 mg/kg, 25 mg/kg, 50 mg/kg, 100 mg/kg, 200 mg/kg, 300 mg/kg, 400 mg/kg, or 500 mg/kg of body weight of the subject. In some embodiments, the invention administers a dose which results in a concentration of the anti-C5-fH fusion protein of at least about any of 1 pM, 10 pM, 100 pM, 1 nM, 10 nM, 100 nM, 1 μM, 2 μM, 3 μM, 5 μM, 10 μM, or 20 μM (e.g., in the plasma) in an individual. Also contemplated are dosage ranges between any of the doses disclosed herein.

In some embodiments, the pharmaceutical compositions useful for practicing the invention may be administered to deliver a dose of no more than about any of 1 ng/kg, 5 ng/kg, 10 ng/kg, 25 ng/kg, 50 ng/kg, 100 ng/kg, 500 ng/kg, 1 μg/kg, 5 μg/kg, 10 μg/kg, 25 μg/kg, 50 μg/kg, 100 μg/kg, 500 μg/kg, 1 mg/kg, 5 mg/kg, 10 mg/kg, 25 mg/kg, 50 mg/kg, 100 mg/kg, 200 mg/kg, 300 mg/kg, 400 mg/kg, or 500 mg/kg of body weight of the subject.

In some embodiments, the invention administers a dose which results in a concentration of the anti-C5-fH fusion protein of no more than about any of 1 pM, 10 pM, 100 pM, 1 nM, 10 nM, 100 nM, 1 μM, 2 μM, 3 μM, 4 μM, 5 μM, 6 μM, 7 μM, 8 μM, 9 μM, 10 μM or 20 μM in an individual, such as in the plasma of an individual. Also contemplated are dosage ranges between any of the doses disclosed herein.

Typically, dosages which may be administered in a method of the invention to a subject, in some embodiments a human, range in amount from 0.5 μg to about 50 mg per kilogram of body weight of the subject. While the precise dosage administered will vary depending upon any number of factors, including but not limited to, the type of subject and type of disease state being treated, the age of the subject and the route of administration. In some embodiments, the dosage of the fusion protein will vary from about 1 μg to about 10 mg per kilogram of body weight of the subject. In some embodiments, the dosage will vary from about 3 μg to about 1 mg per kilogram of body weight of the subject. In some embodiments, the anti-C5-FH fusion protein is administered at about 1 mg/kg to about 5 mg/kg body weight of the subject, such as about 2.5 mg/kg to about 3.5 mg/kg.

The fusion protein may be administered to a subject as frequently as several times daily, or it may be administered less frequently, such as once a day, twice a day, thrice a day, once a week, twice a week, thrice a week, once every two weeks, twice every two weeks, thrice every two weeks, once a month, twice a month, thrice a month, or even less frequently, such as once every several months or even once or a few times a year or less. The frequency of the dose will be readily apparent to the skilled artisan and will depend upon any number of factors, such as, but not limited to, the type and severity of the disease being treated, the type and age of the subject, etc.

In some embodiments, the fusion protein is administered intravenously, for example by infusion over any of 30 minutes, 45 minutes, 1 hour, 2 hours, or longer. In some embodiments, the fusion protein is administered subcutaneously, such as to abdominal wall. In some embodiments, the fusion protein is administered at the dose of about 60 to about 3600 mg (including for example any of 60, 180, 360, 600, 720, 960, 1200, 1440, 1920, 2400, 2880, and 3600 mg, and any numbers in between).

In some embodiments, the fusion protein is administered weekly. In some embodiments, the fusion protein is administered biweekly. In some embodiments, the fusion protein is administered (such as intravenously or subcutaneously) weekly at the dose of about 600 to about 2880 mg (including for example any of 600, 720, 960, 1200, 1440, 1920, 2400, and 2880 mg, and any numbers in between). In some embodiments, the fusion protein is administered (such as intravenously or subcutaneously) biweekly at the dose of about 960 to about 2880 mg (including for example any of 960, 1800, 1920, 2400, and 2880 mg, and any numbers in between).

### EXEMPLARY EMBODIMENTS

Embodiment 1. A method of treating a complement-mediated disease in a human individual, comprising administering to the human individual an effective amount of a fusion protein comprising i) an antibody moiety that specifically binds to human C5 (“anti-C5 antibody moiety”) and ii) a Factor H (FH) or functional fragment thereof.

Embodiment 2. The method of embodiment 1, wherein the complement-mediated disease is selected from the group consisting of paroxysmal nocturnal hemoglobinuria (PNH) syndrome, C3 glomerulopathy (C3G), IgA nephropathy (IgAN), and thrombotic microangiopathy secondary to systemic lupus erythematosus (SLE-TMA).

Embodiment 3. The method of embodiment 2, wherein the complement-mediated disease is PNH.

Embodiment 4. The method of embodiment 2, wherein the complement-mediated disease is C3G.

Embodiment 5. The method of embodiment 2, wherein the complement-mediated disease is IgAN.

Embodiment 6. The method of embodiment 2, wherein the complement-mediated disease is SLE-TMA.

Embodiment 7. The method any of embodiments 1-6, wherein the fusion protein is administered intravenously (IV) or subcutaneously (SC).

Embodiment 8. The method of any one of embodiments 1-7, wherein the fusion protein is administered at a dose of about 60 mg to about 3600 mg.

Embodiment 9. The method of any one of embodiments 1-8, wherein the fusion protein is administered at a single dose.

Embodiment 10. The method of embodiment 9, wherein the fusion protein is administered at a dose of about 60 mg to about 1200 mg.

Embodiment 11. The method of embodiment 9 or 10, wherein the fusion protein is administered at a dose of: about 60 mg IV, about 180 mg IV, about 180 mg SC, about 360 mg IV, about 600 mg IV, about 720 mg SC, or about 1200 mg IV.

Embodiment 12. The method of any one of embodiments 1-8, wherein the fusion protein is administered at multiple doses.

Embodiment 13. The method of embodiment 12, wherein the fusion protein is administered weekly (QW) or biweekly (Q2W).

Embodiment 14. The method of embodiment 12 or 13, wherein the fusion protein is administered at a dose of about 600 mg to about 2880 mg.

Embodiment 15. The method of any one of embodiments 12-14, wherein the fusion protein is administered with an initial phase comprising administering the fusion protein at one or more initial doses, followed by a maintenance phase comprising administering the fusion protein weekly or biweekly for two or more maintenance doses.

Embodiment 16. The method of embodiment 15, wherein the initial dose is about 600 mg to about 3600 mg.

Embodiment 17. The method of embodiment 15 or 16, wherein the initial dose is about 1200 mg to about 3600 mg.

Embodiment 18. The method of any one of embodiments 15-17, wherein the one or more initial doses of the fusion protein are administered IV.

Embodiment 19. The method of any one of embodiments 15-18, wherein the initial phase comprises administering the fusion protein at one initial dose.

Embodiment 20. The method of any one of embodiments 15-19, wherein the maintenance dose is about 600 mg to about 2880 mg.

Embodiment 21. The method of any one of embodiments 15-20, wherein the two or more maintenance doses of the fusion protein are administered weekly.

Embodiment 22. The method of embodiment 21, wherein the maintenance dose is about 600 mg to about 1440 mg.

Embodiment 23. The method of embodiment 21 or 22, wherein the two or more maintenance doses of the fusion protein are each administered at about 600 mg IV to about 1200 mg IV, or at about 720 mg SC to about 1440 mg SC.

Embodiment 24. The method of any one of embodiments 15-20, wherein the two or more maintenance doses of the fusion protein are administered biweekly.

Embodiment 25. The method of embodiment 24, wherein the maintenance dose is about 960 mg to about 2880 mg.

Embodiment 26. The method of embodiment 24 or 25, wherein the two or more maintenance doses of the fusion protein are each administered at about 960 mg SC to about 1920 mg SC, at about 1920 mg SC to about 2880 mg SC, at about 1800 mg SC to about 2880 mg SC, or at about 1800 mg SC to about 2400 mg SC.

Embodiment 27. The method of any one of embodiments 15-26, wherein the maintenance phase is at least about 4 weeks.

Embodiment 28. The method of any one of embodiments 15-27, wherein the maintenance phase is at least about 12 weeks.

Embodiment 29. The method of any one of embodiments 15-28, wherein the maintenance phase comprises administering the fusion protein weekly or biweekly at a first maintenance dose for a first maintenance phase period, followed by administering the fusion protein weekly or biweekly at a second maintenance dose for a second maintenance phase period.

Embodiment 30. The method of embodiment 29,
i) wherein the second maintenance dose is higher than the first maintenance dose; and/or
ii) wherein the second maintenance phase period is longer than the first maintenance phase period.

Embodiment 31. The method of embodiment 29 or 30,
i) wherein the first maintenance dose of the fusion protein is administered at about 720 mg SC or about 600 mg IV weekly, and the second maintenance dose of the fusion protein is administered at about 1440 mg SC or about 1200 mg IV weekly; and/or
ii) wherein the first maintenance phase period is about 4 weeks, and the second maintenance phase period is at least about 44 weeks.

Embodiment 32. The method of any one of embodiments 29-31, wherein the complement-mediated disease is C3G or IgAN.

Embodiment 33. The method of any one of embodiments 29-32, wherein the initial phase comprises administering the fusion protein at an initial dose of about 1200 mg IV on Day 1, and wherein the maintenance phase comprises: i) administering the fusion protein at a first maintenance dose of about 720 mg SC or about 600 mg IV weekly starting Day 8 for about 4 weeks, followed by ii) administering the fusion protein at a second maintenance dose of about 1440 mg SC or about 1200 mg IV weekly starting Day 36 for about 44 weeks.

Embodiment 34. The method of any one of embodiments 15-24, 27, and 28, wherein the initial phase comprises administering the fusion protein at an initial dose of about 1200 mg weekly for one or more doses, and wherein the maintenance phase comprises administering the fusion protein at a maintenance dose of about 720 mg weekly or biweekly for at least two doses.

Embodiment 35. The method of any one of embodiments 15-24, 27, 28, and 34, wherein the initial phase comprises administering the fusion protein at an initial dose of about 1200 mg IV on Day 1, and wherein the maintenance phase comprises administering the fusion protein at a maintenance dose of about 720 mg SC weekly starting Day 8 for 4 weeks.

Embodiment 36. The method of any one of embodiments 12-14, wherein the fusion protein is administered at a dose of about 600 mg IV weekly for 5 weeks.

Embodiment 37. The method of any one of embodiments 15-28, wherein the initial phase comprises administering the fusion protein at an initial dose of about 1200 mg IV to about 3600 mg IV on Day 1, and wherein the maintenance phase comprises administering the fusion protein starting Day 8 at a maintenance dose of:
i) about 600 mg IV to about 1200 mg IV weekly;
ii) about 720 mg SC to about 1440 mg SC weekly;
iii) about 1920 mg SC to about 2880 mg SC biweekly;
iv) about 1800 mg SC to about 2400 mg SC biweekly; or
v) about 960 mg SC to about 2880 mg SC biweekly.

Embodiment 38. The method of any one of embodiments 15-28, 34, and 37, wherein the maintenance phase is at least about 12 weeks for weekly maintenance dosing, or at least about 13 weeks for biweekly maintenance dosing.

Embodiment 39. The method of any one of embodiments 15-28, 34, 37, and 38, wherein the maintenance phase is at least about 24 weeks for weekly maintenance dosing, or at least about 25 weeks for biweekly maintenance dosing.

Embodiment 40. The method of any one of embodiments 15-28, 34, and 37-39, wherein the maintenance phase is at least about 48 weeks for weekly maintenance dosing, or at least about 49 weeks for biweekly maintenance dosing.

Embodiment 41. The method of any one of embodiments 15-28, and 37-40, wherein the complement-mediated disease is PNH.

Embodiment 42. The method of embodiment 41, wherein the initial phase comprises administering the fusion protein at an initial dose of about 1200 mg IV to about 3600 mg IV on Day 1, and wherein the maintenance phase comprises administering the fusion protein starting Day 8 at a maintenance dose of:
i) about 600 mg IV to about 1200 mg IV weekly for at least about 12 weeks;
ii) about 720 mg SC to about 1440 mg SC weekly for at least about 12 weeks;
iii) about 1920 mg SC to about 2880 mg SC biweekly for at least about 13 weeks; or
iv) about 960 mg SC to about 2880 mg SC biweekly for at least about 13 weeks.

Embodiment 43. The method of embodiment 41 or 42, wherein the initial phase comprises administering the fusion protein at an initial dose of about 1200 mg IV on Day 1, and wherein the maintenance phase comprises administering the fusion protein starting Day 8 at a maintenance dose of about 720 mg SC or about 600 mg IV weekly for about 12 weeks.

Embodiment 44. The method of embodiment 43, wherein the initial phase comprises administering the fusion protein at an initial dose of about 1200 mg IV on Day 1, and wherein the maintenance phase comprises administering the fusion protein starting Day 8 at a maintenance dose of about 720 mg SC weekly for about 12 weeks.

Embodiment 45. The method of embodiment 41 or 42, wherein the initial phase comprises administering the fusion protein at an initial dose of about 3600 mg IV on Day 1, and wherein the maintenance phase comprises administering the fusion protein starting Day 8 at a maintenance dose of about 2880 mg SC biweekly for about 13 weeks.

Embodiment 46. The method of embodiment 41 or 42, wherein the initial phase comprises administering the fusion protein at an initial dose of about 1200 mg IV to about 2400 mg IV on Day 1, and wherein the maintenance phase comprises administering the fusion protein starting Day 8 at a maintenance dose of about 960 mg SC to about 2880 mg SC biweekly for about 13 weeks.

Embodiment 47. The method of embodiment 46, wherein the initial phase comprises administering the fusion protein at an initial dose of about 2400 mg IV on Day 1, and wherein the maintenance phase comprises administering the fusion protein starting Day 8 at a maintenance dose of about 960 mg SC to about 2880 mg SC biweekly for about 13 weeks.

Embodiment 48. The method of embodiment 46, wherein the initial phase comprises administering the fusion protein at an initial dose of about 1200 mg IV to about 2400 mg IV on Day 1, and wherein the maintenance phase comprises administering the fusion protein starting Day 8 at a maintenance dose of about 1920 mg SC biweekly for about 13 weeks.

Embodiment 49. The method of any one of embodiments 46-48, wherein the initial phase comprises administering the fusion protein at an initial dose of about 2400 mg IV on Day 1, and wherein the maintenance phase comprises administering the fusion protein starting Day 8 at a maintenance dose of about 1920 mg SC biweekly for about 13 weeks.

Embodiment 50. The method of any one of embodiments 15-28, and 37-40, wherein the complement-mediated disease is SLE-TMA.

Embodiment 51. The method of embodiment 50, wherein the initial phase comprises administering the fusion protein at an initial dose of about 1200 mg IV to about 3600 mg IV on Day 1, and wherein the maintenance phase comprises administering the fusion protein starting Day 8 at a maintenance dose of:
i) about 720 mg SC to about 1440 mg SC weekly for at least about 24 weeks; or
ii) about 1920 mg SC to about 2880 mg SC biweekly for at least about 25 weeks.

Embodiment 52. The method of embodiment 50 or 51, wherein the initial phase comprises administering the fusion protein at an initial dose of about 1200 mg IV on Day 1, and wherein the maintenance phase comprises administering the fusion protein starting Day 8 at a maintenance dose of about 960 mg SC weekly for about 24 weeks.

Embodiment 53. The method of any one of embodiments 15-28, and 37-40, wherein the complement-mediated disease is C3G or IgAN.

Embodiment 54. The method of embodiment 53, wherein the initial phase comprises administering the fusion protein at an initial dose of about 1200 mg IV to about 3600 mg IV on Day 1, and wherein the maintenance phase comprises administering the fusion protein starting Day 8 at a maintenance dose of:
i) about 600 mg IV to about 1200 mg IV weekly for at least about 48 weeks;
ii) about 720 mg SC to about 1440 mg SC weekly for at least about 48 weeks;
iii) about 1920 mg SC to about 2880 mg SC biweekly for at least about 49 weeks; or
iv) about 1800 mg SC to about 2400 mg SC biweekly for at least about 49 weeks.

Embodiment 55. The method of embodiment 53 or 54, wherein the initial phase comprises administering the fusion protein at an initial dose of about 1200 mg IV on Day 1, and wherein the maintenance phase comprises administering the fusion protein starting Day 8 at a maintenance dose of about 720 mg SC or about 600 mg IV weekly for about 48 weeks.

Embodiment 56. The method of embodiment 53 or 54, wherein the initial phase comprises administering the fusion protein at an initial dose of about 1200 mg IV on Day 1, and wherein the maintenance phase comprises administering the fusion protein starting Day 8 at a maintenance dose of about 1440 mg SC or about 1200 mg IV weekly for about 48 weeks.

Embodiment 57. The method of embodiment 53 or 54, wherein the initial phase comprises administering the fusion protein at an initial dose of about 2400 mg IV to about 3600 mg IV on Day 1, and wherein the maintenance phase comprises administering the fusion protein starting Day 8 at a maintenance dose of about 1920 mg SC to about 2880 mg SC biweekly for about 49 weeks.

Embodiment 58. The method of any one of embodiments 1-57, wherein the binding of the anti-C5 antibody moiety to human C5 is pH-dependent, and wherein the anti-C5 antibody moiety binds more strongly to human C5 at a neutral pH than it does at an acidic pH.

Embodiment 59. The method of any one of embodiments 1-58, wherein the anti-C5 antibody moiety is a full-length antibody, a Fab, a Fab′, a F(ab)2, a F(ab′)2, an scFv, or a combination thereof.

Embodiment 60. The method of any one of embodiments 1-59, wherein the anti-C5 antibody moiety is a full-length antibody (“anti-C5 full-length antibody”).

Embodiment 61. The method of embodiment 60, wherein the anti-C5 full-length antibody comprises an Fc fragment derived from human IgG4.

Embodiment 62. The method of embodiment 61, wherein the Fc fragment comprises the amino acid sequence of any of SEQ ID NOs: 32, 33, and 61.

Embodiment 63. The method of embodiment 61 or 62, wherein the Fc fragment comprises the amino acid sequence of SEQ ID NO: 61.

Embodiment 64. The method of any one of embodiments 60-63, wherein the anti-C5 full-length antibody comprises a heavy chain and a light chain, and wherein:
(i) the heavy chain comprises the amino acid sequence of SEQ ID NO: 119, and the light chain comprises the amino acid sequence of SEQ ID NO: 74;
(ii) the heavy chain comprises the amino acid sequence of SEQ ID NO: 121, and the light chain comprises the amino acid sequence of SEQ ID NO: 74;
(iii) the heavy chain comprises the amino acid sequence of SEQ ID NO: 123, and the light chain comprises the amino acid sequence of SEQ ID NO: 74;
(iv) the heavy chain comprises the amino acid sequence of SEQ ID NO: 125, and the light chain comprises the amino acid sequence of SEQ ID NO: 74;
(v) the heavy chain comprises the amino acid sequence of SEQ ID NO: 120, and the light chain comprises the amino acid sequence of SEQ ID NO: 90;
(vi) the heavy chain comprises the amino acid sequence of SEQ ID NO: 122, and the light chain comprises the amino acid sequence of SEQ ID NO: 90;
(vii) the heavy chain comprises the amino acid sequence of SEQ ID NO: 124, and the light chain comprises the amino acid sequence of SEQ ID NO: 90; or
(viii) the heavy chain comprises the amino acid sequence of SEQ ID NO: 126, and the light chain comprises the amino acid sequence of SEQ ID NO: 90.

Embodiment 65. The method of any one of embodiments 1-64, wherein the fusion protein inhibits C3 activation.

Embodiment 66. The method of any one of embodiments 1-65, wherein the functional fragment of FH comprises short consensus repeat (SCR) domains 1-5 of FH.

Embodiment 67. The method of embodiment 66, wherein the functional fragment of FH comprises the amino acid sequence of SEQ ID NO: 85.

Embodiment 68. The method of any one of embodiments 52-67, wherein the fusion protein comprises a first FH or functional fragment thereof and a second FH or functional fragment thereof, wherein the first FH or functional fragment thereof is fused to the C-terminus of a first heavy chain of the anti-C5 full-length antibody, and the second FH or functional fragment thereof is fused to the C-terminus of a second heavy chain of the anti-C5 full-length antibody.

Embodiment 69. The method of embodiment 68, wherein:
(i) each heavy chain fused to FH or functional fragment thereof comprises the amino acid sequence of SEQ ID NO: 72, each light chain comprises the amino acid sequence of SEQ ID NO: 74;
(ii) each heavy chain fused to FH or functional fragment thereof comprises the amino acid sequence of SEQ ID NO: 76, each light chain comprises the amino acid sequence of SEQ ID NO: 74;
(iii) each heavy chain fused to FH or functional fragment thereof comprises the amino acid sequence of SEQ ID NO: 78, each light chain comprises the amino acid sequence of SEQ ID NO: 74;
(iv) each heavy chain fused to FH or functional fragment thereof comprises the amino acid sequence of SEQ ID NO: 80, each light chain comprises the amino acid sequence of SEQ ID NO: 74;
(v) each heavy chain fused to FH or functional fragment thereof comprises the amino acid sequence of SEQ ID NO: 89, each light chain comprises the amino acid sequence of SEQ ID NO: 90;
(vi) each heavy chain fused to FH or functional fragment thereof comprises the amino acid sequence of SEQ ID NO: 116, each light chain comprises the amino acid sequence of SEQ ID NO: 90;
(vii) each heavy chain fused to FH or functional fragment thereof comprises the amino acid sequence of SEQ ID NO: 117, each light chain comprises the amino acid sequence of SEQ ID NO: 90; or
(viii) each heavy chain fused to FH or functional fragment thereof comprises the amino acid sequence of SEQ ID NO: 118, each light chain comprises the amino acid sequence of SEQ ID NO: 90.

Embodiment 70. The method of any one of embodiments 1-69, wherein the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. The examples below are intended to be purely exemplary of the application and should therefore not be considered to limit the application in any way.

All publications and patent applications mentioned in this specification are herein incorporated by reference to the same extent as if each individual publication or patent application was specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1: Design and Characterization of Anti-C5-FH Fusion Protein, a Bifunctional Anti-C5 mAb and FH1-5 Fusion Protein that Synergistically Inhibits Alternative and Terminal Pathways of Complement Activation

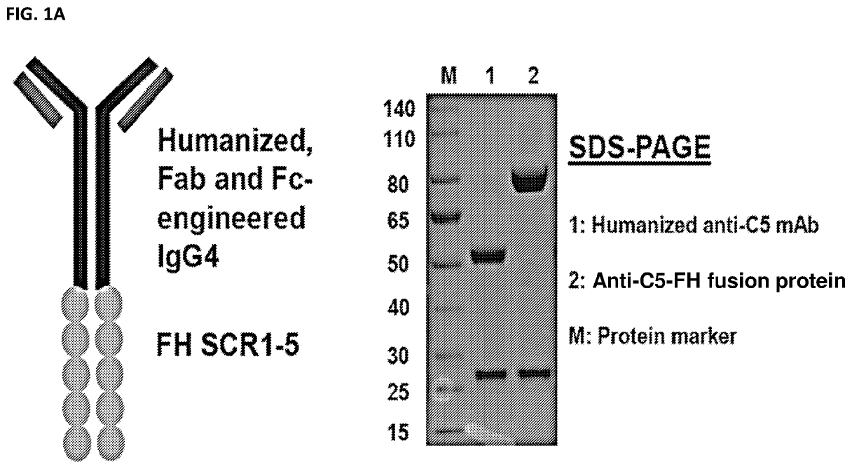

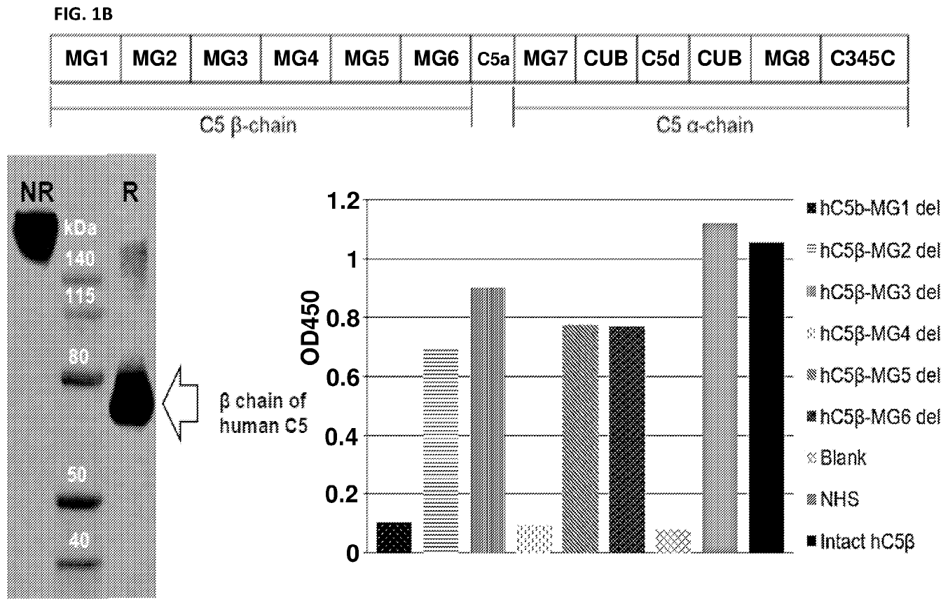

An exemplary anti-C5-FH fusion protein was developed by humanization and engineering of murine anti-human C5 monoclonal antibody (mAb) 2G1-3 (see US20200385481 for 2G1-3, the content of which is incorporated herein in its entirety), Fab- and Fc-engineered IgG4 (IgG4-PLA, SEQ ID NO: 61), and fusion with FH SCR1-5 (SEQ ID NO: 85). This anti-C5-FH fusion protein is named as “FMEH-IgG4-PLA-FH.” SDS-PAGE separation of humanized anti-C5 mAb and anti-C5-FH fusion the corresponding bands separated at ˜50 kDa and ˜80 kDa, respectively (FIG. 1A).

To determine whether 2G1-3 bound to a different epitope from Eculizumab, a commercially available C5 mAb that recognizes the α-chain of C5, a Western blot was run for bound 2G1-3 under non-reduced and reduced conditions. While 2G1-3 bound to non-reduced (NR) human C5, it bound to a band corresponding to the β-chain of human C5 under reducing (R) condition (FIG. 1B). To establish the domain within the β-chain of human C5 that was critical for 2G1-3 binding, culture supernatants from transfected HEK cells with human C5 β-chain deletion mutants lacking individual MG domains (MG1-MG6) were collected and analyzed by Sandwich ELISA. 2G1-3 was used as the capture antibody and SKY59 (humanized anti-C5 long-lasting mAb; see, e.g., Fukuzawa et al., Sci Rep. 2017; 7(1):1080) was used as the detection antibody. Culture supernatants from untransfected HEK cells served as the negative control while normal human serum (NHS) along with intact human C5 β-chain (hC5β) served as the positive controls. 2G1-3 binding to the MG1 domain could not be assessed because the detection mAb SKY59 bound to an epitope in the MG1 domain. As shown in FIG. 1B, the MG4 domain was critical for 2G1-3 binding to the human C5 β-chain.

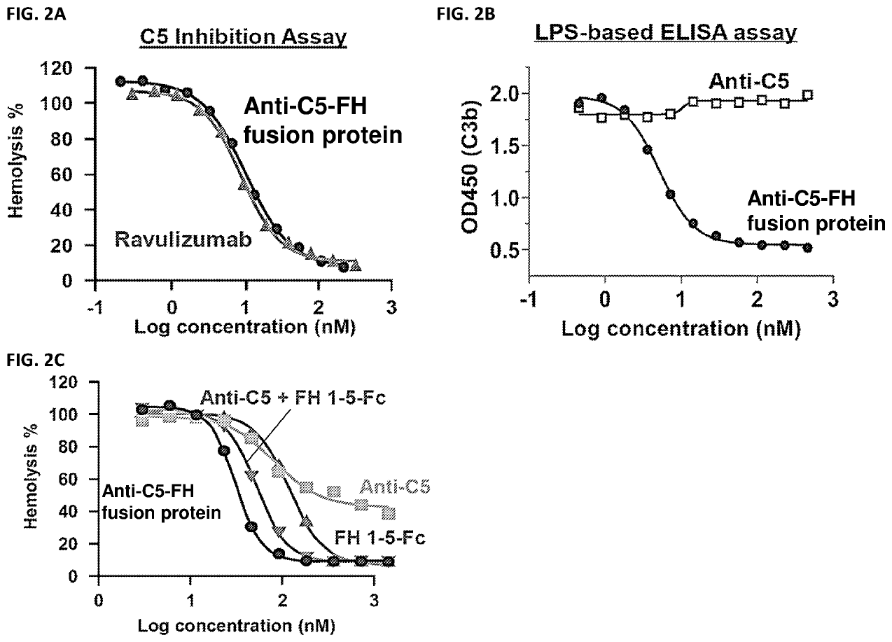

The anti-C5-FH fusion protein was tested for its potency in inhibiting C5 in a sheep RBC lysis assay. Compared to Ravulizumab, an FDA-approved monoclonal antibody inhibitor of C5, the anti-C5-FH fusion protein is as potent as Ravulizumab in inhibiting CP-triggered terminal pathway complement activation (FIG. 2A). Furthermore, based on an LPS-based ELISA assay, unlike the anti-C5 mAb, the anti-C5-FH fusion protein inhibited AP complement (FIG. 2B). As shown in a rabbit RBC lysis assay (FIG. 2C), the anti-C5-FH fusion protein was more potent than anti-C5 mAb alone, AP regulator (FH1-5-Fc (FH SCR domains 1-5 fused to Fc)) alone, or anti-C5 mAb and AP regulator (FH1-5-Fc) together (anti-C5+FH1-5-Fc).

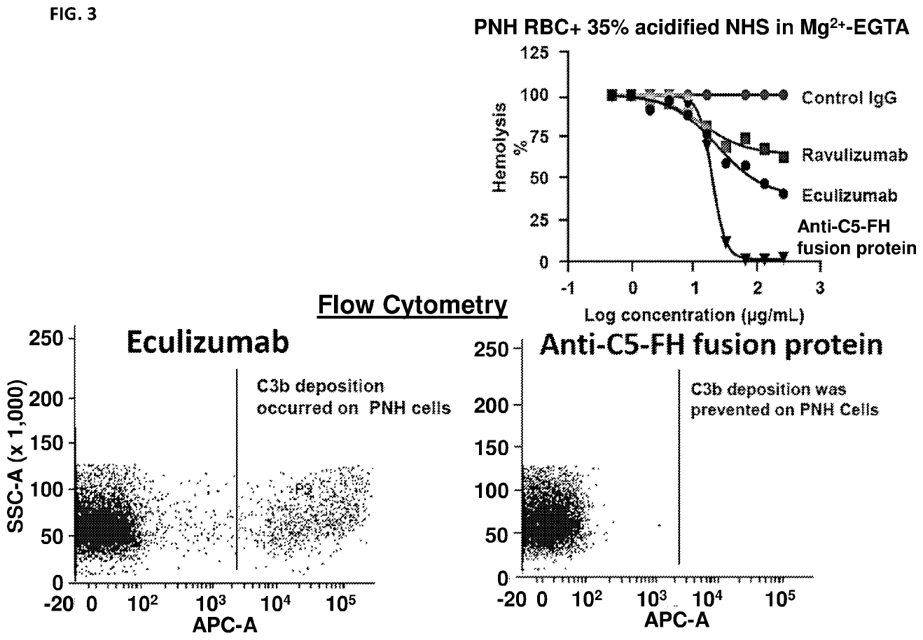

The anti-C5-FH fusion protein was more potent than anti-C5 mAbs, such as Eculizumab and Ravulizumab, in inhibiting the lysis of human paroxysmal nocturnal hemoglobinuria (PNH) RBCs. In a PNH RBCs lysis assay with 35% acidified NHS in Mg2+-EGTA, Eculizumab and Ravulizumab were not able to completely block PNH RBC lysis in a modified Ham's test ex vivo and significant residual hemolysis remained. In contrast, the anti-C5-FH fusion protein was able to fully block PNH RBC lysis and overcame the residual hemolysis (FIG. 3). Moreover, as shown in flow cytometry results in FIG. 3, the anti-C5-FH fusion protein effectively blocked activated C3 fragment opsonization of PNH RBCs, unlike Eculizumab, which had no effect, as expected.

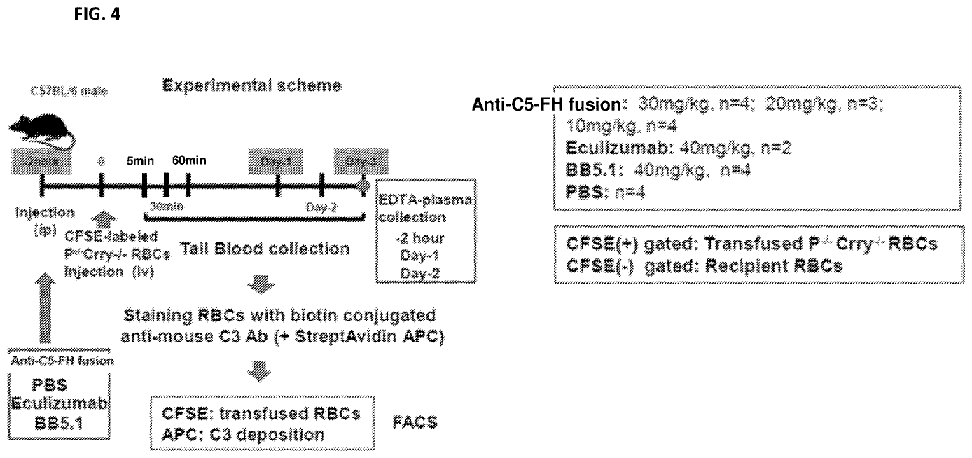

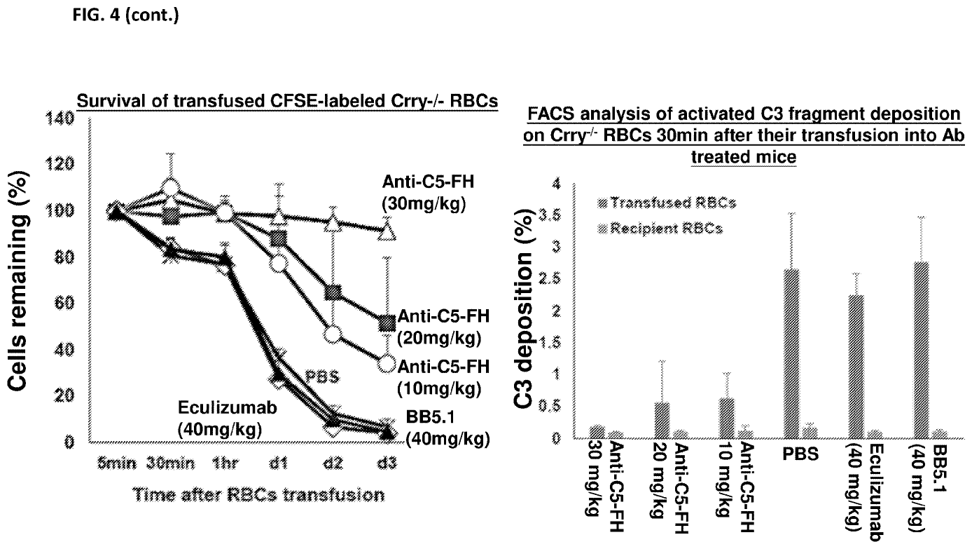

The anti-C5-FH fusion protein dose-dependently inhibited extravascular hemolysis (EVH) in a mouse model of EVH. C57BL/6 males were injected (intraperitoneal injection, i.p.) with 30 mg/kg anti-C5-FH fusion protein (n=4), 20 mg/kg anti-C5-FH fusion protein (n =3), 10 mg/kg anti-C5-FH fusion protein (n=4), 40 mg/kg Eculizumab (n=2), 40 mg/kg BB5.1, an anti-C5 mAb (n=4), or PBS (n=4) 2 hours prior to a CFSE-labeled P−/−Crry−/− RBCs intravenous injection (i.v.). Tail blood was collected at 5 min, 30 min, 60 min, 1 day, 2 days, and 3 days after the i.v. injection. CFSE-labeled transfused RBCs were analyzed by FACs for cell survival. The anti-C5-FH fusion protein prevented EVH in a dose-dependent manner. Injection of mice with 30 mg/kg of anti-C5-FH fusion protein resulted in 100% survival of transfused CFSE-labeled Crry−/− RBCs by day 3, while injection of 20 mg/kg of anti-C5-FH fusion protein and 10 mg/kg of anti-C5-FH fusion protein resulted in 50% and 40% cell survival by day 3, respectively (FIG. 4). Eculizumab and BB5.1 did not prevent EVH, similar to the PBS control (FIG. 4). RBCs were stained with a biotin-conjugated anti-mouse C3 antibody and a Streptavidin-APC and analyzed for C3 deposition by FACs. FACs analysis showed opsonization of the transfused RBCs by activated C3 fragments was inhibited in a dose-dependent manner by the anti-C5-FH fusion protein, but not by Eculizumab or BB5.1 (FIG. 4).

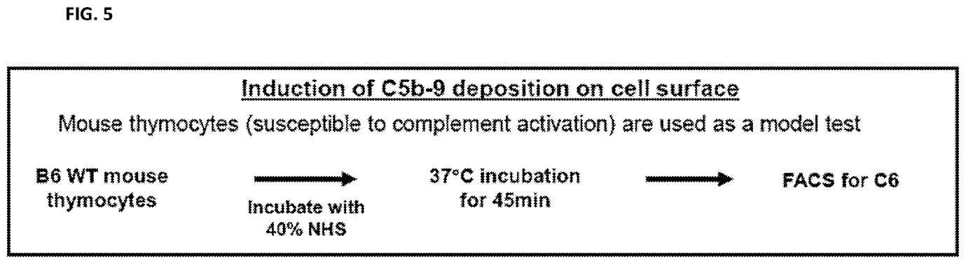

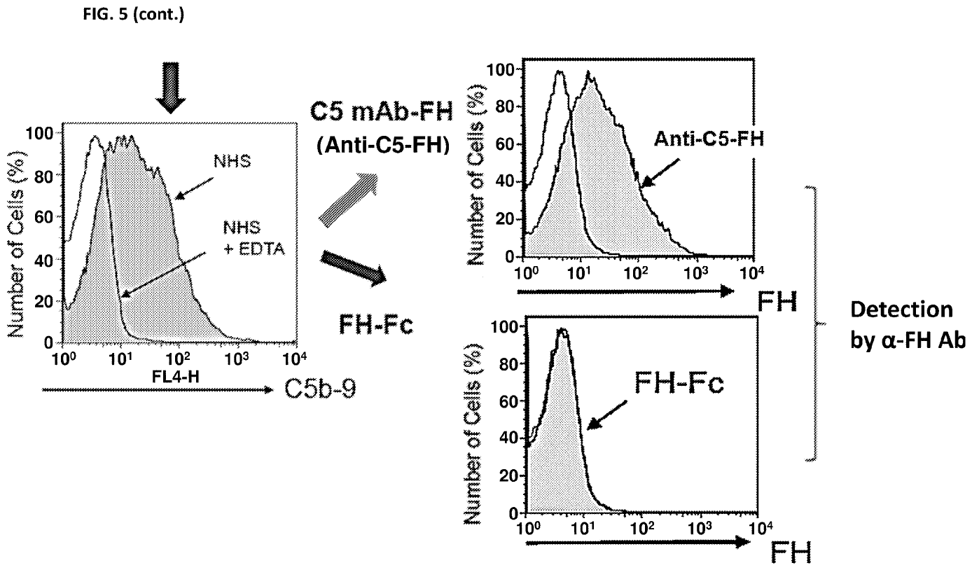

Mouse thymocytes, which are susceptible to complement activation, were used to test for the induction of C5b-9 deposition on the cell surface. C57BL/6 wild-type mouse thymocytes were incubated with 40% NHS at 37° C. for 45 min and were analyzed by FACs for C5. As shown in FIG. 5, unlike the FH1-5-Fc control (FH SCR domains 1-5 fused with an Fc domain), the anti-C5-FH fusion protein bound to cells with C5b-9 deposition, which suggested the anti-C5-FH fusion protein possessed cells- or tissue-targeting property with C5b-9 deposition.

### Conclusion

Anti-C5-FH fusion protein is a novel bi-functional complement inhibitor of the alternative and terminal complement pathways. It was composed of a humanized and extensively engineered (Fab and Fc) anti-C5 mAb fused to SCR1-5 of factor H. Anti-C5-FH fusion protein was more potent than Eculizumab or Ravulizumab in inhibiting rabbit and PNH RBC lysis and was able to block C3b fragment opsonization of PNH RBCs and EVH. Anti-C5-FH fusion protein bound to C5b and had tissue-targeting property for cells or tissues with C5b-9 deposition by virtue of its C5b-binding affinity. Anti-C5-FH fusion protein was potentially more efficacious than either proximal (alternative pathway) or terminal pathway (C5) inhibitors in indications where both pathways were pathogenic.

### Example 2: Therapeutic Efficacy of a Murine Anti-C5-FH Fusion Protein in a Mouse Model of Rapidly Progressing Lethal C3 Glomerulopathy (C3G)

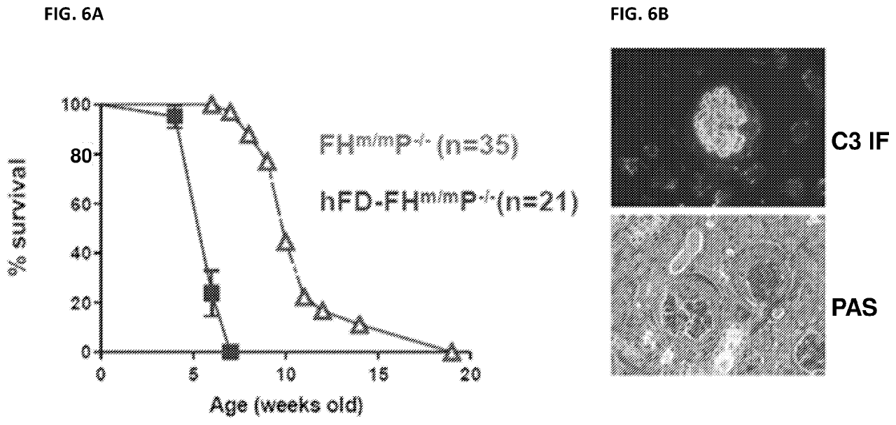

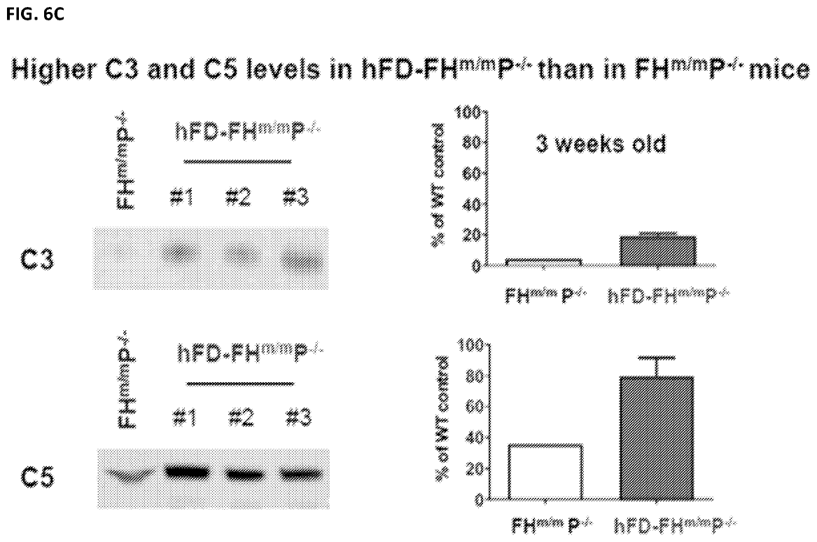

FHm/mP−/− mice, a lethal mouse C3 glomerulopathy (C3G) model in which an anti-mouse C5 mAb (BB5.1) had mixed efficacy on its survival, was generated to test the therapeutic efficacy of a murine anti-mouse C5-FH fusion protein. The bi-functional anti-mouse C5-FH fusion protein was generated by fusing mouse FH SCR domains 1-5 to the C-terminus of heavy chains of a BB5.1 variant anti-mouse C5 mAb. Factor D (FD) was humanized in FHm/mP−/− mice by crossing FHm/mP−/− mice with human FD knock-in mice (hFD). The resultant hFD-FHm/mP−/− mice (n=21) not only recapitulated the C3G phenotype (FIG. 6B), but also developed a more aggressive form of C3G, with 100% mortality occurring before 7 weeks of age, rather than the typical 12-14 weeks of age for FHm/mP−/− mice (n=35) (FIG. 6A). As shown in Western blots and their respective quantifications in FIG. 6C, higher levels of C3 and C5 were present in hFD-FHm/mP−/− mice at 3 weeks old, compared to that of FHm/mP−/− mice.

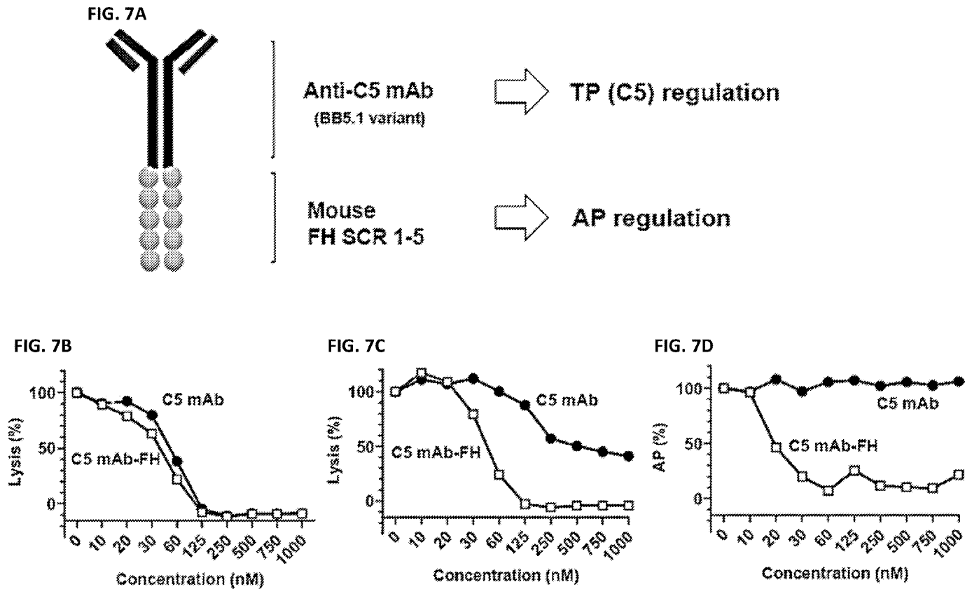

The murine anti-C5-FH fusion protein (FIG. 7A) was tested against BB5.1 for potency in different assays. In a sheep RBC lysis assay with 50% mouse plasma, the murine anti-C5-FH fusion protein and BB5.1 had similar potency in inhibiting terminal pathway complement activation triggered via the classical pathway (FIG. 7B). In a rabbit RBC lysis assay with 50% mouse plasma, the murine anti-C5-FH fusion protein was more potent than BB5.1 in inhibiting terminal pathway complement activation triggered via the alternative pathway (FIG. 7C). In an LPS-based ELISA assay, the murine anti-C5-FH fusion protein inhibited LPS-induced C3b deposition via AP complement activation, unlike BB5.1, which did not inhibit C3b deposition (FIG. 7D).

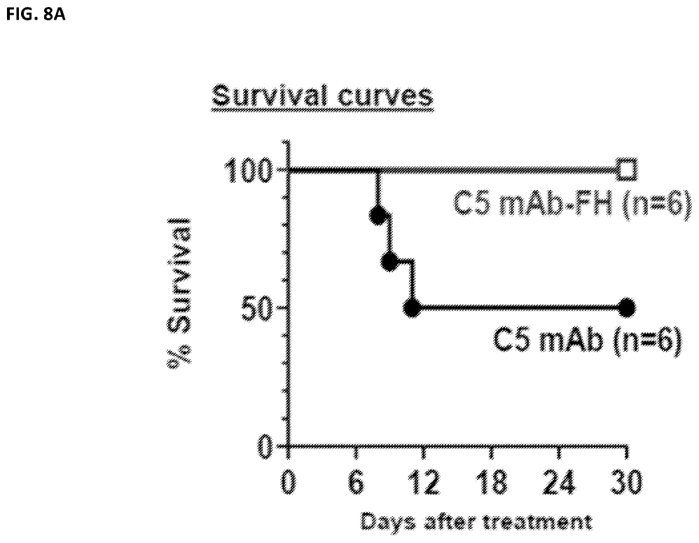

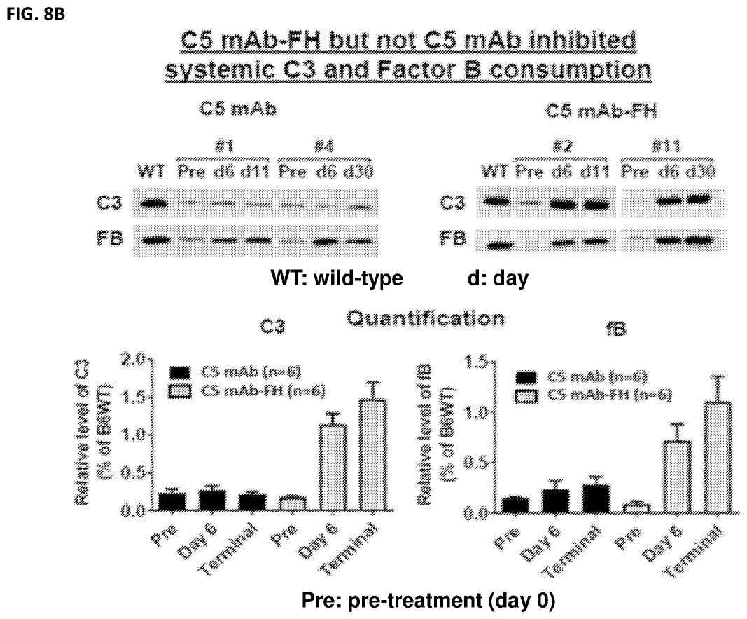

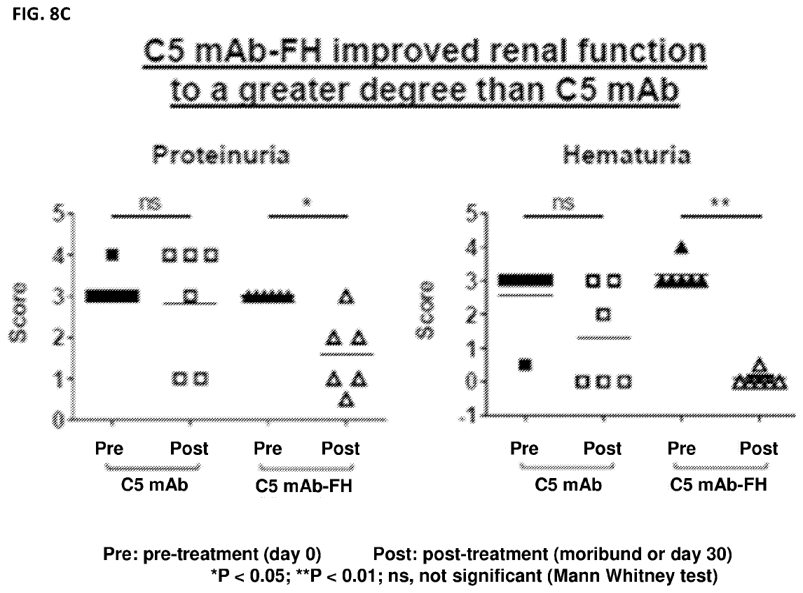

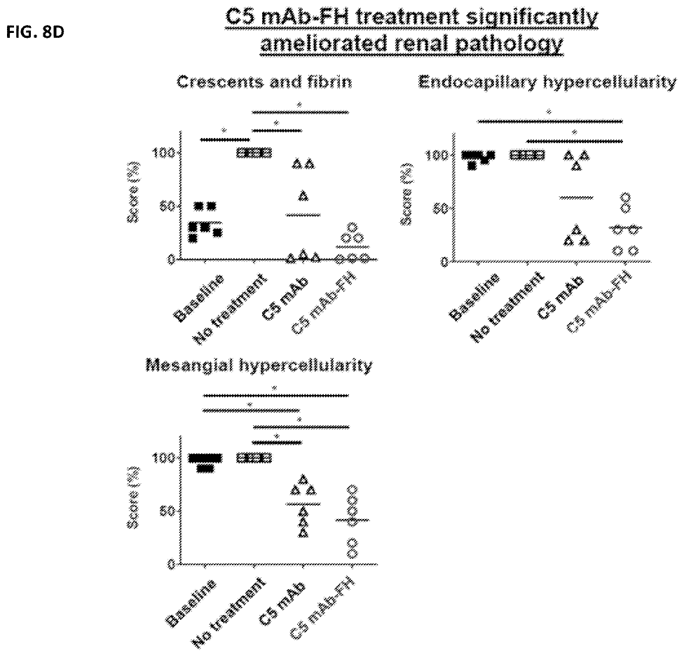

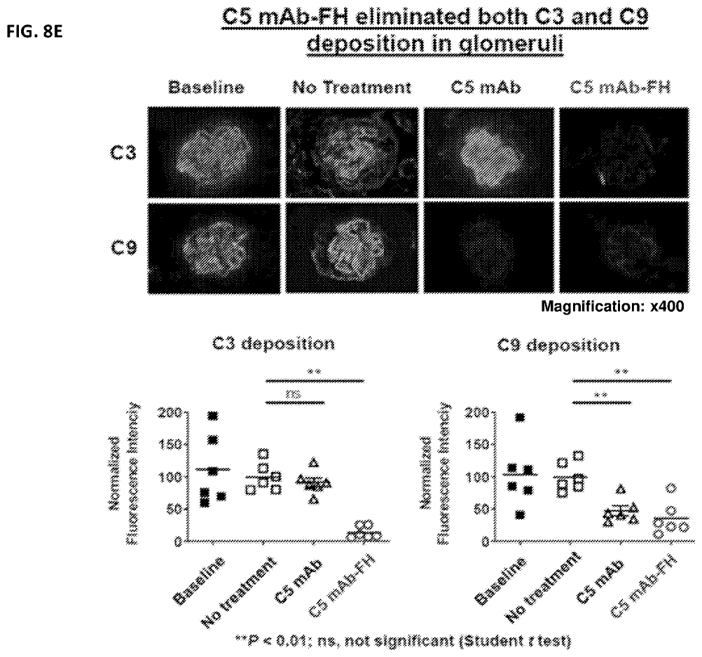

The therapeutic efficacy of BB5.1 and the murine anti-C5-FH fusion protein was tested in hFD-FHm/mP−/− mice screened for development of 3+ proteinuria and hematuria. 40 mg/kg of the BB5.1 (n=6 males, 4-6 weeks old) or the murine anti-C5-FH fusion protein (n=6 males, 3-6 weeks old) was injected (i.p.) every 3 days for a total of 10 doses (30-day treatment). All mice treated with the murine anti-C5-FH fusion protein survived the 30-day treatment whereas only 50% of mice treated with BB5.1 survived (FIG. 8A). The murine anti-C5-FH fusion protein, but not BB5.1, inhibited systemic C3 and factor B consumption. Protein levels of C3 and factor B from hFD-FHm/mP−/− mice injected with the murine anti-C5-FH fusion protein were higher on days 6 and 30, compared to pre-treatment day 0 (FIG. 8B). However, hFD-FHm/mP−/− mice injected with BB5.1 showed comparable protein levels of C3 and only slightly higher protein levels of factor B at days 6 and 30 of treatment, compared to pre-treatment day 0 (FIG. 8B). Additionally, the murine anti-C5-FH fusion protein improved renal function to a greater degree than BB5.1. Following post-treatment at either day 30 or moribund state, treatment with BB5.1 did not significantly improve renal function, whereas treatment with the murine anti-C5-FH fusion protein significantly decreased the scores for both proteinuria and hematuria (FIG. 8C).

To assess the effect of the murine anti-C5-FH fusion protein on renal pathology, kidneys were collected from non-treated and treated mice. All non-treated mice died, and their kidneys were collected when moribund. More than 200 glomeruli in kidney sections of each mouse were examined and scored for glomeruli that showed pathology. When the kidney sections were assessed for crescents and fibrin deposition, endocapillary hypercellularity, and mesangial hypercellularity, BB5.1 significantly reduced the pathologies by 50%, compared to no treatment (FIG. 8D). However, murine anti-C5-FH fusion protein treatment exhibited better amelioration of renal pathology than BB5.1, as only 15% of examined kidney sections showed crescents and fibrin, 25% showed endocapillary hypercellularity, and 40% showed mesangial hypercellularity, compared to the no treatment control (FIG. 8D). Thus, both drugs significantly ameliorated mesangial hypercellularity over baseline and crescents and fibrin deposition and endocapillary hypercellularity over the non-treated cohort, but the murine anti-C5-FH fusion protein further reduced proteinuria, hematuria, and glomerular endocapillary hypercellularity over baseline, compared to BB5.1. Upon immunofluorescence staining for C3 and C9, the murine anti-C5-FH fusion protein prevented both glomerular C3 and C9 deposition whereas BB5.1 only prevented glomerular C9 deposition, as shown by the lack of staining and corresponding quantification in FIG. 8E.

### Example 3: a Phase 1, First-In-Human, Safety, Tolerability, Immunogenicity, PK and PD Study of an Anti-C5-FH Fusion Protein in Escalating Single and Multiple Doses in Healthy Subjects

An anti-C5-FH fusion protein as described in Example 1 (FMEH-IgG4-PLA-FH) for the treatment of Paroxysmal Nocturnal Hemoglobinuria was tested in a randomized, double-blind, placebo-controlled study in healthy human volunteers. Safety, tolerability, anti-drug antibody (ADA) development, pharmacokinetic (PK), and pharmacodynamic (PD) were assessed in single ascending dose (SAD) and multiple ascending dose (MAD) cohorts. The SAD doses ranged from 60 mg to 1200 mg and were administered subcutaneously (SC) or intravenously (IV). The MAD cohort received either 1) 600 mg IV Day 1 followed by 600 mg IV once weekly for 4 weeks or 2) 1200 mg IV Day 1 followed by 720 mg SC once weekly for 4 weeks. To evaluate the function of the anti-C5-FH fusion protein as a bifunctional complement inhibitor, PD markers including free C5, C3b deposition, and rRBC inhibition were evaluated. Free C5 and C3b were measures of anti-C5 and FH activities and rRBC inhibition was a measure of combined AP/TP inhibition by the anti-C5-FH fusion protein. Total C5 and FH levels were also measured.

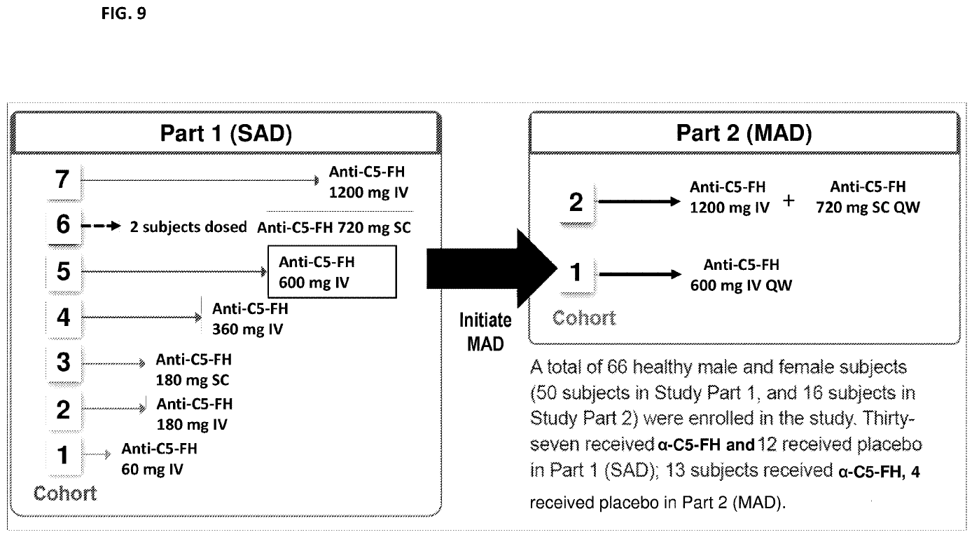

The study was conducted in 2 parts: Part 1, the single ascending dose (SAD) is the first in human (FIH) study of the anti-C5-FH fusion protein and Part 2, multiple ascending dose (MAD). A total of 66 healthy male and female subjects (50 subjects in Study Part 1 and 16 subjects in Study Part 2) were enrolled in the study. 37 received the anti-C5-FH fusion protein and 12 received a placebo in Part 1 (SAD) while 13 subjects received the anti-C5-FH fusion protein and 4 received a placebo Part 2 (MAD) (FIG. 9). The study schema outlined in FIG. 9 illustrate the dosing regimen for each cohort. Also see clinicaltrials.gov NCT05490017.

### Arms and Interventions

Participants in the experimental SAD cohorts received the anti-C5-FH fusion protein intravenous (IV) dose approximately for 1 hour or a subcutaneous (SC) dose. Participants in the experimental MAD cohorts also received the anti-C5-FH fusion protein intravenous (IV) dose approximately for 1 hour or a subcutaneous (SC) dose. Participants in the placebo comparator cohorts received a matching placebo which was the anti-C5-FH fusion protein vehicle containing sodium phosphate, sodium chloride, and L-Lysine Hydrochloride (L-Lys-HCL).

### Primary Outcome Measures

Primary outcome measures included the following: 1) number of participants reporting Treatment Emergent Adverse Events (TEAEs) [Time Frame: Up to Day 85]; 2) number of participants reporting Treatment Emergent Serious Adverse Events (TESAEs) [Time Frame: Up to Day 85]; 3) number of participants with Dose-limiting toxicities (DLT) [Time Frame: Up to Day 85]; and 4) number of participants reporting AEs of Special interests (AESIs) [Time Frame: Up to Day 85]. An Adverse event (AE) is defined as any unfavorable and unintended sign including an abnormal laboratory finding), symptom, or disease or any worsening of a pre-existing condition temporally associated with the use of a study drug, whether or not related to study drug. A TEAE is defined as any AE that started or worsened in severity on or after the first dose of study treatment. A DLT is defined as any adverse event considered by the investigator to be anti-C5-FH fusion-protein-related with a severity greater than or equal to (>=) National Cancer Institute (NCI) Common Terminology Criteria for Adverse Events (CTCAE) v5.0 Grade 3 which also represents a shift from baseline clinical status of >1 NCI CTCAE grade. A hypersensitivity/administration reaction occurring with a severity of Grade 2 despite the use of pre-medications will also be designated as a DLT. Number of participants with AESIs including infections and local or systemic administration reactions was assessed.

### Secondary Outcome Measures

Secondary outcome measures included the following: 1) maximum concentration (Cmax) of anti-C5-FH fusion protein [Time Frame: Up to Day 29]; 2) area under the concentration-time profile (AUC) of anti-C5-FH fusion protein [Time Frame: Up to Day 29]; 3) change from baseline in total and free serum C5 levels [Time Frame: Baseline and up to Day 29]; and 4) change from baseline in rabbit red blood cell (RBC) assay [Time Frame: Baseline and up to Day 29].

### Other Outcome Measures

Other outcome measures included the following: 1) changes in serum free C5 levels to Minimum concentration (Cmin) correlation [Time Frame: Baseline and up to Day 29]; 2) changes in serum free C5 levels to AUC correlation [Time Frame: Baseline and up to Day 29]; 3) changes in C3b activity to Cmax correlation [Time Frame: Baseline and up to Day 29]; 4) changes in C3b activity to Cmin correlation [Time Frame: Baseline and up to Day 29]; 5) changes in C3b activity to AUC correlation [Time Frame: Baseline and up to Day 29]; 6) changes in rabbit RBC lysis to Cmax correlation [Time Frame: Baseline and up to Day 29]; 7) changes in rabbit RBC lysis to Cmin correlation [Time Frame: Baseline and up to Day 29]; 8) changes in rabbit RBC lysis to AUC correlation [Time Frame: Baseline and up to Day 29]; 9) immunogenicity of anti-C5-FH fusion protein [Time Frame: Up to Day 29]; 10) maximum tolerated dose (MTD) of anti-C5-FH fusion protein [Time Frame: Up to Day 29]; 11) optimal biologic dose (OBD) of anti-C5-FH fusion protein [Time Frame: Up to Day 29]; 12) number of participants with clinically significant changes in laboratory values, electrocardiograms (ECGs), physical examinations, and vital signs [Time Frame: Up to Day 29]; 13) changes in serum free complement component C5 levels to Cmax correlation [Time Frame: Baseline and up to Day 29]; 14) dose optimization of anti-C5-FH fusion protein [Time Frame: Up to Day 29]; 15) systemic clearance (C1) of anti-C5-FH fusion protein [Time Frame: Up to Day 29]; 16) elimination half-life (t½) of anti-C5-FH fusion protein [Time Frame: Up to Day 29]; 17) change from baseline in complement component of C3b activity assay [Time Frame: Baseline and up to Day 29]; 18) absolute bioavailability of anti-C5-FH fusion protein administered SC (F) [Time Frame: Up to Day 29]; and 19) change from baseline in Factor H (FH) serum levels [Time Frame: Baseline and up to Day 29].

### Eligibility Criteria

Adults, including healthy volunteers ages 18 years to 55 years of all sexes were eligible for the study.

### Inclusion Criteria

Inclusion criteria included, but were not limited to the following: 1) weight of >40 kilograms (kg) and <120 kg at Screening; 2) in good general health, determined by no clinically significant findings in the opinion of the Investigator from medical history, physical examination, 12-lead ECG, clinical laboratory findings, and vital signs at Screening and Check-in; 3) hemoglobin, hematocrit, white blood cell count, absolute neutrophil count, and platelet count results within the normal range at the Screening Visit; participants with Gilbert's disease with associated abnormalities of liver function tests are eligible for enrollment. Tests may be repeated at the discretion of the Investigator to confirm abnormalities; 4) creatinine clearance based on the Cockcroft-Gault equation of >=80 milliliters per minute (ml/min); 5) females of childbearing potential and males must have practiced effective contraception from Screening until 28 days after the end of study (EOS) visit; and 5) females of childbearing potential must have had a negative pregnancy test at Screening and within 24 hours prior to dosing of study drug; for post-menopausal subjects, a blood sample was also tested for follicle stimulating hormone to confirm post-menopausal status.

### Exclusion Criteria

Exclusion criteria included, but were not limited to the following: 1) any clinically significant underlying illness in the opinion of the Investigator; 2) any history or sign of significant chronic active or recurrent infection, or screening laboratory evidence consistent with a significant chronic active or recurrent infection requiring treatment with antibacterials, antivirals, or antifungals; 3) treatment of any infection with IV (within 30 days of Screening) or oral (within 14 days of Screening) antibacterials, antivirals, or antifungals; 4) history of clinically significant hematologic or bone marrow disease or blood dyscrasias; 5) history of meningococcal infection; 6) history of tuberculosis; 7) history of asplenia (functional or anatomical); 8) prior exposure to anti-C5-FH fusion protein; 9) known allergy to penicillin antibiotics or history of allergy or contraindication to required prophylactic antibiotic therapy to be used during the study; 10) known or suspected complement deficiency during screening; 11) positive serology for Hepatitis B virus (HBV), hepatitis C virus (HCV) or human immunodeficiency virus (HIV) at Screening; and 12) history of drug or alcohol abuse within 1 year of Screening in the opinion of the investigator, or a positive test for drugs of abuse or alcohol at Screening or Check-in.

### Demographics

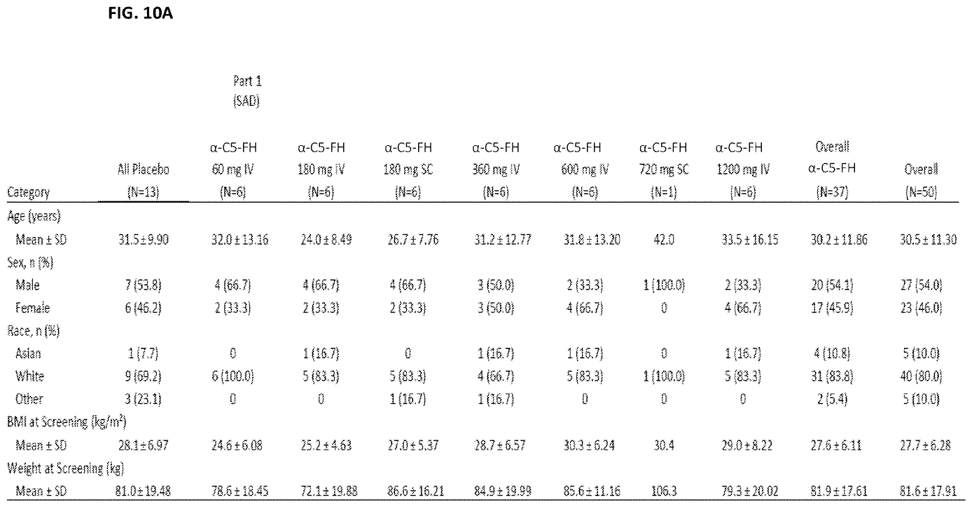

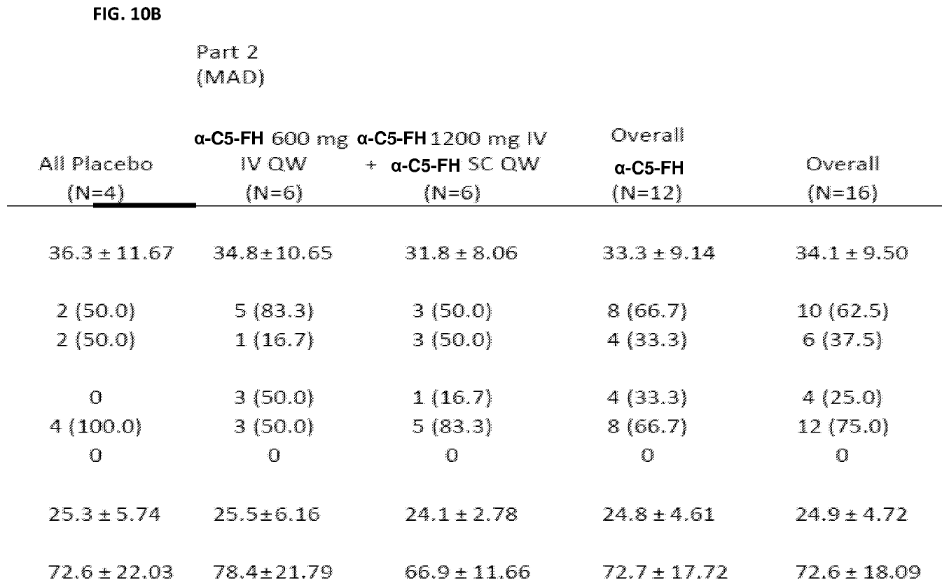

In Part 1 (SAD), demographic characteristics in the placebo group (N=13) were similar to those in the combined anti-C5-FH fusion protein group (N=37) (FIG. 10A). In Part 2 (MAD), all placebo subjects (N=4) were white, while 66.7% (N=8) of anti-C5-FH fusion protein subjects were white. Subjects receiving anti-C5-FH fusion protein 600 mg IV weekly were predominantly male (83.3%) (FIG. 10B). Otherwise, the demographic characteristics were similar in the combined placebo and combined anti-C5-FH fusion protein groups (FIGS. 10A and 10B).

### Safety

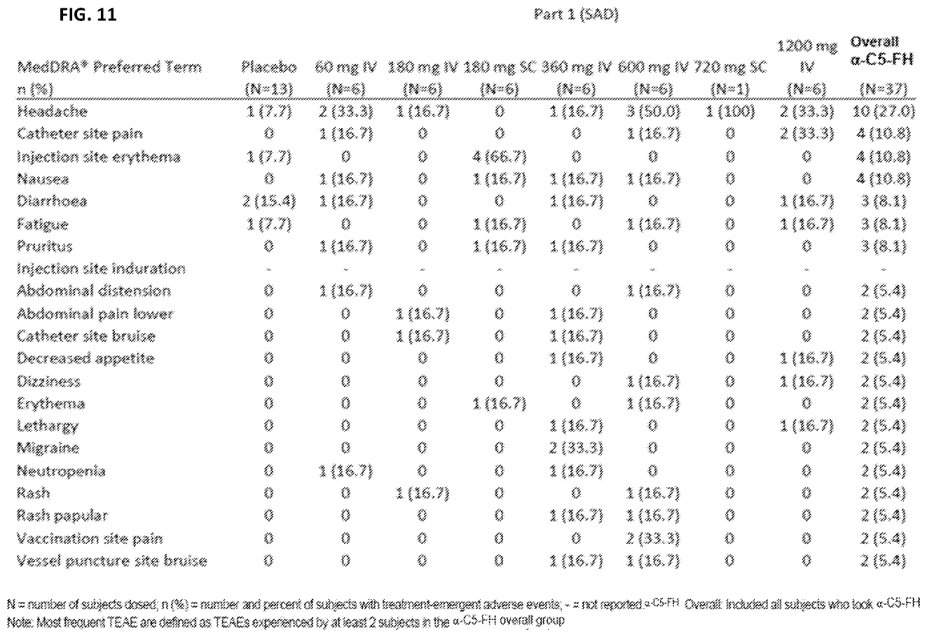

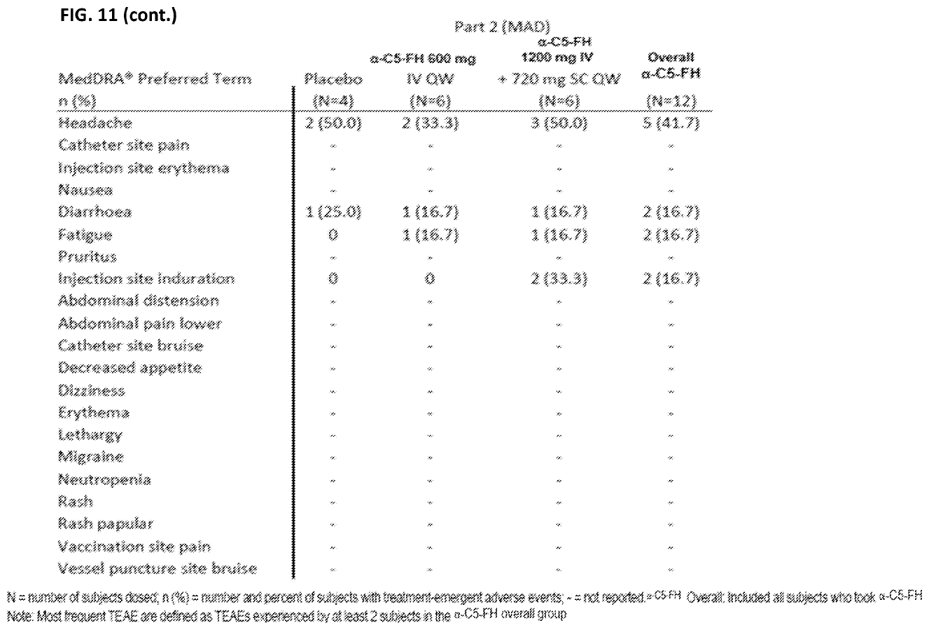

There were no deaths or serious treatment-emergent adverse events (TEAEs) reported in the study; no subjects were discontinued from the study due to drug-related TEAEs. Furthermore, there were no differences in the TEAE profile between subjects who tested positive for anti-C5-FH fusion proteins (14% subjects) and those who were negative for ADA. The majority of the TEAEs were mild (Grade 1) and no severe (Grade 3) TEAEs were reported. No dose-limiting toxicities occurred. There was no clear dose-related trend observed across treatment groups in the incidence of individual TEAE preferred terms, and no drug-related changes were noted in clinical safety laboratory values, vital signs, or ECGs (FIG. 11).

### Pharmacokinetics (PK)

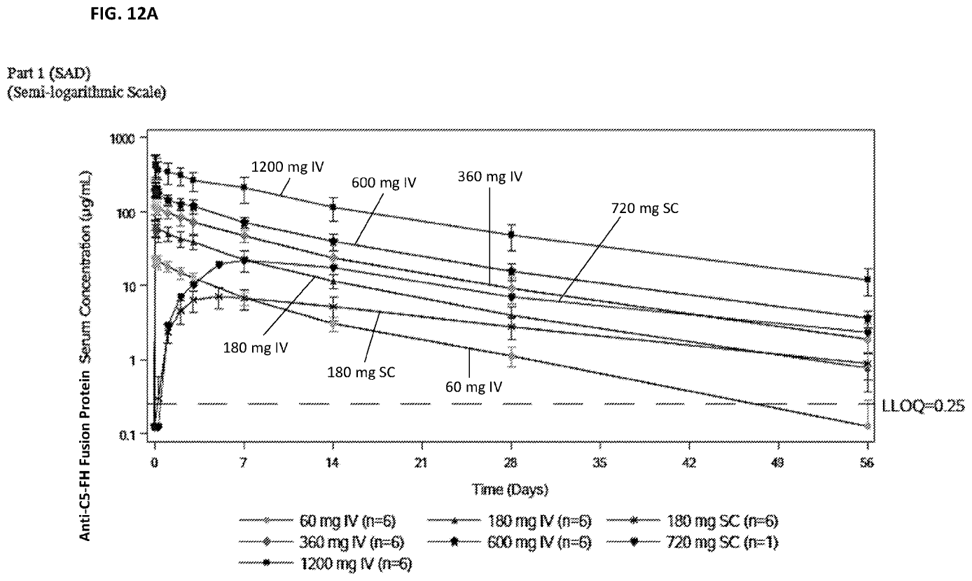

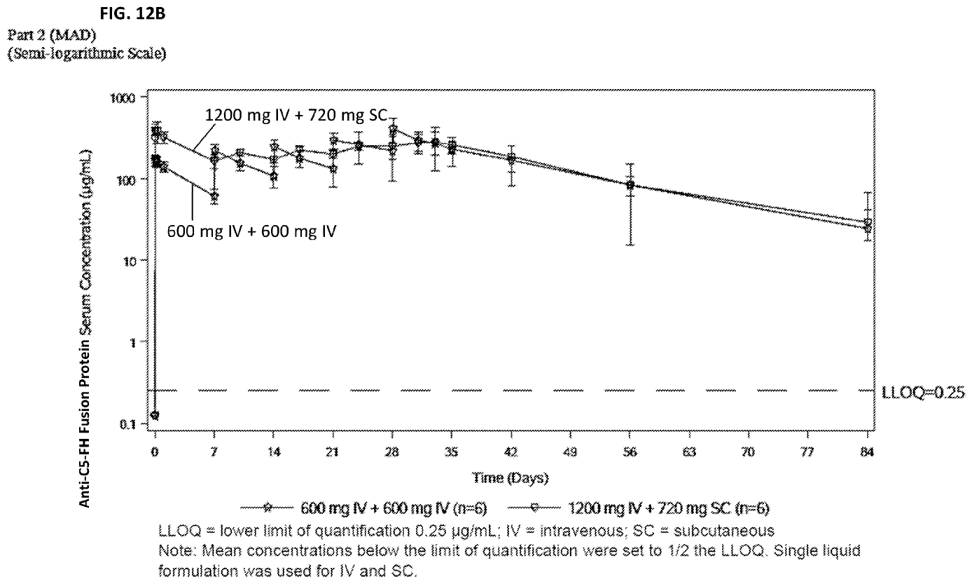

The anti-C5-FH fusion protein PK generally behaved like IgG with approximately dose-proportionality and distributed primarily intravascularly (FIGS. 12A-12B). A single liquid formulation was used for IV (intravenous) and SC (subcutaneous). With 600 mg IV weekly for 5 doses, anti-C5-FH fusion protein peak concentrations increased after each dose, which indicated accumulation. After the last dose at Week 5, the anti-C5-FH fusion protein generally displayed a biphasic profile with a rapid distribution followed by a slow elimination phase, which was measurable up to 8 weeks after the last dose. With 1200 mg IV Day 1+720 mg SC weekly, steady state appeared to be achieved after the fourth dose, and the mean terminal elimination half-life was 15.7 days. Following 4 weekly doses of SC administration, serum anti-C5-FH fusion protein estimated bioavailability was approximately 67% using a population PK modeling approach.

### Pharmacodynamics (PD)

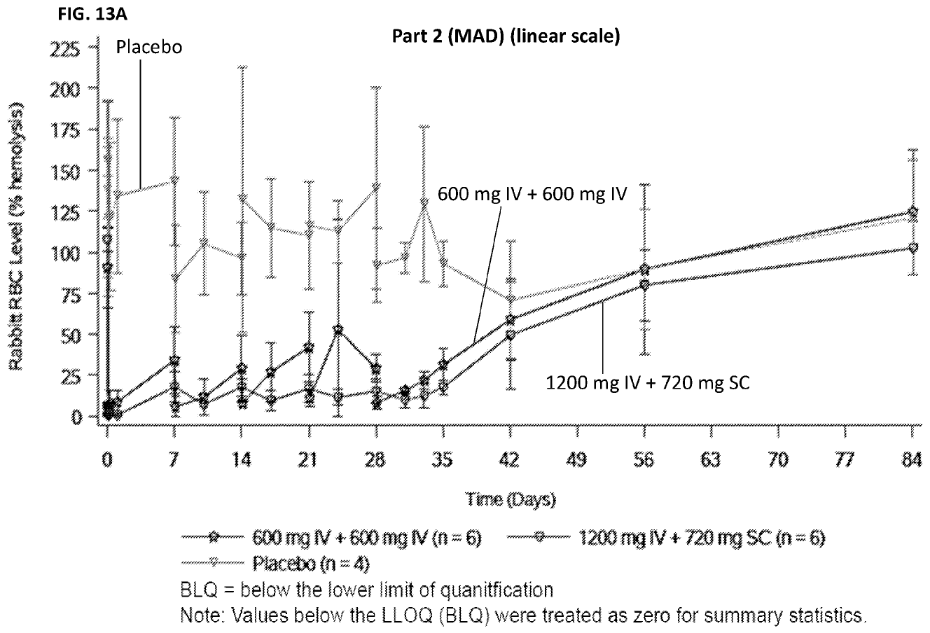

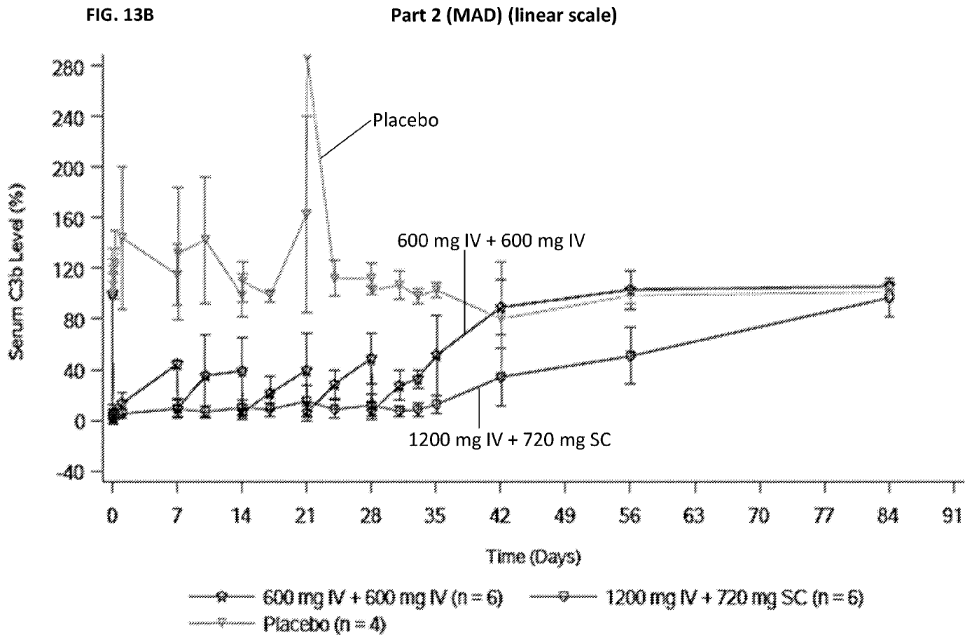

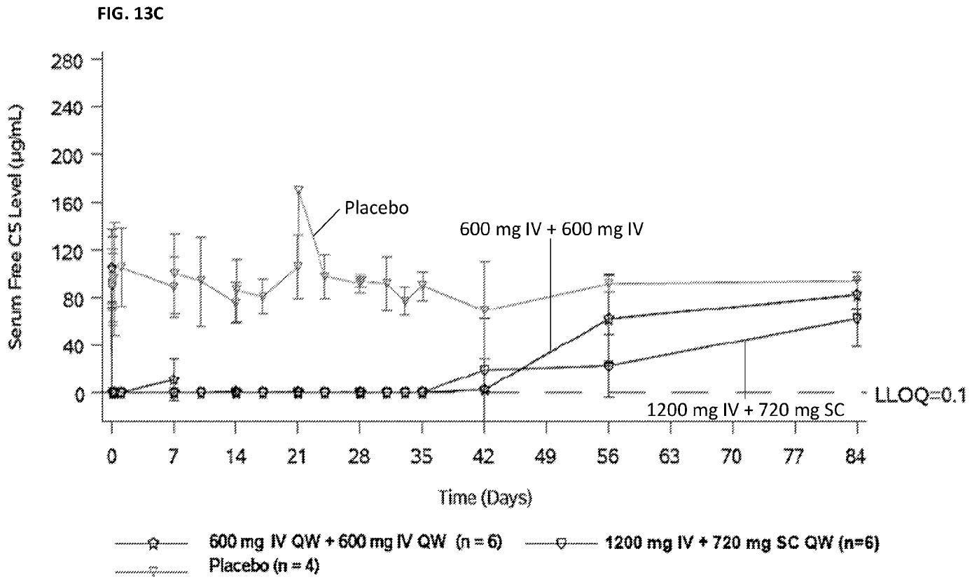

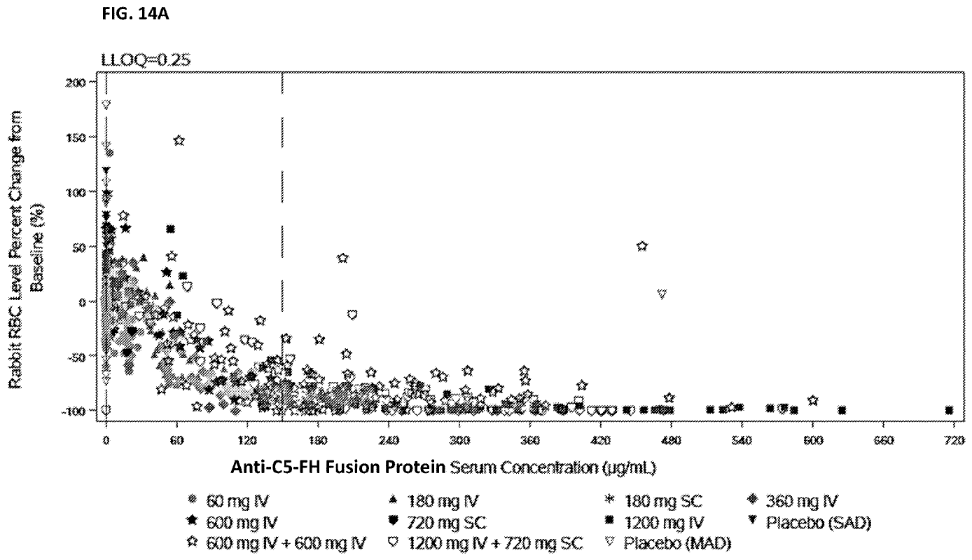

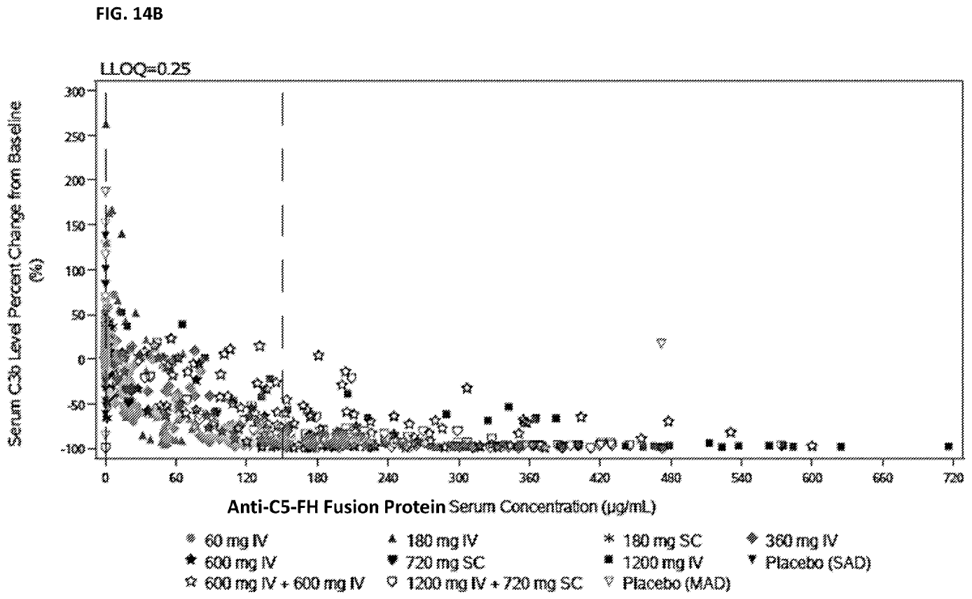

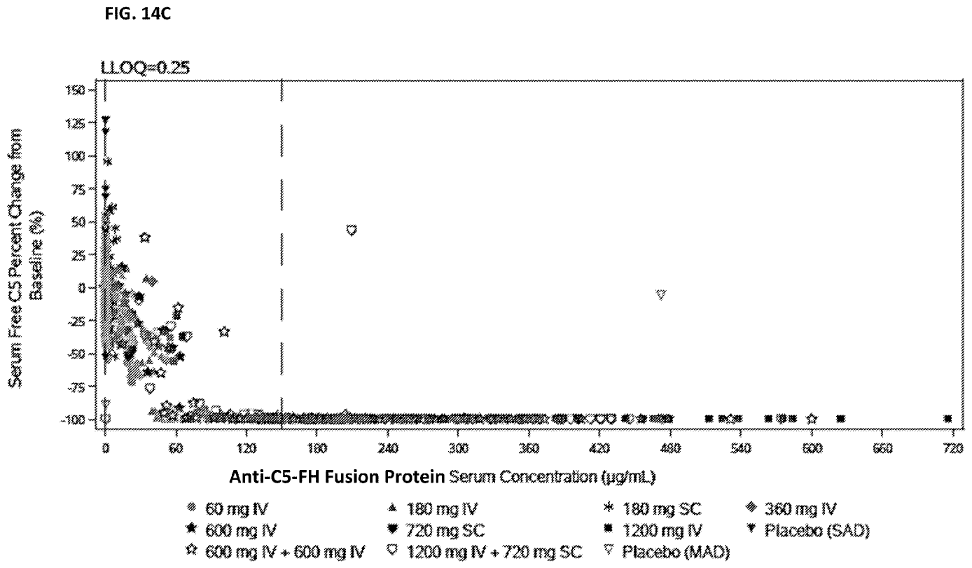

Inhibition of RBC lysis and C3b deposition increased with the concentration of the anti-C5-FH fusion protein and reached at least 80% inhibition from baseline at drug concentrations >150 g/mL. The inhibition on free C5 by anti-C5-FH fusion protein achieved >99.5% at drug concentration >150 g/mL. Free C5 levels were maintained below 0.5 g/mL from Day 1 post-dose to Day 35, which was 8 days following the last dose of anti-C5-FH fusion protein with MAD 1200 mg IV Day 1+720 mg SC weekly for 4 weeks (FIGS. 13A-13C and 14A-14C). There was no significant impact of anti-C5-FH fusion protein dosing on total C5 and factor H levels observed throughout the study.

### Immunogenicity

Seven out of 49 (14%) subjects who received the anti-C5-FH fusion protein tested positive for anti-C5-FH fusion protein antibodies. Two from the Part 1 (SAD) tested positive transiently on Day 15 or 57, with titers ranging from 0 to 60. Five from the Part 2 (MAD) tested positive between Day 57 and Day 85 with titers ranging from 60 to 7680. The presence of ADAs after single or multiple anti-C5-FH fusion protein IV or SC doses did not impact safety.

### Conclusion

The anti-C5-FH fusion protein was safe, well-tolerated, and showed proof of mechanism with potent TP and AP inhibition in this clinical study. The data support planned future clinical trials in complement-mediated kidney diseases.

### Example 4: An Open-Label, Phase 2 Study to Evaluate the Safety, Tolerability, Pharmacokinetics, Pharmacodynamics, and Efficacy of Anti-C5-FH Fusion Protein in Complement Inhibitor-Naïve Subjects with Paroxysmal Nocturnal Hemoglobinuria (PNH)

The purpose of this study is to evaluate the safety, tolerability, immunogenicity, pharmacokinetics, pharmacodynamics, and efficacy of the anti-C5-FH fusion protein as described in Example 1 (FMEH-IgG4-PLA-FH) in complement inhibitor-naïve subjects with PNH. The study will be conducted in 2 parts. Part 1 is a dose-selection study to assess escalating doses and varying dose intervals of the anti-C5-FH fusion protein. Part 2 is a proof-of-concept (POC) study assessing the efficacy of the optimal intravenous (IV) loading dose followed by the optimal maintenance dose and regimen of the anti-C5-FH fusion protein. Also see clinicaltrials.gov NCT05476887.

### Arms and Interventions

Participants in the experimental Part 1 cohorts will receive escalating and varying dose intervals of the anti-C5-FH fusion protein weekly or biweekly. The experimental proof-of-concept cohort for Part 2 will also receive escalating and varying dose intervals of the anti-C5-FH fusion protein weekly or biweekly. All participants will receive a loading dose of the anti-C5-FH fusion protein intravenously (IV) followed by a maintenance dose every week or biweekly.

### Primary Objectives

Part 1 of the primary objective of this study is to assess the safety and tolerability of the anti-C5-FH fusion protein administered as an intravenous (IV) loading dose followed by SC or IV maintenance doses in complement inhibitor-naïve subjects with paroxysmal nocturnal hemoglobinuria (PNH) and to determine the optimal biologic dose (OBD) and regimen of the anti-C5-FH fusion protein in complement inhibitor-naïve subjects with PNH. Part 2 is a proof-of-concept study assessing the efficacy of the optimal intravenous (IV) loading dose followed by the optimal maintenance dose and regimen of anti-C5-FH fusion protein. Part 2 of the primary objective of this study is to assess the efficacy of the anti-C5-FH fusion protein administered as an IV loading dose followed by SC or IV maintenance doses in complement inhibitor-naïve subjects with PNH.

### Secondary Objectives

Part 1 of the secondary objective of this study is to assess the PK of the anti-C5-FH fusion protein administered as an IV loading dose followed by SC or IV maintenance doses in complement inhibitor-naïve subjects with PNH. Part 2 seeks to assess the following: the safety and tolerability of the anti-C5-FH fusion protein administered as an IV loading dose followed by SC or IV maintenance doses in complement inhibitor-naïve subjects with PNH; the pharmacokinetics (PK) of the anti-C5-FH fusion protein administered as an IV loading dose followed by SC or IV maintenance doses in complement inhibitor-naïve subjects with PNH; and the effect of the anti-C5-FH fusion protein administered as an IV loading dose followed by SC or IV maintenance doses in complement inhibitor-naïve subjects with PNH on LDH, hemoglobin and transfusion dependence.

### Exploratory Objectives

Part 1 of the exploratory objective is to assess the efficacy of the anti-C5-FH fusion protein administered as an IV loading dose followed by SC or IV maintenance doses in complement inhibitor-naïve subjects with PNH. Parts 1 and 2 of the exploratory objective aims to assess the following: the pharmacodynamics (PD) and biomarker changes of the anti-C5-FH fusion protein administered as an IV loading dose followed by SC or IV maintenance doses in complement inhibitor-naïve subjects with PNH; the immunogenicity of the anti-C5-FH fusion protein in complement inhibitor-naïve subjects with PNH; and the impact of the anti-C5-FH fusion protein on the quality of life (QoL) of complement inhibitor-naïve subjects with PNH.

### Primary Outcome Measures

Primary outcome measures include the following: 1) Part 1: number of participants with Dose-limiting toxicities (DLT) [Time Frame: Up to Week 4]; 2) Part 2: percentage of participants with >=2 grams per deciliter (g/dL) increase in hemoglobin level from Baseline in the absence of transfusion for weekly dosing [Time Frame: Baseline and at Week 12]; and 3) Part 2: percentage of participants with >=2 g/dL increase in hemoglobin level from Baseline in the absence of transfusion for biweekly dosing [Time Frame: Baseline and at Week 13]. A DLT is defined as any adverse event considered by the investigator to be anti-C5-FH-fusion-protein-related with a severity greater than or equal to (>=) National Cancer Institute (NCI) Common Terminology Criteria for Adverse Events (CTCAE) v5.0 Grade 3 which also represents a shift from baseline clinical status of >1 NCI CTCAE grade. A hypersensitivity/administration reaction occurring with a severity of Grade 2 despite the use of pre-medications will also be designated as a DLT. Blood samples will be collected for the analysis of increase in hemoglobin levels in the absence of transfusion.

### Secondary Outcome Measures

Secondary outcome measures include the following: 1) Part 2: change from Baseline in serum lactate dehydrogenase (LDH) levels for weekly dosing [Time Frame: Baseline and at Week 12]; 2) Part 2: change from Baseline in serum LDH levels for biweekly dosing [Time Frame: Baseline and at Week 13]; 3) Part 2: change from Baseline in hemoglobin level for weekly dosing [Time Frame: Baseline and at Week 12]; 4) Part 2: change from Baseline in the hemoglobin level for biweekly dosing [Time Frame: Baseline and at Week 13]; 5) Part 2: change from Baseline in red blood cell (RBC) transfusion dependence for weekly dosing [Time Frame: Baseline and at Week 12]; 6) Part 2: change in RBC transfusion dependence for biweekly dosing [Time Frame: Baseline and at Week 13]; 7) Parts 1 and 2: number of participants reporting Treatment Emergent Adverse Events (TEAEs) [Time Frame: Up to Week 21]; 8) Parts 1 and 2: number of participants reporting Treatment Emergent Serious Adverse Events (TESAEs) [Time Frame: Up to Week 21]; 9) Parts 1 and 2: number of participants reporting AEs of Special interests (AESIs) [Time Frame: Up to Week 21]; 10) Parts 1 and 2: maximum concentration (Cmax) of anti-C5-FH fusion protein for weekly dosing [Time Frame: Up to Week 20]; 11) Parts 1 and 2: Cmax of anti-C5-FH fusion protein for biweekly dosing [Time Frame: Up to Week 21]; 12) Parts 1 and 2: trough concentration (Ctrough) of the anti-C5-FH fusion protein for weekly dosing [Time Frame: Up to Week 20]; and 13) Ctrough of the anti-C5-FH fusion protein for biweekly dosing [Time Frame: Up to Week 21]. Blood samples will be collected for the analysis of serum LDH and hemoglobin level. The RBC transfusion dependence is the difference in the volume of RBC transfusions per month for the 3 months prior to initiation of investigational product versus the volume of RBC transfusions per month for each month on study. An Adverse event (AE) is defined as any unfavorable and unintended sign (including an abnormal laboratory finding), symptom, or disease or any worsening of a pre-existing condition temporally associated with the use of a study drug, whether or not related to study drug. A TEAE is defined as any AE that started or worsened in severity on or after the first dose of study treatment. A TESAE is defined as any AE that started or worsened in severity on or after the first dose of study treatment.

### Exploratory Outcome Measures

The exploratory endpoint for Part 1 includes the efficacy of anti-C5-FH-fusion protein as assessed by the proportion of subjects at Week 12 (for weekly Dosing) or Week 13 (for biweekly dosing) with a ≥2 g/dL increase in hemoglobin level from baseline in the absence of transfusion. The exploratory endpoints for Parts 1 and 2 include the following: 1) change from baseline in serum lactate dehydrogenase (LDH) levels at Weeks 4 and 8 (for weekly Dosing) or Week 5 and week 9 (for biweekly dosing); 2) change from baseline in mean hemoglobin level at Weeks 4 and 8 (for weekly Dosing) or Week 5 and week 9 (for biweekly dosing); 3) proportion of subjects with breakthrough hemolysis defined as at least 1 new or worsening symptom or sign of intravascular hemolysis (fatigue, hemoglobinuria, abdominal pain, shortness of breath [dyspnea], anemia [hemoglobin <10 g/dL], MAVE including thrombosis, dysphagia, or erectile dysfunction) in the presence of elevated LDH ≥2×the upper limit of normal (ULN), after prior LDH reduction to <1.5×ULN on therapy; 4) change in the proportion of subjects with hemoglobin ≥12 g/dL at Weeks 4, 8, and 12 (for weekly Dosing) or Week 5, 9 and 13 (for biweekly dosing); 5) pharmacodynamics and biomarker changes associated with anti-C5-FH fusion protein administration; 6) immunogenicity of the anti-C5-FH fusion protein; and 7) change in quality of life assessed by the Functional Assessment of Chronic Illness Therapy (FACIT)-Fatigue Score and EQ-5D-3L. The pharmacodynamics and biomarker changes include, but are not limited to the following: 1) change from baseline in C3b activity assay; 2) change from baseline in total and free serum C5 levels; 3) change from baseline in rabbit RBC assay; 4) change from baseline in Factor H serum level; 5) change from baseline in d-dimer; 6) change from baseline in free hemoglobin; 7) change from baseline in serum total and direct bilirubin; 8) change from baseline in serum haptoglobin levels; and 9) change from baseline in reticulocyte count.

### Other Safety Outcome Measures

Other safety outcome measures include the following: type and frequency of changes in clinical laboratory values, safety biomarkers, ECGs, physical examinations, and vital signs.

### Overall Study Design and Plan

This is an open-label, Phase 2 study to evaluate the safety, tolerability, immunogenicity, PK, PD, and efficacy of the anti-C5-FH fusion protein in complement inhibitor-naïve subjects with PNH. Treatment-naïve subjects are defined as those subjects not previously or currently treated with complement-targeting therapeutics. The study will be conducted in 2 parts. Part 1 will assess escalating doses and varying dose intervals of anti-C5-FH fusion protein administered as an IV loading dose followed by SC or IV maintenance doses. Part 2 will be a proof-of-concept (POC) study assessing the efficacy of the optimal IV loading dose followed by the optimal SC or IV maintenance dose and regimen of the anti-C5-FH fusion protein. Part 1 will include up to 3 cohorts of 3 to 6 subjects/cohort dosed for 12 weeks. Part 2 will include 1 cohort of up to 17 subjects receiving the anti-C5-FH fusion protein for 12 weeks with weekly dosing, and 13 weeks with bi-weekly dosing. Intensive PK/PD samples will be collected from 8-12 patients in Parts 1 and 2 who consent to intensive PK/PD evaluation. Sparse PK/PD samples will be collected from the rest of the subjects.

Administration of the anti-C5-FH fusion protein will be initiated in the first dosing cohorts as a weekly dosing regimen according to the Schedule of Events for Weekly Dosing. Thereafter, and pending analysis of safety, PK, or PD data, the dosing regimen may be adjusted as described below to a biweekly regimen.

All subjects completing dosing in this study and demonstrating benefit from the anti-C5-FH fusion protein may be eligible for continued treatment with the anti-C5-FH fusion protein at the optimal biologic dose in a long-term safety extension phase of the trial. The details will be specified in a future protocol amendment.

All subjects participating in this study must provide evidence of prior vaccination against Neisseria meningitidis, Streptococcus pneumoniae, and Hemophilus influenzae at the Screening Visit; subjects not providing evidence of prior vaccination should receive vaccination following initiation of administration of the anti-C5-FH fusion protein but are required to receive treatment with appropriate antibiotic prophylaxis until 2 weeks after completion of vaccination.

Participants in Cohort 1 will receive a loading dose of 1200 mg IV on Day 1 followed by 720 mg SC once weekly (QW) starting on Day 8. Cohort 2 will receive a loading dose of 2400 mg IV or 3600 mg IV on Day 1 followed by 1920 mg SC or 2880 mg SC every two weeks (Q2W) starting on Day 8. Cohort 3 will be determined based on the totality data on safety, efficacy, pharmacokinetics, and pharmacodynamics from Cohorts 1 and 2. The maintenance doses will be administered at least 12 weeks or longer.

### Part 1 (Dose Selection)

Part 1 will utilize a single arm design with sequential dose cohorts. Each dose cohort will have 3 to 6 subjects. The proposed initial anti-C5-FH fusion protein dose, route, and regimen for Part 1 is a 1200 mg IV loading dose followed by a 720 mg SC or 600 mg IV maintenance dose every week; this initial dose and regimen was determined based on results from the preceding Phase 1 clinical study in healthy volunteers. Dose escalation decisions will be based on the data from subjects completing the 4-week DLT assessment period. After the last subject in each cohort has completed the DLT assessment period, an internal data review committee (IDRC) will conduct a review of the available data (safety, tolerability, PK, anti-drug antibodies [ADA], and/or PD) from both the current cohort and cumulative data from all anti-C5-FH fusion protein clinical studies, including cumulative PK/PD modeling, before determining dose and regimen recommendations for advancement to the next dose level.

Each cohort will initially enroll 3 subjects. If 0 of 3 evaluable subjects experiences a DLT, then advancement to the next cohort may occur. The dose and regimen for the next cohort will be based on cumulative safety, PK and PD data from all anti-C5-FH fusion protein clinical studies. It can be lower or higher than that of the current cohort. In addition, if and only if all 3 subjects have a ≥2 g/dL increase in hemoglobin level from baseline in the absence of transfusion up to Week 4, then an additional 3 subjects might be enrolled in the same cohort to confirm the efficacy signal. If 1 of 3 subjects experiences a DLT, then additional 3 subjects will be enrolled in the same cohort for a total of up to 6 evaluable subjects. If 1 out of the 6 evaluable subjects experiences a DLT, then advancement to the next cohort may occur. The dose and regimen for the next cohort will be based on cumulative safety, PK and PD data from all anti-C5-FH fusion protein clinical studies. This dose can be lower or higher than that of the current cohort. If 2 or more subjects in a cohort of up to 6 evaluable subjects experience a DLT, no additional subjects will be enrolled until a complete evaluation of all available data can be conducted and an opinion can be rendered by the IDRC and DSMC as to whether the dose and regimen has exceeded the maximum tolerated dose (MTD). After the third subject in the last cohort in Part 1 completing the DLT period, an optimal biological dose (OBD) for the anti-C5-FH fusion protein will be estimated with all available anti-C5-FH fusion protein safety, PK and PD data in this study and all other anti-C5-FH fusion protein clinical studies for evaluation in Part 2. When OBD is available, ongoing subjects in Part 1 might be switched to OBD at the PI's discretion.

### Part 2 (Proof-of-Concept)

Part 2 is to confirm proof-of-concept by studying the optimal dosing regimen identified in Part 1 in 1 cohort of complement inhibitor-naïve subjects with PNH. Part 2 is planned to include up to 17 subjects. The percent of subjects treated with anti-C5-FH fusion protein showing an increase from baseline in hemoglobin level of at least 2 g/dL at the end of the treatment period (12 weeks for weekly dosing or 13 weeks or bi-weekly dosing) will be compared to a reference background rate of 15%. Assuming the true percent of anti-C5-FH fusion protein-treated subjects demonstrating this increase in hemoglobin is 55%, at least 15 completers will be needed to obtain a power of 90% using an exact binomial one-sample test and a one-sided significance level of 2.5%. Further assuming the dropout rate in the study is 10%, 17 subjects are planned to be enrolled in Part 2. Written informed consent for study participation will be obtained before any study-related procedures or assessments are performed. All potential subjects will be screened for potential participation, and those meeting all eligibility criteria will be offered participation in the study.

Subject participation in both Part 1 and 2 of the study will be conducted in the following 3 defined periods: screen, treatment, and safety follow-up periods.

### Screening Period

The Screening Period will begin when the informed consent form (ICF) is signed. During this period, subjects will undergo baseline assessments to determine eligibility for study participation. The Screening Period duration will be up to 35 days for each of Parts 1 and 2; it will end after all evaluations required to meet eligibility have been completed. If a subject meets all eligibility criteria, they will be offered enrollment into the study.

### Treatment Period

The Treatment Period will begin on Day 1 with administration of the first dose of the anti-C5-FH fusion protein and have a duration of 12 weeks for weekly maintenance dosing regimen and 13 weeks for biweekly maintenance dosing regimen. During the Treatment Period, an initial IV loading dose of the anti-C5-FH fusion protein will be administered to the study subject, followed by SC or IV maintenance doses at the prescribed dosing interval. The initial dose, route and regimen for Part 1 is proposed as a 1200 mg IV loading dose followed by a 720 mg SC or 600 mg IV maintenance dose every week; this initial dose and regimen was determined based on results from the preceding Phase 1 clinical study of the anti-C5-FH fusion protein in healthy volunteers. The precise dose and regimen for subsequent cohorts and for Part 2 (POC) will be determined based on the cumulative data (safety, tolerability, PK, ADA, and/or PD) from all anti-C5-FH fusion protein clinical studies and nonclinical safety studies. Subjects will return to the study site for follow-up evaluations according to a prespecified schedule during the Treatment Period. Following the Treatment Period, subjects, who don't participate in the long-term safety extension phase, will enter the Safety Follow-up Period.

### Safety Follow-Up Period

The Safety Follow-up Period will have a 56-day duration, culminating with an end-of-study (EOS) visit.

Safety will be assessed at each study visit, and assessments of pharmacokinetics, pharmacodynamics, and the potential development of ADAs will be assessed at specific timepoints.

If subjects do not complete all study visits, or terminate early from the study, they will be asked to return to the study site for an Early Termination (ET) Visit. If the subject discontinued from the study during the treatment period, the ET visit should be scheduled within 7 days of the last dose of study drug administration.

If a subject has a DLT during the Treatment Period, study drug dosing will be discontinued for that subject. Subjects experiencing a DLT will be asked to remain in the study and complete the study visits during the safety follow-up period. These subjects will not be replaced.

If a subject terminates early for a reason other than DLT during the Treatment Period, the subject may be replaced.

Any subject experiencing breakthrough hemolysis may receive an additional dose of the anti-C5-FH fusion protein at their current maintenance dose level if deemed necessary by investigators. Subjects not responding to this additional anti-C5-FH fusion protein dose may receive an additional anti-C5-FH fusion protein dose at least 3 days after the first. Subjects should remain on their original maintenance dosing schedule, provided that the next maintenance dose is more than 3 days from the last anti-C5-FH fusion protein dose; if the next maintenance dose is scheduled for less than 3 days from the last anti-C5-FH fusion protein dose, the maintenance dose should be delayed until 3 days after the last anti-C5-FH fusion protein dose. Any subject with continued breakthrough hemolysis not responsive to 2 additional anti-C5-FH fusion protein doses should be removed from study.

A program level DSMC will periodically convene and review all available safety data during the study along with the safety data from other ongoing anti-C5-FH fusion protein studies. After its evaluation, the DSMC may recommend study continuation (with or without modification) or termination. The DSMC may also recommend de-escalation to lower doses and/or regimens. If escalation is terminated, the next-lower dose may be declared the MTD. Alternatively, an additional cohort at an intermediate dose may be added to better define the MTD and/or OBD.

### Dose Limiting Toxicity, Maximum Tolerated Dose, Optimal Biological Dose, Dose Escalation Stopping Rules and Individual Subject Stopping Rules

A DLT is defined as any related AE with a National Cancer Institute Common Terminology Criteria for Adverse Events (NCI CTCAE) 5.0 Grade ≥3 which also represents a shift from baseline clinical status of >1 NCI CTCAE grade. A hypersensitivity/administration reaction occurring with a severity of Grade 2 despite the use of pre-medications will also be designated as a DLT. The MTD is defined as the anti-C5-FH fusion protein dose and regimen (anti-C5-FH fusion protein exposure) below the dose at which 2 or more of 6 evaluable subjects receiving the anti-C5-FH fusion protein in a cohort experience a DLT, as confirmed by the DSMC. The OBD is defined as the lowest dose achieving the target PD effect or demonstrating similar effects on PD biomarkers of the anti-C5-FH fusion protein. OBD will be lower than MTD and will be determined by PK/PD modeling and simulation with data from Part 1.

The study may be stopped at the discretion of the Sponsor based on recommendations of the DSMC. The study may also be stopped pending IDRC or program level DSMC's evaluation if any of the following occur: any study drug-related death, two CTCAE Grade 4 TEAEs that are deemed related to study drug, two or more cases of hypersensitivity reaction of Grade 3 or higher, or two or more cases of bacteremia related to encapsulated bacteria. Dosing of anti-C5-FH fusion protein will be permanently discontinued in a subject if any of the following occurs: any DLT, subject withdraws consent, subject becomes pregnant, subject is unable to comply with the protocol requirements, sponsor terminates the study, study dosing cessation is mandated by a regulatory authority. In all cases, all necessary measures will be taken to ensure appropriate safety follow-up of all subjects in the study.

### Number of Subjects Planned

A total of up to 35 subjects with PNH are planned to be enrolled in the study, including up to 18 (up to 6 per cohort) in Part 1 and up to 17 in Part 2 (POC).

### Study Duration

Up to 26 weeks for each subject is expected.

### Eligibility Criteria

Adults ages 18 years and older of all sexes are eligible for the study. Healthy volunteers are not accepted.

### Inclusion Criteria

Inclusion criteria include, but are not limited to the following: 1) documented diagnosis of PNH confirmed by flow cytometry evaluation of white blood cells and red blood cells, with granulocyte or monocyte clone size of >=10 percent (%) within 6 months of the Screening visit; 2) presence of 1 or more PNH-related signs or symptoms within 3 months of initiation of Screening; 3) LDH >=2.0×ULN at screening; 4) hemoglobin <=10.0 g/dL at screening; 5) females of childbearing potential and males must practice effective contraception from Screening until 28 days after the end of study (EOS) visit; 6) females of childbearing potential must have a negative pregnancy test at Screening and within 24 hours prior to dosing of study drug; 7) BMI of <35 kg/m²; 8) must provide evidence of prior vaccination against Neisseria meningitidis at the Screening Visit (Subjects not providing evidence of prior vaccination should receive vaccination following initiation of administration of the anti-C5-FH fusion protein, but are required to receive treatment with appropriate antibiotic prophylaxis until 2 weeks after vaccination. Subjects who refuse vaccination are not eligible for this study); 9) vaccination against Streptococcus pneumoniae and Hemophilus influenzae should be received if patients have not done so prior to screening visit and vaccines are accessible (If administration of the anti-C5-FH fusion protein is initiated within 2 weeks of vaccination, appropriate antibiotics should be given for prophylaxis); and 10) able to provide Informed Consent.

### Exclusion Criteria

Exclusion criteria include, but are not limited to the following: 1) any clinically significant poorly controlled underlying illness other than PNH per discretion of investigators; 2) treatment of any infection with IV (within 30 days of Screening) or oral (within 14 days of Screening) antibiotics, antivirals, or antifungals; 3) history of meningococcal infection; 4) history of untreated tuberculosis; 5) history of splenectomy; 6) positive serology for Hepatitis C Virus (HCV) ribonucleic acid (RNA) or human immunodeficiency virus (HIV) at Screening; 7) history of bone marrow or stem cell transplantation; 8) absolute neutrophil count (ANC) <500 cells per microliter (cells/μL); 9) reticulocyte count <100×10³ cells/μL; 10) platelet count <30,000 cells/μL; 11) history of systemic autoimmune disease; 12) estimated glomerular filtration rate (eGFR) <30 milliliters/minute/1.73 meter square (mL/min/1.73 m²) calculated by Chronic Kidney Disease Epidemiology Collaboration (CKD-EPI) equation; 13) known allergy to penicillin antibiotics; 14) history of drug or alcohol abuse within 1 year of Screening; 15) received any type of live attenuated vaccine <1 month prior to Screening or is planning to receive any such live attenuated vaccine over the course of the study; 16) use of rituximab within the last 3 months or any complement inhibitors prior to screening; 17) use of steroids (topical use is allowed), EPO, iron supplements, folic acid, vitamin B12, androgen, HIF-PHIs within 4 weeks and immunosuppressive agents within 3 months prior to screening (if doses of above drugs could be maintained stable at this period, it is acceptable); 18) history of malignancy, except adequately treated basal cell carcinoma or in situ carcinoma of the uterine cervix; 19) bone marrow failure and/or candidate for bone marrow transplantation; 20) any female who is pregnant or breastfeeding, or any female who is planning to become pregnant during the study and follow-up period; 21) any condition that, in the investigator's opinion, may compromise study participation, present a safety risk to the subject, or may confound the interpretation of the study results; 22) a QT duration corrected for heart rate by Fridericia's formula (QTcF) >450 millisecond (msec) for males and >470 msec for females based on either single or averaged QTcF values of triplicate ECGs obtained over a 3-minute interval; and 24) currently enrolled in another investigational device or drug study, or less than 30 days since ending another investigational device or drug study.

### Description of Investigational Drug

The anti-C5-FH fusion protein drug product is a liquid solution formulated at a concentration of 120 mg/mL in a phosphate buffer containing sodium phosphate, sodium chloride, L-Lys-HCL, and PS80 at pH of 6.0. The anti-C5-FH fusion protein is provided in 2 mL vials with a fill volume of 1.85 mL for a deliverable volume of 1.5 mL. The anti-C5-FH fusion protein drug product should be stored at 2° C. to 8° C. protected from light.

### Study Drug Dosage Preparation

For IV administration, the anti-C5-FH fusion protein drug product is diluted in an infusion bag containing ˜100 mL of 0.9% sodium chloride for infusion.

For SC administration, the anti-C5-FH fusion protein drug product can be directly injected manually or via a syringe pump to the abdomen. SC injections should not be administered within 3 cm of the umbilicus. Each scheduled SC dose of study drug will be rotated around the 4 abdominal quadrants.

Study drug administration should be conducted with the subject in a seated or recumbent position; subjects should remain in that position for a minimum of 30 minutes. Subjects should refrain from consuming food or water for a minimum of 30 minutes following anti-C5-FH fusion protein administration.

### Dose Modification, Dose Delays, and Dose Schedule Deviations

After OBD is estimated, ongoing subjects in Part 1 may be switched to OBD at PI's discretion. Otherwise, study drug dose level modifications or dosing administration deviations of >48 hours are not permitted during the Treatment Period.

### Example 5: Study to Evaluate Safety, Tolerability and Pharmacodynamics of the Anti-C5-FH Fusion Protein in Participants with Thrombotic Microangiopathy Secondary to Systemic Lupus Erythematosus

This study will evaluate the safety, tolerability, pharmacokinetics (PK), pharmacodynamics (PD), and efficacy of the anti-C5-FH fusion protein as described in Example 1 (FMEH-IgG4-PLA-FH) in participants with systemic lupus erythematosus (SLE)-Thrombotic microangiopathy (TMA). The study consists of 2 parts: Part 1 (Dose Optimization) and Part 2 (Proof of Concept). All participants will receive the anti-C5-FH fusion protein in combination with standard of care (SOC) for SLE-TMA. Also see ClinicalTrials.gov NCT05504187.

### Arms and Interventions

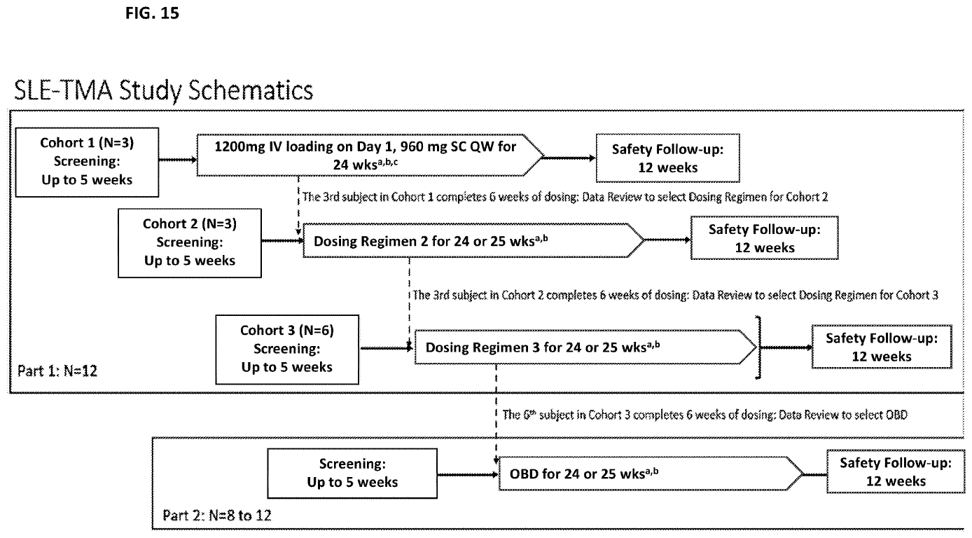

For experimental Part 1, participants in the dose optimization cohort 1 will receive Dose 1. Participants will be administered the anti-C5-FH fusion protein as a weekly maintenance dose for 24 Weeks. After the last participant completes 6 weeks of treatment, all available data, including safety, PK, PD, and modeling results, will be reviewed by the Internal Data Review Committee (IDRC) to determine Dosing Regimen 2. For experimental Part 1, participants in dose optimization cohort 2 will receive Dose 2. Participants will be administered with the anti-C5-FH fusion protein dose regimen 2 for 24 Weeks. After the last participant completes 6 weeks of treatment, all available data, including safety, PK, PD, and modeling results, will be reviewed by the IDRC to determine Dosing Regimen 3. For experimental Part 1, participants in dose optimization cohort 3 will receive Dose 3. Participants will be administered with the anti-C5-FH fusion protein dose regimen 2 for 24 Weeks. After the last participant completes 6 weeks of treatment, all available data, including safety, PK, PD, and modeling results, will be reviewed by IDRC to determine the Optimal biologic dose (OBD) for Part 2. For experimental Part 2, the OBD cohort will receive the OBD, Dose 4. Participants will be administered with the anti-C5-FH fusion protein OBD for 24 Weeks. The initial single loading dose will range from 1200 mg IV to 2400 mg IV, followed by a maintenance dose range of 1) 720 mg SC to 1440 mg SC once weekly (QW) for at least 24 weeks or 2) 1920 mg SC to 2880 mg SC every two weeks (Q2W) for at least 24 weeks. The study schema is shown in FIG. 15.

### Primary Objective

The primary objective is to assess the efficacy of anti-C5-FH fusion protein administered as an intravenous (IV) loading dose followed by subcutaneous (SC) maintenance doses in subjects with thrombotic microangiopathy secondary to systemic lupus erythematosus (SLE-TMA).

### Secondary Objective

The secondary objective is to assess the safety and tolerability of the anti-C5-FH fusion protein administered as an IV loading dose followed by SC maintenance doses in subjects with SLE-TMA.

### Exploratory Objectives

The exploratory objectives include the following: 1) determine the optimal biologic dose (OBD) and regimen of the anti-C5-FH fusion protein in subjects with SLE-TMA; 2) assess the pharmacokinetics (PK) of the anti-C5-FH fusion protein administered as an IV loading dose followed by SC maintenance doses in subjects with SLE-TMA; 3) assess the pharmacodynamics (PD) and biomarker changes of anti-C5-FH fusion protein administered as an IV loading dose followed by SC maintenance doses in subjects with SLE-TMA; 4) assess the PK/PD relationship of the anti-C5-FH fusion protein in subjects with SLE-TMA; and 5) assess the immunogenicity of the anti-C5-FH fusion protein in subjects with SLE-TMA.

### Primary Outcome Measures

Primary outcomes measures include the percent change from Baseline in platelet count [Time Frame: Baseline (Day 1) and up to Week 12] and the percent change from Baseline in serum lactate dehydrogenase (LDH) levels [Time Frame: Baseline (Day 1) and up to Week 12].

### Secondary Outcome Measures

The secondary outcome measures include the number of participants with Treatment-emergent adverse events (TEAEs), treatment-emergent serious adverse events (TESAEs) and Adverse events of special interest (AESIs) [Time Frame: Up to 24 weeks] and the number of participants with change in clinical laboratory values, Electrocardiograms (ECGs), physical examinations, and vital signs [Time Frame: Up to 24 weeks].

### Exploratory Outcome Measures

The exploratory endpoint measures include the following: 1) the percent change of estimated glomerular filtration rate (eGFR) from Baseline to Week 24; 2) the percent change in urine protein/creatinine ratio (UPCR) from Baseline to Week 24; 3) time to the first hematological response, defined as platelet count >100,000/μL accompanied by normalized LDH; 4) time to improvement in platelet count of at least 25% from Baseline; 5) time to improvement in platelet count of at least 50% from Baseline; 6) time to improvement in platelet count of at least 75% from Baseline; 7) percent of subjects with improvement in platelet count at 6 weeks of at least 25% from Baseline; 8) percent of subjects with improvement in platelet count at 6 weeks of at least 50% from Baseline; 9) percent of subjects with improvement in platelet count at 6 weeks of at least 75% from Baseline; 10) change from Baseline to week 24 in haptoglobin, hemoglobin, albumin, 24-hour proteinuria, bilirubin, and reticulocyte count; 11) change in dialysis status throughout the course of treatment; 12) change in red blood cell (RBC) or platelet transfusion requirement (number of transfusions and total units required) through 24 weeks; 13) overall survival at 24 weeks; 14) rate of extra-renal complications related to TMA as determined by the investigator; 15) percentage of subjects with resolution of schistocytosis at final follow-up; 16) markers of healthcare utilization and morbidity including but not limited to: duration of inpatient hospitalization (days) during the study, duration of ICU stay during the study, and quality of life (QoL; The Functional Assessment of Chronic Illness Therapy-Fatigue [FACIT-F]); 17) incidence of investigator-identified treatment failure after at least 6 weeks of anti-C5-FH fusion protein administration as defined by any of the following conditions: doubling of creatinine from Baseline in the setting of renal-related SAEs or corresponding to >1 grade increase in Common Terminology Criteria for Adverse Events (CTCAE), worsening of renal functioning requiring new dialysis, worsening of renal or hematologic functioning requiring use of another complement inhibitor at the discretion of the investigator, worsening of renal, hematological, neurologic, or other functions as a result of TMA, requiring use of IVIG, belimumab, or rituximab at the discretion of the investigator, major extra-renal adverse event secondary to TMA, and subject death due to TMA, TMA-related complications, or study drug as determined by the investigator; 18) change in Systemic Lupus Erythematosus Disease Activity Index (SLEDAI) score from Baseline; 19) in subjects undergoing renal biopsy during the study, the histological findings will be collected and compared to the most recent prior renal biopsy, if available; 20) presence of autoantibodies to Factor H; 21) PK parameters of the anti-C5-FH fusion protein, including but not limited to: maximum concentration (Cmax), trough concentration (Ctrough), area under the concentration-time profile (AUC), serum clearance (CL), and elimination half-life (ti/2); 22) pharmacodynamics and biomarker changes of the anti-C5-FH fusion protein, including but not limited to: change from Baseline in serum C3b and free and total C5, change from Baseline in rabbit RBC assay (alternative complement pathway [AP] and terminal complement pathway [TP] activity), change in factor H endogenous serum level, and change in serum C5b-9; and 23) immunogenicity of the anti-C5-FH fusion protein.

### Other Safety Outcome Measures

Other safety outcome measures include changes in clinical laboratory values, electrocardiograms (ECGs), physical examinations, and vital signs.

### Overall Study Design and Plan

This is an open-label, Phase 2 study to evaluate the safety, tolerability, PK, PD, and efficacy of anti-C5-FH fusion protein in subjects with SLE-TMA. All subjects enrolled will receive anti-C5-FH fusion protein during the treatment period in combination with standard of care (SOC) for SLE-TMA. The study will consist of 2 parts. Results from Part 1 (Dose Optimization) will be used to estimate the OBD, a fixed optimum biologic dosing regimen including the loading, induction, and maintenance dose levels and dosing frequencies. In Part 2 (Proof of Concept), a cohort of subjects will receive the OBD. The predicted maximum exposure given to subjects in this study will not exceed 10200 day·μg/mL, the highest exposure that was well-tolerated in the 26-week GLP toxicology study in monkeys.

### Part 1 (Dose Optimization)

Three cohorts of 3 to 6 subjects per cohort will be enrolled in Part 1. In each cohort, subjects will be dosed with the anti-C5-FH fusion protein for 24 weeks.

In Cohort 1, three subjects will be enrolled and receive a 1200 mg IV loading dose on Day 1 followed by 960 mg SC (bioavailability of SC/IV is˜55%) weekly maintenance dose starting on Day 8 for 24 weeks. After the last subject completes 6 weeks of treatment, all available data, including safety, PK, PD, and modeling results, will be reviewed by the Internal Data Review Committee (IDRC) to determine Dosing Regimen 2. This regimen will include the loading, induction, and maintenance dose levels and dosing frequencies for use in Cohort 2.

In Cohort 2, three subjects will be enrolled and receive Dosing Regimen 2 for 24 weeks. After the last subject completes 6 weeks of treatment, all available data, including safety, PK, PD, and modeling results, will be reviewed by the IDRC to determine Dosing Regimen 3. This regimen will include the loading, induction, and maintenance dose levels and dosing frequencies for Cohort 3.

In Cohort 3, six subjects will be enrolled and receive Dosing Regimen 3 for 24 weeks.

After the last subject completes 6 weeks of treatment, all available data, including safety, PK, PD, and modeling results, will be reviewed by IDRC to determine the OBD for Part 2.

For all 3 cohorts in Part 1, as the newest maintenance dose becomes available, is approved by IDRC, and after consultation with the Sponsor, the new regimen may be administered to the subjects who are still in the treatment period at that time.

The predicted maximum exposure given to subjects should not exceed 10200 day μg/mL, the highest dose that was well-tolerated in the 26-week GLP toxicology study in monkeys.

### Part 2 (Proof-of-Concept)

Upon identification of the OBD, and at the discretion of the Sponsor, up to 12 additional subjects will be enrolled to receive the anti-C5-FH fusion protein OBD for 24 weeks.

All subjects completing dosing in this study and demonstrating benefit from the anti-C5-FH fusion protein may be eligible for continued treatment with the anti-C5-FH fusion protein.

### Definition of Standard of Care and Procedures for Rescue Therapy

Subjects receiving the anti-C5-FH fusion protein in this study will continue to receive SOC therapy for SLE-TMA. SOC includes any combination of the following: IV or PO corticosteroids, cyclophosphamide induction with/without azathioprine maintenance therapy, calcineurin inhibitors, or mycophenolate mofetil. All dosing regimens will be determined by the treating physician. SOC will exclude other complement inhibitors, IVIG, and rituximab.

The study will allow for rescue therapy via plasma exchange, plasmapheresis, and plasma infusion which will require a supplemental dose of the anti-C5-FH fusion protein via IV. The need for rescue therapy due to inadequate response to SOC and the anti-C5-FH fusion protein will be determined by the treating physician.

All subjects completing dosing in this study and demonstrating benefit from the anti-C5-FH fusion protein may be eligible for continued treatment with the anti-C5-FH fusion protein.

All subjects participating in this study who have not received vaccinations against Neisseria meningitidis, Streptococcus pneumoniae, and Haemophilus influenzae previously will receive prophylactic antibiotics starting no later than concurrently with the first dose of the anti-C5-FH fusion protein. Subjects will continue to receive this antibiotic prophylaxis treatment until 2 weeks after vaccination. Subjects not willing to be vaccinated are not eligible to participate in the study.

### Initial Dosing Regimen Rationale

Anti-C5-FH fusion protein is a bi-functional complement inhibitor fusion protein that is comprising a terminal C5 complement inhibitor portion and an alternative complement pathway regulator portion. The initial dose, route, and regimen for Cohort 1 in Part 1 is proposed to be a 1200 mg IV loading dose followed by a 960 mg SC dose weekly. This initial dose is based on PK/PD modeling and simulations using PK/PD data from healthy volunteers. It represents a 5.2-fold and 4.1-fold safety margin for Cmax and AUC, respectively, at steady-state for the predicted human exposure relative to that observed at the highest tolerated dose in the 26-week GLP toxicology study in monkeys.

### Dosing Regimen Selection Process: Part 1 (Dose Optimization)

At each dose selection step described above, the IDRC will conduct a review of all available safety, tolerability, PK, PD, and cumulative PK/PD modeling data from both the current cohort and cumulative data from all anti-C5-FH fusion protein clinical studies before recommending the dose regimen for the next cohorts.

### Dosing Regimen Selection Process: Part 2 (Proof-of-Concept)

The proof-of-concept portion of the protocol will consist of up to 12 subjects treated at the identified optimal dose level described above.

Written informed consent for study participation will be obtained before any study-related procedures or assessments are performed. All potential subjects will be screened for potential participation, and those meeting all eligibility criteria will be offered participation in the study.

Subject participation in the study will be conducted in the following 3 defined periods:

### Screening Period

The Screening Period will begin when the informed consent form (ICF) is signed. During this period, subjects will undergo baseline assessments to determine eligibility for study participation. The Screening Period duration will be up to 35 days, but given the severity of SLE-TMA, it is anticipated to be much shorter. It will end after all assessments to check eligibility criteria have been completed. If a subject meets all eligibility criteria, they will be offered enrollment into the study.

### Treatment Period

The Treatment Period will begin on Day 1 with administration per protocol procedures of the first dose of the anti-C5-FH fusion protein and have a duration of 24 weeks. During the Treatment Period, an initial IV loading dose of the anti-C5-FH fusion protein will be administered to the study subject on Day 1, followed by SC maintenance doses of anti-C5-FH fusion protein at the prescribed dosing interval. Subjects not admitted to hospital will return to the study site as needed for dosing and other study procedures according to a prespecified schedule during the Treatment Period. Following the Treatment Period, subjects will enter the Safety Follow-up Period.

### Safety Follow-Up Period

The Safety Follow-up Period will have an 84-day duration, culminating with an end-of-study (EOS) visit. This correlates with 5 half-lives of the anti-C5-FH fusion protein and a 12-week follow-up.

Safety will be assessed at each study visit, and assessments of PK and the potential development of anti-drug antibodies (ADA) will be assessed at specific timepoints.

If subjects do not complete all study visits, or discontinue early from the study or study drug, they will be asked to return to the study site for an Early Termination (ET) Visit within 7 days of the last dose of study drug administration.

Subjects who discontinue from the study drug due to drug-related SAEs will be asked to remain in the study and complete the study visits through the EOS visit.

If a subject discontinues early for a reason other than a safety-related finding during the Treatment Period, the subject may be replaced. All subjects who discontinue the study prematurely should be followed for safety through EOS, or if unwilling, they should at least have an ET visit.

An external Data and Safety Monitoring Committee (DSMC) will periodically convene and review all available clinical and laboratory data during the study according to the DSMC Charter. After its evaluation, the DSMC may recommend study continuation (with or without modification) or termination of a dose and/or regimen; the DSMC may also recommend de-escalation to lower doses and/or regimens.

### Internal Data Review Committee, Data and Safety Monitoring Committee, Optimal Biological Dose, Study Stopping Rules, and Individual Subject Stopping Rules

An IDRC will be formed with representatives from the Sponsor. The IDRC will be responsible for selecting dosing regimens at the predefined milestones.

A program DSMC will be formed and periodically convene and review all available data on the anti-C5-FH fusion protein according to the DSMC Charter. After its evaluation, the DSMC may recommend study continuation (with or without modification) or termination of a dose and/or regimen. In all cases, the final decisions concerning any DSMC recommendations rest with the Sponsor.

The OBD will be determined based on efficacy, safety, PK, PD, and cumulative PK/PD modeling data from Part 1 by the IDRC. Efficacy as assessed by improvement in platelets and LDH at 6 weeks in combination with safety and PD biomarkers (free C5, rabbit RBC [rRBC] lysis, etc.) will be the primary criteria guiding the determination of the OBD.

The study may be stopped at the discretion of the Sponsor and/or based on recommendations of the DSMC. The study may also be stopped pending DSMC evaluation of all available safety, PK, and PD data if any of the following occur: any study drug-related death; two CTCAE Grade 4 TEAEs that are deemed related to study drug; two or more cases of hypersensitivity reaction of Grade 3 or higher; two or more cases of bacteremia related to encapsulated bacteria; new information or other evaluation regarding the safety or efficacy of the trial medication that indicates a change in the known risk/benefit profile for the compound, such that the risk/benefit is no longer acceptable for subjects participating in the trial; significant violation of Good Clinical Practice (GCP) that compromises the ability to achieve the primary trial objectives or compromises subject safety; sponsor terminates the study; or regulatory authority mandates a study dosing cessation.

Dosing of the anti-C5-FH fusion protein will be permanently discontinued in a subject if any of the following occurs: subject withdraws consent; subject lost to follow-up; any serious adverse event, clinically significant adverse event, severe laboratory abnormality, intercurrent illness, or other medical condition that indicates to the investigator that for safety or tolerability reasons it is in the best interest of the subject to discontinue study intervention; worsening of renal function at least 6 weeks after anti-C5-FH fusion protein initiation requiring new dialysis; pregnancy; subject is unable to comply with the study requirements; sponsor, Institutional Review Board (IRB), IEC, or regulatory agency terminates the study; a dose-limiting toxicity, defined as clinical deterioration as assessed by the investigator due to an adverse event considered by the investigator to be anti-C5-FH-fusion-protein-related with a severity ≥National Cancer Institute (NCI) CTCAE v5.0 Grade 3 which also represents a shift from baseline clinical status of ≥1 NCI CTCAE grade; or a regulatory authority mandates a study dosing cessation.

### Number of Subjects Planned

A total of up to approximately 24 subjects with SLE-TMA are planned to be enrolled in the study, including approximately 12 subjects (3 to 6 per cohort) in Part 1 and up to approximately 12 in Part 2.

### Study Duration

Up to 40 weeks for each subject is expected.

### Eligibility Criteria

Adults ages 18 years to 65 years of all sexes are eligible for the study. Healthy volunteers are not accepted.

### Inclusion Criteria

Inclusion criteria include, but are not limited to the following: 1) meets criteria for SLE per the 2019 European League Against Rheumatism (EULAR)/American College of Rheumatology (ACR) criteria; 2) decrease in platelet count to <100,000/μL AND representing at least a 25% decrease from their pre-study platelet count. Pre-study platelet count is defined as the platelet count within 6 months prior to study screening, or the median of all platelet counts if more than 1 measurement was taken during those 6 months (If no platelet values from before screening are available, a platelet count of <100,000/μL at screening AND a renal biopsy within 6 months with evidence of TMA will be sufficient.); 3) LDH >2×the upper limit of normal (ULN); 4) presence of schistocytes on peripheral blood smear within 14 days of Screening; 5) abnormal renal function as defined by creatinine above the ULN or proteinuria as defined below. Subjects requiring dialysis within 4 weeks of screening for acute kidney injury due to SLE-TMA are eligible (Urine protein >1.0 g/24h; OR UPCR ≥1.0 g/g (or ≥113 mg/mmol) on 2 separate assessments during the Screening Period; these assessments should be separated by at least 3 days and should have a difference of <20% comparing the higher to the lower value); 6) females of childbearing potential and males must agree to practice effective contraception from Screening until 28 days after the EOS visit; 7) females of childbearing potential must have a negative pregnancy test at Screening and/or within 24 hours prior to first dosing of the anti-C5-FH fusion protein; 8) must provide evidence of prior vaccination against Neisseria meningitidis, Streptococcus pneumoniae, and Hemophilus influenzae at the Screening Visit; subjects not providing evidence of prior vaccination should receive vaccination following initiation of administration of the anti-C5-FH fusion protein but are required to receive treatment with appropriate antibiotic prophylaxis until 2 weeks after vaccination; 9) willing and able to provide informed consent; and 10) evidence of microangiopathic hemolytic anemia.

### Exclusion Criteria

Exclusion criteria include, but are not limited to the following: 1) diagnosis of other TMA syndromes, including but not limited to ADAMTS13-deficiency-mediated TMA, metabolism-mediated TMA, Shiga-toxin-mediated TMA, coagulation-mediated TMA, hematopoietic stem cell transplantation-mediated TMA, and drug-mediated TMA; 2) a renal biopsy within 7 days of screening that shows exclusively chronic changes of TMA, as defined by mucoid changes and onion skin lesions of arterioles and/or arteries, WITHOUT any acute components as defined by at least 1 fibrin microthrombus in glomeruli, small arterioles, and/or arteries; 3) any history or sign in the 6 months prior to screening of significant chronic active or recurrent infection or screening laboratory evidence consistent with a significant chronic active or recurrent infection requiring treatment with antibiotics, antivirals, or antifungals; 4) positive Coombs test at the time of TMA diagnosis; 5) treatment of any infection with IV (within 30 days of Screening) or oral (within 14 days of Screening) antibiotics, antivirals, or antifungals; 6) positive nasopharyngeal swab for Neisseria meningitidis at Screening or a prior history of meningitis; 7) history of meningococcal infection; 8) untreated tuberculosis; 9) positive serology for HCV or HIV; 10) history of splenectomy; 11) known allergy to penicillin antibiotics; 12) known or suspected immunodeficiency disease, including hereditary complement deficiency; 13) history of transplant including heart, lung, small bowel, pancreas, liver, kidney, bone marrow, or stem cell transplant; 14) absolute neutrophil count <1000 cells/mm³; 15) eGFR <30 mL/min/1.73 m2 with the exception of subjects requiring acute dialysis within 4 weeks of screening for the new diagnosis of SLE-TMA; 16) platelet count <30,000/mm³; 17) history of drug or alcohol abuse within 1 year of Screening; 18) received any type of live attenuated vaccine <1 month prior to Screening or is planning to receive any such live attenuated vaccine over the course of the study; 19) use of any complement inhibitors within 5 half-lives of the individual therapy; 20) use of IVIG within 7 days of anti-C5-FH fusion protein initiation; 21) use of rituximab within 3 months of anti-C5-FH fusion protein initiation; 22) use of belimumab within 3 months of anti-C5-FH fusion protein initiation; 23) history of malignancy, except adequately treated basal cell carcinoma or in situ carcinoma of the uterine cervix; 24) any female who is pregnant or breastfeeding, or any female who is planning to become pregnant during the study or follow-up period; 25) any condition such as active symptomatic COVID infection that, in the investigator's opinion, may compromise study participation, present a safety risk to the subject, or may confound the interpretation of the study results; 26) a QT duration corrected for heart rate by Fridericia's formula (QTcF) >450 millisecond (msec) for males or >470 msec for females based on either single or averaged QTcF values of triplicate ECGs obtained over a 3-minute interval; 27) currently enrolled in another investigational device or drug study, or less than 30 days since ending another investigational device or drug study; and 28) unwilling to get vaccinated against Neisseria meningitidis, Streptococcus pneumoniae, and Hemophilus influenzae.

### Description of Investigational Drug

The anti-C5-FH fusion protein drug product is a liquid solution formulated at a concentration of 120 mg/mL in a phosphate buffer containing sodium phosphate, sodium chloride, polysorbate 80, and L-Lys-HCL, at pH of 6.0. The anti-C5-FH fusion protein is provided in 2 mL vials with a fill volume of 1.85 mL for a deliverable volume of 1.5 mL. The anti-C5-FH fusion protein drug product should be stored at 2° C. to 8° C. protected from light.

### Study Drug Dosage Preparation

For IV administration, the anti-C5-FH fusion protein drug product is diluted in an infusion bag containing 100 mL of 0.9% sodium chloride for infusion.

For SC administration, the anti-C5-FH fusion protein is administered without further formulation and administered into the abdominal wall using standard techniques for SC administration.

### Example 6: An Open-label, Phase 2 Study to Evaluate the Efficacy, Safety, Pharmacokinetics, and Pharmacodynamics of the Anti-C5-FH Fusion Protein in Subjects with IgA Nephropathy (IgAN) and Complement 3 Glomerulopathy (C3G)

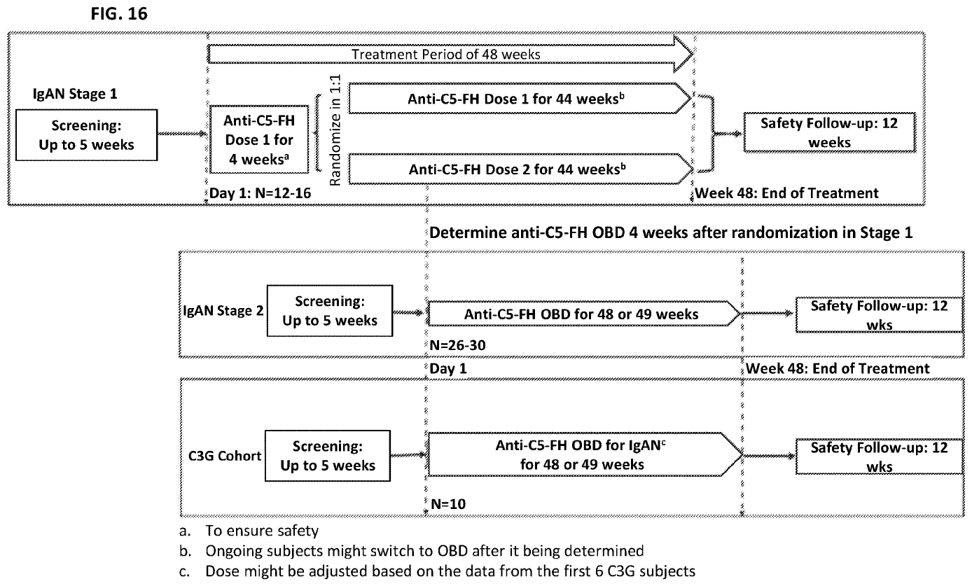

The purpose of this study is to evaluate the efficacy, safety, pharmacokinetics (PK), and pharmacodynamics (PD) of the anti-C5-FH fusion protein as described in Example 1 (FMEH-IgG4-PLA-FH) in participants with IgAN and C3G. The study will start with enrolling the IgAN cohort. Approximately 42 participants with IgAN will be enrolled in 2 stages: Stage 1 will be used to collect safety, immunogenicity, PK, and PD data to select the optimal biologic dose (OBD) of the anti-C5-FH fusion protein for IgAN. Stage 2 will be used to collect safety, immunogenicity, PK, PD, and efficacy data at the OBD dose of the anti-C5-FH fusion protein. As soon as the OBD for IgAN is determined, eligible participants with C3G will be enrolled and dosed at the OBD for IgAN for a minimum of 48 weeks for weekly maintenance dosing and a minimum of 47 weeks for biweekly maintenance dosing. Approximately 10 participants with C3G will be enrolled. Also see ClinicalTrials.gov NCT05517980. The study schema is shown in FIG. 16.

### Arms and Interventions

For the stage 1 experimental IgAN cohort (Dose 1), participants will be randomized to receive weekly or biweekly maintenance doses of the anti-C5-FH fusion protein at Dose 1. Participants in Stage 1 will also have the opportunity to be switched to the OBD if they are still in the treatment period. For the stage 1 experimental IgAN cohort (Dose 2), participants will be randomized to receive weekly or biweekly maintenance doses of the anti-C5-FH fusion protein at Dose 2. Participants in Stage 1 will also have the opportunity to be switched to the OBD if they are still in the treatment period. For the stage 2 experimental IgAN cohort, participants will receive weekly or biweekly maintenance doses of the anti-C5-FH fusion protein at the OBD. For experimental C3G cohort, participants will receive weekly or biweekly maintenance doses of the anti-C5-FH fusion protein at the OBD. All participants will receive loading and/or weekly maintenance intravenous (IV) or subcutaneous (SC) doses of the anti-C5-FH fusion protein. The initial single loading dose will range from 1200 mg IV to 3600 mg IV, followed by a maintenance dose range of 1) 720 mg SC to 1440 mg SC once weekly (QW) for at least 48 weeks or 2) 1920 mg SC to 2880 mg SC every two weeks (Q2W) for at least 49 weeks.

### Primary Objective

The primary objective is to assess the efficacy of the anti-C5-FH fusion protein administered as an intravenous (IV) loading dose followed by IV or subcutaneous (SC) maintenance doses in subjects with IgA nephropathy (IgAN) and complement 3 glomerulopathy (C3G).

### Secondary Objectives

The secondary objectives are to assess the safety and tolerability of the anti-C5-FH fusion protein administered as an IV loading dose followed by SC or IV maintenance doses in subjects with IgAN and C3G and to assess the pharmacodynamics (PD) and biomarker changes associated with the anti-C5-FH fusion protein administered as an IV loading dose followed by SC or IV maintenance doses in subjects with IgAN and C3G.

### Exploratory Objectives

The exploratory objectives include the following: 1) determine the optimal biologic dose(s) (OBD(s) and regimen(s) of the anti-C5-FH fusion protein in subjects with IgAN and C3G; 2) assess the pharmacokinetics (PK) of the anti-C5-FH fusion protein administered as an IV loading dose followed by SC or IV maintenance doses in subjects with IgAN and C3G; 3) assess the PK/PD relationship of the anti-C5-FH fusion protein in subjects with IgAN and C3G; 4) assess the immunogenicity of the anti-C5-FH fusion protein in subjects with IgAN and C3G; and 5) assess the impact of the anti-C5-FH fusion protein on the quality of life (QoL) of subjects with IgAN and C3G.

### Primary Outcome Measure

The primary outcome is measured by the efficacy of the anti-C5-FH fusion protein as assessed by the percent change from baseline in 24-hour urinary protein creatinine ratio (UPCR) at 24 (C3G), and 48 (IgAN) weeks for weekly maintenance dosing and at 23 (C3G) and 47 (IgAN) weeks for biweekly maintenance dosing. The UPCR will be calculated as percent change in protein (Pr)/Creatinine (Cr).

### Secondary Outcome Measures

Secondary outcome measures include the following: 1) safety and tolerability of the anti-C5-FH fusion protein (as assessed by type and frequency of treatment-emergent adverse events (TEAEs), type and frequency of treatment-emergent serious adverse events (TESAEs), and/or type and frequency of adverse events of special interest (AESIs) including infections and local or systemic administration reactions); 2) pharmacodynamics and biomarker changes associated with anti-C5-FH fusion protein administration (including, but not limited to change from baseline in Rabbit RBC assay, change from baseline in C3b activity assay, and/or change from baseline in free serum C5 levels); and 3) efficacy of the anti-C5-FH fusion protein (as assessed by change in eGFR at 24 (C3G) and 48 (IgAN) weeks for weekly maintenance dosing and at 23 (C3G) and 47 (IgAN) weeks for biweekly maintenance dosing).

### Exploratory Outcome Measures

The exploratory endpoint measures include the following: 1) efficacy of the anti-C5-FH fusion protein; 2) pharmacokinetic parameters of the anti-C5-FH fusion protein; 3) pharmacodynamics and biomarker changes associated with anti-C5-FH fusion protein administration; 4) immunogenicity of the anti-C5-FH fusion protein; 5) change in quality of life assessed by the Functional Assessment of Chronic Illness Therapy (FACIT)-Fatigue Score and Kidney Disease Quality of Life (KDQoL) scale; and 6) change in histology and histopathology in subjects undergoing repeat renal biopsy. Efficacy of the anti-C5-FH fusion protein is assessed by change from baseline in UPCR at Weeks 2, 4, 6, 8, 12, 24, 36 and 48 for weekly maintenance dosing and at weeks 1, 3, 5, 7, 11, 23, 35 and 47 for biweekly maintenance dosing; change from baseline in 24-hour urine protein excretion at Weeks 12, 24, 36, and 48 for weekly maintenance dosing and at weeks 11, 23, 35 and 47 for biweekly maintenance dosing; change in eGFR at Weeks 2, 4, 6, 8, 12 and 36 for weekly maintenance dosing and at weeks 1, 3, 5, 7, 11 and 35 for biweekly maintenance dosing; change in hematuria; change from baseline in average weekly corticosteroid use; and/or time to institution of rescue medication. Pharmacokinetic parameters of the anti-C5-FH fusion protein include, but are not limited to maximum concentration (Cmax), trough concentration (Ctrough), and urine PK parameters such as urine concentration of the anti-C5-FH fusion protein. Pharmacodynamics and biomarker changes associated with anti-C5-FH fusion protein administration include, but are not limited to change from baseline in total serum C5 levels, change from baseline in serum and urine Factor H level; and change from baseline in urine C5b-9 level.

### Other Safety Outcome Measures

Other safety outcome measures include changes in clinical laboratory values, electrocardiograms (ECGs), physical examinations, and vital signs.

### Overall Study Design and Plan

This is an open-label, Phase 2 study to evaluate the efficacy, safety, pharmacokinetics, and pharmacodynamics of the anti-C5-FH fusion protein in subjects with IgA nephropathy (IgAN) and complement 3 glomerulopathy (C3G). The study will start with enrolling the IgAN cohort. Approximately 42 subjects with IgAN will be enrolled in 2 stages. Stage 1 will be used to collect safety, immunogenicity, PK, and PD data to select the optimal biologic (OBD) dose of the anti-C5-FH fusion protein for IgAN. Stage 2 will be used to collect safety, immunogenicity, PK, PD, and efficacy data at the OBD dose of the anti-C5-FH fusion protein.

The proposed anti-C5-FH fusion protein starting dose, route, and regimen for subjects with IgAN in Stage 1 is a 1200 mg IV loading dose followed by a 720 mg SC or 600 mg IV weekly maintenance dose (Dose 1). This dose and regimen were determined based on results from all preceding clinical studies with the anti-C5-FH fusion protein. Twelve to sixteen subjects will be enrolled in Stage 1 at Dose 1 for at least 4 weeks to confirm this dose is well-tolerated and enables PK/PD modeling. At the end of Week 4, subjects will be randomized to either continue with Dose 1 or receive anti-C5-FH fusion protein 1440 mg SC or 1200 mg IV weekly (Dose 2) for an additional 44 weeks. The total treatment period for each subject in Stage 1 will be 48 weeks. Dose 2 may be adjusted based on data available prior to randomization. The expected exposure for Dose 2 will not exceed the highest exposure observed in the 26-week GLP toxicology study.

After the 12th subject in Stage 1 finishes the first 8 weeks of treatment, a data review will be performed to select the OBD of the anti-C5-FH fusion protein for IgAN by an internal data review committee (IDRC). All available safety, PK, and PD data, and corresponding modeling and simulation results, of the anti-C5-FH fusion protein from all enrolled subjects will be used. If the OBD for IgAN cannot be determined at this time, another data review will be performed when the 16th subject in Stage 1 finishes the first 8 weeks of treatment to select the OBD. Upon selection of the OBD, the enrollment for Stage 2 will start. All subjects in Stage 2 will receive the OBD of the anti-C5-FH fusion protein for a minimum of 48 weeks for weekly maintenance dosing and a minimum of 47 weeks for biweekly maintenance dosing. All subjects in Stage 1 will also have the opportunity to be switched to the OBD if they are still in the treatment period. Safety, PK, and PD endpoints will be measured during the treatment period.

As soon as the OBD for IgAN is determined, eligible subjects with C3G will be enrolled and dosed at the OBD for IgAN for a minimum of 48 weeks for weekly maintenance dosing and a minimum of 47 weeks for biweekly maintenance dosing. Approximately 10 subjects with C3G will be enrolled. Adjustment of the dose, route, and/or regimen may be performed for subjects with C3G based on safety, PK, and PD data at Week 12 from the first 3 subjects with C3G (Dose 3); the adjusted dose will be given to all subsequently enrolled subjects with C3G. All ongoing subjects will also have the opportunity to be switched to the adjusted dose if they are still in the treatment period.

All subjects participating in this study must provide evidence of prior vaccination against Neisseria meningitidis, Streptococcus pneumoniae, and Hemophilus influenzae at the Screening Visit; subjects not providing evidence of prior vaccination must receive vaccination to all three pathogens and are required to receive concomitant treatment with appropriate antibiotic prophylaxis during any period of coadministration with the anti-C5-FH fusion protein until at least 2 weeks after completion of vaccination. Subjects who refuse vaccination are not eligible for this study.

Subject participation will be conducted in the following 3 defined periods:

### Screening Period

The Screening Period will begin when the informed consent form (ICF) is signed. During this period, subjects will undergo screening assessments to determine eligibility for study participation. The Screening Period duration will be up to 35 days; it will end after all evaluations required to meet eligibility have been completed. If a subject meets all eligibility criteria, they will be offered enrollment into the study. A renal biopsy documentation for eligibility is required; however, repeat renal biopsy will be requested of all subjects during the screening period but will not be required.

### Treatment Period

The Treatment Period will begin on Day 1 with administration of the first dose of the anti-C5-FH fusion protein and have a duration of 48 weeks in Stage 1 and could have a duration of 47 weeks in Stage 2 if biweekly maintenance dosing is adopted. During the Treatment Period, an initial IV loading dose of the anti-C5-FH fusion protein will be administered to the study subject, followed by maintenance doses at the prescribed dose and dosing interval. Each cohort will receive maintenance dosing by only one route, either SC or IV (only if SC is not available), and the route will not change. The initial dose, route and regimen for Stage 1 is proposed as a 1200 mg IV loading dose followed by a 720 mg SC (or 600 mg IV) maintenance dose every week; this initial dose and regimen were determined based on results from all preceding clinical studies with the anti-C5-FH fusion protein. Potential modification of the dose, route, and/or regimen during the study will be determined based on cumulative data (safety, tolerability, PK, ADA, and PD) from all anti-C5-FH fusion protein clinical studies. The expected exposure during the treatment period will not exceed the highest exposure observed in the 26-weeks GLP toxicology study. Subjects will return to the study site for follow-up evaluations according to a prespecified schedule during the Treatment Period. Following the Treatment Period, subjects will enter the Safety Follow-up Period.

### Safety Follow-Up Period

The Safety Follow-up Period will have a 56-day duration, culminating with an end-of-study (EOS) visit. A renal biopsy will be requested of all subjects during the safety follow-up period but will not be required.

Safety will be assessed at each study visit, and assessments of pharmacokinetics, pharmacodynamics, and the potential development of ADAs will be assessed at specific timepoints and followed until resolution or stabilization.

If subjects do not complete all study visits, or terminate early from the study, they will be asked to return to the study site for an Early Termination (ET) Visit within 14 days of the last dose of study drug.

If a subject has a DLT during the first 8 weeks of Treatment Period in Stage 1, study drug will be discontinued for that subject. Subjects experiencing a DLT will be asked to remain in the study and complete study visits during the safety follow-up period. These subjects will not be replaced.

If a subject terminates the study early for a reason other than toxicity during the first 8 weeks of the Treatment Period, an additional subject may be enrolled. Study drug dose level modifications or dosing administration deviations of >48 hours are not permitted during the Treatment Period.

Any subject experiencing flare of their underlying disease, as determined by the study physician, may receive rescue medication according to local standard of care for their disease. Subjects should remain on their maintenance dosing schedule of the anti-C5-FH fusion protein. Any subject with continued flare of their underlying disease not responsive to rescue medication, as determined by the study physician, should be discontinued from the study.

An anti-C5-FH fusion protein program level DSMC will periodically convene and review all available clinical and laboratory data during the study according to the DSMC charter. After its evaluation, the DSMC may recommend study continuation (with or without modification) or termination of a dose and/or regimen and/or disease subgroup; the DSMC may also recommend de-escalation to lower doses and/or regimens.

### Dose Limiting Toxicity, Optimal Biological Dose, Dose Escalation Stopping Rules and Individual Subject Stopping Rules

A DLT is defined as any related AE with an NCI CTCAE 5.0 Grade ≥3 which also represents a shift from baseline clinical status of >1 NCI CTCAE grade, with the exception of isolated laboratory abnormalities that result in no clinically meaningful sequelae. A hypersensitivity/administration reaction occurring with a severity of Grade 2 despite the use of pre-medications will also be designated as a DLT. DLT will be evaluated during the first 8 weeks in Stage 1.

The OBD is defined as the lowest dose achieving the target PD activity of the anti-C5-FH fusion protein or the lower of two doses achieving similar PD activity.

If potentially clinically significant differences in safety, PK, and/or PD are noted between IgAN and C3G subjects, additional subjects at a modified OBD and regimen may be enrolled in C3G cohort; the enrollment of these additional subjects will be at the discretion of the IDRC.

The study may be stopped at the discretion of the Sponsor based on recommendations of the DSMC. The study may also be stopped pending IDRC/DSMC evaluation of all available safety, PK, and PD data if any of the following occur: any study drug-related death, two CTCAE Grade 4 TEAEs that are deemed related to study drug, two or more cases of hypersensitivity reaction of Grade 3 or higher, or two or more cases of bacteremia related to encapsulated bacteria.

Dosing of the anti-C5-FH fusion protein will be permanently discontinued in a subject if any of the following occurs: any DLT (see definition above), subject withdraws consent, pregnancy, subject is unable to comply with the study requirements, sponsor terminates the study, or a regulatory authority mandates a study dosing cessation.

In all cases, all necessary measures will be taken to ensure appropriate safety follow-up of all subjects in the study.

### Number of Subjects Planned

Approximately 52 subjects are planned to be enrolled in the study in order to have a total of approximately 42 subjects with IgAN and approximately 10 subjects with C3G subjects. A 10% dropout rate is anticipated for this study.

### Study Duration

Up to 61 weeks for each subject is expected.

### Eligibility Criteria

Adults ages 18 years to 75 years of all sexes are eligible for the study. Healthy volunteers are not accepted.

### Inclusion Criteria

Inclusion criteria include, but are not limited to the following: 1) weight of >35 kilograms (kg) at Screening; 2) body mass index (BMI) of <35 kilograms per square meter (kg/m²); 3) UPCR >1.5 grams per gram (g/g) by 24-hour urine collection at Screening; 4) documented diagnosis and clinical status of IgAN or C3G.; 5) females of childbearing potential and males must practice effective contraception from Screening until 28 days after the end of study (EOS) visit; 6) females of childbearing potential must have a negative pregnancy test at Screening and within 1 day prior to dosing of study drug; 7) vaccination; and 8) able to provide informed consent. A documented diagnosis and clinical status of IgAN includes the following: 1) diagnosis of IgAN verified by biopsy taken within the past 12 months; 2) on stable regimen of angiotensin converting enzyme or angiotensin blocking agents for 12 weeks and/or sodium-glucose cotransporter-2 (SGLT2) inhibitors for 6 weeks at Screening. A documented diagnosis and clinical status of C3G includes the following: 1) diagnosis of C3G verified by biopsy taken within the past 12 months; 2) on stable regimen of angiotensin converting enzyme or angiotensin blocking agents for 12 weeks and/or SGLT2 inhibitors for 6 weeks at Screening. Regarding vaccination, participants 1) must provide evidence of prior vaccination against Neisseria meningitidis at the Screening Visit; subjects not providing evidence of prior vaccination must receive the vaccination. Subjects who refuse the vaccination are not eligible for this study; 2) if subjects have not received vaccinations against Streptococcus pneumoniae and Hemophilus influenzae at the screening visit and the vaccines are available, they should receive the vaccinations; and 3) for all the three vaccines, if the vaccination is not completed at least 2 weeks prior to the first anti-C5-FH fusion protein administration, appropriate antibiotic prophylaxis should be given until at least 2 weeks after completion of vaccination.

### Exclusion Criteria

Exclusion criteria include, but are not limited to the following: 1) any clinically significant, poorly controlled underlying illness other than IgAN or C3G, as determined by the investigator; 2) any history or sign of significant chronic active or recurrent infection within 6 months of screening or screening laboratory evidence consistent with a significant chronic active or recurrent infection requiring treatment with antibiotics, antivirals, or antifungals; 3) treatment of any infection with IV (within 30 days of Screening) or oral (within 14 days of Screening) antibiotics, antivirals, or antifungals; 4) history of infections with encapsulated organisms; 5) history of untreated tuberculosis; 6) known allergy to penicillin antibiotics; 7) known or suspected immunodeficiency disease, including hereditary complement deficiency; 8) positive serology for hepatitis C virus (HCV) ribonucleic acid (RNA) or human immunodeficiency virus (HIV) at Screening; 9) history of bone marrow or stem cell transplantation; 10) absolute neutrophil count (ANC) <500 cells per microliter (cells/μL); 11) eGFR <30 milliliters per minute per 1.73 square meter (mL/min/1.73 m²) using the Chronic Kidney Disease Epidemiology Collaboration (CKD-EPI) formula; 12) presence of crescent formation in >50 percent (%) of glomeruli assessed on renal biopsy; 13) nephrotic syndrome; 13) rapidly progressive glomerulonephritis, defined as a fall in eGFR of >30 mL/min/1.73 m² within 24 weeks prior to the Screening Visit; 14) receiving renal replacement therapy or anticipated to require renal replacement therapy during the duration of the study; 15) history of drug or alcohol abuse within 1 year of screening; 16) received any type of live attenuated vaccine <4 weeks prior to screening or is planning to receive any such live attenuated vaccine over the course of the study; 17) use of rituximab within the last 12 weeks or any prior use of complement inhibitors; 18) use of systemic corticosteroids >10 mg per day (prednisone or equivalent) within 4 weeks prior to screening or immunosuppressive agents (e.g., mycophenolate mofetil, hydroxychloroquine, cyclosporin etc.) within 3 months prior to screening; 19) history of malignancy, except adequately treated basal cell carcinoma or in situ carcinoma of the uterine cervix; 20) any female who is pregnant or breastfeeding, or any female who is planning to become pregnant during the study and follow-up period; 21) any condition that, in the investigator's opinion, may compromise study participation, present a safety risk to the subject, or may confound the interpretation of the study results; 22) a QT duration corrected for heart rate by Fridericia's formula (QTcF) >450 millisecond (msec) for males and >470 msec for females based on either single or averaged QTcF values of triplicate ECGs obtained over a 3-minute interval; 23) currently enrolled in another investigational device or drug study, or less than 30 days (or 5 half-lives for drugs) since ending another investigational device or drug study; and 24) diagnosed with secondary forms of IgAN as defined by the investigator (e.g., Henoch Schönlein purpura, IgA vasculitis).

### Description of Investigational Drug

The anti-C5-FH fusion protein drug product is a liquid solution formulated at a concentration of 120 mg/mL in a phosphate buffer containing sodium phosphate, sodium chloride, and L-Lys-HCL, and PS80 at pH of 6.0. The anti-C5-FH fusion protein is provided in 2 mL vials with a fill volume of 1.85 mL for a deliverable volume of 1.5 mL. The anti-C5-FH fusion protein drug product should be stored at 2° C. to 8° C. protected from light.

### Study Drug Dosage Preparation

For IV administration, the anti-C5-FH fusion protein drug product is diluted in an infusion bag containing 100 mL of 0.9% sodium chloride for infusion.

For SC administration, the anti-C5-FH fusion protein is administered without further formulation and administered into the abdominal wall using standard techniques for SC administration.

### Example 7: Interim Results of a Phase 2 Study of the Anti-C5-FH Fusion Protein in Complement Inhibitor-Naïve PNH Patients

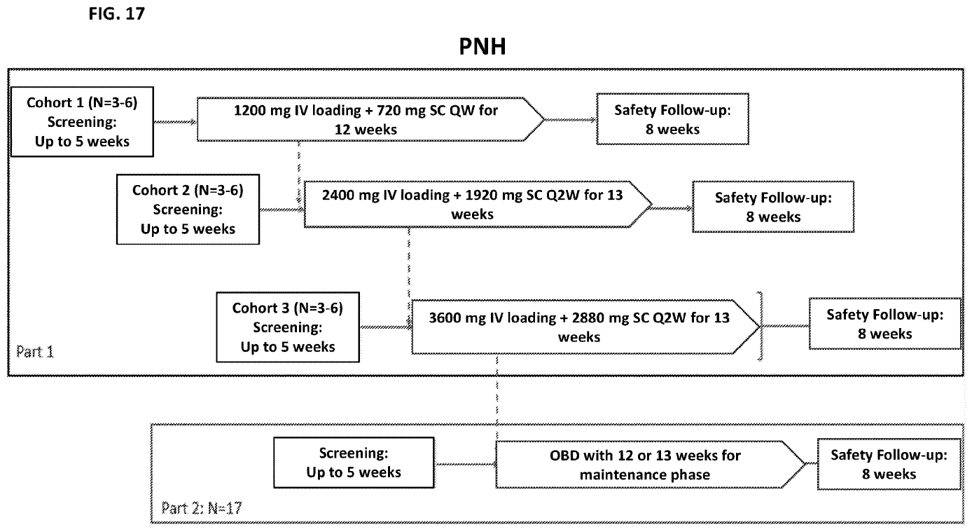

The purpose of this study was to evaluate the efficacy, safety, tolerability, pharmacokinetics, and pharmacodynamics (PD) of the anti-C5-FH-fusion protein in complement inhibitor-naïve PNH patients. The primary objectives of the study were to determine the optimal biologic dose and to assess the clinical endpoints of complement-dependent intravascular hemolysis (IVH) and extravascular hemolysis (EVH) control, including lactate dehydrogenase (LDH), hemoglobin levels (Hgb), transfusion avoidance, and FACIT-fatigue scores. Three to six subjects per cohort were enrolled in up to three cohorts. Patients received an initial IV loading dose followed by weekly (QW) or biweekly (Q2W) SC maintenance doses. Also see Example 4 and FIG. 17 for study design. The first part of the study consisted of two cohorts, with an enrollment of 5 patients in Cohort 1 and 6 patients in Cohort 2. The study included a 5-week screening period, followed by a treatment period of 12 or 13 weeks of the anti-C5-FH-fusion protein dosing regimen. During the screening period, patients underwent baseline assessments to determine eligibility for study participation. The specific dosing regimens for each cohort were as follows: Cohort 1: 1,200 mg IV (Day 1)+720 mg SC QW (starting Day 8) for 12 weeks; and Cohort 2: 2,400 mg IV (Day 1)+1,920 mg SC Q2W (starting Day 8) for 13 weeks.

### Results

Preliminary results were collected from 11 patients (3 males and 8 females, mean age 33.95 years). These include 5 patients from Cohort 1 (1,200 mg IV loading+720 mg SC QW maintenance) treated for 12 weeks and 6 patients from Cohort 2 (2,400 mg IV loading+1,920 mg SC Q2W maintenance) treated for 13 weeks. The study schema is shown in FIG. 17. Baseline LDH and Hgb were 1,865 U/L and 6.55 g/dL for Cohort 1 patients, and 1,597 U/L and 7.77 g/dL for Cohort 2 patients, respectively. All patients experienced a rapid and sustained reduction in LDH levels up to the data cutoff point, with a mean reduction of 83% and 86% in the respective cohorts. Additionally, all 11 patients achieved ≥2 g/dL Hgb increase from baseline (average 4.3 g/dL and 4.7 g/dL increase for Cohort 1 and 2, respectively), and 20% (1/5) in Cohort 1 and 83% (5/6) in Cohort 2 achieved Hgb normalization (>12 g/dL) and improved Absolute Reticulocyte Count, bilirubin, and haptoglobin levels. Following a median of 6 transfusions in Cohort 1 and 12.5 transfusions in Cohort 2 prior to the treatment, none of the 11 patients required additional transfusions or experienced thromboembolic events following the administration of the anti-C5-FH-fusion protein. Patients in both cohorts also had significant improvement in FACIT-fatigue scores (15.0±5.39 and 16.0±8.72 for Cohort 1 and 2, respectively). Dose-dependent reductions in two PD biomarkers, C3b deposition and plasma free C5 levels, were observed, confirming dual mechanism alternative pathway (AP) and terminal pathway (TP) inhibition. Anti-C5-FH-fusion protein was safe and well-tolerated, with no SAEs, deaths, or TEAEs that led to drug discontinuation or study withdrawal. The most frequently reported TEAEs (≥2 patients) were mild or moderate and transient injection site induration, headache, and COVID-19, all were promptly resolved. No infections with encapsulated organisms were observed.

### Conclusion

The interim results from this Phase 2 study suggested that anti-C5-FH-fusion protein was a safe and highly effective therapy for controlling EVH and IVH with dose-dependent efficacy in naïve PNH patients. The results also provided clinical proof-of-concept that a bifunctional AP and TP inhibitor can address the potential unmet medical needs of single pathway inhibitors and obviate the need of cumbersome and expensive combination therapies, as recently proposed (see, e.g., Notaro et al., N Engl J Med. 2022 Jul. 14; 387(2):160-166).

### Example 8: Interim Results of a Phase 2 Study of the Anti-C5-FH Fusion Protein in Complement Inhibitor-Naïve PNH Patients

The purpose of this study was to evaluate the efficacy, safety, tolerability, pharmacokinetics (PK), and pharmacodynamics (PD) of the anti-C5-FH-fusion protein in complement inhibitor-naïve PNH patients. The primary objectives of the study were to evaluate key clinical markers including lactate dehydrogenase (LDH) and hemoglobin (Hgb) levels, transfusion requirements, and FACIT-fatigue scores. Also see Example 4 and FIG. 17 for study design. The first part of the study consisted of three cohorts, with an enrollment of 6 patients per cohort. The study included an initial treatment period of 12 or 13 weeks of the anti-C5-FH-fusion protein dosing regimen, followed by a 9-month extension treatment period. The specific dosing regimens for each cohort were as follows:
Cohort 1: 1,200 mg IV (Day 1)+720 mg SC QW (starting Day 8) for 12 weeks;
Cohort 2: 2,400 mg IV (Day 1)+1,920 mg SC Q2W (starting Day 8) for 13 weeks;
Cohort 3: 3,600 mg IV (Day 1)+2,880 mg SC Q2W (starting Day 8) for 13 weeks; and each cohort was then followed by 1,920 mg SC Q2W for 9 months during an extension phase.

### Results

Interim results included data from 15 patients who completed 16/17 weeks of treatment (6 patients each from Cohort 1 and 2, and 3 patients from Cohort 3). Among these patients (4 males and 11 females, mean (±SD) age 33(±12) years), a median of 3 transfusions were required 12 months prior to the study and 9 patients were previously diagnosed with aplastic anemia. Baseline mean (SD) Hgb and LDH levels were 7.0 (±1.5) g/dL and 1,824 (±512) U/L, respectively.

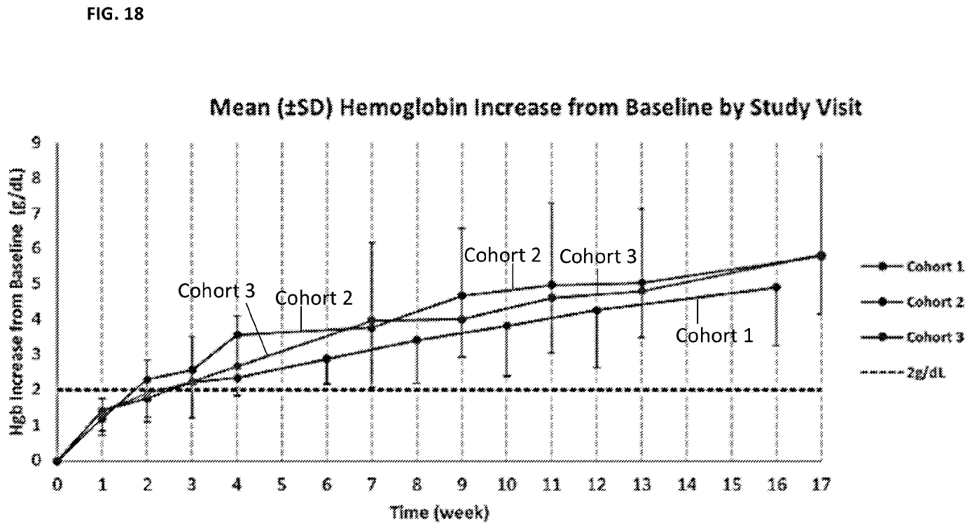

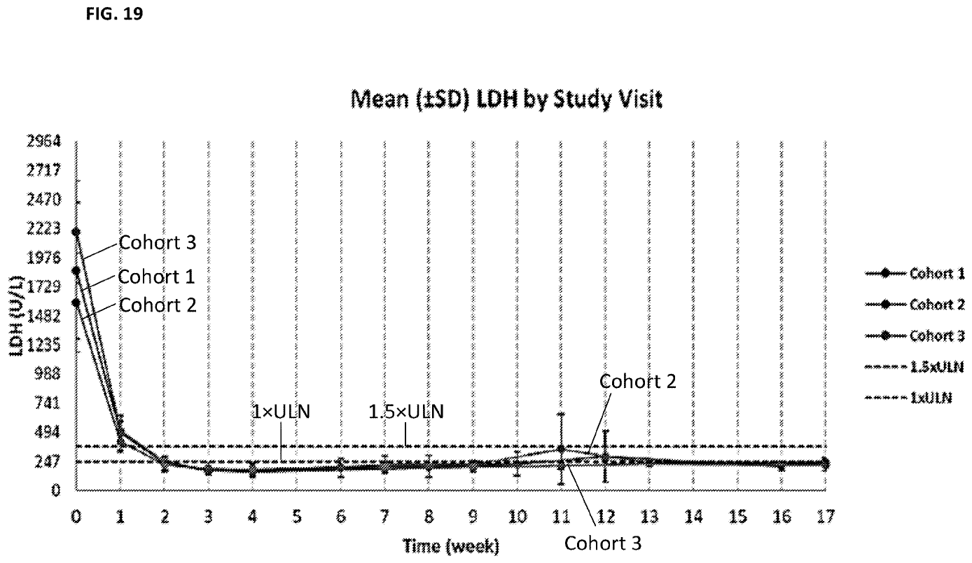

By week 16/17, all 15 patients demonstrated positive clinical improvements. Mean (SD) Hgb levels increased by 4.9 (±1.7) g/dL, 5.8 (±1.6) g/dL, and 5.8 (±2.8) g/dL over baseline (FIG. 18), and mean (SD) LDH levels reduced by 88.0 (±3.67) %, 83.5 (±7.45) %, and 89.5 (±4.05) % over baseline (FIG. 19) for Cohorts 1, 2 and 3, respectively. Additionally, 9/15 (60%) patients achieved hemoglobin levels ≥12 g/dL (3/6, 5/6, and 1/3 for Cohorts 1, 2 and 3, respectively), and all 15 patients had LDH levels below 1.5×ULN (upper limit of normal). None of the 15 patients required transfusions or encountered any thromboembolic events after receiving the anti-C5-FH-fusion protein treatment. Positive changes were observed in absolute reticulocyte count and bilirubin levels, along with significant enhancements in FACIT-fatigue scores seen in all 3 cohorts, with mean (SD) increases of 13.8 (±7.91), 12.8 (±9.15), and 9.3 (±10.69) for Cohorts 1, 2, and 3, respectively. Dose-dependent reductions in two PD biomarkers, C3b deposition and free C5 levels, further confirmed anti-C5-FH-fusion protein's dual mechanism of AP and TP inhibition.

Anti-C5-FH-fusion protein was well tolerated, with no serious adverse events or treatment-emergent adverse events (TEAEs) that led to drug discontinuation or study withdrawal. 10/15 (67%) patients reported at least one mild or moderate TEAE, with no observed dose dependency. The most frequently reported TEAEs included transient injection site induration, headache, and COVID-19 infection, all of which were promptly or duly resolved. A single occurrence of clinical breakthrough hemolysis, observed in the lowest dose cohort and concurrent with an episode of gastroenteritis, was quickly resolved after an extra dose.

### Conclusion

The interim results of this phase 2 study demonstrate the safety and efficacy of the anti-C5-FH-fusion protein in effectively controlling IVH and EVH in complement inhibitor-naïve PNH patients. The findings provide compelling clinical evidence that a bifunctional inhibitor targeting the AP and TP can properly address the current treatment gaps associated with single complement pathway inhibitors, potentially eliminating the need for cumbersome and costly combination therapies (see, e.g., Notaro et al., N Engl J Med. 2022; 387:160-6).

1. A method of treating a complement-mediated disease in a human individual, comprising administering to the human individual an effective amount of a fusion protein comprising i) an antibody moiety that specifically binds to human C5 (“anti-C5 antibody moiety”) and ii) a Factor H (FH) or functional fragment thereof;
wherein the fusion protein is administered with an initial phase comprising administering the fusion protein at one or more initial doses, followed by a maintenance phase comprising administering the fusion protein for two or more maintenance doses;
wherein the initial phase comprises administering the fusion protein at an initial dose of about 600 mg IV to about 3600 mg IV on Day 1, and wherein the maintenance phase comprises administering the fusion protein starting Day 8 at a maintenance dose of:
i) about 600 mg IV to about 1200 mg IV weekly,
ii) about 720 mg SC to about 1440 mg SC weekly,
iii) about 1920 mg SC to about 2880 mg SC biweekly,
iv) about 1800 mg SC to about 2400 mg SC biweekly, or
v) about 960 mg SC to about 2880 mg SC biweekly; and
wherein the anti-C5 antibody moiety comprises a VH-CDR1 comprising the amino acid sequence of SEQ ID NO: 86, a VH-CDR2 comprising the amino acid sequence of SEQ ID NO: 57, a VH-CDR3 comprising the amino acid sequence of SEQ ID NO: 49, a VL-CDR1 comprising the amino acid sequence of SEQ ID NO: 23, a VL-CDR2 comprising the amino acid sequence of SEQ ID NO: 9, and a VL-CDR3 comprising the amino acid sequence of SEQ ID NO: 96.

2. The method of claim 1, wherein the complement-mediated disease is selected from the group consisting of paroxysmal nocturnal hemoglobinuria (PNH) syndrome, C3 glomerulopathy (C3G), IgA nephropathy (IgAN), and thrombotic microangiopathy secondary to systemic lupus erythematosus (SLE-TMA).

3-10. (canceled)

11. The method of claim 1, wherein the maintenance phase is at least about 12 weeks for weekly maintenance dosing, or at least about 13 weeks for biweekly maintenance dosing.

12. The method of claim 1, wherein the complement-mediated disease is PNH.

13. The method of claim 12, wherein the initial phase comprises administering the fusion protein at an initial dose of about 1200 mg IV to about 3600 mg IV on Day 1, and wherein the maintenance phase comprises administering the fusion protein starting Day 8 at a maintenance dose of:
i) about 600 mg IV to about 1200 mg IV weekly for at least about 12 weeks;
ii) about 720 mg SC to about 1440 mg SC weekly for at least about 12 weeks;
iii) about 1920 mg SC to about 2880 mg SC biweekly for at least about 13 weeks; or
iv) about 960 mg SC to about 2880 mg SC biweekly for at least about 13 weeks.

14. The method of claim 13,
(i) wherein the initial phase comprises administering the fusion protein at an initial dose of about 1200 mg IV on Day 1, and wherein the maintenance phase comprises administering the fusion protein starting Day 8 at a maintenance dose of about 720 mg SC or about 600 mg IV weekly for about 12 weeks;
(ii) wherein the initial phase comprises administering the fusion protein at an initial dose of about 1200 mg IV to about 2400 mg IV on Day 1, and wherein the maintenance phase comprises administering the fusion protein starting Day 8 at a maintenance dose of about 960 mg SC to about 2880 mg SC biweekly for about 13 weeks;
(iii) wherein the initial phase comprises administering the fusion protein at an initial dose of about 2400 mg IV on Day 1, and wherein the maintenance phase comprises administering the fusion protein starting Day 8 at a maintenance dose of about 960 mg SC to about 2880 mg SC biweekly for about 13 weeks;
(iv) wherein the initial phase comprises administering the fusion protein at an initial dose of about 1200 mg IV to about 2400 mg IV on Day 1, and wherein the maintenance phase comprises administering the fusion protein starting Day 8 at a maintenance dose of about 1920 mg SC biweekly for about 13 weeks; or
(v) wherein the initial phase comprises administering the fusion protein at an initial dose of about 3600 mg IV on Day 1, and wherein the maintenance phase comprises administering the fusion protein starting Day 8 at a maintenance dose of about 1920 mg SC to about 2880 mg SC biweekly for about 13 weeks.

15. The method of claim 14,
(i) wherein the initial phase comprises administering the fusion protein at an initial dose of about 1200 mg IV on Day 1, and wherein the maintenance phase comprises administering the fusion protein starting Day 8 at a maintenance dose of about 720 mg SC weekly for about 12 weeks;
(ii) wherein the initial phase comprises administering the fusion protein at an initial dose of about 2400 mg IV on Day 1, and wherein the maintenance phase comprises administering the fusion protein starting Day 8 at a maintenance dose of about 1920 mg SC biweekly for about 13 weeks; or
(iii) wherein the initial phase comprises administering the fusion protein at an initial dose of about 3600 mg IV on Day 1, and wherein the maintenance phase comprises administering the fusion protein starting Day 8 at a maintenance dose of about 2880 mg SC biweekly for about 13 weeks.

16. The method of claim 12, wherein the fusion protein is further administered with an extension phase comprising administering the fusion protein at one or more extension doses after the maintenance phase.

17. The method of claim 16, wherein the extension phase comprises administering the fusion protein at an extension dose of about 1920 mg SC biweekly for about 9 months.

18. The method of claim 1, wherein the complement-mediated disease is SLE-TMA.

19. The method of claim 18, wherein the initial phase comprises administering the fusion protein at an initial dose of about 1200 mg IV to about 3600 mg IV on Day 1, and wherein the maintenance phase comprises:
i) administering the fusion protein starting Day 8 at a maintenance dose of about 720 mg SC to about 1440 mg SC weekly for at least about 24 weeks;
ii) administering the fusion protein starting Day 8 at a maintenance dose of about 1920 mg SC to about 2880 mg SC biweekly for at least about 25 weeks;
iii) administering the fusion protein starting Day 8 at a first maintenance dose of about 600 mg IV to about 1200 mg IV weekly for a first maintenance phase period, followed by administering the fusion protein at a second maintenance dose of about 720 mg SC to about 1440 mg SC weekly for a second maintenance phase period, and wherein the maintenance phase is at least about 24 weeks; or
iv) administering the fusion protein starting Day 8 at a first maintenance dose of about 600 mg IV to about 1200 mg IV weekly for a first maintenance phase period, followed by administering the fusion protein at a second maintenance dose of about 1920 mg SC to about 2880 mg SC biweekly for a second maintenance phase period, and wherein the maintenance phase is at least about 25 weeks.

20. (canceled)

21. The method of claim 1, wherein the complement-mediated disease is C3G or IgAN.

22. The method of claim 21, wherein the initial phase comprises administering the fusion protein at an initial dose of about 600 mg IV to about 3600 mg IV on Day 1, and wherein the maintenance phase comprises administering the fusion protein starting Day 8 at a maintenance dose of:
i) about 600 mg IV to about 1200 mg IV weekly for at least about 48 weeks;
ii) about 720 mg SC to about 1440 mg SC weekly for at least about 48 weeks;
iii) about 1920 mg SC to about 2880 mg SC biweekly for at least about 49 weeks; or
iv) about 1800 mg SC to about 2400 mg SC biweekly for at least about 49 weeks.

23-25. (canceled)

26. The method of claim 1, wherein the anti-C5 antibody moiety is a full-length antibody (“anti-C5 full-length antibody”).

27-29. (canceled)

30. The method of claim 26, wherein the anti-C5 full-length antibody comprises a heavy chain and a light chain, and wherein
the heavy chain comprises the amino acid sequence of SEQ ID NO: 119, and the light chain comprises the amino acid sequence of SEQ ID NO: 74.

31. The method of claim 1, wherein the functional fragment of FH comprises short consensus repeat (SCR) domains 1-5 of FH.

32. The method of claim 31, wherein the functional fragment of FH comprises the amino acid sequence of SEQ ID NO: 85.

33. The method of claim 26, wherein the fusion protein comprises a first FH or functional fragment thereof and a second FH or functional fragment thereof, wherein the first FH or functional fragment thereof is fused to the C-terminus of a first heavy chain of the anti-C5 full-length antibody, and the second FH or functional fragment thereof is fused to the C-terminus of a second heavy chain of the anti-C5 full-length antibody.

34. The method of claim 33, whereineach heavy chain fused to FH or functional fragment thereof comprises the amino acid sequence of SEQ ID NO: 89, each light chain comprises the amino acid sequence of SEQ ID NO: 90.

35. The method of claim 1, wherein the fusion protein is formulated in a pharmaceutical composition, wherein the pharmaceutical composition comprises about 120 mg/mL of the fusion protein, sodium phosphate, sodium chloride, L-Lys-HCL, and polysorbate 80, pH of about 6.0.
